(19) United States
(12) Patent Application Publication
(10) Pub. No.: US 2008/0247955 A1
(43) Pub. Date: Oct. 9, 2008

(54) Inflammation treatment, detection and monitoring via TREM-1

(76) Inventors: Jun Kuai, Lexington, MA (US); Ken Dower, Medford, MA (US); Jeffrey Lawrence Feldman, Arlington, MA (US); Debra Denene Pittman, Windham, MA (US); Moitreyee Chatterjee-Kishore, Kildeer, IL (US); David Gunther Winkler, Arlington, MA (US); Lih-Ling Lin, Concord, MA (US); Scott Alan Jelinsky, Acton, MA (US); Cara Williams, Methuen, MA (US)

Correspondence Address: WYETH;PATENT LAW GROUP, 5 GIRALDA FARMS, MADISON, NJ, 07940, US

(21) Appl. No.: 12/009,166

(22) Filed: Jan. 16, 2008

Related U.S. Application Data
(60) Provisional application No. 60/880,804, filed on Jan. 16, 2007.
(60) Provisional application No. 60/904,264, filed on Feb. 28, 2007.
(60) Provisional application No. 60/923,131, filed on Apr. 11, 2007.
(60) Provisional application No. 61/001,687, filed on Nov. 2, 2007.

(51) Int. Cl. A61K 49/00; A61K 31/7088; A61K 39/00; C07K 16/00; A61K 38/02; G01N 33/53; A61K 39/395; C07H 21/02; A61P 37/06

(52) U.S. Cl. 424/9.2; 514/44; 424/184.1; 530/388.1; 424/141.1; 536/23.1; 435/7.2; 435/7.1; 514/12

(57) ABSTRACT

The present invention provides methods of treating inflammatory diseases/disorders in a subject by inhibiting/antagonizing TREM-1 expression/activity/signal transduction and/or DAP12/TyroBP expression and/or activity. Methods of detecting the presence of inflammatory disease in a subject by detecting TREM-1 and/or DAP12/TyroBP expression and/or activity in the subject or a sample obtained therefrom, wherein increased expression or activity is indicative of the inflammatory disease are also included. The present invention further provides methods for assessing the efficacy of a TREM-1-modulating agent administered to a patient by detecting levels of secreted phosphoprotein 1 (SPP1) and/or one or more other biomarkers in the patient or in a sample from the patient.

### REFERENCE TO RELATED APPLICATIONS

This application claims the benefit of U.S. Provisional Patent Application No. 61/001,687, filed Nov. 2, 2007, U.S. Provisional Patent Application No. 60/923,131, filed Apr. 11, 2007, U.S. Provisional Patent Application No. 60/904,264, filed Feb. 28, 2007, and U.S. Provisional Patent Application No. 60/880,804, filed Jan. 16, 2007, the entire disclosures of each of which are hereby incorporated by reference herein.

### BACKGROUND

Rheumatoid arthritis (RA) is an autoimmune inflammatory disease that affects about 1-2% of the population (Feldman (2002) Nature Rev. Immunol. 2(5):364-371; Mount et al. (2005) Nature Rev. Drug Discovery 4(1):11-12). RA is characterized by chronic inflammation and destruction of bone and cartilage in diarthrodial joints. Disease onset is typically between 25 and 50 years of age, and one in three patients becomes severely disabled within 20 years.

Although the precise etiology of RA is unknown, it has been proposed that the initiating event in RA involves an infectious event or environmental exposure (Firestein (2005) J. Clin. Rheumatology 11(3 Supp.):S39-44). Local induction of innate immunity may activate cells in the synovial lining and prime the area for subsequent adaptive immune responses in genetically susceptible individuals (Firestein (2005) J. Clin. Rheumatology 11(3 Supp.):S39-44). The synovium of established RA patients is characterized by marked synovial intimal lining hyperplasia, increased vascularity, and accumulation of inflammatory cells in the sublining. Biopsies of RA synovium have revealed spontaneous production of a number of pro-inflammatory cytokines, such as TNF-α, IL-1β, IL-6, and granulocyte-macrophage colony-stimulating factor (GM-CSF) (Feldman et al. (1996) Ann. Rev. Immunol. 14:397-440). Antibodies that neutralize TNF-α activity abrogate the production of multiple cytokines, a finding that led to the discovery of novel anti-TNFα therapies to treat RA. Recent clinical findings highlight the heterogeneity of RA, and suggest that additional factors may contribute to the pathogenesis of the disease.

Triggering receptor expressed on myeloid cell-1 (TREM-1) is a recently identified immunoglobulin-like cell surface receptor mainly expressed on neutrophils and a subset of CD14^{high} monocytes (Colonna et al. (2000) Seminars in Immunol. 12(2):121-27). TREM-1 has a short intracellular domain, and TREM-1 signaling is mediated through adaptor protein DAP12/TyproBP. DAP12/TyroBP is a transmembrane protein with an immunoreceptor tyrosine-based activation motif (ITAM) and functions as an adaptor protein, associating with TREM-1 and other transmembrane receptors.

TREM-1 expression is up-regulated during acute inflammation and by various Toll Like Receptor (TLR) ligands (Bouchon et al. (2001) Nature 410(6832):1103-07; Bleharski et al. (2003) J. Immunol. 170(7):3812-18; Murakami et al. (2006) 54(2):455-62). For instance, TREM-1 has been implicated in the acute inflammation associated with sepsis (Colonna (2003) Nat. Rev. Immun. 3(6):445-53). The expression of cell-surface and soluble TREM-1 is increased in sepsis in a manner that correlates with disease severity (Gibot et al. (2005) New England J. Med. 350(5):451-58; Gibot et al. (2005) Critical Care Med. 33(4):792-96). In a monosodium urate (MSU) induced inflammation model of gout, TREM-1 expression is rapidly induced in the infiltrated peritoneal macrophages and neutrophils. Moreover, activation of TREM-1 stimulates production of multiple pro-inflammatory cytokines and chemokines.

Furthermore, the synergistic effects of TREM-1 with TLRs and Nod-like receptors in production of these cytokines amplify the inflammatory response (Bouchon et al. (2001) Nature 410(6832):1103-07; Bleharski et al. (2003) J. Immunol. 170(7):3812-18; Netea et al. (2006) J. Leukocyte Biol. 80(6): 1454-61). These data suggest that increased TREM-1 expression and migration of the TREM-1 expressing cells to the site of inflammation may contribute to acute inflammation through amplification of the inflammatory responses.

A role for TREM-1 in acute inflammation was further demonstrated by the protection of mice from the lethality of LPS- or bacteria-induced septic shock using the TREM-1 ectodomain-Fc fusion or a synthetic peptide of TREM-1 ectodomain (Bouchon et al. (2001) Nature 410(6832):1103-07; Gibot et al. (2004) J. Exp. Med. 200(11):1419-26). TREM-1-Fc also protects against zymosan-A induced granuloma formation, which suggests that TREM-1 may play a role in chronic inflammation as well as acute inflammation (Nochi et al. (2003) Am. J. Path. 162(4):1191-201). Furthermore, accumulating evidence indicates that circulating levels of a soluble form of TREM-1 is a biomarker for multiple inflammatory disorders, including sepsis, pneumonia, acute pancreatitis, and peptic ulcer disease (Gibot et al. (2005) Intensive Care Med. 31(4):594-97; Gibot et al. (2004) New England J. Med. 350(5):451-58; Wang et al. (2004) World J. of Gastroenterology 10(18):2744-46; Koussoulas et al. (2006) Eur. J. Gastroenterology & Hepatology 18(4):375-79).

### SUMMARY OF THE INVENTION

The present invention is based, in part, on the discovery that the overexpression of TREM-1 and/or DAP12/TyroBP is associated with the presence of autoimmune and/or inflammatory disease. Thus, TREM-1 and/or Dap12/TyroBP are novel therapeutic targets for the treatment or prevention of autoimmune and inflammatory disorders. In one aspect, the invention relates to the use of TREM-1 and/or DAP12/TyroBP antagonists to treat or prevent an inflammatory disorder. Antagonists that can be used in the invention include, for example, antibodies (including, e.g., antibody fragments, single chain Fv, single domain antibodies derived from any species, including, for example, human, mouse, camel, llama, shark, goat, rabbit and bovine, as described more fully below); soluble receptors (including truncated receptors, natural soluble receptors, or fusion proteins comprising a receptor (or a fragment thereof) fused to a second protein, such as an Fc portion of an immunoglobulin); peptide inhibitors; small molecules; ligand fusions; and binding proteins. TREM-1 and DAP12/TyroBP are effective biomarkers for RA because these genes are overexpressed in individuals afflicted with RA. TREM-1 is a cellular receptor expressed on specific cell types, such as neutrophils and a subset of monocytes, and TREM-1 also exists in a soluble form. TREM-1 signaling is mediated through an adaptor protein, DAP12/TyroBP. Activation of TREM-1 induces production of proinflammatory cytokines and chemokines. Thus, elevated expression of TREM-1 may cause or contribute to the inflammation observed in RA, asthma, and other inflammatory diseases, such as chronic inflammatory diseases and respiratory inflammatory disorders/diseases. Accordingly, TREM-1 and/or DAP12/TyroBP are promising therapeutic targets for treating, modulating and/or preventing the symptoms associated with RA and other inflammatory disorders.

In one aspect, the present invention provides a method of treating inflammatory disease, such as, for example, chronic inflammatory disease (e.g., RA) or respiratory disorder/disease (e.g., asthma), by reducing TREM-1-mediated signal transduction. Reducing TREM-1-mediated signal transduction can include modulating, inhibiting, and/or antagonizing the TREM-1 receptor and/or other molecules involved in TREM-1 signal transduction (e.g., DAP12/TyroBP), thereby lessening, treating, preventing, alleviating, and/or ameliorating symptoms associated with TREM-1 mediated inflammation. In some embodiments, TREM-1 protein expression is reduced by inhibiting TREM-1 transcription; by selectively cleaving endogenous TREM-1 mRNA; or by inhibiting translation of endogenous TREM-1 mRNA. For example, TREM-1 protein expression can be reduced by administering an interfering RNA, such as an shRNA (e.g., an shRNA encoded by any of SEQ ID NOs:9-22) or an siRNA (e.g., any of SEQ ID NOs:23-26). In other embodiments, TREM-1 activation is inhibited by administering a small molecule, a peptide mimetic, a peptide inhibitor, a ligand fusion protein, an antibody or antibody fragment that binds TREM-1, an antibody or antibody fragment that binds TREM-1 ligand, a soluble TREM-1 receptor or ligand-binding portion thereof, or a soluble TREM-1 receptor fusion protein. Additional embodiments of the invention provide methods for treating inflammatory disease, such as, for example, chronic inflammatory disease (e.g., RA) or respiratory disorder/disease (e.g., asthma), by directly inhibiting non-TREM members (e.g., the TREM-1 accessory protein DAP12/TyroBP) of the TREM-1 signaling pathway. In some embodiments, TREM-1-mediated signal transduction is reduced in a human subject by inducing an immune response to endogenous TREM-1 or DAP12/TyroBP protein in the subject. For example, an immunogenic composition comprising an adjuvant and TREM-1 or DAP12/TyroBP protein or an immunogenic fragment thereof can be administered to the subject to provoke an immune response to the endogenous protein.

A further aspect of the invention provides for an antibody or antibody fragment that binds to TREM-1 without activating the receptor. The antibody or antibody fragment can be, for example, monoclonal. Additional embodiments of the invention provide for methods of treating a subject (e.g., a human subject) which include the step of administering to the subject a therapeutically effective quantity of an antibody or antibody fragment that binds to TREM-1 without activating the receptor.

In another aspect, the invention provides for an shRNA encoded by any of SEQ ID NOs:9-22.

The present invention provides that activation of TREM-1 results in the differential expression of a number of genes, such as secreted phosphoprotein 1 (SPP1), which can be used as markers for TREM-1 activity. Therefore, in another aspect, the invention provides markers which are specific for and indicative of TREM-1 activity. Changes in the level of one or more of these markers correlate with changes in TREM-1 activity. Thus, the invention also provides methods for assessing TREM-1 activity, and/or the efficacy of a TREM-1-modulating agent administered to a patient (e.g., a human patient) in need of such treatment, by detecting the level of one or more of these markers. For example, secreted phosphoprotein 1 (SPP1; also known as osteopontin (OPN), bone sialoprotein I (BSPI), early T-lymphocyte activation 1 (ETA-1), or MGC110940) levels can be detected in the patient or in a sample from the patient, and changes in SPP1 levels correlate with changes in TREM-1 signaling. SPP1 levels can be detected in the patient or in any clinically relevant sample from the patient, such as a body fluid sample (e.g., serum, synovial fluid, tracheobronchial fluid, sputum). In one embodiment, the method includes the further step of comparing SPP1 levels to a reference level, wherein an increase in SPP1 levels as compared to the reference level can be indicative of an increase in TREM-1 activity, and wherein a decrease SPP1 levels as compared to the reference level can be indicative of a decrease in TREM-1 activity. The reference level can be, for example, SPP1 levels detected in the patient or in a sample from the patient at a time prior to administration of the TREM-1-modulating agent. Additional markers which can be used to assess TREM-1 activity according to the present invention are described more fully below, and in, for example, FIG. 8A.

In a further aspect, the invention provides a method of screening for candidate agents capable of modulating TREM-1 signaling. The method includes contacting a TREM-1-expressing cell with a candidate agent and assessing the secreted phosphoprotein 1 (SPP1) levels of the TREM-1-expressing cell to determine whether the candidate agent modulates TREM-1 activation. Candidate agents which can be screened in accordance with the invention include, for example, an interfering RNA, a small molecule, a peptide mimetic, a peptide inhibitor, a ligand fusion protein, an antibody or fragment thereof that binds TREM-1, an antibody or fragment thereof that binds TREM-1 ligand, a soluble TREM-1 receptor, a soluble TREM-1 receptor fusion protein, and combinations thereof. In other embodiments, the method includes contacting the TREM-1-expressing cell with a TREM-1 activator (e.g., a crosslinking antibody). In yet other embodiments, the method can include comparing the assessed SPP1 levels with a reference level. An increase in SPP1 levels as compared to the reference level can be indicative of an increase in TREM-1 signaling, and a decrease SPP1 levels as compared to the reference level can be indicative of a decrease in TREM-1 signaling. In one embodiment, the reference level corresponds to SPP1 levels of the TREM-1-expressing cell assessed at a time prior to contacting the TREM-1-expressing cell with the candidate agent. Additional markers which can be used to assess TREM-1 activity according to the present invention are described more fully below, and in, for example, FIG. 8A.

Another aspect of the invention provides a method of monitoring a patient treated for inflammation or chronic inflammation. The method includes administering a TREM-1 modulating agent to a patient (e.g., a human patient) in need thereof, detecting secreted phosphoprotein 1 (SPP1) levels in the patient or in a sample from the patient, and comparing the detected SPP1 levels with a reference level. SPP1 levels can be detected in the patient or in a sample from the patient, such as a body fluid sample (e.g., serum, synovial fluid, tracheobronchial fluid, sputum). A reduction in SPP1 levels as compared to the reference level is indicative of a reduction in TREM-1 mediated inflammation, and no change or an increase in SPP1 levels as compared to the reference level may indicate that there has been no change or an increase in TREM-1 mediated inflammation, respectively. In some embodiments, the reference level corresponds to SPP1 levels detected in the patient or in a sample from the patient at a time prior to or concurrent with administration of the TREM-1-modulating agent. In other embodiments, the reference level corresponds to SPP1 levels in a control subject (e.g., a human) or a sample from the control subject, where the control subject is known not to have chronic inflammation. Additional markers which can be used to assess TREM-1 activity according to the present invention are described more fully below, and in, for example, FIG. 8A.

In another aspect, the invention relates to a method of detecting the presence of inflammatory disease, such as, for example, chronic inflammatory disease (e.g., RA) or respiratory disorder/disease (e.g., asthma), in a subject (e.g., a human subject). Inflammatory diseases include, for example, arthritis (including rheumatoid arthritis, juvenile rheumatoid arthritis, osteoarthritis, psoriatic arthritis, lupus-associated arthritis or ankylosing spondylitis), scleroderma, systemic lupus erythematosis, vasculitis, multiple sclerosis, autoimmune thyroiditis, dermatitis (including atopic dermatitis and eczematous dermatitis), autoimmune skin diseases, myasthenia gravis, inflammatory bowel disease (IBD), Crohn's disease, colitis, ulcerative colitis, diabetes mellitus (type I); inflammatory conditions of, e.g., the skin (e.g., psoriasis, acute and chronic urticaria (hives)), cardiovascular system (e.g., atherosclerosis), nervous system (e.g., Alzheimer's disease, amyotrophic lateral sclerosis), liver (e.g., hepatitis), kidney (e.g., nephritis) and pancreas (e.g., pancreatitis); cardiovascular disorders, e.g., cholesterol metabolic disorders, oxygen free radical injury; disorders associated with wound healing; respiratory disorders, e.g., asthma and COPD (e.g., cystic fibrosis); acute inflammatory conditions (e.g., endotoxemia, septicemia, septic shock, toxic shock syndrome and infectious disease); transplant rejection and allergy (e.g., anaphylaxis, angioedema, atopy, insect sting allergies, allergic rhinitis). Additional conditions which can be detected in accordance with the present invention include ischemia. The method includes the step of detecting TREM-1 (e.g., membrane-bound TREM-1, soluble TREM-1) or DAP12/TyroBP expression or activity in a subject or in a sample obtained from the subject. (In this application, “or” can mean “and/or”; thus, the method can include detection of both TREM-1 and DAP12/TyroBP and can include detection of expression and activity, if so desired.) Samples useful in the practice of this and other methods of the invention are any samples in which TREM-1 and/or DAP12/TyroBP can be detected, including, for example, samples including joint tissue, synovial fluid, synovial membranes, or any other clinically relevant body fluid or tissue, whether, for example, circulating (e.g., blood, plasma, or lymph) or localized at a site of chronic inflammation, in a tissue of the immune system, or in a tissue or fluid previously exposed to a site of chronic inflammation. Detection of elevated TREM-1 or DAP12/TyroBP expression or activity is indicative of the presence of the inflammatory disease, such as, for example, chronic inflammatory disease, such as, for example, RA. The method can include the additional step of comparing TREM-1 or DAP12/TyroBP expression or activity in a subject, or a sample derived from the subject, with a known reference level. The outcome of the comparison (e.g., increased expression or activity) is indicative of the presence of the inflammatory disease. The reference level can, for example, be indicative of the presence of the inflammatory disease, or of a threshold for differentiating between normal and increased expression.

The invention also provides a method for detecting the presence of inflammatory disease, such as, for example, chronic inflammatory disease, such as, for example, RA or asthma, in a subject (e.g., a human subject) by: detecting TREM-1 or DAP12/TyroBP expression in a subject or a sample from the subject; and comparing TREM-1 or DAP12/TyroBP expression in the subject or the sample to a reference level. The outcome of the comparison (e.g., increased expression) is indicative of the presence of the inflammatory disease. The reference level can be indicative of the presence of the inflammatory disease, or of a threshold for differentiating between normal and increased expression, etc.

In another aspect, the invention provides a method of monitoring inflammatory disease, such as, for example, chronic inflammatory disease (e.g., RA) or respiratory disorder/disease (e.g., asthma), in a subject (e.g., a human subject). The method benefits from an appreciation that changes in TREM-1 or DAP12/TyroBP expression or activity in a subject over time (as determined at different times in the subject or as determined in samples obtained from the subject at different times) can be used as an indication of changes in disease status. The method includes detecting TREM-1 or DAP12/TyroBP expression or activity in the subject at two or more different times (sometimes referred to herein as a first time and a second, later time) or in samples obtained from the subject at two or more different times and comparing the expression or activity observed. A decrease in TREM-1 or DAP12/TyroBP expression or activity over time can be indicative of a reduction in the inflammatory disease, whereas an increase in TREM-1 or DAP12/TyroBP expression or activity over time can be indicative of an increase in the inflammatory disease.

The monitoring method is also useful to evaluate a treatment for inflammatory disease, such as, for example, chronic inflammatory disease (e.g., RA) or respiratory disorder/disease (e.g., asthma), in a subject (e.g., a human subject). A treatment is administered prior to the second, later time of detecting TREM-1 or DAP12/TyroBP expression or activity in the subject or prior to taking the second, later sample. The treatment can be administered before or after the monitoring method has begun (before or after the first time of detection in the subject or the taking of the first sample from the subject). Moreover, the course of treatment can be modified based upon the comparison of TREM-1 or DAP12/TyroBP expression or activity at a first time or in a first sample with TREM-1 or DAP12/TyroBP expression at a second, later time or in a second sample.

These and other aspects and embodiments of the invention are also described in the following sections of the application, which are provided to highlight specific embodiments of the invention and are not intended to limit the invention, the scope of which is limited only by the issued claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a bar graph showing expression of TREM-1 and DAP12/TyroBP in synovium from RA patients.

FIG. 2 is a bar graph showing TREM-1 and DAP12/TyroBP mRNA expression in the murine collagen induced arthritis (CIA) model.

FIG. 3A shows a representative image of immunohistochemistry staining of human TREM-1 in a section of an RA positive synovium with a TREM-1-positive index >1000.

FIG. 3B shows a representative image of immunohistochemistry staining of human TREM-1 in an osteoarthritis (OA) control section with a TREM-1-positive index <20.

FIG. 4 is a graph showing levels of soluble TREM-1 detected by ELISA in human plasma samples obtained from RA and control (HVOS) patients.

FIG. 5 shows an exemplary Western blot depicting a time-course of mitogen activated protein kinase (MAPK) activation after crosslinking of TREM-1.

FIG. 6 shows an exemplary ANOVA heat map clustering analysis of genes regulated >2-fold with p<0.01. Individual donors are shown on the left, average mean intensity is shown on the right.

FIG. 7 shows an exemplary scatter plot of all present genes (“calls”). In fold-changes plotted for TREM-1 (x-axis) and LPS (y-axis), with down-regulation converted to negative values. Selected genes are highlighted. The 45° axis demarcates genes comparably regulated by both treatments.

FIG. 8A shows a table listing exemplary genes that are up-regulated >4-fold upon TREM-1 activation.

FIG. 8B shows a table listing exemplary genes that are up-regulated >4-fold upon treatment with LPS.

FIG. 9A shows a table listing exemplary genes that are commonly up-regulated >4-fold; i.e., genes which are up-regulated both upon TREM-1 activation and upon treatment with LPS.

FIG. 9B shows a table listing exemplary genes that are down-regulated >4-fold either upon TREM-1 activation or upon treatment with LPS.

FIGS. 10A-B show the results of an exemplary phagocytosis assay with GFP-expressing human monocytic THP-1 cells. 1 μM beads appear in red. FIG. 10A shows morphological changes in THP-1 cells after treatment. FIG. 10B shows that treatment with α-TREM-1 and LPS induces microsphere phagocytosis.

FIGS. 11A-F are graphs showing exemplary time-course ELISAs. FIG. 11A shows GM-CSF, FIG. 11B shows M-CSF, FIG. 11C shows G-CSF, FIG. 11D shows INHBA (inhibin, beta A (activin A, activin AB alpha polypeptide)), FIG. 11E shows SPP1, FIG. 11F shows IL-23.

FIG. 12 is a series of bar graphs showing that crosslinking of TREM-1 induces production of multiple cytokines in an RA tissue sample in a dose dependent manner.

FIGS. 13A-B are charts showing production multiple cytokine in tissue samples prepared from three different donors. FIG. 13A shows a comparison of spontaneous cytokine production in three donor samples. FIG. 13B shows a comparison of cytokine production upon crosslinking of TREM-1 in three donor samples.

FIG. 14 is a bar graph showing increased TREM-1 expression in K/BxN paws.

FIG. 15 is a graph showing ankle thickening in mTREM-1-hFC transgenic mice and wildtype mice in response to K/BxN serum transfer.

FIG. 16 is a graph showing ankle thickening in mTREM-1-hFC transgenic mice on Day 14, in response to K/BxN serum transfer.

FIG. 17 is a graph showing ear swelling after anti-IgE antibody challenge in transgenic mice expressing a mTREM-1-hFc fusion protein.

FIG. 18 is a graph showing ear swelling after anti-IgE antibody challenge in mice pretreated with mTREM-1-mFc protein.

FIG. 19 is a graph showing dose response of ear swelling after anti-IgE antibody challenge in mice pretreated with mTREM-1-mFc protein.

FIG. 20 is a graph showing ear swelling in TREM-1 knockout mice after anti-IgE antibody challenge.

FIG. 21 is a bar graph showing TREM-1 expression by RT-PCR after shRNA or siRNA knockdown.

FIGS. 22A-B show representative Western blots depicting TREM-1 expression after lentiviral shRNA knockdown of TREM-1 in TREM-1 over-expressing cell lines.

FIG. 23 is a table summarizing the results from global gene expression profiling of purified human monocytes using Affymetrix® Human Genome U133_plus 2.0 arrays (see Example 6).

FIG. 24 is a table further summarizing the results from global gene expression profiling of purified human monocytes using Affymetrix® Human Genome U133_plus 2.0 arrays (see Example 6).

### DETAILED DESCRIPTION OF THE INVENTION

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides.

As used herein, “RA positive,” “RA sample,” and “RA tissue” refer to a subject, or any tissue, fluid, or other sample derived from a subject with rheumatoid arthritis. “RA negative” refers to a subject, or any tissue, fluid, or other sample derived from an unaffected subject.

As used herein, the term “RA” refers to rheumatoid arthritis. As used herein, the term “OA” refers to osteoarthritis.

The term “antibody” includes intact molecules as well as functional fragments thereof, such as Fab, Fab′, F(ab′)₂, Fc, Fd, Fd′, Fv, single chain antibodies (scFv for example), single variable domain antibodies (Dab), diabodies (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibodies of the present invention can be monoclonal or polyclonal. The antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function). Typically, the antibody specifically binds to a predetermined antigen, e.g., an antigen associated with a disorder, e.g., a neurodegenerative, metabolic, inflammatory, autoimmune and/or a malignant disorder.

Antibodies of the present invention can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In one aspect of the invention, a single domain antibody can be derived from a variable region of the immunoglobulin found in fish, such as, for example, that which is derived from the immunoglobulin isotype known as Novel Antigen Receptor (NAR) found in the serum of shark. Methods of producing single domain antibodies derived from a variable region of NAR (“IgNARs”) are described in WO 03/014161 and Streltsov (2005) Protein Sci. 14:2901-2909. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The invention also contemplates the use of Small Modular ImmunoPharmaceuticals (“SMIPs™”) which typically refers to binding domain-immunoglobulin fusion proteins including a binding domain polypeptide that is fused or otherwise connected to an immunoglobulin hinge or hinge-acting region polypeptide, which in turn is fused or otherwise connected to a region comprising one or more native or engineered constant regions from an immunoglobulin heavy chain, other than CH1, for example, the CH2 and CH3 regions of IgG and IgA, or the CH3 and CH4 regions of IgE (see e.g., U.S. 2005/0136049 by Ledbetter, J. et al. for a more complete description). The binding domain-immunoglobulin fusion protein can further include a region that includes a native or engineered immunoglobulin heavy chain CH2 constant region polypeptide (or CH3 in the case of a construct derived in whole or in part from IgE) that is fused or otherwise connected to the hinge region polypeptide and a native or engineered immunoglobulin heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE). Typically, such binding domain-immunoglobulin fusion proteins are capable of at least one immunological activity selected from the group consisting of antibody dependent cell-mediated cytotoxicity, complement fixation, and/or binding to a target, for example, a target antigen.

The term “antisense” as used herein refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines natural sequences produced by the cell to form duplexes. These duplexes then block either the further transcription or translation. The designation “negative” is sometimes used in reference to the antisense strand, and “positive” is sometimes used in reference to the sense strand.

As used herein, the terms “subject” and “patient” refers to any human or nonhuman mammal.

As used herein, the term “detect” and all other forms of the root word “detect” refer to the ascertainment of the presence or absence of one or more targets, quantitation of one or more targets, or determination of the presence or absence of a threshold value of one or more biomarkers.

As used herein, the term “joint tissue” refers to any tissue or fluid derived from a joint area, including, by way of non-limiting example, tendons, ligaments, and synovial membranes.

As used herein, the term “inflammatory disease” includes, by way of non-limiting example, arthritis (including rheumatoid arthritis, juvenile rheumatoid arthritis, osteoarthritis, psoriatic arthritis, lupus-associated arthritis or ankylosing spondylitis), scleroderma, systemic lupus erythematosis, vasculitis, multiple sclerosis, autoimmune thyroiditis, dermatitis (including atopic dermatitis and eczematous dermatitis), autoimmune skin diseases, myasthenia gravis, inflammatory bowel disease (IBD), Crohn's disease, colitis, ulcerative colitis, diabetes mellitus (type I); inflammatory conditions of, e.g., the skin (e.g., psoriasis, acute and chronic urticaria (hives)), cardiovascular system (e.g., atherosclerosis), nervous system (e.g., Alzheimer's disease, amyotrophic lateral sclerosis), liver (e.g., hepatitis), kidney (e.g., nephritis) and pancreas (e.g., pancreatitis); cardiovascular disorders, e.g., cholesterol metabolic disorders, oxygen free radical injury; disorders associated with wound healing; respiratory disorders, e.g., asthma and COPD (e.g., cystic fibrosis); acute inflammatory conditions (e.g., endotoxemia, septicemia, septic shock, toxic shock syndrome and infectious disease); transplant rejection and allergy (e.g., anaphylaxis, angioedema, atopy, insect sting allergies, allergic rhinitis).

As used herein, the term “chronic inflammatory disease” refers to any disease where the inflammatory response is of prolonged duration (e.g., weeks, months, or even indefinitely) and whose extended time course is provoked by persistence of the causative stimulus to inflammation in the tissue. The term “chronic inflammatory disease” includes, for example, rheumatoid arthritis.

As used herein, the term “indicative” (e.g., indicative of the inflammatory disease) means a sign or indication or factor to be considered, as opposed to being definitive proof in and of itself. Generally, increased TREM-1 expression levels correlate with an increased likelihood of inflammatory disease (e.g., RA); thus, increased TREM-1 expression is indicative of the presence of the inflammatory disease (e.g., RA). Likewise, normal TREM-1 expression levels generally correlate with an increased likelihood of the absence of the inflammatory disease (e.g., RA).

As used herein, the term “PCR” refers to polymerase chain reaction.

The present invention includes the identification of TREM-1 and DAP12/TyroBP as biomarkers for inflammatory disease, such as, for example, chronic inflammatory disease, and, more specifically, as biomarkers for RA. A comparison of transcriptional profiling of RA and normal synovial tissues revealed that TREM-1 mRNA expression was up-regulated 6.5 fold above normal in human RA samples, and DAP12/TyroBP mRNA expression was up-regulated 2 fold above normal in human RA samples (see Example 1). In a collagen induced arthritis (CIA) model, TREM-1 mRNA was up-regulated 132 fold in CIA paws as compared to paws from normal mice, and DAP12/TyroBP mRNA was up-regulated 8.21 fold in CIA paws as compared to paws from normal mice (see Example 2). By immunohistochemistry, human RA synovial samples contained an increased number of TREM-1-expressing cells (see Example 3). Moreover, activation of TREM-1 in synovial cultures induced pro-inflammatory cytokine and cytokine production in a dosage-dependent manner (see Example 9). Further, soluble TREM-1 levels are elevated in human clinical plasma samples from RA patients as compared to control patients (see Example 4). The identity of TREM-1 and DAP12/TyroBP as biomarkers for RA and the ability of TREM-1 to induce a pro-inflammatory response make TREM-1 and members of the TREM-1 signaling pathway ideal therapeutic targets for inflammatory disease, such as, for example, chronic inflammatory disease, especially RA.

### Detection of Gene Expression

The present invention provides methods for detecting and monitoring inflammatory disease, such as, for example, chronic inflammatory disease by detecting or quantitating the expression or activity of TREM-1 or DAP12/TyroBP. Many methods of detection of a protein, nucleic acid, or activity level of interest, with or without quantitation, are well known and can be used in the practice of the invention.

Target gene transcripts can be detected using numerous techniques that are well known in the art. Some useful nucleic acid detection systems involve preparing a purified nucleic acid fraction of a sample, and subjecting the sample to a direct detection assay or an amplification process followed by a detection assay, such as an assay of TREM-1 mRNA in a joint tissue sample. Amplification can be achieved, for example, by polymerase chain reaction (PCR), reverse transcriptase (RT) and coupled RT-PCR. Detection of a nucleic acid can be accomplished, for example, by probing the purified nucleic acid fraction with a probe that hybridizes to the nucleic acid of interest, and in many instances detection involves an amplification as well. Northern blots, dot blots, microarrays, quantitative PCR, and quantitative RT-PCR are all well known methods for detecting a nucleic acid in a sample. Nucleic acids also can be amplified by ligase chain reaction, strand displacement amplification, self-sustained sequence replication or nucleic acid sequence-based amplification. See, for example, Lewis (1992) Genetic Engineering News 12(9):1; Guatelli et al. (1990) Proc. Natl. Acad. Sci. USA 87:1874-1878; and Weiss (1991) Science 254:1292. Nucleic acids can also be detected by sequencing; the sequencing can use a primer specific to the target nucleic acid (e.g., a TREM-1 cDNA sequence) or a primer to an adaptor sequence attached to the target nucleic acid. Sequencing of randomly selected mRNA or cDNA sequences can provide an indication of the relative expression of a biomarker as indicated by the percentage of all sequenced transcripts containing nucleic acid sequence corresponding to the biomarker (e.g., to a TREM-1 cDNA or mRNA sequence). Alternatively, a nucleic acid can be detected in situ, such as by hybridization, without extraction or purification.

Target proteins can be detected, for example, immunologically using one or more antibodies. In immunological assays, an antibody having specific binding affinity for a biomarker or a secondary antibody that binds to such an antibody can be labeled, either directly or indirectly. The antibody need not be complete: an antibody variable domain or an artificial analog thereof, such as a single chain antibody, is sufficient. Suitable labels include, without limitation, radionuclides (e.g., 125I, 131I, 35S, 3H, 32P, 33P, or 14C), fluorescent moieties (e.g., fluorescein, FITC, PerCP, rhodamine, or PE), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). Antibodies can be indirectly labeled by conjugation with biotin then detected with avidin or streptavidin labeled with a molecule described above. Methods of detecting or quantifying a label depend on the nature of the label and are known in the art. Examples of detectors include, without limitation, x-ray film, radioactivity counters, scintillation counters, spectrophotometers, calorimeters, fluorometers, luminometers, and densitometers. Combinations of these approaches (including “multi-layer” assays) familiar to those in the art can be used to enhance the sensitivity of assays.

Immunological assays for detecting a target protein can be performed in a variety of known formats, including sandwich assays, competition assays (competitive RIA), or bridge immunoassays. See, for example, U.S. Pat. Nos. 5,296,347; 4,233,402; 4,098,876; and 4,034,074. Methods of detecting a target protein generally include contacting a biological sample with an antibody that binds to the protein and detecting binding of the protein to the antibody. For example, an antibody having specific binding affinity for TREM-1 can be immobilized on a solid substrate by any of a variety of methods known in the art and then exposed to the biological sample. Binding of TREM-1 to the antibody on the solid substrate can be detected by exploiting the phenomenon of surface plasmon resonance, which results in a change in the intensity of surface plasmon resonance upon binding that can be detected qualitatively or quantitatively by an appropriate instrument, e.g., a Biacore® apparatus (Biacore International AB, Rapsgatan, Sweden). Alternatively, the antibody can be labeled and detected as described above. A standard curve using known quantities of a protein can be generated to aid in the quantitation of biomarker levels.

In other embodiments, a “sandwich” assay in which a capture antibody is immobilized on a solid substrate is used to detect the level of a target protein. The solid substrate can be contacted with the biological sample such that any target protein in the sample can bind to the immobilized antibody. The level of the target protein bound to the antibody can be determined using a “detection” antibody having specific binding affinity for the target protein and the methods described above. It is understood that in these sandwich assays, the capture antibody should not bind to the same epitope (or range of epitopes in the case of a polyclonal antibody) as the detection antibody. Thus, if a monoclonal antibody is used as a capture antibody, the detection antibody can be another monoclonal antibody that binds to an epitope that is either completely physically separated from or only partially overlaps with the epitope to which the capture monoclonal antibody binds, or a polyclonal antibody that binds to epitopes other than or in addition to that to which the capture monoclonal antibody binds. If a polyclonal antibody is used as a capture antibody, the detection antibody can be either a monoclonal antibody that binds to an epitope that is either completely physically separated from or partially overlaps with any of the epitopes to which the capture polyclonal antibody binds, or a polyclonal antibody that binds to epitopes other than or in addition to that to which the capture polyclonal antibody binds. Sandwich assays can be performed as sandwich ELISA assays, sandwich Western blotting assays, or sandwich immunomagnetic detection assays.

Suitable solid substrates to which an antibody (e.g., a capture antibody) can be bound include, without limitation, microtiter plates, tubes, membranes such as nylon or nitrocellulose membranes, and beads or particles (e.g., agarose, cellulose, glass, polystyrene, polyacrylamide, magnetic, or magnetizable beads or particles). Magnetic or magnetizable particles can be particularly useful when an automated immunoassay system is used.

Other techniques for detecting target polypeptides include mass-spectrophotometric techniques such as electrospray ionization (ESI), and matrix-assisted laser desorption-ionization (MALDI). See, for example, Gevaert et al. (2001) Electrophoresis 22(9):1645-51; Chaurand et al. (1999) J. Am. Soc. Mass Spectrom. 10(2):91-103. Mass spectrometers useful for such applications are available from Applied Biosystems (Foster City, Calif.); Bruker Daltronics (Billerica, Mass.); and GE Healthcare (Piscataway, N.J.).

It will be appreciated that the expression of any target gene transcript or target protein according to the present invention can be readily detected using one or more of the above techniques.

### Detection of TREM-1 Activity

The activity of TREM-1 or DAP12/TyroBP can be assessed, for instance, by assessing the expression levels of one or more (e.g., two or more than two, more than three, more than four, more than five, more than ten, or more than twenty) TREM-1 responsive genes. The expression levels can be absolute or relative, e.g., as compared to a control sample or a reference level. Differential gene expression can be determined by transcriptional profiling of a test sample and, optionally, a control sample. The reference level can be a transcriptional profile corresponding to a sample of known disease state. A positive control can be, for example, a sample wherein TREM-1 and/or DAP12/TyroBP have been intentionally over-expressed in one or more cells, a sample of cells in which endogenous or recombinantly-expressed TREM-1 or DAP12/TyroBP have been activated by, for example, addition of a cross-linking antibody, or a sample obtained from a subject having an inflammatory disease or chronic inflammatory disease (e.g., RA) of a known severity. A negative control can be, for example, a sample wherein TREM-1 and/or DAP12/TyroBP have not been expressed or activated or a sample from a subject without the inflammatory disease or chronic inflammatory disease (e.g., RA).

Numerous protocols are available for using nucleic acid microarrays for transcriptional profiling. Exemplary protocols include those provided by Affymetrix in connection with the use of its GeneChip® arrays. Samples amenable to nucleic acid microarray hybridization can be prepared from any human cell or tissue. Where a nucleic acid microarray includes probes for non-human drug target genes, samples can be prepared for cells or tissues of the corresponding non-human species.

The sample for hybridization to a nucleic acid microarray can be either RNA (e.g., mRNA or cRNA) or DNA (e.g., cDNA). Various methods are available for isolating RNA from tissues. These methods include, but are not limited to, RNeasy® kits (provided by Qiagen, Hilden, Germany), MasterPure™ kits (provided by Epicentre Technologies), and Trizol® (provided by Gibco BRL, Carlsbad, Calif.). The RNA isolation protocols provided by Affymetrix can also be used.

In one embodiment, isolated RNA is amplified or labeled before being hybridized to a nucleic acid microarray. Suitable RNA amplification methods include, but are not limited to, reverse transcriptase PCR, isothermal amplification, ligase chain reaction, and Qbeta replicase method. The amplification products can be either cDNA or cRNA. In one embodiment, the isolated mRNA is reverse transcribed to cDNA using a reverse transcriptase and a primer consisting of oligo d(T) and a sequence encoding the phage T7 promoter. The cDNA is single stranded. The second strand of the cDNA can be synthesized using a DNA polymerase, combined with an RNase to break up the DNA/RNA hybrid. After synthesis of the double stranded cDNA, T7 RNA polymerase is added to transcribe cRNA from the second strand of the doubled stranded cDNA. In one embodiment, the originally isolated RNA can be hybridized to a nucleic acid microarray without amplification.

cDNA, cRNA, or other nucleic acid samples can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical or chemical means. The labeling moieties include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

Hybridization reactions can be performed in absolute or differential hybridization formats. In the absolute hybridization format, polynucleotides derived from one sample are hybridized to the probes in a nucleic acid microarray. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to a nucleic acid microarray. The nucleic acid microarray is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

Signals gathered from the nucleic acid microarrays can be analyzed using commercially available software, such as those provided by Affymetrix or Agilent Technologies. Controls for scan sensitivity, probe labeling, and cDNA or cRNA quantitation, can be included in the hybridization experiments. Hybridization signals can be scaled or normalized before being subject to further analysis. For instance, hybridization signals for each individual probe can be normalized to take into account variations in hybridization intensities when more than one microarray is used under similar test conditions. Hybridization signals can also be normalized using the intensities derived from internal normalization controls contained on each microarray. In addition, genes with relatively consistent expression levels across the samples can be used to normalize the expression levels of other genes. In one embodiment, probes for certain maintenance genes are included in the nucleic acid microarray. These genes are chosen because they show stable levels of expression across a diverse set of tissues. Hybridization signals can be normalized or scaled based on the expression levels of these maintenance genes.

### Monitoring and Evaluation of Disease or Treatment

The present invention provides methods for monitoring inflammatory disease, such as, for example, chronic inflammatory disease, such as, for example, RA, in a subject. Progression of an inflammatory disease in a subject can be monitored by measuring a level of expression of mRNA corresponding to, or protein encoded by, or activity of, one or more biomarkers, such as TREM-1 or DAP12/TyroBP. Target gene mRNA or protein expression levels can be detected in vivo or in samples taken from, for example, joint tissue, synovial fluid, synovial membranes, or any other clinically relevant source. The level of expression of mRNA and/or protein corresponding to the target gene can be detected by standard methods as described above. Disease state in a subject can be monitored (e.g., for amelioration, aggravation, or reoccurrence of disease) by comparing levels of target gene protein or RNA in the subject to the subject's baseline level of target protein or RNA. For instance, TREM-1 expression levels in the subject at a first time can be compared with TREM-1 expression levels in the subject at a second, later time. An increase in the level of expression of TREM-1 mRNA or protein over time is indicative of the progression of the inflammatory disease. A decrease in the level of expression of TREM-1 mRNA or protein over time is indicative of the reduction of the inflammatory disease.

The levels of, for instance, TREM-1 or DAP12/TyroBP protein or RNA in a subject also can be used to monitor the effectiveness of treatment. Typically, the subject's baseline level of a target protein or RNA is obtained (e.g., before treatment) and compared to the level of the target protein or RNA at various time points after or between treatments (e.g., one or more days, weeks, or months after treatment). The result of the comparison may reveal the effectiveness of past treatment, and future treatment can be modified accordingly. For instance, a decrease in TREM-1 protein or RNA levels relative to previously detected levels generally indicates a positive response to a treatment regimen, and thus similar treatment should be continued. Similarly, disease state in a subject can be monitored (e.g., for amelioration, aggravation, or reoccurrence of disease) by comparing levels of a target protein or RNA in the subject to the subject's baseline level, or to prior detected levels, of a target protein or RNA.

### Treatment

The present invention provides methods for treating inflammatory disease, such as, for example, chronic inflammatory disease (e.g., RA) or respiratory disorder/disease (e.g., asthma), by inhibiting and/or antagonizing TREM-1-mediated signal transduction. Inhibiting and/or antagonizing TREM-1-mediated signal transduction can be accomplished by directly inhibiting TREM-1 or by inhibiting and/or antagonizing non-TREM-1 members of the TREM-1 signaling pathway, such as the TREM-1 accessory protein DAP12/TyroBP. Suitable inhibitors and/or antagonists can, for example, decrease the expression of a nucleic acid encoding TREM-1, decrease levels of the TREM-1 protein, or inhibit TREM-1 activity. Examples of inhibitors and/or antagonists include, by way of non-limiting example: antisense oligonucleotides; interfering RNAs; antibodies to TREM-1; antibodies to TREM-1 ligand; competitors for TREM-1 ligand binding sites, including TREM-1 receptors and ligand-binding fragments thereof, soluble truncated TREM-1 receptors, and soluble TREM-1 receptor fusion proteins, such as, for example, a TREM-1 fusion containing the Fc portion of an IgG immunoglobulin, ligand fusion proteins; peptide mimetics; peptide inhibitors; small molecules; and combinations thereof.

### Antisense Oligonucleotides

Antisense oligonucleotides can be used to inhibit TREM-1, DAP12/TyroBP, or any other member of the TREM-1 or DAP12/TyroBP signaling pathways by decreasing mRNA and protein levels of these targets. Antisense suppression refers to administration or in situ generation of nucleic acid sequences or their derivatives that specifically hybridize or bind under cellular conditions, with the cellular mRNA and/or genomic DNA encoding one or more of the subject target alleles so as to inhibit expression of that target allele, e.g. by inhibiting transcription and/or translation. The binding may be by conventional base pair complementarity, or, for example, in the case of binding to DNA duplexes, through specific interactions in the major groove of the double helix. In general, antisense suppression refers to the range of techniques generally employed in the art, and includes any suppression which relies on specific binding to nucleic acid sequences. An antisense construct of the present invention can be delivered, for example, as an expression plasmid which, when transcribed in the cell, produces RNA that is complementary to at least a unique portion of the cellular mRNA that encodes a target sequence or target allele of an endogenous gene. Alternatively, the antisense construct can be a nucleic acid that is generated ex vivo and which, when introduced into the cell, causes inhibition of expression by hybridizing with the mRNA and/or genomic sequences of a target allele of an endogenous gene. Such nucleic acids are preferably modified oligonucleotides that are resistant to endogenous nucleases, e.g., exonucleases and/or endonucleases, and are therefore stable in vivo. Modifications, such as phosphorothioates, have been made to nucleic acids to increase their resistance to nuclease degradation, binding affinity and uptake. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775).

The antisense nucleic acids can be DNA or RNA or chimeric mixtures or derivatives or “modified versions thereof,” single-stranded or double-stranded. As referred to herein, “modified versions thereof” refers to nucleic acids that are modified, e.g., at a base moiety, sugar moiety, or phosphate backbone, for example, to improve stability, halflife, hybridization, effectiveness, etc. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. The nucleic acid may include other appended groups such as peptides (e.g., for targeting host cell receptors in vivo), or agents facilitating transport across the cell membrane (see, e.g., PCT Publication No. WO 88/09810, published Dec. 15, 1988) or the blood-brain barrier (see, e.g., PCT Publication No. WO 89/10134, published Apr. 25, 1988), hybridization-triggered cleavage agents or intercalating agents. To this end, the oligonucleotide may be conjugated to another molecule, e.g., a peptide, hybridization triggered cross-linking agent, transport agent, hybridization-triggered cleavage agent, etc.

The antisense nucleic acid can optionally include at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N-6-isopentenyladenine, 1-methylguanine, 1-methylinosine, 2,2-dimethylguanine, 2-methyladenine, 2-methylguanine, 3-methylcytosine, 5-methylcytosine, N-6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopente-nyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5oxyacetic acid methylester, uracil-5-oxyacetic acid (v), -5-methyl-2-thiouracil, 3-(3-amino-3-N-2-carboxypropyl) uracil, (acp3)w, and 2,6-diaminopurine.

Methods for synthesizing antisense oligonucleotides are known, including solid phase synthesis techniques. Equipment for such synthesis is commercially available from several vendors including, for example, Applied Biosystems (Foster City, Calif.). Alternatively, expression vectors that contain a regulatory element that directs production of an antisense transcript can be used to produce antisense molecules.

It is understood in the art that the sequence of an antisense oligonucleotide need not be 100% complementary to that of its target nucleic acid to be hybridizable under physiological conditions. Antisense oligonucleotides hybridize under physiological conditions when binding of the oligonucleotide to the TREM-1 nucleic acid interferes with the normal function of the TREM-1 nucleic acid, and non-specific binding to non-target sequences is minimal.

### RNAi

The present invention further contemplates the use of RNA interference (RNAi) to inhibit the expression of TREM-1, DAP12/TyroBP, or any other member of the TREM-1 or DAP12/TyroBP signaling pathways. The RNAi molecules of the present invention can be designed to specifically inhibit TREM-1, DAP12/TyroBP, or any other member of the TREM-1 or DAP12/TyroBP signaling pathways. Any type of RNAi sequence can be used for the present invention. Non-limiting examples include short interfering RNA (siRNA) molecules or short hairpin RNA (shRNA). A variety of algorithms are available for RNAi sequence design. In one embodiment, the target sequences for siRNA include about 18, 19, 20 or more nucleotides. 2dT's can be added to the 3′ end during siRNA synthesis, creating an “AA” overhang. In many instances, the GC content of a target sequence is between 35% and 55%, and the sequence does not include any four consecutive A or T (i.e., AAAA or TTTT), three consecutive G or C (i.e., GGG or CCC), or seven “GC” in a row. More stringent criteria can also be employed. For instance, the GC content of a target sequence can be limited to between 45% and 55%, and any sequence having three consecutive identical bases (i.e., GGG, CCC, TTT, or AAA) or a palindrome sequence with 5 or more bases can be excluded. Furthermore, the target sequence can be selected to have low sequence homology to other variants or genes. The effectiveness of an RNAi molecule can be evaluated by introducing or expressing the RNAi sequence in a cell that expresses the targeted gene products. A substantial change in the mRNA or protein level of the targeted gene products is indicative of the effectiveness of the RNAi molecule in inhibiting the expression of that gene. Methods for expressing RNAi molecules in a cell are well known in the art, and include, for example, lentivirus vectors.

### Immunogenic Compositions

Compositions provoking an immune response to TREM-1 or DAP12/TyroBP can be used to reduce TREM-1 signaling. The compositions can include TREM-1 or DAP12/TyroBP protein or a fragment or variant thereof (e.g., a variant or a fragment of which has enhanced binding to a human MHC molecule) useful in provoking an immune response to human TREM-1 or human DAP12/TyroBP. The protein, fragment or variant can be supplied as an isolated polypeptide or with additional peptide sequence as, for example, in a fusion protein or a conjugate with another polypeptide such as a carrier protein. In some embodiments, a nucleic acid encoding the protein, fragment or variant is provided in the immunogenic composition in lieu of providing the protein, fragment or variant itself.

An immunogenic composition preferably also contains an adjuvant. An adjuvant can be any substance that enhances or potentiates an immune response (antibody and/or cell-mediated) to an exogenous antigen. Examples of immunostimulants include aluminum salts; biodegradable microspheres (e.g., polylactic galactide); liposomes (into which the compound is incorporated); cytokines (such as, for example, GM-CSF or IL-2, IL-7, or IL-12, or nucleic acids encoding them); and CpG polynucleotides.

As noted above, a vaccine can contain DNA encoding TREM-1 or DAP12/TyroBP protein or a portion or variant thereof and can also contain DNA encoding an adjuvant protein such as a cytokine, such that the polypeptide or polypeptides are generated in vivo. The DNA can be present within any of a variety of delivery systems known to those of ordinary skill in the art, including nucleic acid expression vectors, gene delivery vectors, and bacteria expression systems. Numerous gene delivery techniques are well-known in the art. Appropriate nucleic acid expression systems contain the necessary DNA sequences for expression in the subject (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In one embodiment, the DNA is introduced using a viral expression system (e.g., vaccinia or other pox virus, retrovirus, or adenovirus), which may involve the use of a non-pathogenic (defective), replication competent virus. Techniques for incorporating DNA into such expression systems are well-known to those of ordinary skill in the art. The DNA can also be “naked,” as described, for example, in Ulmer et al. (1993) Science 259:1745-1749. The uptake of naked DNA can be increased by coating the DNA onto biodegradable beads, which are efficiently transported into the cells. A vaccine may comprise both a polynucleotide and a polypeptide component. Such vaccines may provide for an enhanced immune response.

### Ligand Binding Competitors

TREM-1 signaling can also be inhibited by administration of a competitor for binding to the ligand for TREM-1. This can be accomplished, for example, by administration of a soluble fragment of a TREM-1 extracellular domain, optionally coupled to a carrier protein, such as, for example, an IgG immunoglobulin known in the art. For example, administration of a TREM-1-Fc fusion protein using a TREM-1 fragment and a human IgG1 Fc portion has been described and shown to be effective to protect against microbial sepsis (see, for example, U.S. Patent Application Publication No. 2005-0260670, herein incorporated by reference in its entirety). The IgG Fc portion of the fusion protein may be derived from any IgG subclass (e.g., IgG1, IgG2, IgG3, and IgG4). Methods of making TREM-1/IgG fusions proteins are well known. For example, Bouchon et al. (2000) Am. Assoc. Immun. 164(10):499.1-95 describes and teaches how to produce a soluble TREM-1-Fc fusion protein. It is now expected, based on the association of TREM-1 and DAP12/TyroBP with RA, that similar administration of a suitable TREM-1 fragment in RA patients should reduce the severity of the disease. Importantly, treatment of a human does not necessarily require administration of a fragment of wild-type human TREM-1: other TREM-1 fragments from other mammals can be used, and one or more amino acid substitutions can be incorporated, so long as the fragment retains the ability to compete with endogenous human TREM-1 for binding to ligand.

### Binding Partners

A further means for treating inflammatory disease, such as, for example, RA and asthma, includes administration of a binding agent, such as a protein, a peptide and/or an antibody or a portion thereof (e.g., a Fab, F(ab′)₂, Fv or a single chain Fv fragment), that interacts with, e.g., binds to and/or neutralizes, a therapeutic target. Therapeutic targets of the present invention include, for example, TREM-1, TREM-1 ligand, DAP12/TyroBP, and any other member of the TREM-1 signaling pathway. Administration of an anti-TREM-1 binding agent, for example, an anti-TREM-1 antibody, to an RA or asthma patient can reduce the symptoms of the disease by inhibiting and/or antagonizing TREM-1 or DAP12/TyroBP activity or TREM-1 signaling. The antibody can be an isolated antibody. In one embodiment, the antibody is an antagonistic antibody. In another embodiment, the antibody is a neutralizing antibody. In a further embodiment, the antibody modulates, reduces and/or inhibits one or more TREM-1 associated activities, including, but not limited to, modulating, reducing and/or inhibiting TREM-1 interaction with TREM-1 ligand and/or DAP12/TyroBP; modulating, reducing and/or inhibiting TREM-1 mediated signal transduction; modulating, reducing and/or inhibiting expression of TREM-1 activated pro-inflammatory cytokines and/or chemokines; and modulating, reducing and/or inhibiting the expression of TREM-1 activated genes, such as, for example, SPP1. Anti-TREM-1 antibodies of the invention can include, for example, antibodies that specifically bind TREM-1, and/or antibodies that bind the membrane-bound form of the TREM-1 receptor without activating the TREM-1 receptor. In certain embodiments the antibody or fragment thereof is directly or indirectly labeled with a detectable substance to facilitate detection of the bound or unbound antibody. Suitable detectable substances include, for example, enzymes, prosthetic groups, fluorescent materials, luminescent materials and radioactive material. Methods of making and methods of screening antibodies such as those useful for practicing the invention are well known in the art (see, e.g., Harlow et al. (1988) Antibodies: A Laboratory Manual, (New York: Cold Spring Harbor Laboratory). Anti-TREM-1 antibodies of the invention can also include single domain antibodies derived from any species. Alternate binding domain polypeptides, such as, for example, a SMIP™, can also be used to inhibit and/or antagonize TREM-1 or DAP12/TyroBP activity or TREM-1 signaling. The antibodies, or fragments thereof, can also be used in diagnosing, monitoring, and/or preventing in a subject an inflammatory disease, such as, for example, RA and asthma.

### EXAMPLES

### Example 1

### Transcriptional Profiling Analysis of TREM-1 and DAP12/TyroBP

Global analysis of messenger RNA by gene expression analysis has been successfully applied to many diseases to identify genes that contribute to disease pathogenesis and that are potential targets for novel therapies (Battiwalla et al. (2005) Genes and Immunity 6(5):388-97). Using gene expression analysis, TREM-1 and DAP12/TyroBP were identified as a potential target for therapies for inflammation diseases, such as, for example, RA.

Briefly, twenty-four synovial tissue samples were obtained from RA patients during surgery as defined by American College of Rheumatology criteria, with prior approval from the local research ethics committee. Of these twenty-four samples, ten samples were obtained from joint synovium and fourteen samples were obtained from tenosynovium. Eight uninvolved (i.e., normal) synovial tissues were obtained from non-RA patients. The uninvolved synovial tissues were isolated from three patients who required amputation due to blunt trauma and the synovia were isolated from sites distant from the points of trauma. Synovial samples from RA patients and non-RA patients were harvested and immediately flash frozen in liquid nitrogen and stored at −80° C. until processed. Total RNA was isolated and analyzed using Affymetrix® (Santa Clara, Calif.) HG_U95A and B (human samples) GeneChip® oligonucleotide microarrays. Expression measurements from the arrays were generated by the Affymetrix® MAS4 algorithm, and normalized to estimates of transcripts per million by reference to spiked-in standards (Hill et al. (2001) Genome Biol. 2(12):RESEARCH0055).

RNA Isolation

Frozen samples were disrupted and lysed in tissue lysis buffer (RNAgents® Kit, Promega, Madison, Wis.) with a PowerGen™ 700 homogenizer (Fisher Scientific, Pittsburgh, Pa.). Total RNA was isolated with a modification of the manufacturer's recommendations. Briefly, RNA was precipitated with the addition of isopropanol and washed twice with cold 75% ethanol. The pellet was dissolved in RNeasy® minikit sample lysis buffer (RLT) and RNA was purified according to the manufacturer's recommendations (Qiagen, Hilden, Germany). For each sample, total RNA was quantitated from a measure of UV absorption at 260 μm, and an aliquot was analyzed with an Agilent® 2100 Bioanalyzer™ (Agilent Technologies, Santa Clara, Calif.) to determine RNA integrity.

### Preparation of Hybridization Solutions for Oligonucleotide Array Analysis

Double-stranded cDNA was prepared from 5 μg of total RNA using the SuperScript® Choice™ kit (Invitrogen, Carlsbad, Calif.) and 33 pMol of oligo-dT primer containing a T7 RNA polymerase promoter (Proligo, LLC, Boulder, Colo.). First strand cDNA synthesis was initiated with the addition of the following kit components: first strand buffer at 1×, DTT at 10 mM, dNTPs at 500 μM, Superscript® RT II at 400 U, and RNAse inhibitor at 40 U. The reaction proceeded at 47° C. for 1 hour. Second strand synthesis proceeded with the addition of the following kit components: second strand buffer at 1×, additional dNTPs at 200 μM, E. coli DNA polymerase I at 40 U, E. coli RNaseH at 2 U, E. coli DNA ligase at 10 U. The reaction proceeded at 15.8° C. for 2 hr. T4 DNA polymerase (New England BioLabs, Beverly, Mass.), at a final concentration of 6 U, was added for the last five minutes of the second strand reaction. Double stranded cDNA was purified using a solid-phase, reversible immobilization technique, and collected in a volume of 20 μl of 10 mM Tris acetate, pH 7.8. Purified cDNA (10 μl) was transcribed with the BioArray™ High Yield™ RNA Transcript Labeling Kit (T7), following the manufacturer's protocol (Enzo, Farmingdale, N.Y.). Biotin-labeled, anti-sense cRNA was purified using an RNeasy® mini kit as described by the manufacturer (Qiagen, Hilden, Germany). The cRNA yield was determined from a measure of UV absorption at 260 nm.

Oligonucleotide Microarray Hybridization Procedures

To improve hybridization efficiencies, 15 μg of cRNA was fragmented. The fragmented cRNA probes were used to create an oligonucleotide microarray hybridization solution as suggested by the manufacturer (Affymetrix, Santa Clara, Calif.). Hybridization solutions contained a mix of eleven prokaryotic RNAs, each at a different known concentration, which were used to create an internal standard curve for each microarray and was interpolated to determine the frequencies of detected genes. Hybridization solutions were pre-hybridized to two glass beads (Fisher Scientific, Pittsburgh, Pa.) at 45° C. overnight. The hybridization solution was removed to a clean tube and heated for 1-2 min at 95° C. and microcentrifuged at maximum speed for 2 minutes to pellet insoluble debris. Labeled cRNA solutions were hybridized to Affymetrix® (Santa Clara, Calif.) Hg_U95Av2 & B GeneChip® oligonucleotide microarrays on which sequences for 25,128 human gene sequences were arrayed. Following hybridization, cRNA solutions were recovered and microarrays were washed and prepared for scanning according to Affymetrix protocols. Raw fluorescence data were collected and reduced with the use of the GeneChip® 3.2 application (Affymetrix, Santa Clara, Calif.).

Analysis of Expression Profiling Data

The primary data were processed using the hybrid scaled frequency normalization described by Hill et al. (2001) Genome Biol. 2(12):RESEARCH0055. The scaled frequency data were reduced and analyzed using GeneSpringGX™ v7.3 (Agilent Technologies, Santa Clara, Calif.). Two types of analyses were performed. In the first, all diseased samples were compared against all normal samples. In the second, the data were subdivided based upon site of disease, such that joint RA synovia were normalized to the average values of the control joint synovial samples, and tenosynovial RA samples were normalized to the average values of the control tenosynovial samples.

To identify transcripts that were associated with RA, gene transcript scaled frequencies from each of the diseased samples were normalized to the average of all of the non-rheumatoid gene transcript frequencies. Data were reduced by filtering for gene transcripts that had either increased or decreased levels of expression relative to the average of the controls. In addition to the data normalization steps, several statistical analyses were performed on the filtered data. False discovery rates (FDR) and Bonferroni family-wise error rates (FWER) were determined using a cutoff p-value of 0.05. In addition, an unsupervised hierarchical cluster analysis and a k-nearest neighbor analysis of the data set were performed in GeneSpring™. The resulting data set was visualized with an unsupervised hierarchical cluster analysis.

In a second analysis, the joint and tendon samples were analyzed separately. Joint synovial and tenosynovial samples were normalized to the averages of their respective site-specific controls. The same filtration and statistical parameters were applied to each of analysis as described above. The two resulting data sets were combined using a Venn analysis and were subjected to an unsupervised hierarchical cluster analysis of the genes and samples. An analysis using k-nearest neighbor was used to discriminate between the four parameters: disease, non-disease, joint, and tendon. The process for this analysis is described below. The genes with the best discrimination were then subjected to an unsupervised hierarchical cluster analysis of the samples and genes.

Statistical analysis of expression data was executed on log-2 transformed expression measurements. Fold-changes between groups of samples were computed by taking the difference of the means of the log-2 expression levels in each group, and back-transforming the resulting log-fold-change to the linear scale. The significance of differential expression between groups was determined by a permutation test. Briefly, an F-statistic was computed for each probeset in each comparison of interest, using a within-group error estimate derived from pooling the error estimates of probesets with similar intensity levels. The observed F-statistics were referred to a null distribution of identically calculated F-statistics from the same dataset after random permutation of the sample labels. The p-value for differential expression was defined as the fraction of permutation F-statistics that were greater than the observed F-statistic for each probeset (Edington (1995) Randomization Tests (New York: Marcel Dekker); Zar (1999) Biostatistical Analysis (New Jersey: Simon & Shuster)).

Results

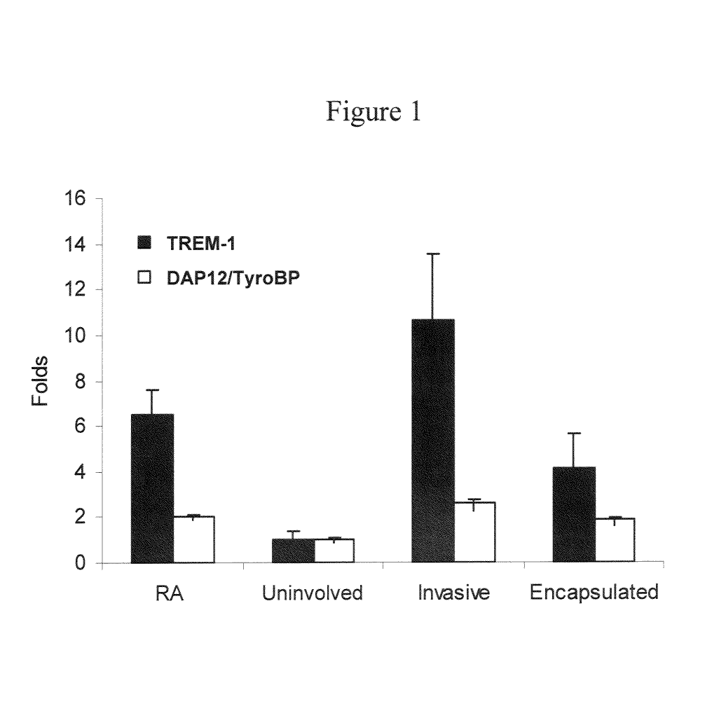

Gene expression analysis of synovial biopsies revealed that TREM-1 and DAP12/TyroBP mRNA expression is significantly up-regulated in RA patients. FIG. 1 is a bar graph showing expression of TREM-1 and DAP12/TyroBP in synovium from RA patients. The fold changes of TREM-1 expression were normalized to normal synovium specimens. TREM-1 mRNA was up-regulated 6.5 fold (p-value of 1.98×10⁻⁶) in RA positive samples (n=24) as compared to uninvolved samples (n=8) (FIG. 1). Moreover, DAP12/TyroBP mRNA was up-regulated 2 fold (p-value of 7.83×10⁻⁴) in RA positive samples (n=24) as compared to uninvolved samples (n=8) (FIG. 1).

In addition to being up-regulated in RA, TREM-1 and DAP12/TyroBP mRNA expression levels vary with the severity of RA. Fourteen tendon samples from RA patients were divided into two clinically defined disease subtypes, invasive and encapsulated, invasive RA being the more progressive form. TREM-1 mRNA was up-regulated 2.64 fold (p-value of 1.36×10⁻⁴) in invasive tenosynovium samples (n=7) as compared to encapsulated tenosynovium samples (n=7) (FIG. 1). Likewise, DAP12/Tyro12 mRNA was up-regulated 1.4 fold (p-value of 1.67×10⁻²) in invasive samples (n=7) as compared to encapsulated samples (n=7) (FIG. 1).

A comparison of cell-specific gene expression in monocytes, neutrophils, and macrophages indicates that inflammatory cell infiltration is only partly responsible for increased TREM-1 expression levels in RA positive synovial tissues. In order to identify whether the increased TREM-1 expression was due to the infiltration of TREM-1 positive inflammatory cells in the RA synovium, we looked globally at the expression levels of genes that were specifically expressed in monocytes (182 genes), neutrophils (328 genes) and macrophages (34 genes). The mean expression levels of these genes range from 1.22 to 1.59, with large standard deviations. The increased expression of TREM-1 was mainly caused by the up-regulation of TREM-1 gene expression rather than by a large increase in cell infiltration.

### Example 2

### Quantitative Real-Time PCR of TREM-1 and DAP121TyroBP

TREM-1 and DAP12/TyroBP mRNA are overexpressed in a collagen-induced arthritis model. Collagen-induced arthritis (CIA) was performed in male DBA/1 mice (Jackson Laboratories, Bar Harbor, Me.) using bovine collagen type II (Chondrex, Redmond, Wash.). Mice were monitored for disease progression at least three times a week. Individual limbs were assigned a clinical score based on the following index: (0) normal; (1) visible erythema accompanied by one to two swollen digits; (2) pronounced erythema, characterized by paw swelling and/or multi digit swelling; (3) massive swelling extending into ankle or wrist joint; and (4) difficulty in use of limb or joint rigidity. The sum of all limb scores for any given mouse yields a potential maximum total body score of 16. Animals were euthanized and tissues were harvested at various disease stages. RNA from disease animals was prepared from three score 3 paws and one score 4 paw. RNA extracted from normal animals was prepared from four score 0 paws. TREM-1 mRNA was quantified using the following primers and probe:

Forward primer

(SEQ ID NO:1)

CAGATGTGTTCACTCCTGTCATCA (413-436);
Reverse primer

(SEQ ID NO:2)

CTGGGTGAGTATTTTGTGGTAATAAGG (494-468);
Probe

(SEQ ID NO:3)

CCTATTACAAGGCTGACAGAGCGTCCCA (439-466).

A standard curve was generated using known concentrations of mTREM-1. DAP12/TyroBP mRNA was quantified using the following primers and probe:

| | |
|---|---|
| Forward primer | |
| CCTGGTCTCCCGAGGTCAA (255-273); | (SEQ ID NO:4) |
| Reverse primer | |
| GGCGACTCAGTCTCAGCAATG (323-302); | (SEQ ID NO:5) |
| Probe | |
| TTGTTTCCGGGTCCCTTCCGCT (300-279). | (SEQ ID NO:6) |

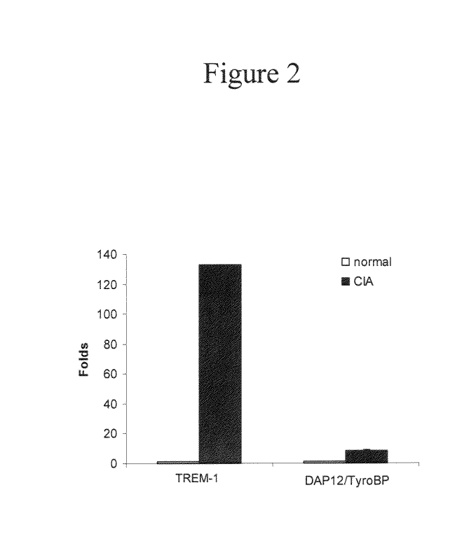

In order to calculate fold-changes in expression, TREM-1 RNA levels and DAP12/TyroBP RNA levels were normalized to GAPDH mRNA. By RT-PCR, TREM-1 mRNA was up-regulated 132 fold in CIA paws as compared to paws from normal mice (FIG. 2), while DAP12/TyroBP was up-regulated 8.21 fold (FIG. 2). These results further confirm that expression of TREM-1 and proteins associated with TREM-1 signaling are elevated in the sites of RA disease.

### Example 3

### TREM-1 Expression by Immunohistochemistry

Immunohistochemistry of various synovial samples was performed to determine whether TREM-1 expression was increased at the protein level. Briefly, five RA synovial samples were obtained from patients during surgery through the Kennedy Institute of Rheumatology and two OA tissues were obtained from New England Baptist Hospital. Tissues were fixed in formalin and embedded in paraffin. Immunohistochemistry was performed on 4 μm tissue sections. Sections were first de-paraffinized in xylene and rehydrated in a graded ethanol series. After washing with PBS, antigens were retrieved and cells were permeablized with Tween 20. Samples were immunostained with mouse anti-TREM-1 antibody (R&D Systems, Minneapolis, Minn.) in a Biogenex™ i6000™ system according to a standard protocol. The secondary antibody and detection reagent used were from a Mach3™-mouse probe HRP polymer kit (Biocare, Concord, Calif.). Cells positive for immunohistochemistry staining were defined as those with brown pigments. For each slide, ten 200× fields of view adjacent to the synovial surface were randomly selected and immunohistochemistry-positive cells were counted and totaled as an immunohistochemistry-positive cell index.

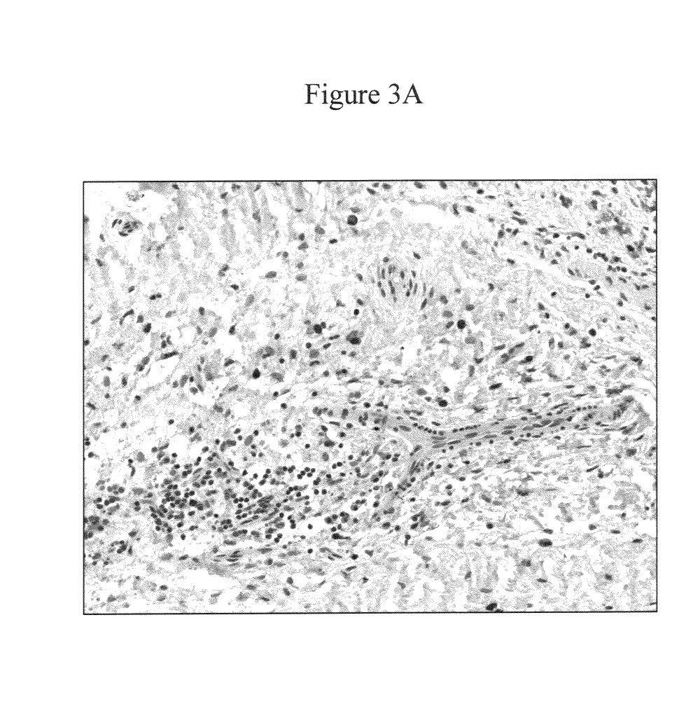

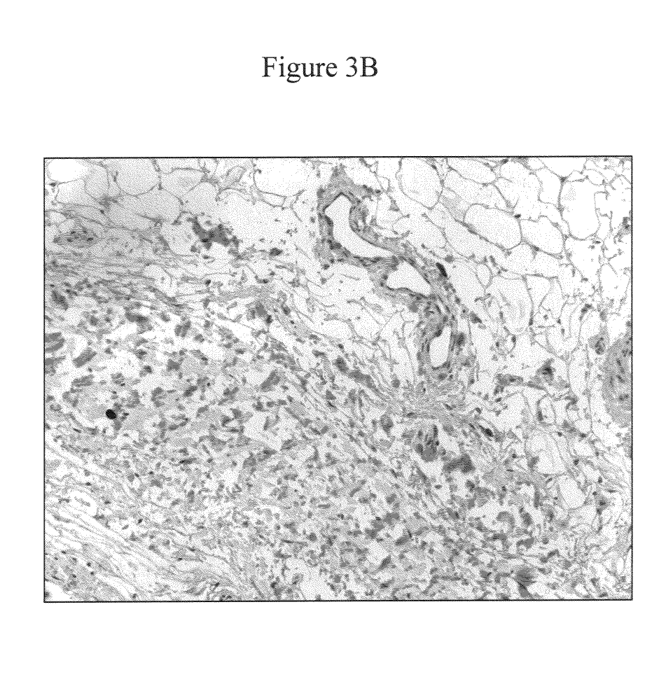

Immunohistochemistry on sections from five RA patients revealed high TREM-1 levels in three out of five patients, with extremely high levels observed in one patient, RA4 (Table 1). Two of the RA samples (RA1 and RA2) had low TREM-1 levels and were similar to control samples (OA1 and OA2) from osteoarthritis patients (Table 1). FIG. 3A depicts one representative field of an anti-TREM-1 labeled RA synovial tissue sample. FIG. 3B depicts one representative field of an anti-TREM-1 labeled synovial tissue sample from a control, OA patient.

To identify the cell type of TREM-1 positive cells in RA samples, double immunohistochemistry of TREM-1 and CD163, CD14, CD68 or myeloperoxidase was performed sequentially by staining with TREM-1 antibody first, followed by CD163 (10D16 from Labvision, Freemont, Calif.), CD14 (Labvision, Freemont, Calif.), CD68 (PG-M1 from Biocare, Concord, Calif.), or myeloperoxidase (Abcam, Cambridge, UK) antibodies, respectively. None of the sections stained with myeloperoxidase suggesting the absence of neutrophils. Surprisingly, only sections from RA5 stained with CD68, but these sections did not co-stain with TREM-1. A small portion (3-10%) of TREM-1 positive cells from RA3 and RA5 were co-stained with CD14 or CD163. The difference in the presence of these markers and TREM-1 in the five RA synovium sections reflected the heterogeneity of the disease.

TABLE 1
Index of TREM-1 positive cells in RA and control (OA)
synovium samples.

| | | IHC positive cell |
|---|---|---|
| Sample | Patient samples | index |
| 1 | RA1 | 27 |
| 2 | RA2 | 20 |
| 3 | RA3 | 129 |
| 4 | RA4 | >1000 |
| 5 | RA5 | 297 |
| 6 | OA1 | 27 |
| 7 | OA2 | 5 |

### Example 4

### Detection of Soluble TREM-1 in Human Clinical Plasma Samples

An enzyme-linked immunosorbent assay (ELISA) was performed to demonstrate that TREM-1 protein levels are both detectable and elevated in human RA samples. Plasma from RA patients was obtained from a phase two, double-blinded, placebo-controlled, parallel, randomized, multicenter, out-patient, comparative study in subjects with active RA and an inadequate response to stable dosages of methotrexate (MTX) (7.5 to 20 mg once weekly). Subjects were enrolled at 81 sites worldwide. At selected sites, 32 subjects who agreed to participate in the voluntary sample collection for exploratory biomarker studies provided blood samples. Data reported here is from plasma samples taken on day 1 (predose). The control group plasma was collected from subjects enrolled in a healthy volunteer multicenter, prospective, non-interventional observational study. Each clinical site's institutional review board or ethics committee approved these studies, and no procedures were performed before obtaining informed consent from each patient.

An ELISA protocol for detecting soluble TREM-1 in human clinical plasma samples was adapted from a DuoSet® ELISA Development System, which is commercially available from R&D Systems (Minneapolis, Minn.; catalog number DY1278). The adapted ELISA protocol reduced false positives and improved the linear dynamic range of the standard curve. Briefly, ELISA was performed in a sandwich format with 4.0 ug/ml of capture antibody and 200 ng/ml of detection antibody. The plasma samples were diluted 1:2 fold in GF1 buffer, which is commercially available from Meso Scale Discovery (Gaithersburg, Md.; catalog number R54BB-3). The standard was also diluted with 1:2 dilution of neat plasma in GF1 buffer. The limit of the detection was 1.37 pg/ml using a four parameter curve fit (XL-Fit IDBS, Burlington, Mass.) with R² of 0.999 in the range of 1.37 to 1000 pg/ml.

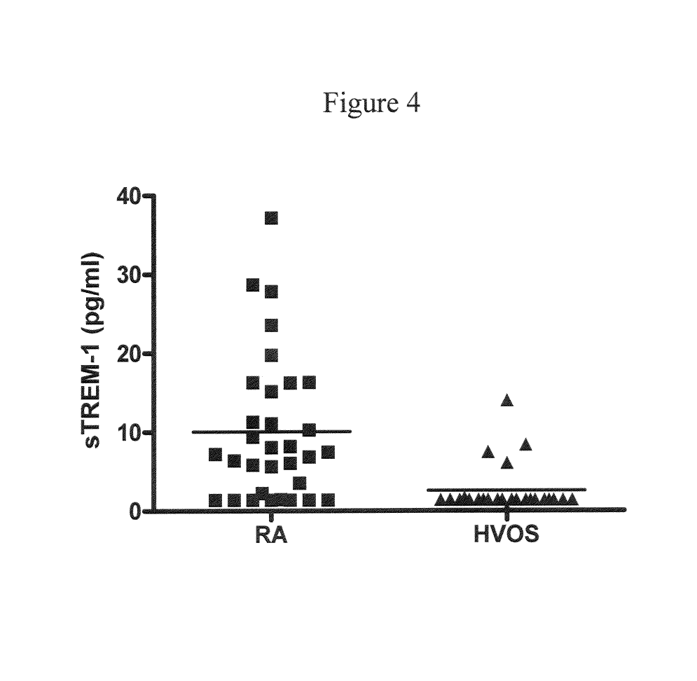

Using the above ELISA method, 32 samples from RA patients and 25 samples from healthy volunteers (HVOS) were tested. FIG. 4 is a graph showing protein levels of soluble TREM-1 detected by ELISA in human plasma samples obtained from RA and control patients (HVOS). As seen in FIG. 4, the average amount of soluble TREM-1 in RA plasma was 10.04±1.626 (n=32) pg/ml, while the average amount of soluble TREM-1 in healthy volunteers (HVOS) was 2.549±0.6253 (n=25) pg/ml. The level of soluble TREM-1 in RA plasma is higher (more than three fold) than that of healthy volunteers, with a p-value of <0.0001 (unpaired t test). Thus, detection of increased levels of human soluble TREM-1 in plasma correlates with and is indicative of RA.

In addition, a significant association between elevated plasma TREM-1 levels and elevated levels of rheumatoid factor was detected.

### Example 5

### Activation of Mitogen Activated Protein Kinases After Crosslinking of TREM-1

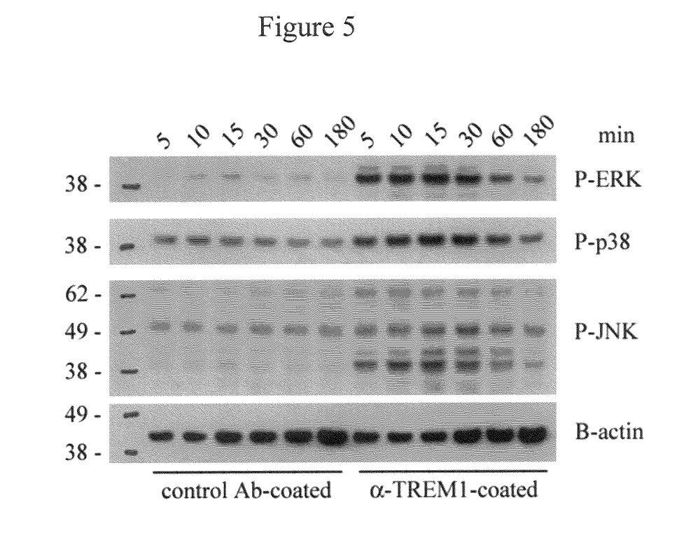

Purified human monocytes were seeded into wells pre-coated with either isotype-matched control antibody or α-TREM-1 cross-linking antibody to determine whether TREM-1 receptor activation triggers the activation of mitogen activated protein kinases (MAPKs). Briefly, human leukopacks (Buffycoats) from healthy volunteers were obtained from the Massachusetts General Hospital Clinical Hematology Laboratory (Boston, Mass.). Buffycoats were stored at 4° C. overnight for cell isolation the following day. Monocytes were isolated by negative selection using RosetteSep® (StemCell Technologies, Vancouver, BC; 15068) as per the manufacturer's protocol by density centrifugation over Histopaque® (SIGMA, H8889). All incubations were at 37° C. in a tissue culture incubator maintained at 5% CO₂. Purified monocytes were maintained in RPMI 1640 medium supplemented with 10% heat-inactivated fetal bovine serum. Tissue culture-treated plates were treated with a 5 μg/ml solution of α-TREM-1 crosslinking antibody (R&D Systems, Minneapolis, Minn.; MAB1278) in PBS overnight in a tissue culture incubator. Control wells were similarly treated with an isotype-matched antibody to E. tenella (Wyeth, Madison, N.J.). Wells were washed twice with PBS immediately prior to cell addition. Western blots were performed using standard protocols over a time course of 5 to 180 minutes (see FIG. 5). α-phospho-ERK, α-phospho-p38, and α-phospho-JNK antibodies were purchased from Cell Signaling Technologies (Danvers, Mass.; 9101, 9211 and 9251, respectively). α-actin antibody was purchased from Sigma (St. Louis, Mo.; A2103). As seen in FIG. 5, activation of TREM-1 with a crosslinking antibody results in broad activation of MAPKs. p38 and JNK were previously unknown to be responsive to TREM-1 activation. These broad pro-inflammatory responses were corroborated by global gene expression changes arising from TREM-1 activation in purified human monocytes (see Example 10).

### Example 6

### Transcriptional Profiling Analysis After α-TREM-1 and LPS Treatments

Transcription profiling analysis was employed to identify genes that are differentially expressed upon activation of the TREM-1 receptor. Using transcriptional profiling, secreted phosphoprotein 1 (SPP1; also known as osteopontin (OPN), bone sialoprotein I (BSPI), early T-lymphocyte activation 1 (ETA-1), or MGC110940) was identified as a marker specific for TREM-1 activation, as opposed to being an obligatory pro-inflammatory readout.

Tissue Culture Preparation and RNA Isolation

Purified human monocytes from multiple donors were prepared as described in Example 9 and were seeded into tissue-culture treated wells. Tissue-culture treated wells were either untreated (for untreated control and lipopolysaccharide (LPS) treatment), or pre-coated with either isotype-control antibody or α-TREM-1 crosslinking antibody as described in Example 9. For LPS treatments, gel filtration chromatography purified LPS from S. enterica (Sigma, St. Louis, Mo.; L2262) was added to a final concentration of 1 ng/ml. Subsequently, 5×10⁶ monocytes were plated into untreated or antibody-coated 12-well tissue culture-treated plates. After 2 hours treatment, total RNA was isolated using Qiagen® QIAshredder™ columns and RNeasy® Mini Kits (Valencia, Calif.; 79654 and 74104, respectively) as per the manufacturer's protocols. A 2 hour time-point was chosen to minimize the contribution of secondary and/or differentiation effects, while generating gene expression changes suitable for high-confidence analysis. Total RNA yields ranged from 1-6 μg. Total RNA was further purified by DNase treatment, followed by phenol-chloroform extraction and ethanol precipitation, using standard protocols. Microarray analysis was performed using Affymetrix® HG_U133 2.0 Plus arrays according to established protocols. For each array, all probe sets were normalized to a mean intensity value of 100. Default GeneChip® Operating System (GCOS) statistical values were used for all analyses. Monocytes from a total of 11 healthy donors were analyzed.

Analysis of Expression Profiling Data

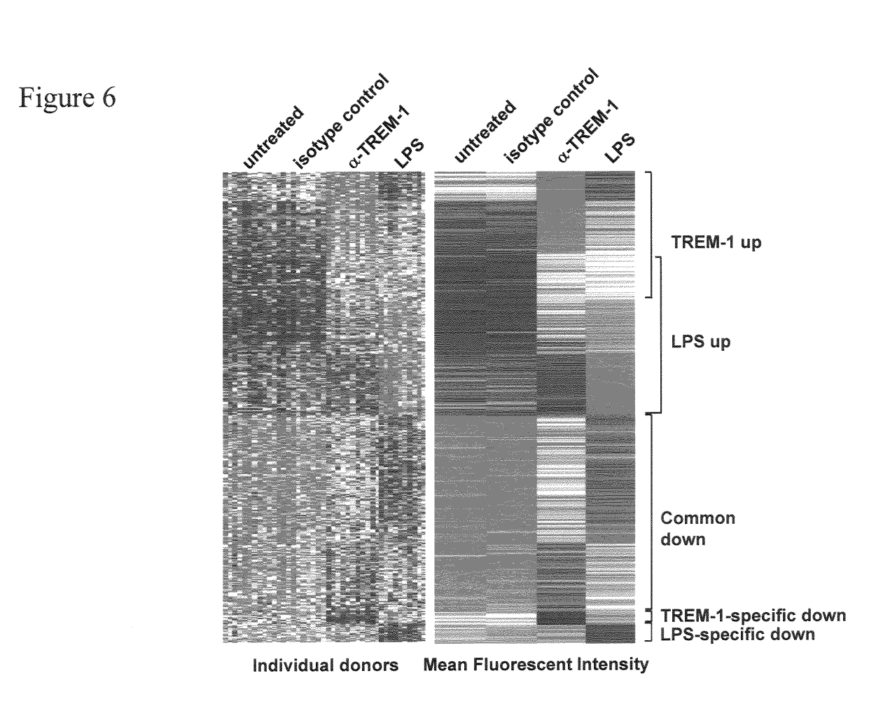

Qualifiers present at >50 signal and called present in >66% of treatment groups were considered for analysis. Normalized signal values were transformed to log₂ values prior to ANOVA analysis. Qualifiers with p<0.01 and ≧2-fold change (FC) in expression between any treatment groups were used to generate the heat-map in FIG. 6 and for subsequent analyses. Fold reductions are reported as negative fold-changes. For genes represented by multiple qualifiers, the qualifier with the highest average intensity in the untreated sample was chosen for further analysis.

FIG. 6 shows a heat-map clustering analysis of the transcriptional profiling data. In FIG. 6, individual donors are shown on the left (each column represents an individual donor) and average mean intensity is shown on the right; each row represents an individual qualifier. Fluorescent intensities are shown. α-TREM-1-treated samples were compared to control antibody-treated samples and LPS-treated samples were compared to no treatment. A hierarchical clustering algorithm was used to group qualifiers with similar patterns of expression. In FIG. 6, bracketed regions indicate heat map regions corresponding to qualifiers up-regulated by TREM-1 activation, up-regulated by LPS, common down-regulated, down-regulated by TREM-1 activation, or down-regulated by LPS.

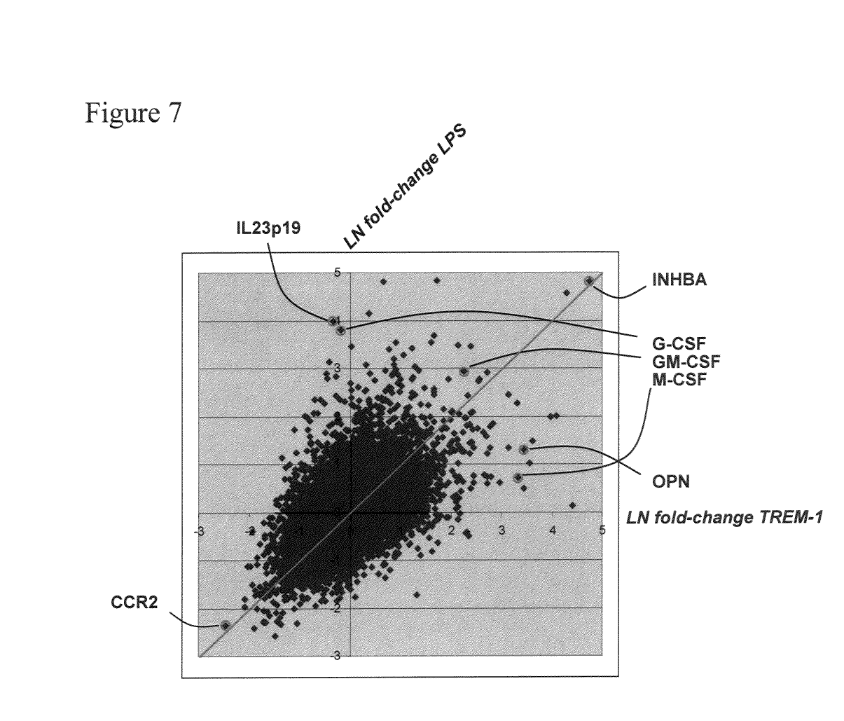

FIG. 7 shows an exemplary scatter plot of all present calls. For scatter plot analysis, In fold-changes plotted for TREM-1 (x-axis) and LPS (y-axis), with down-regulation converted to negative values. In FCs (α-TREM-1/control antibody and LPS/no treatment) for all present calls were calculated and plotted, with down-regulated genes plotted as ln(−1/FC). Selected genes are highlighted. The 45° axis demarcates genes comparably regulated by both treatments.

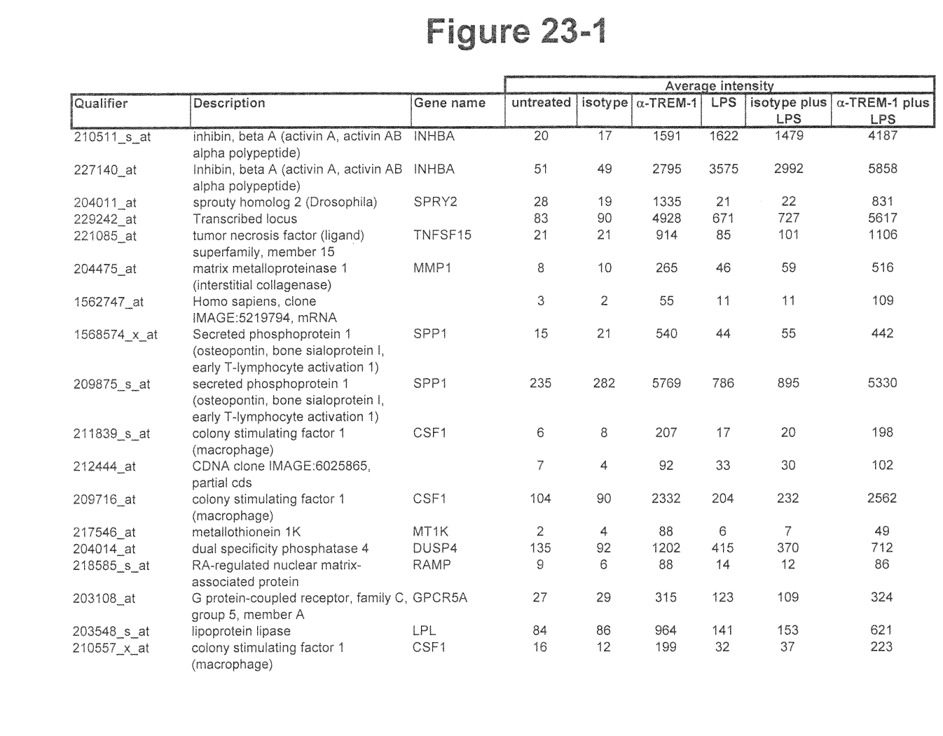

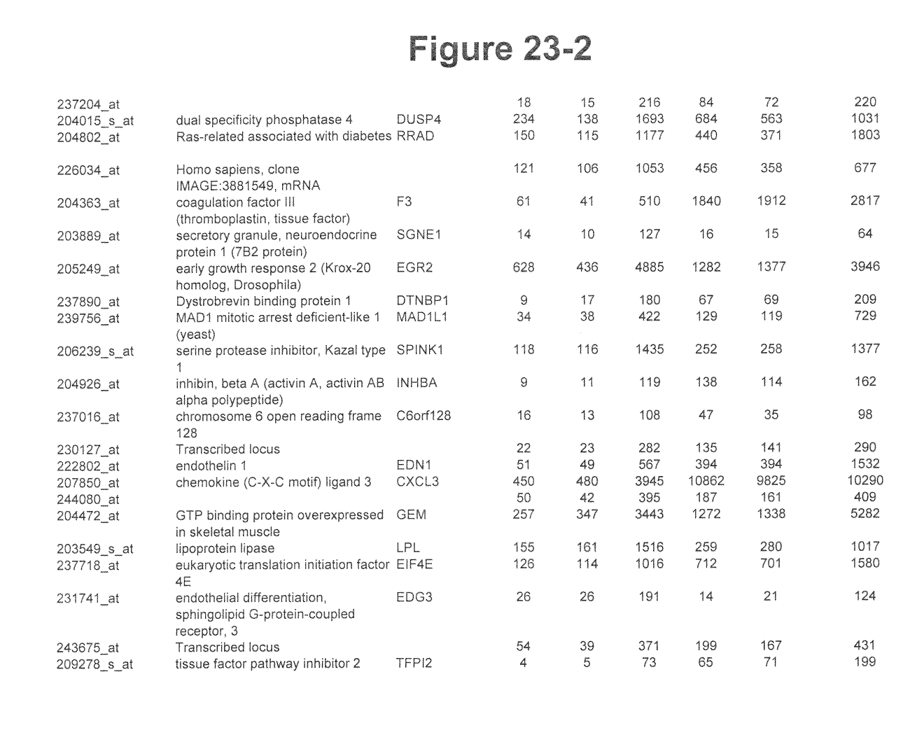

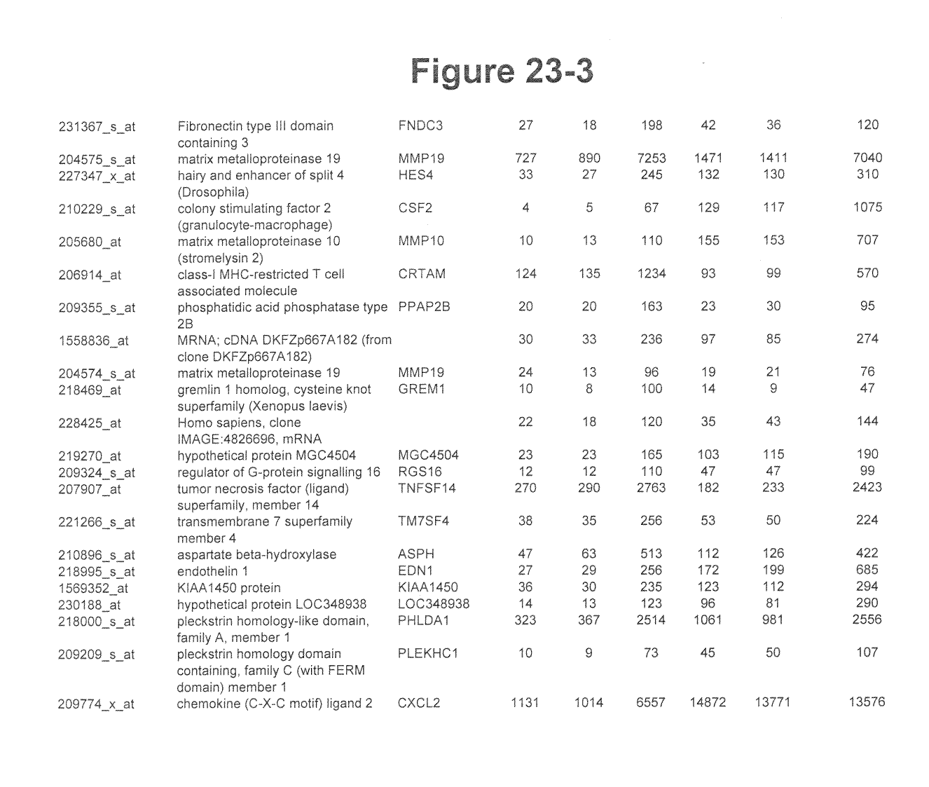

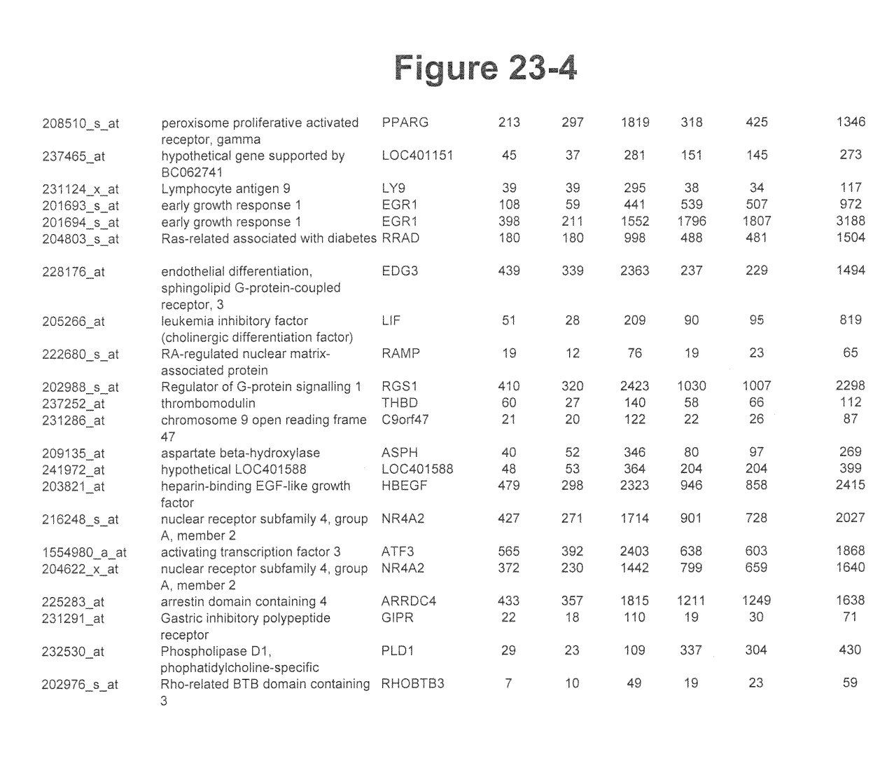

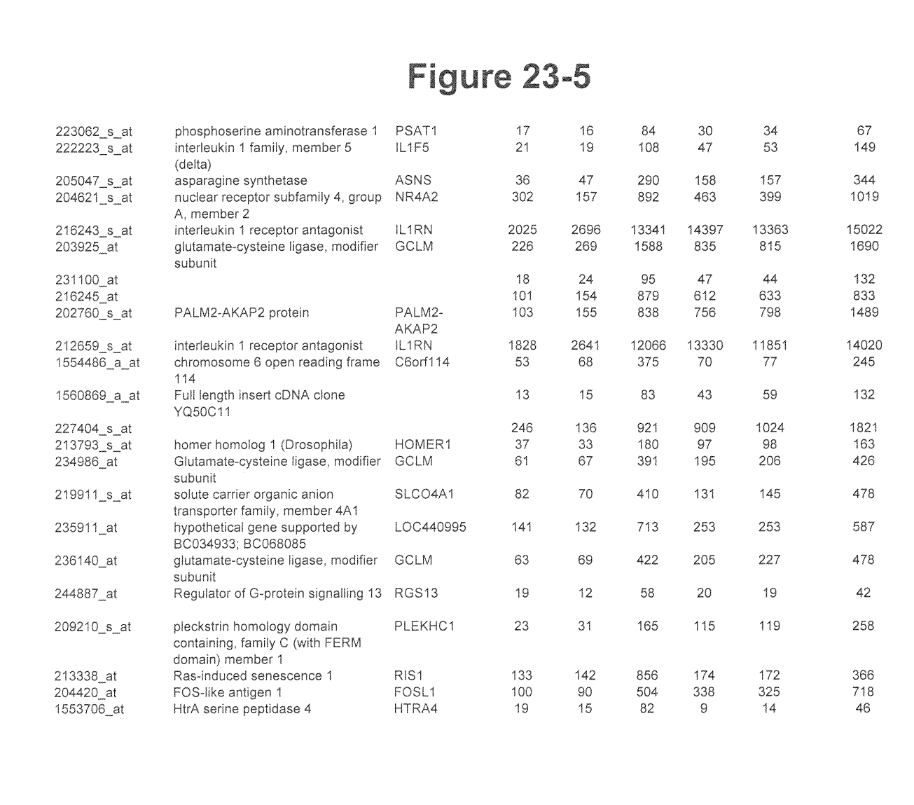

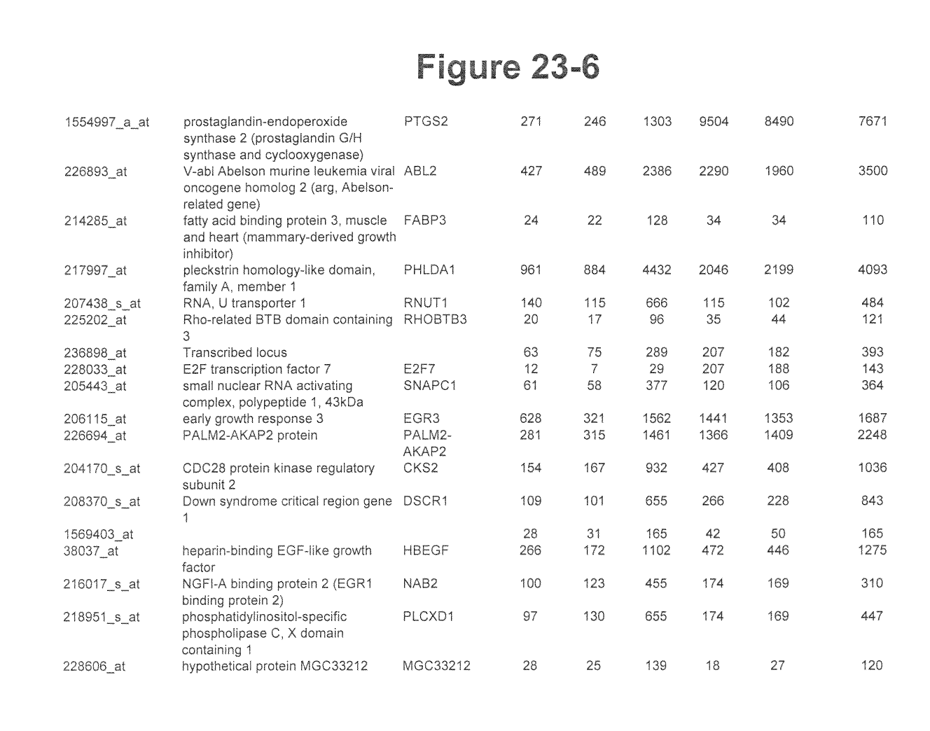

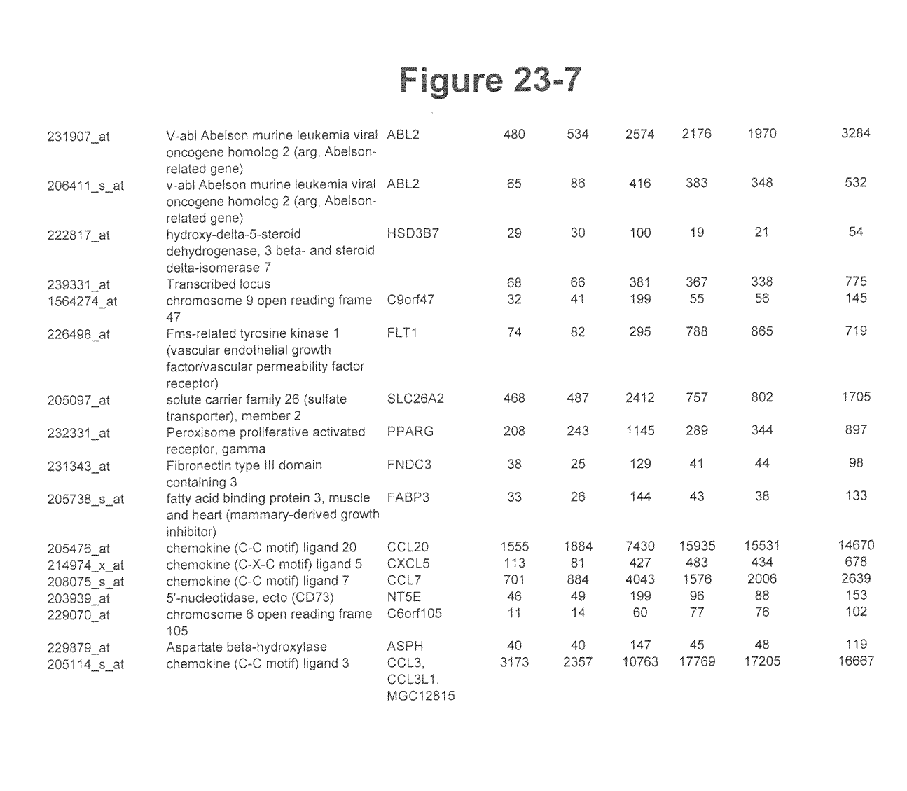

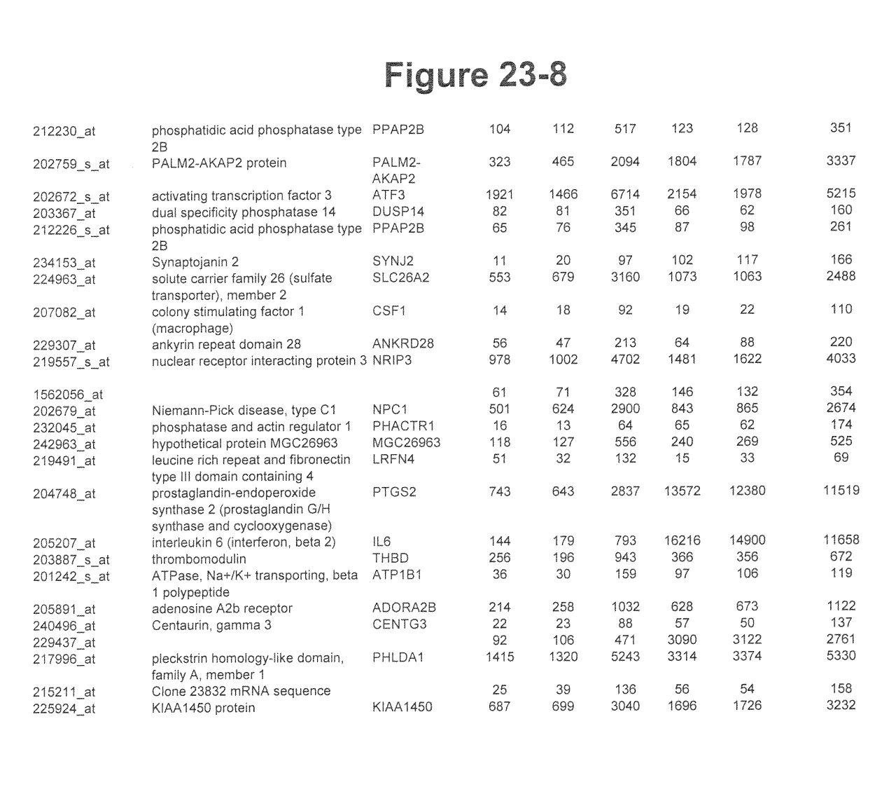

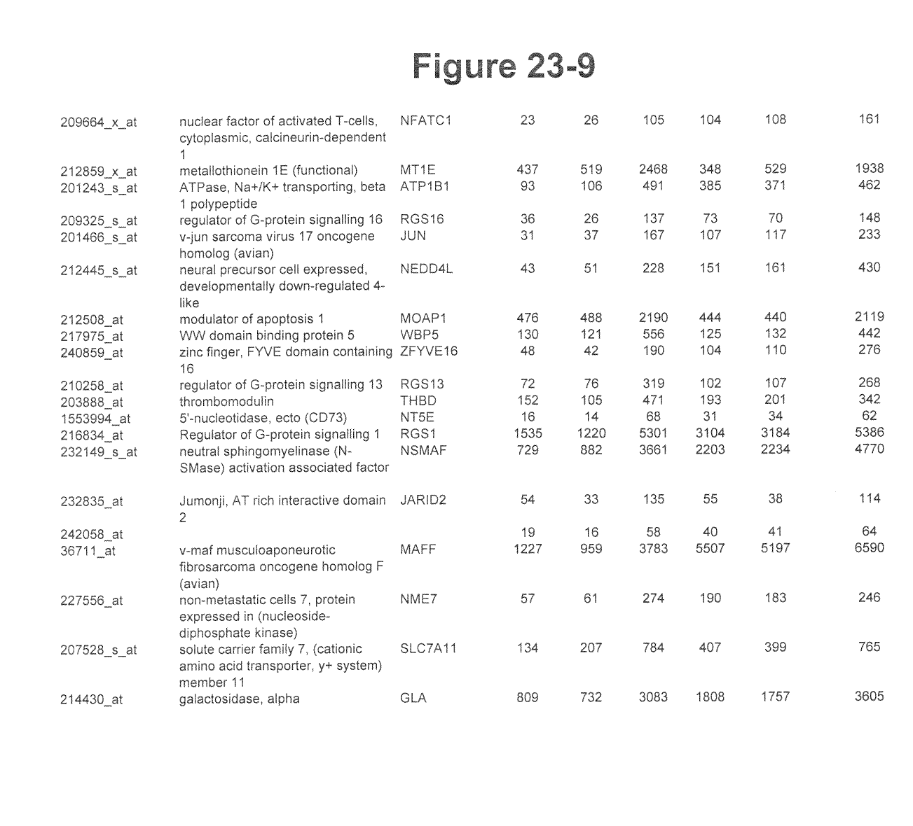

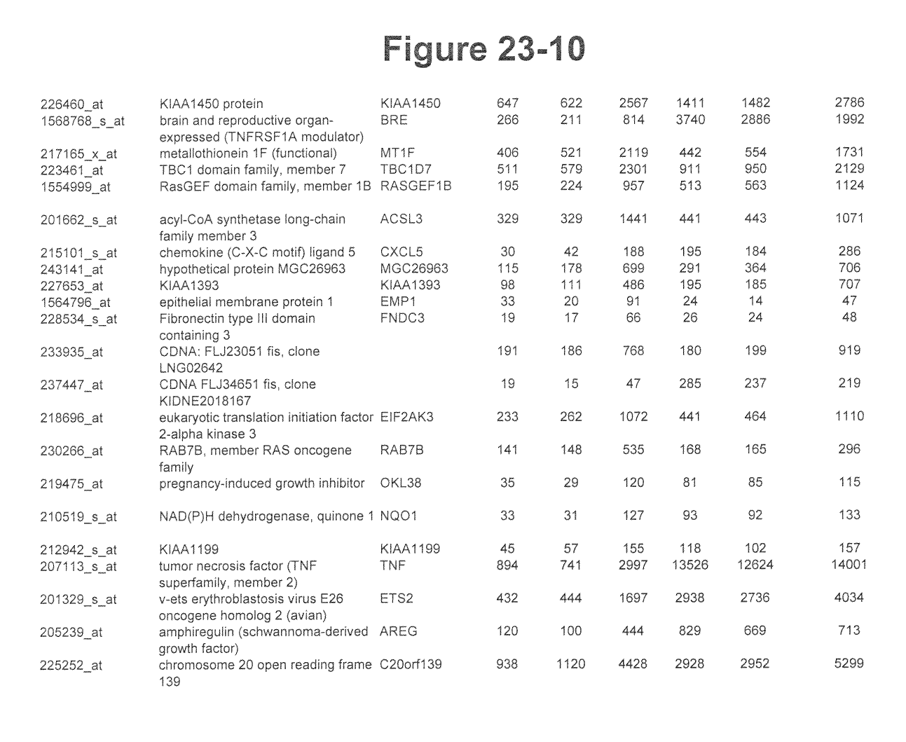

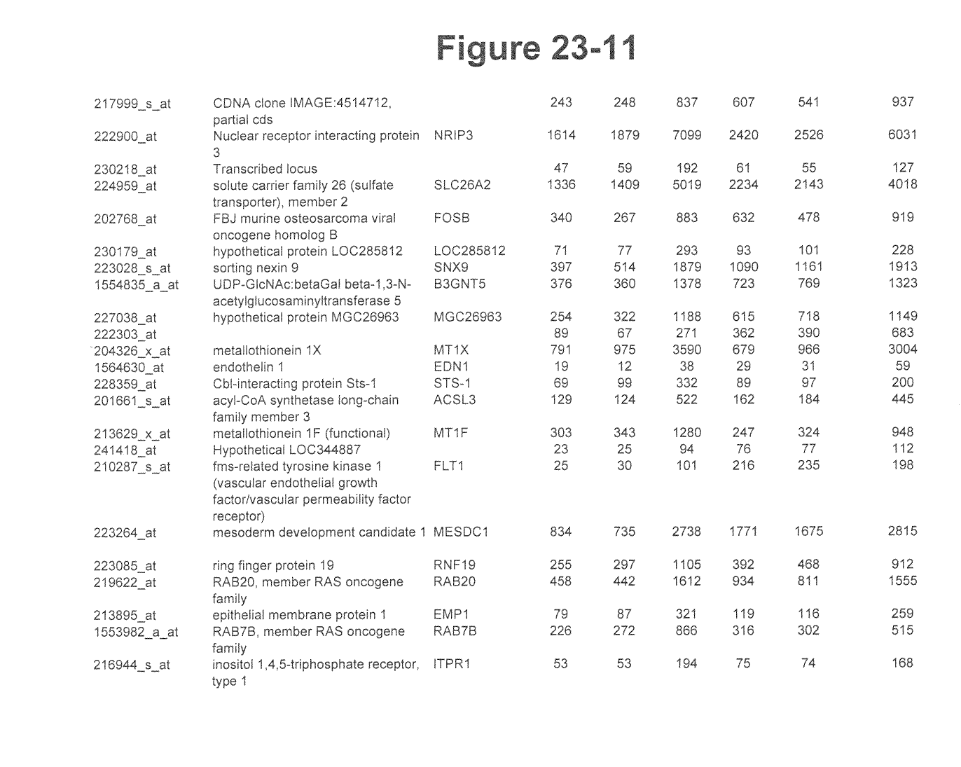

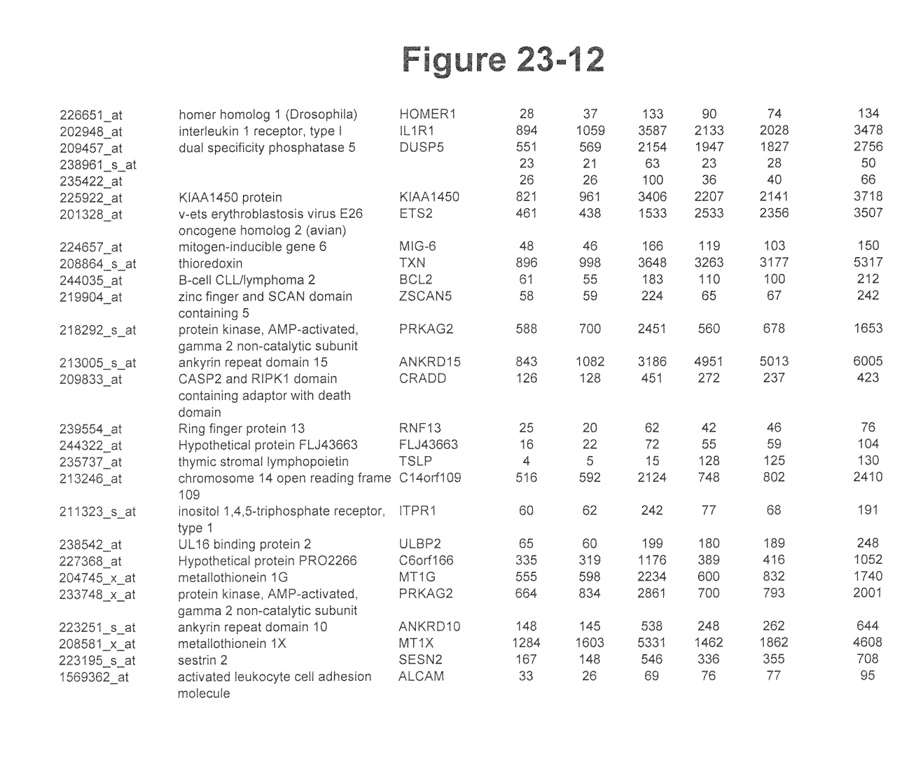

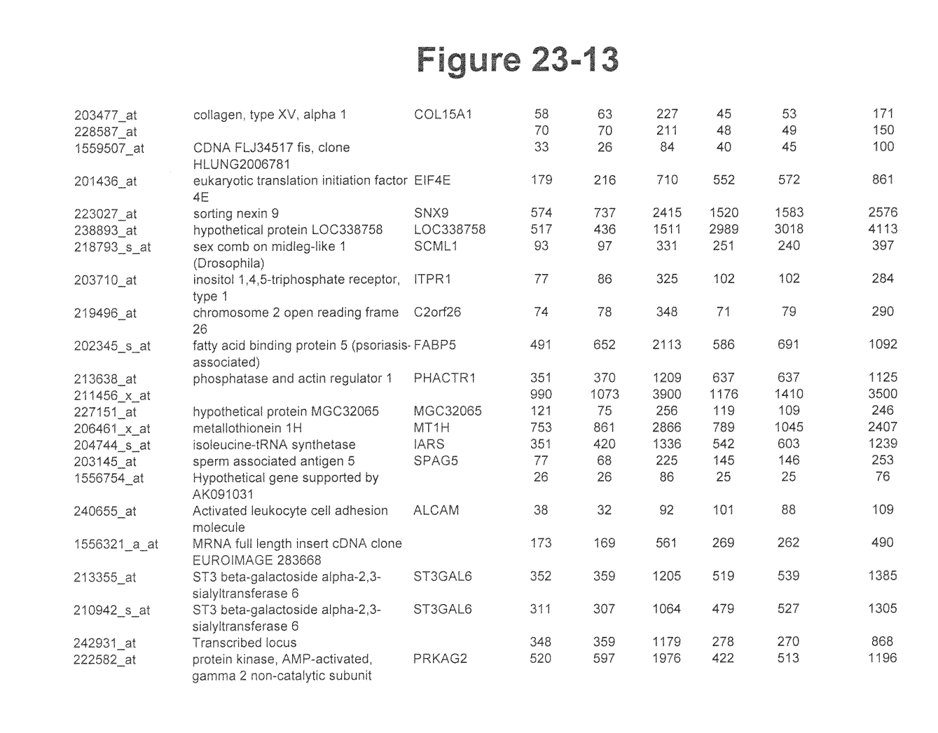

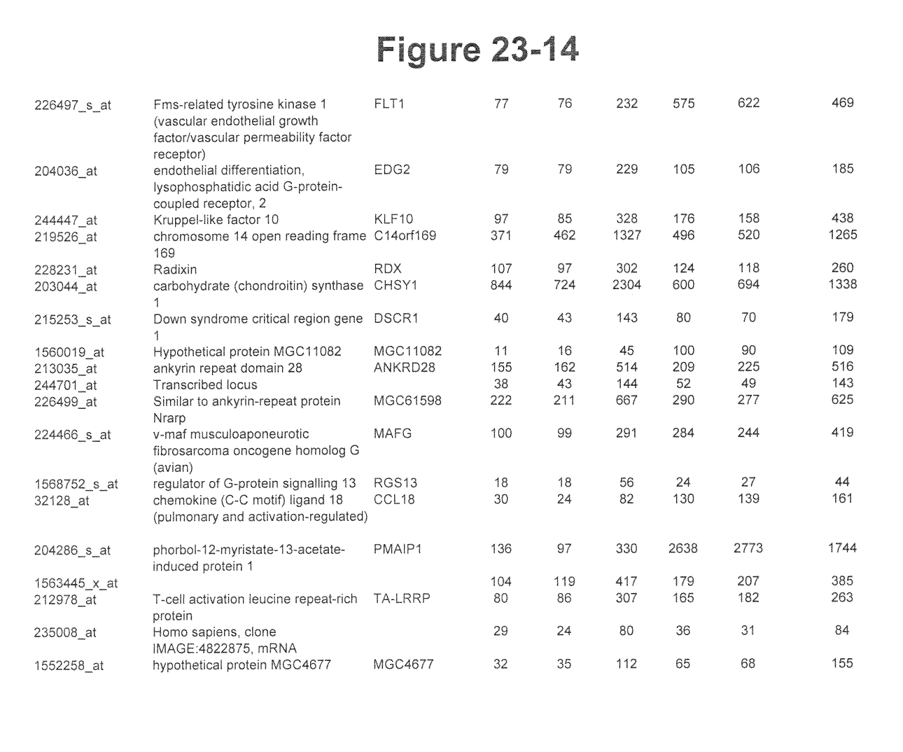

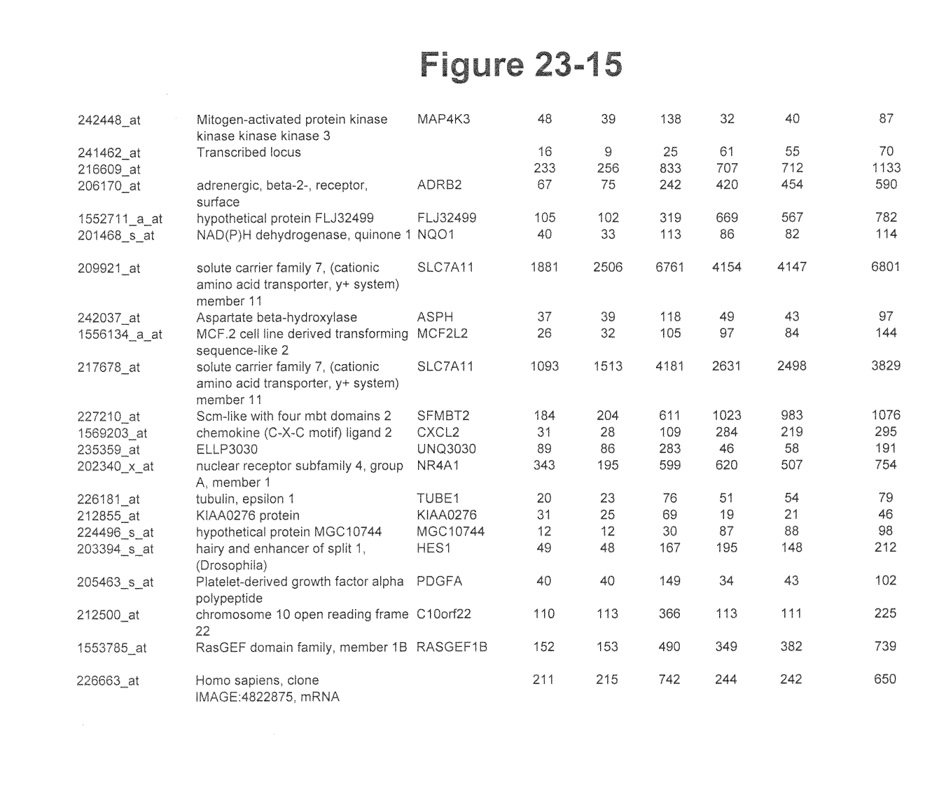

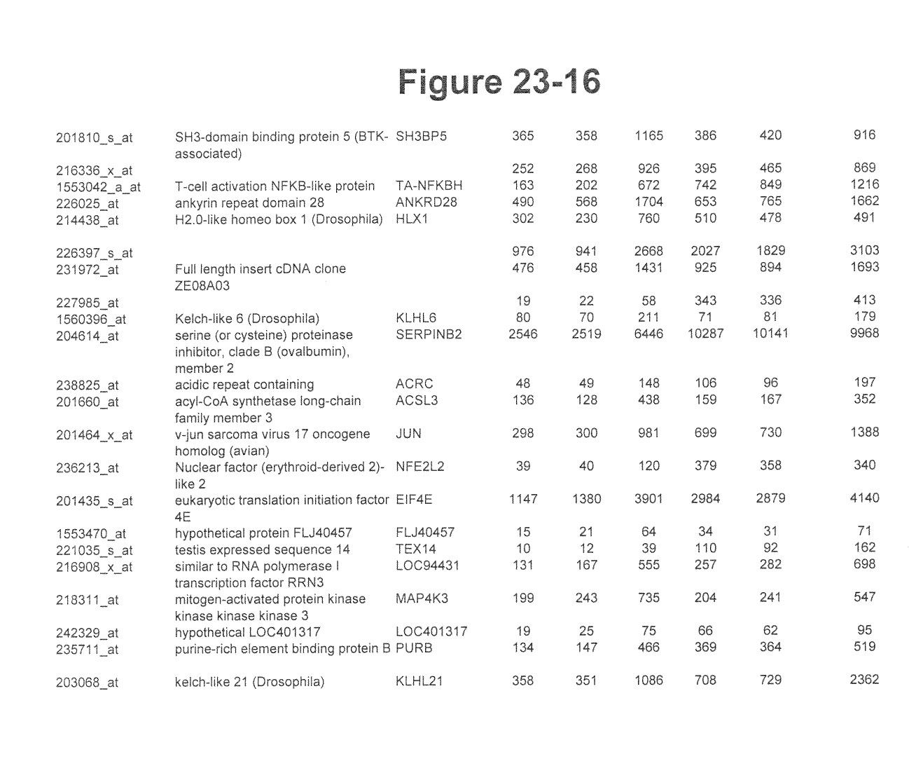

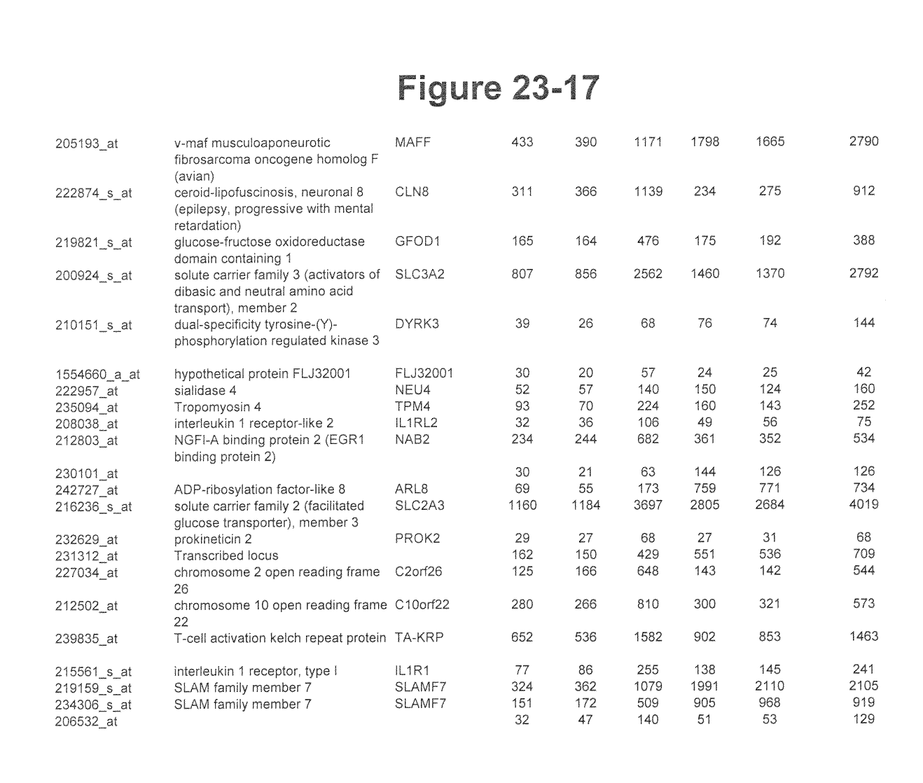

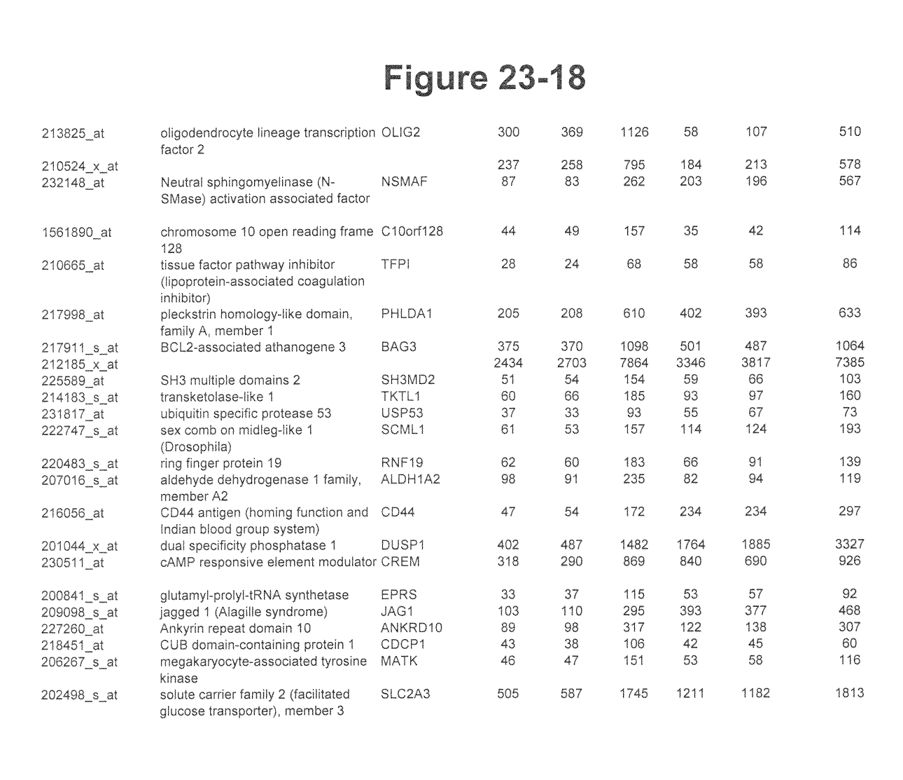

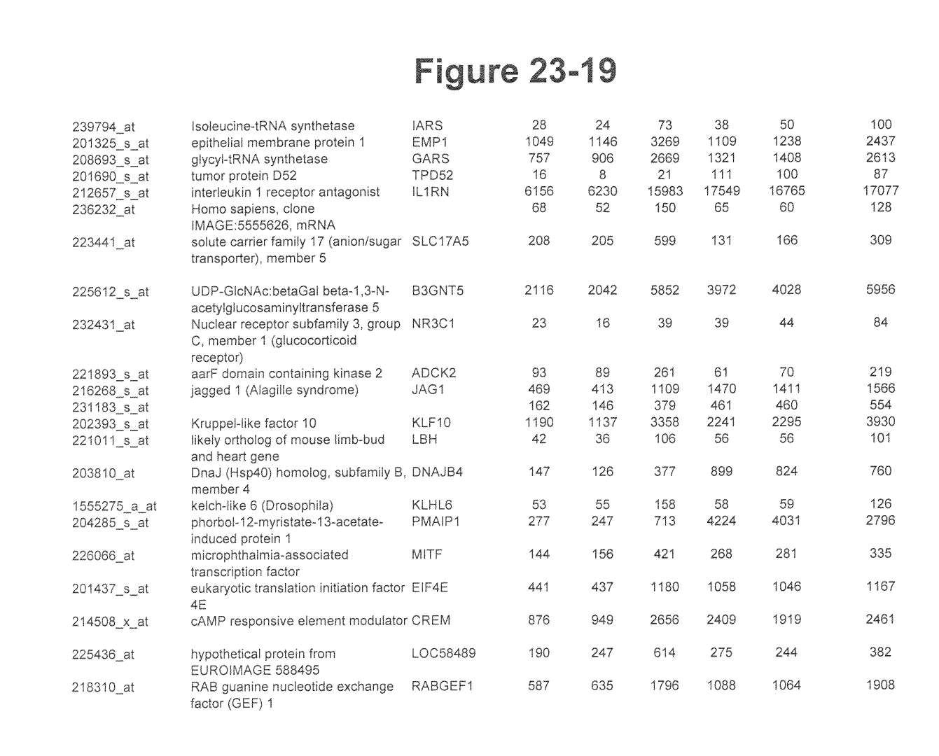

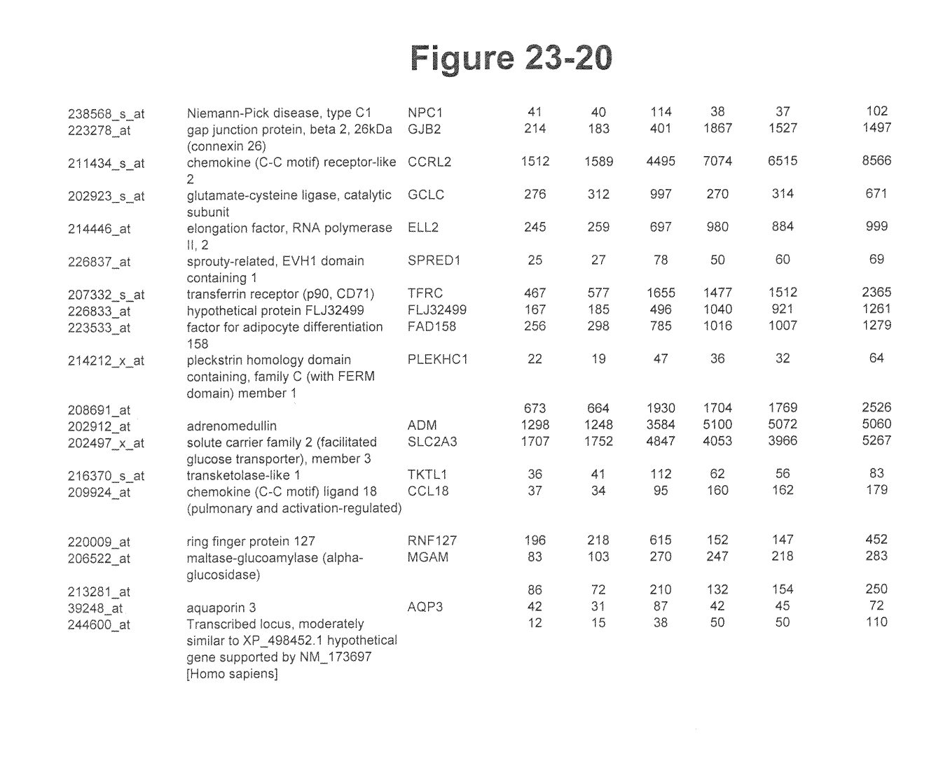

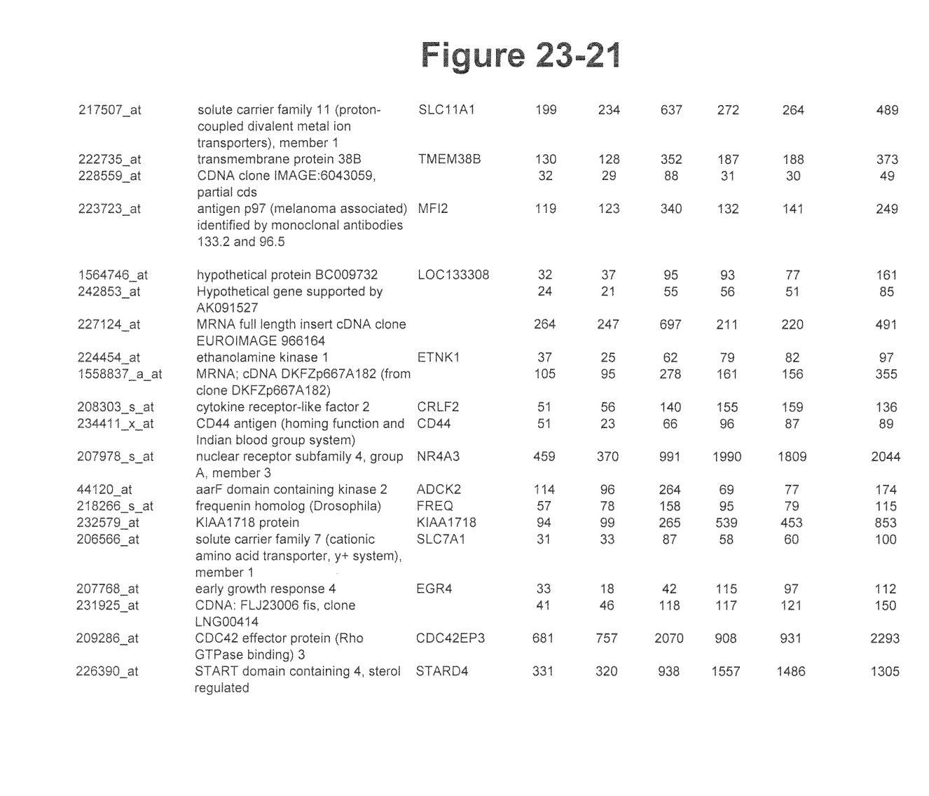

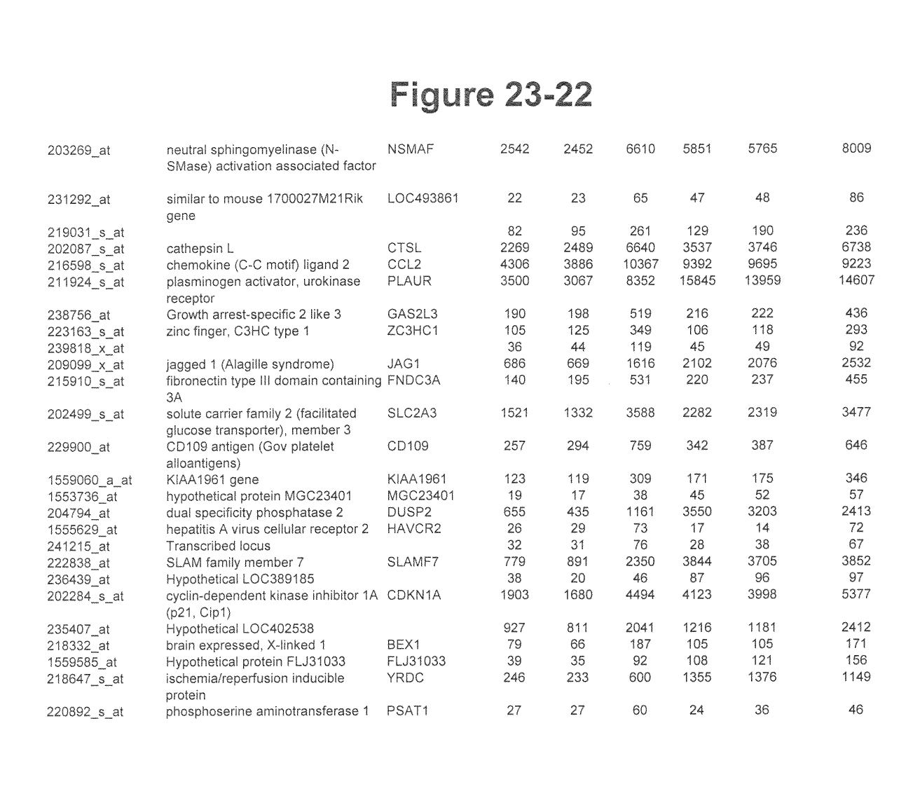

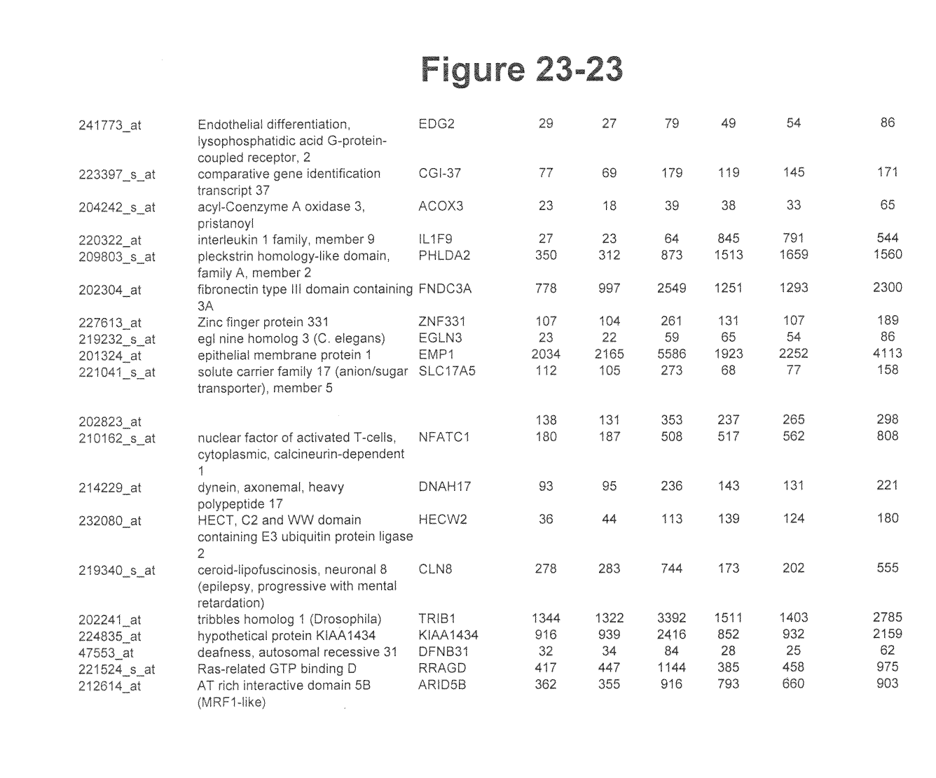

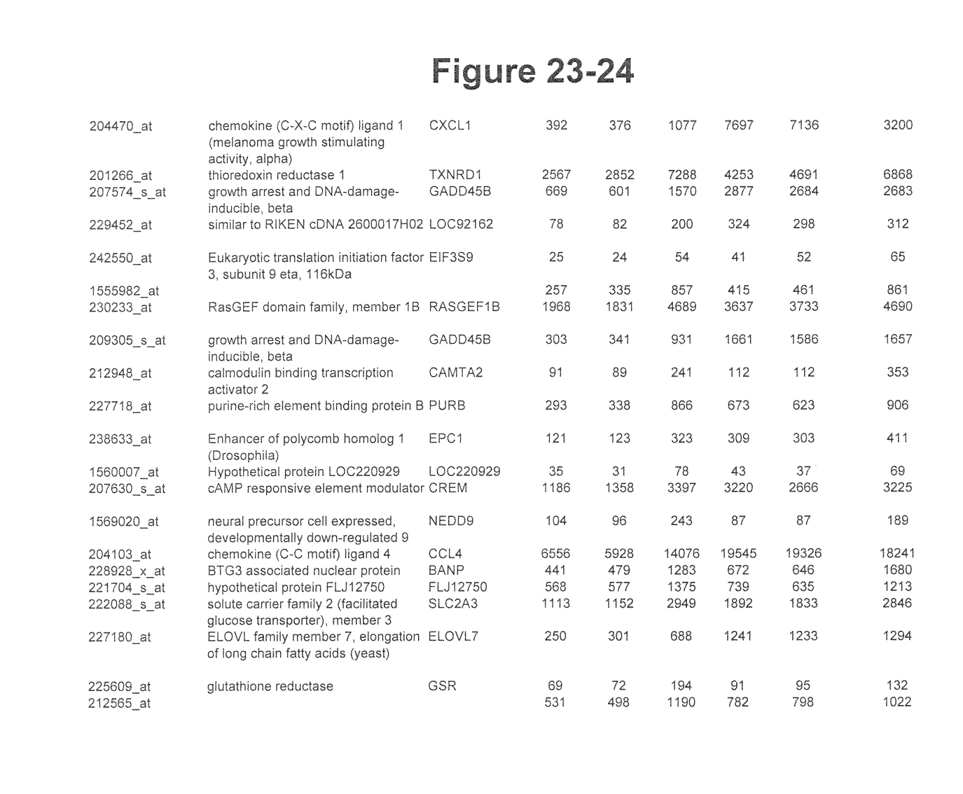

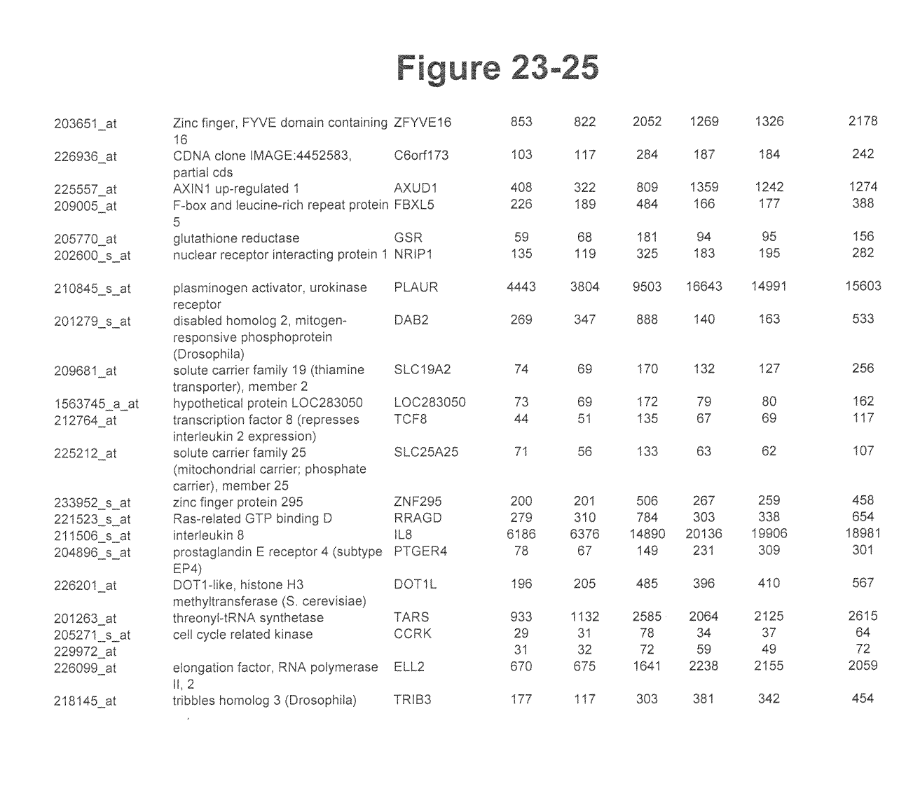

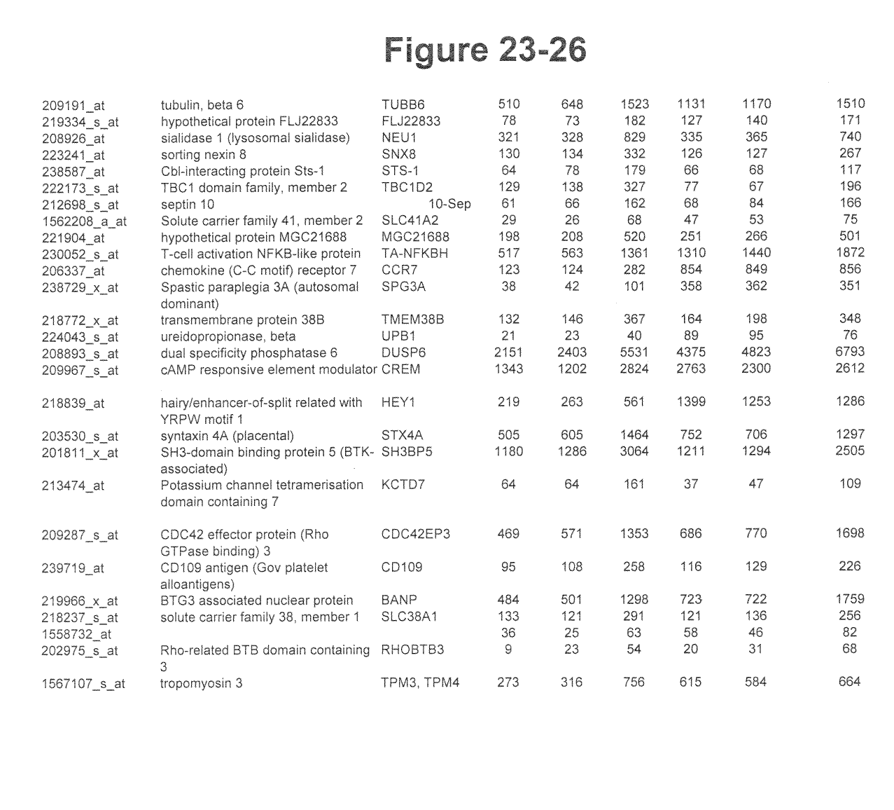

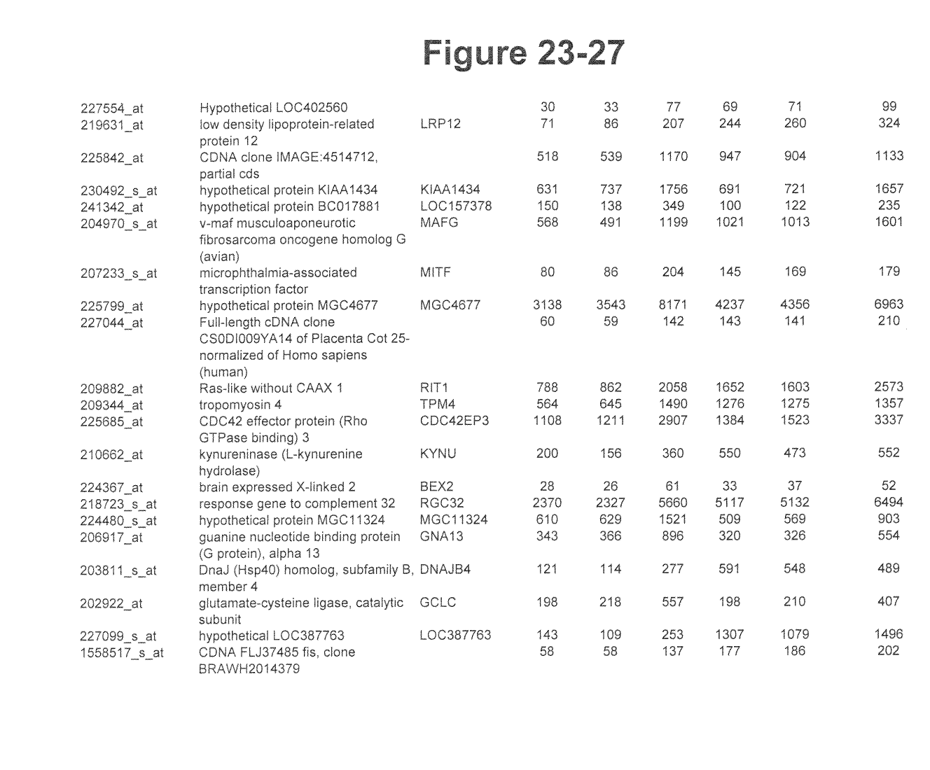

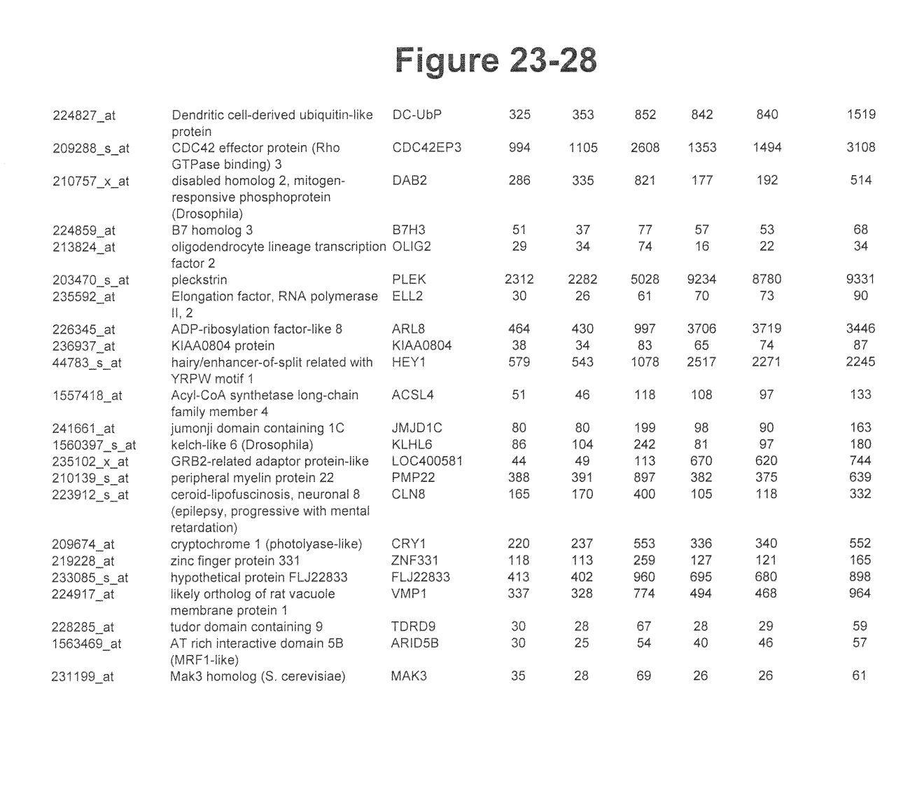

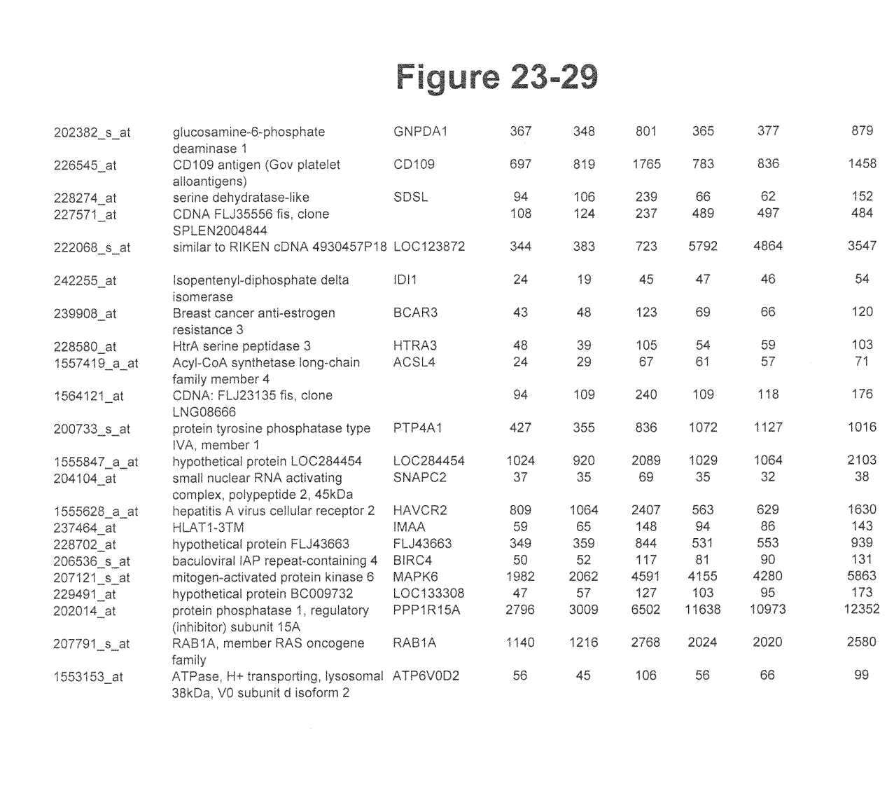

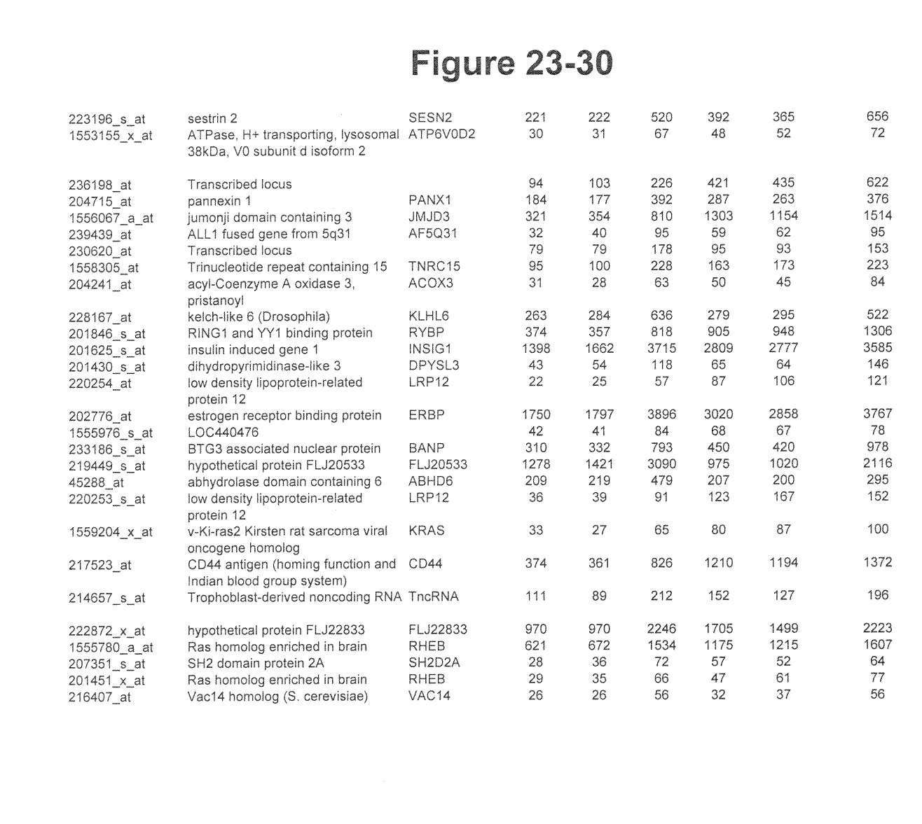

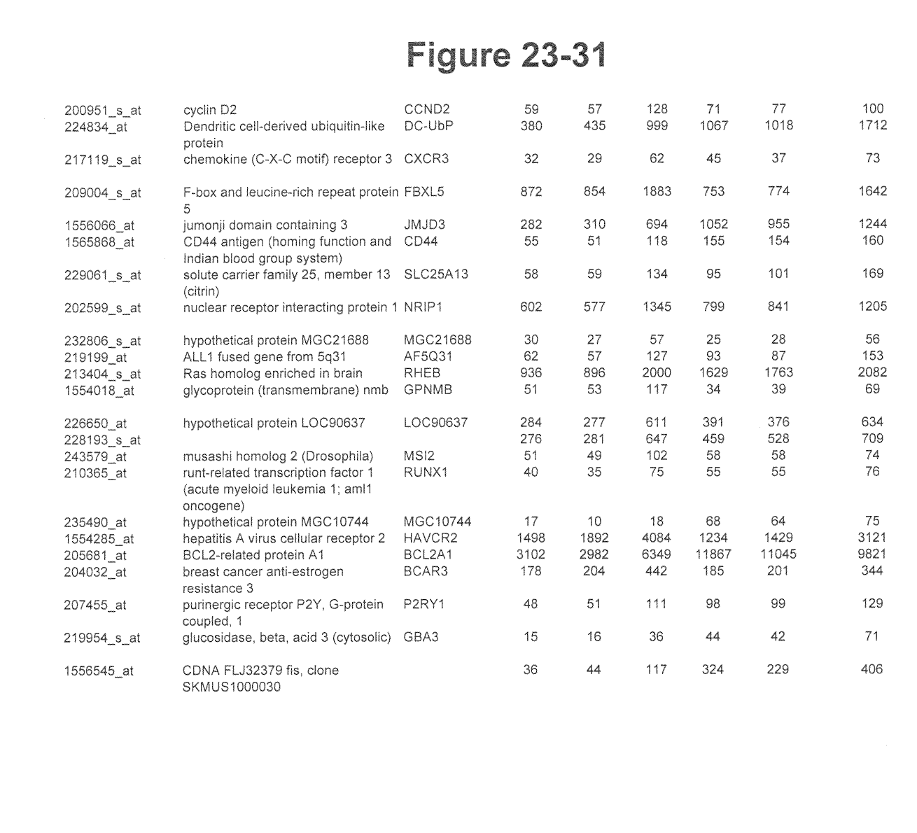

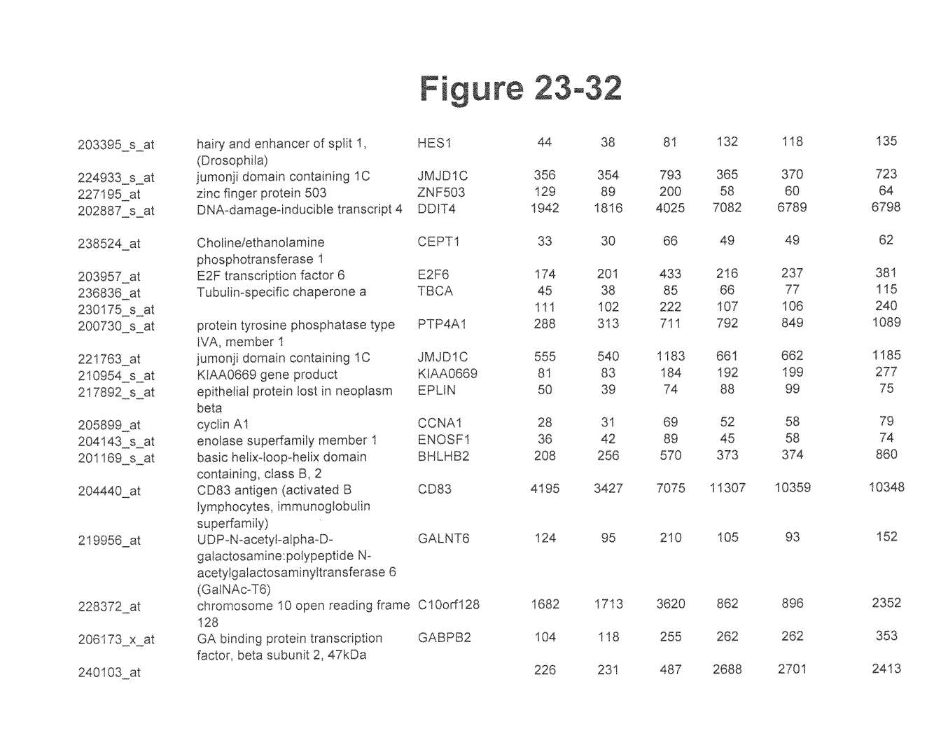

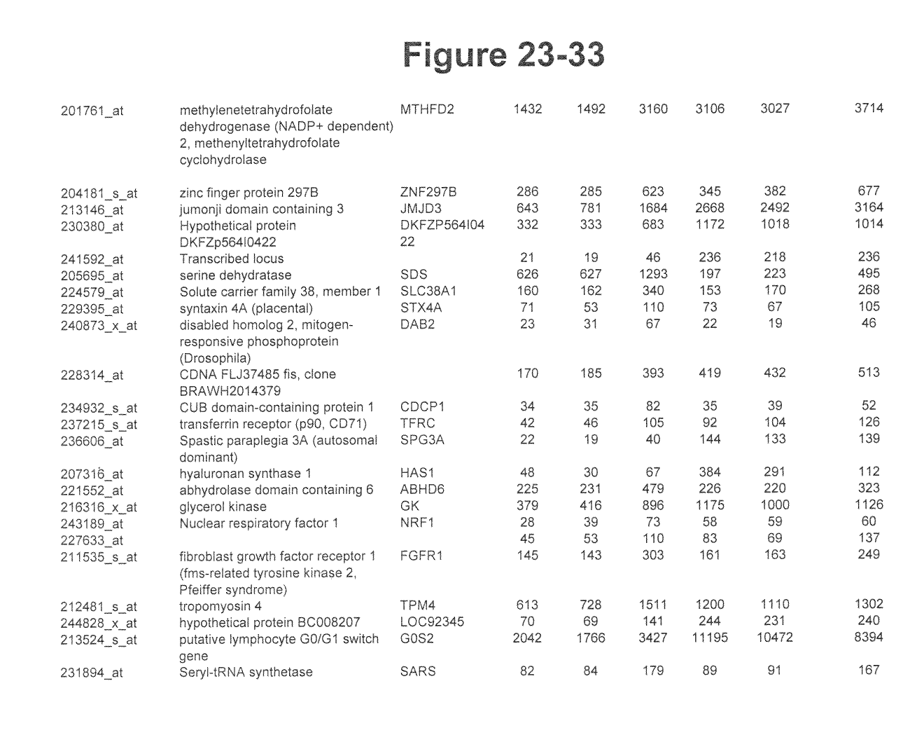

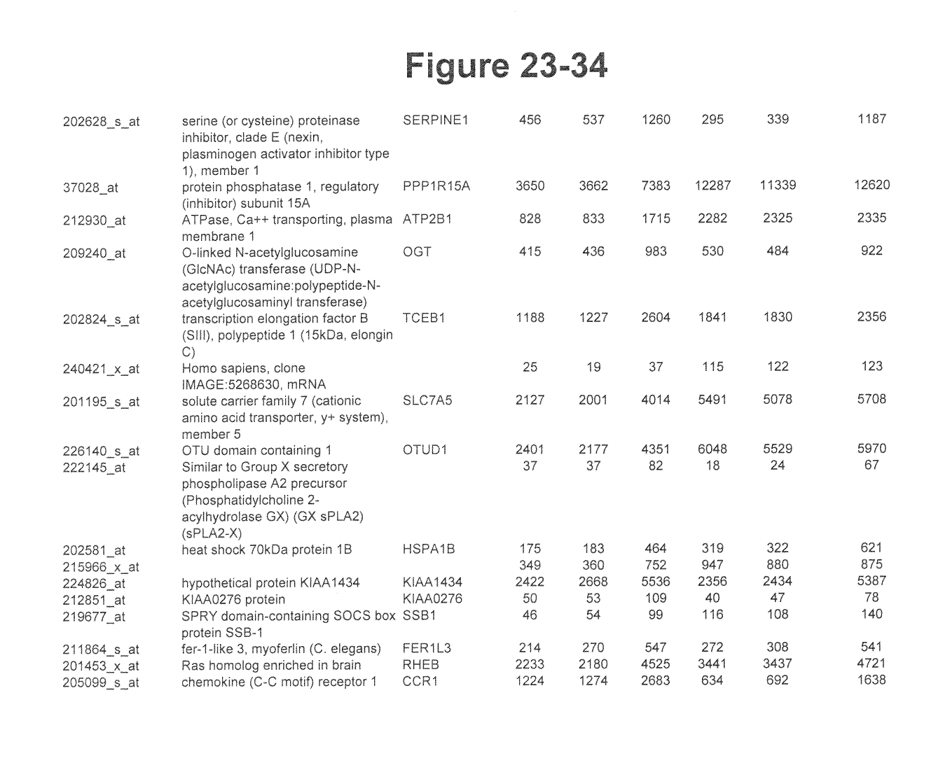

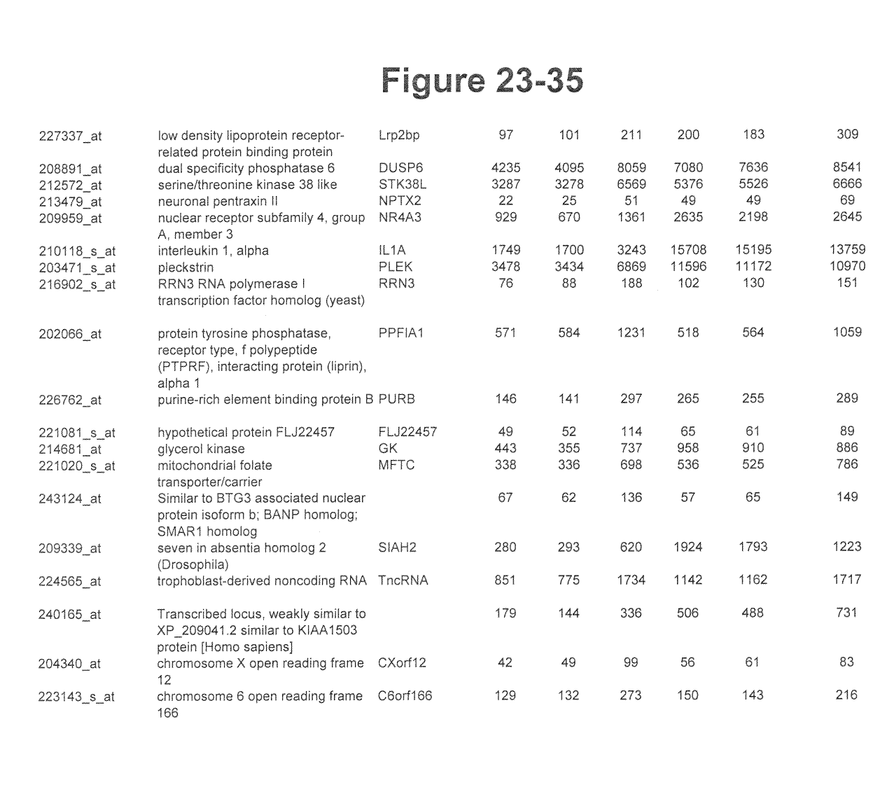

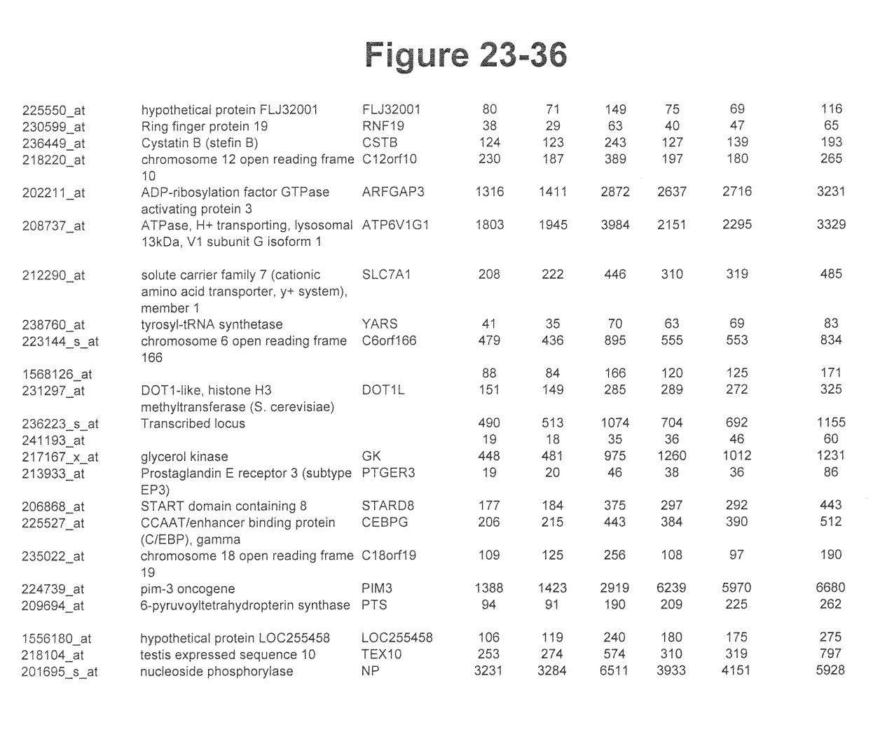

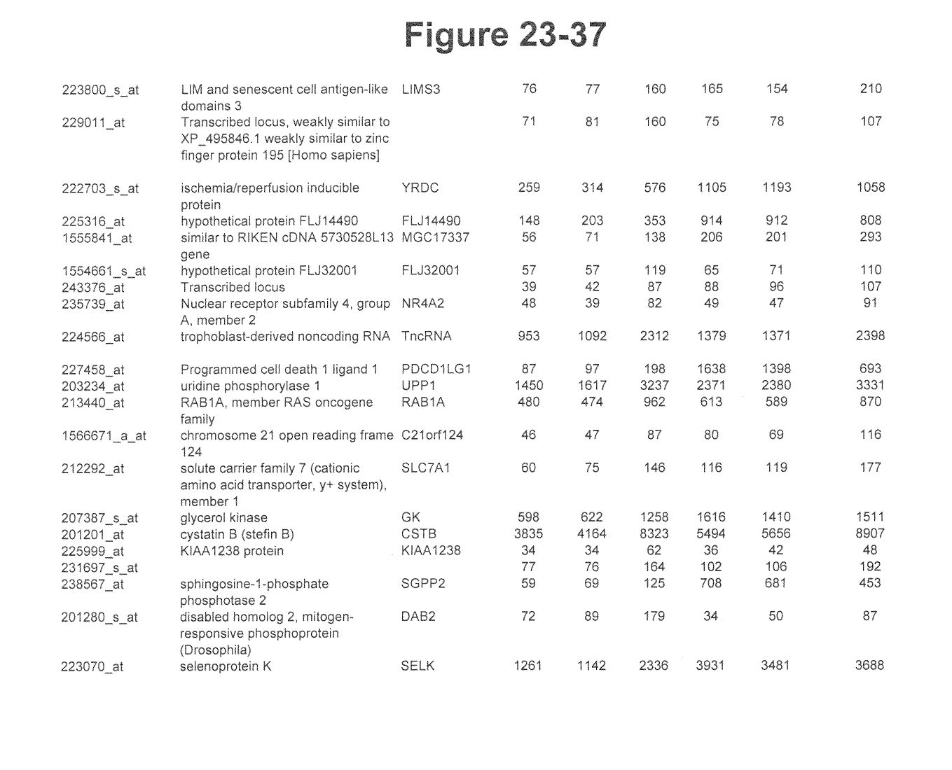

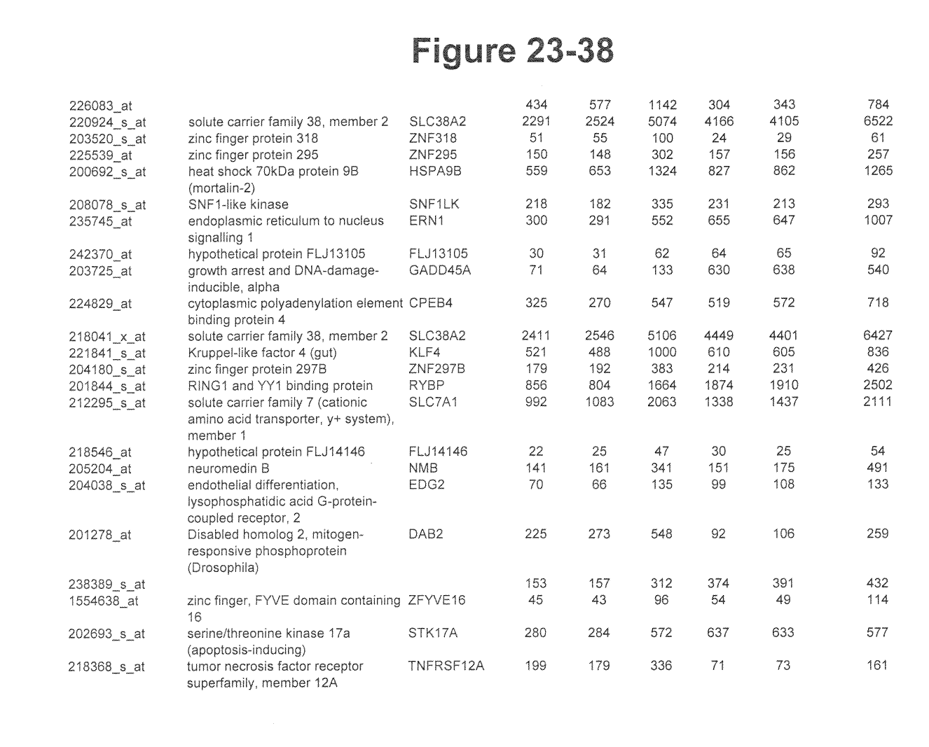

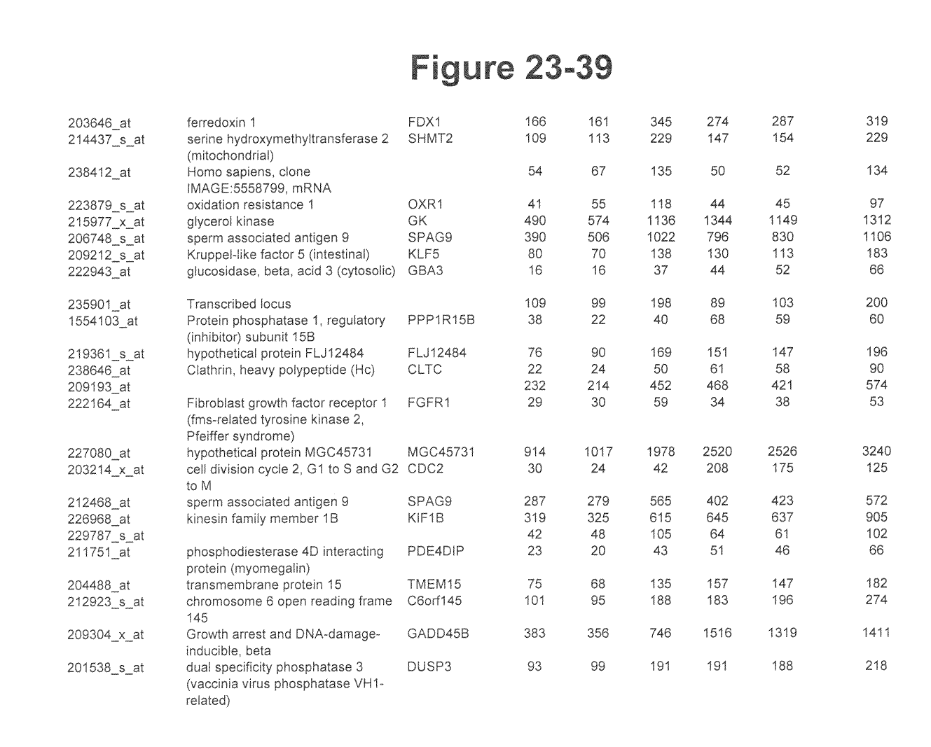

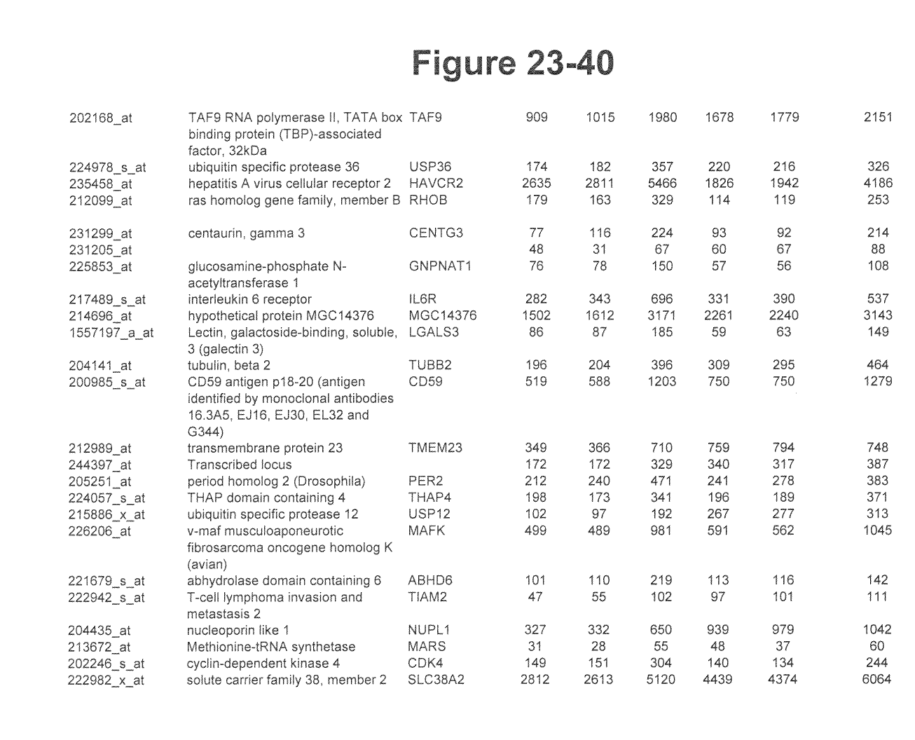

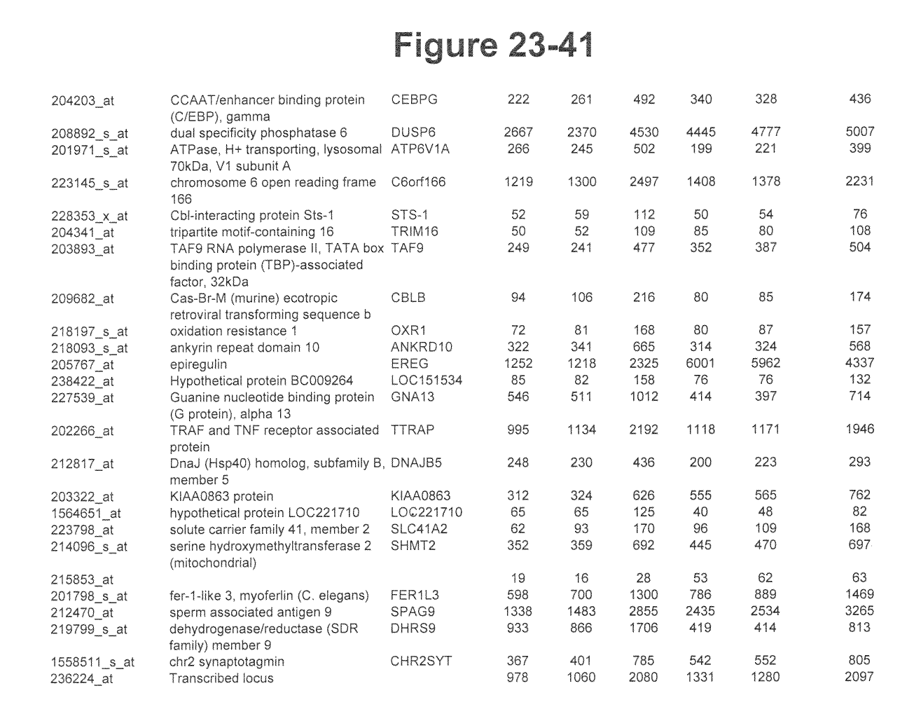

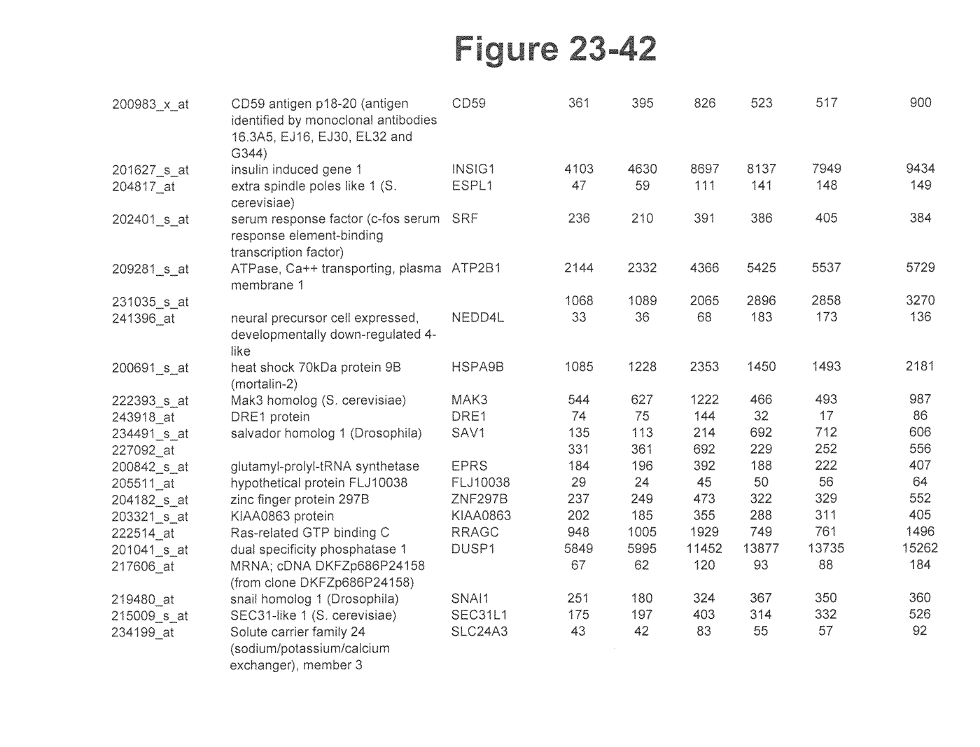

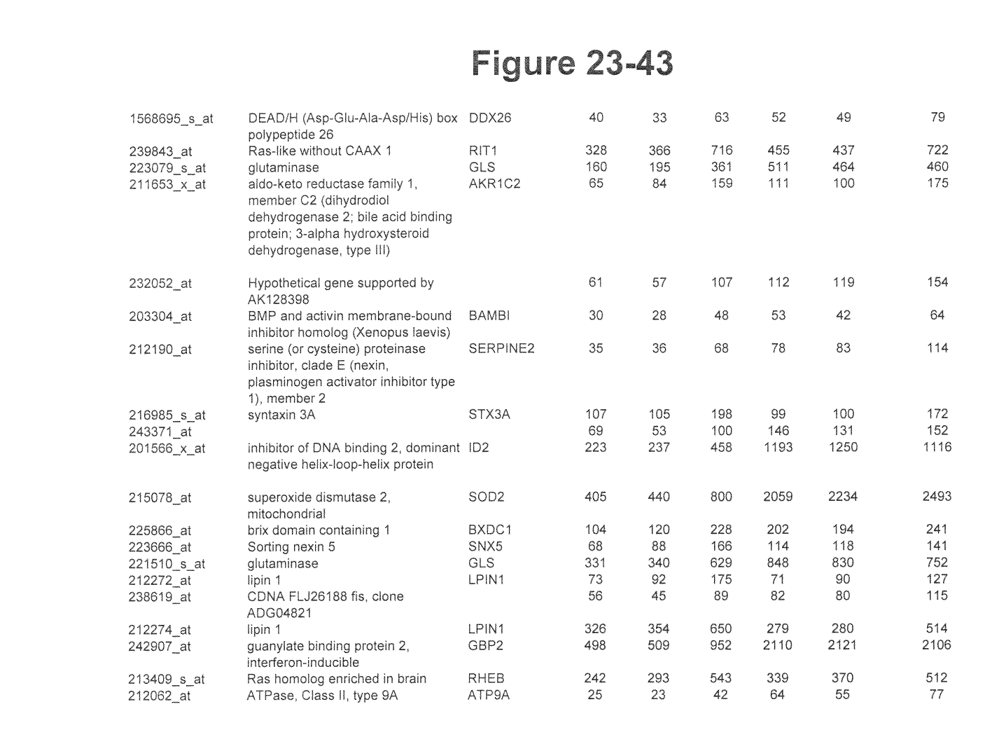

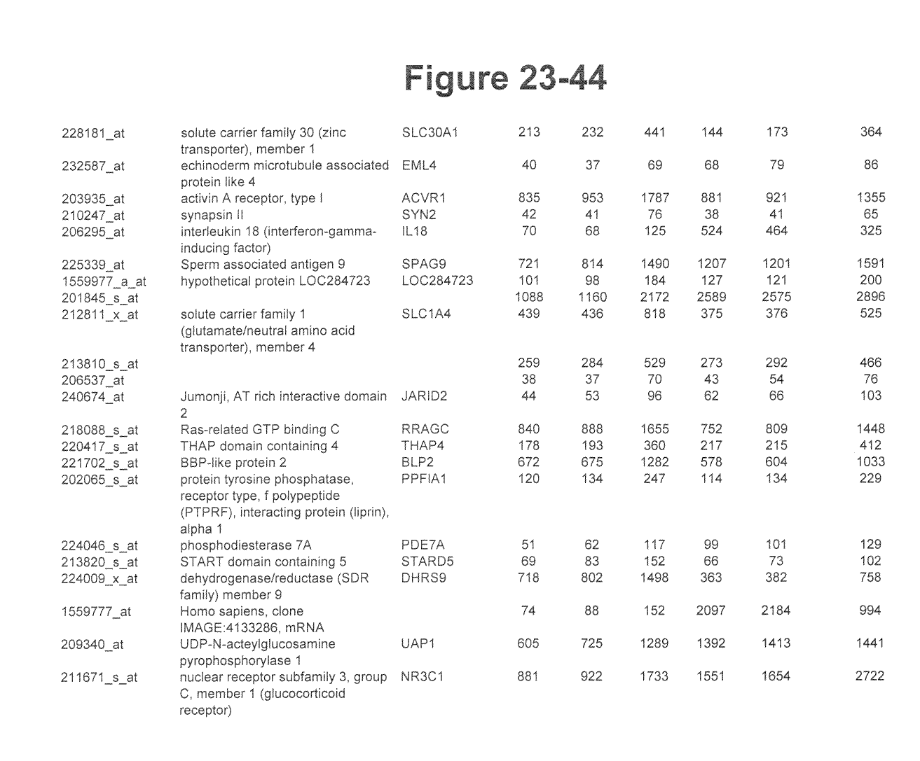

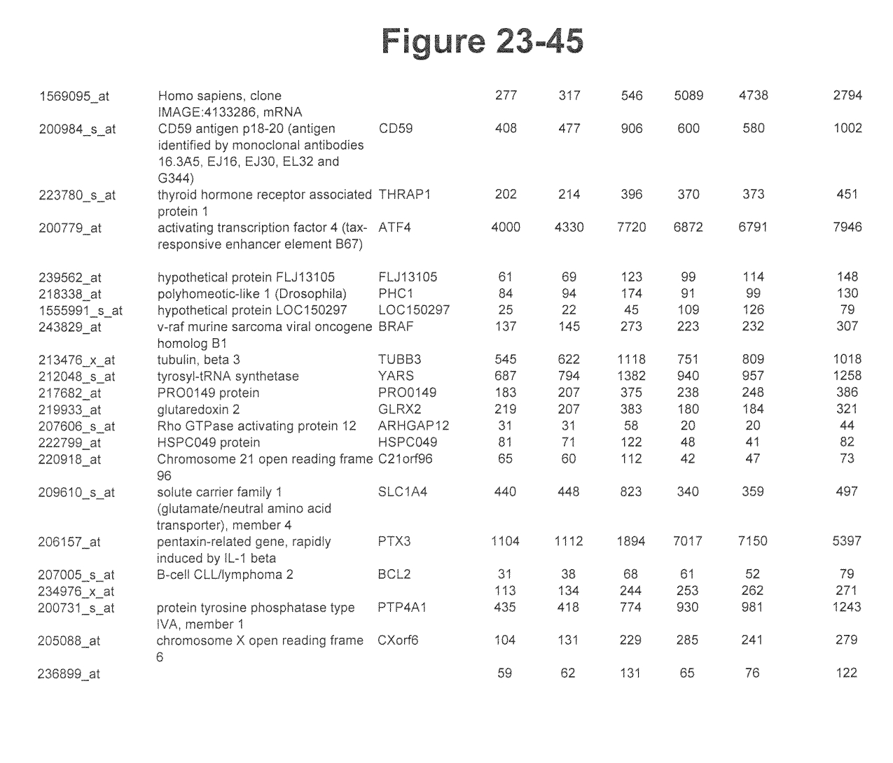

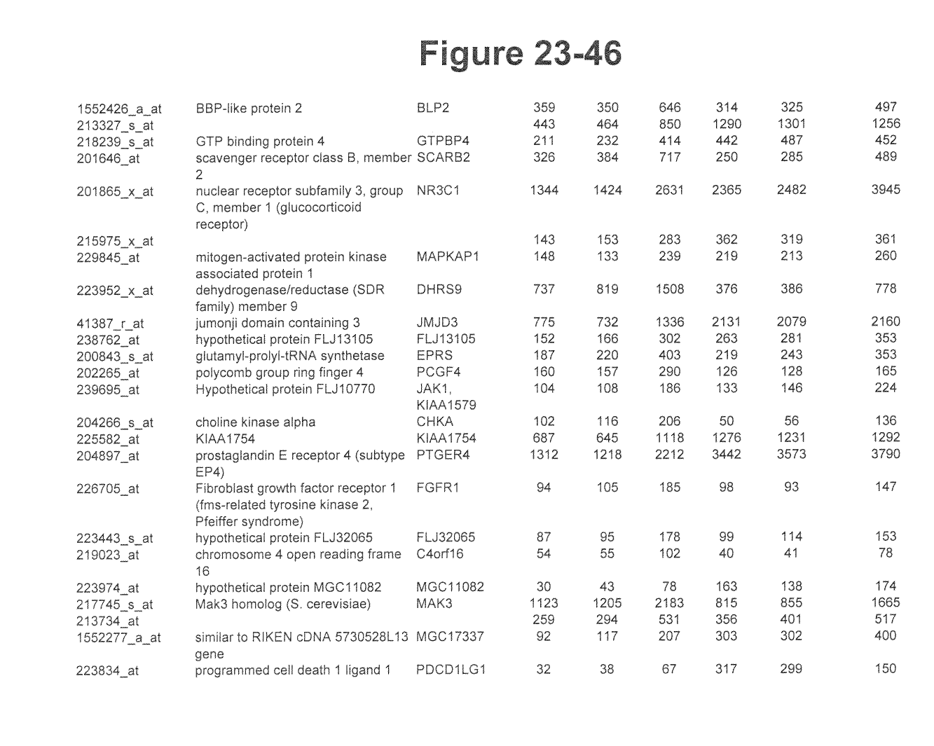

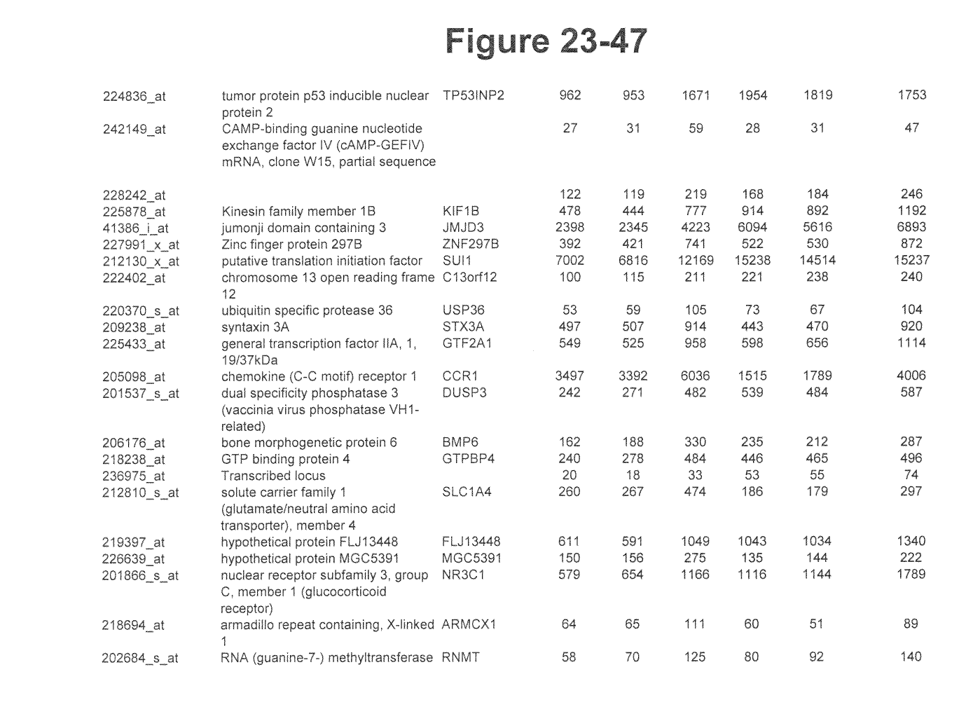

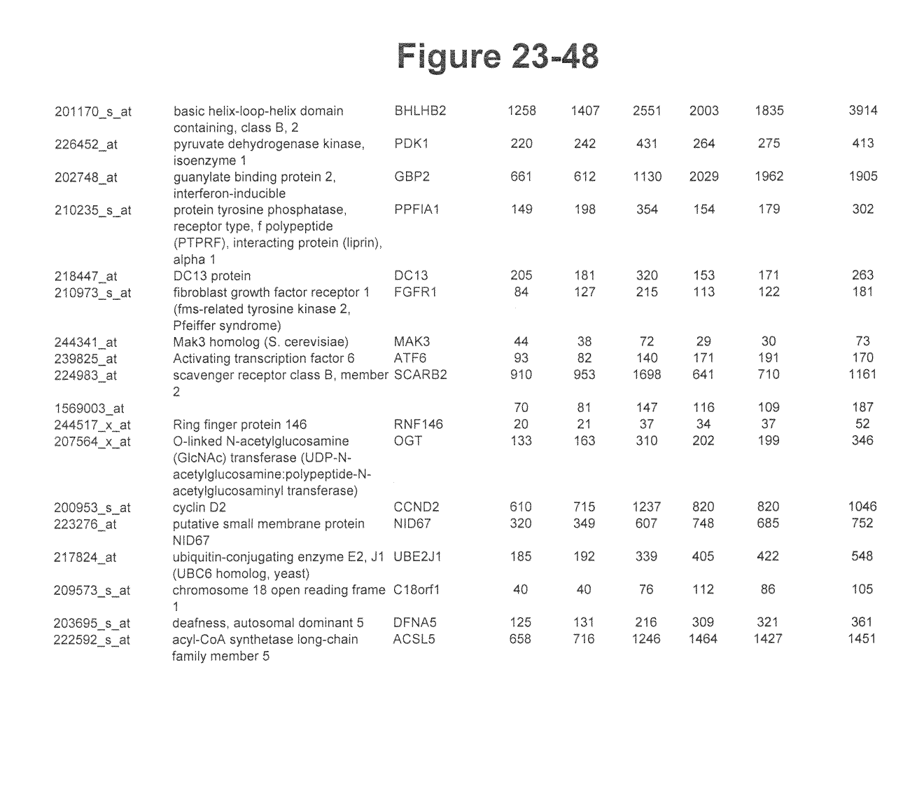

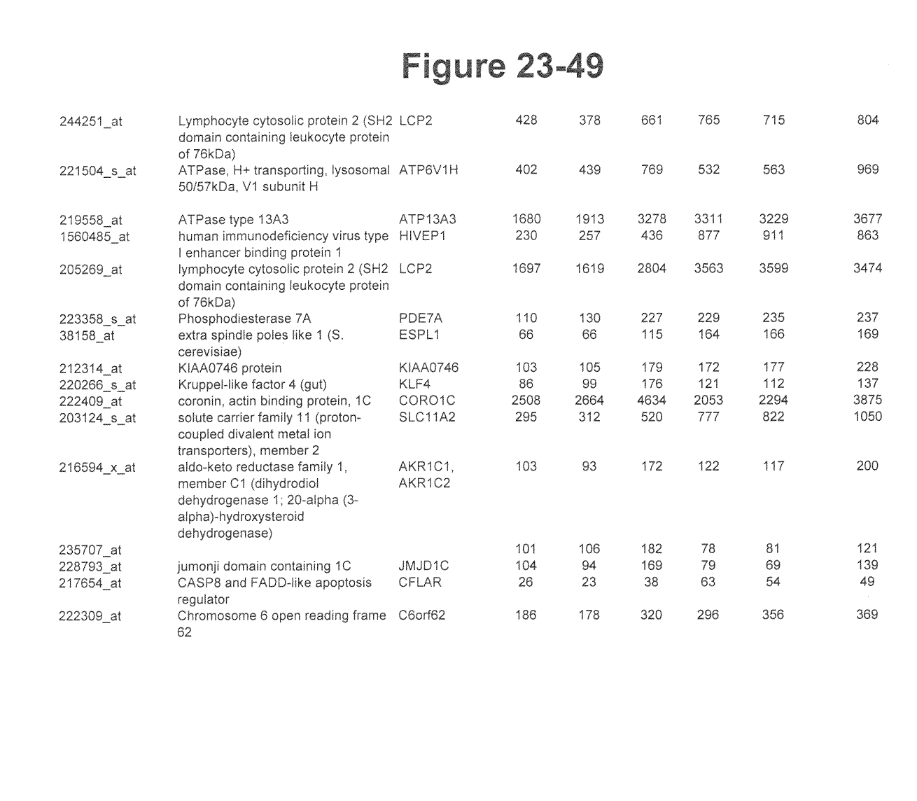

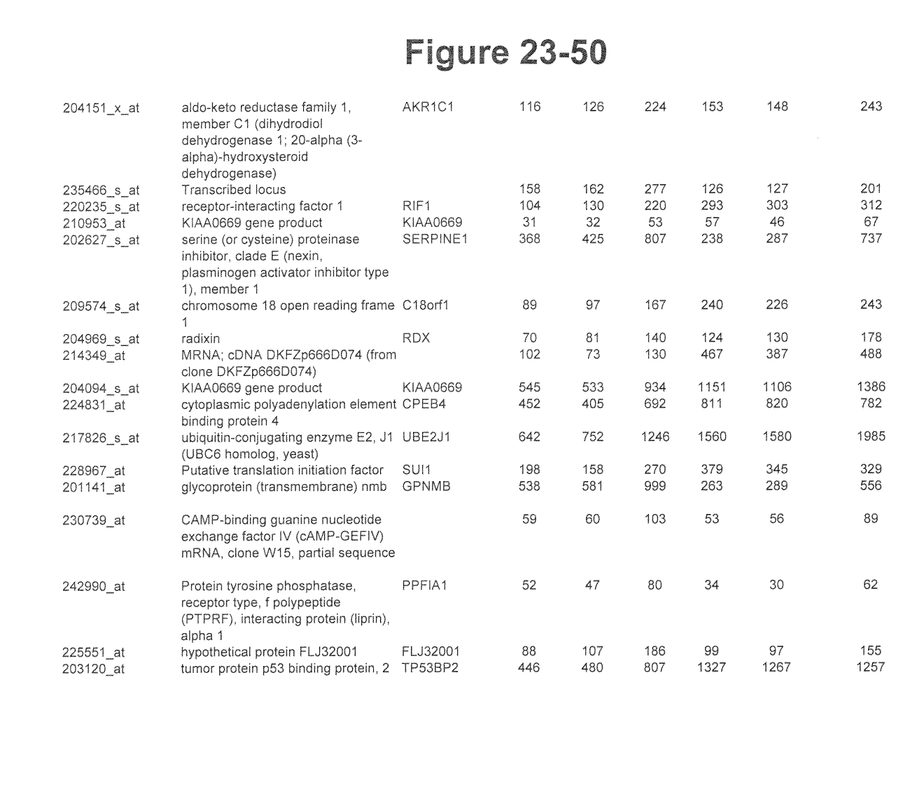

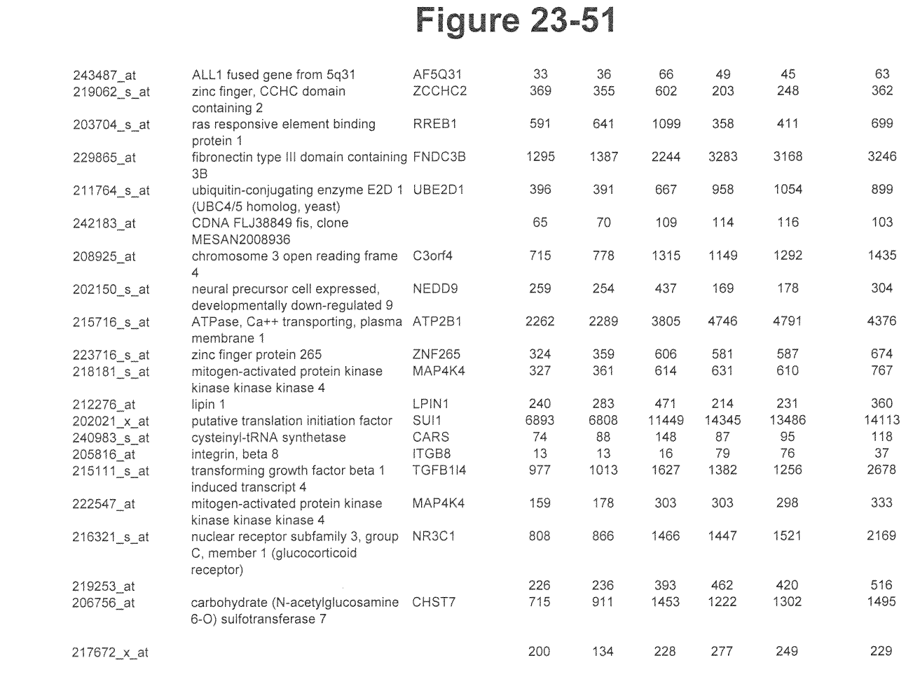

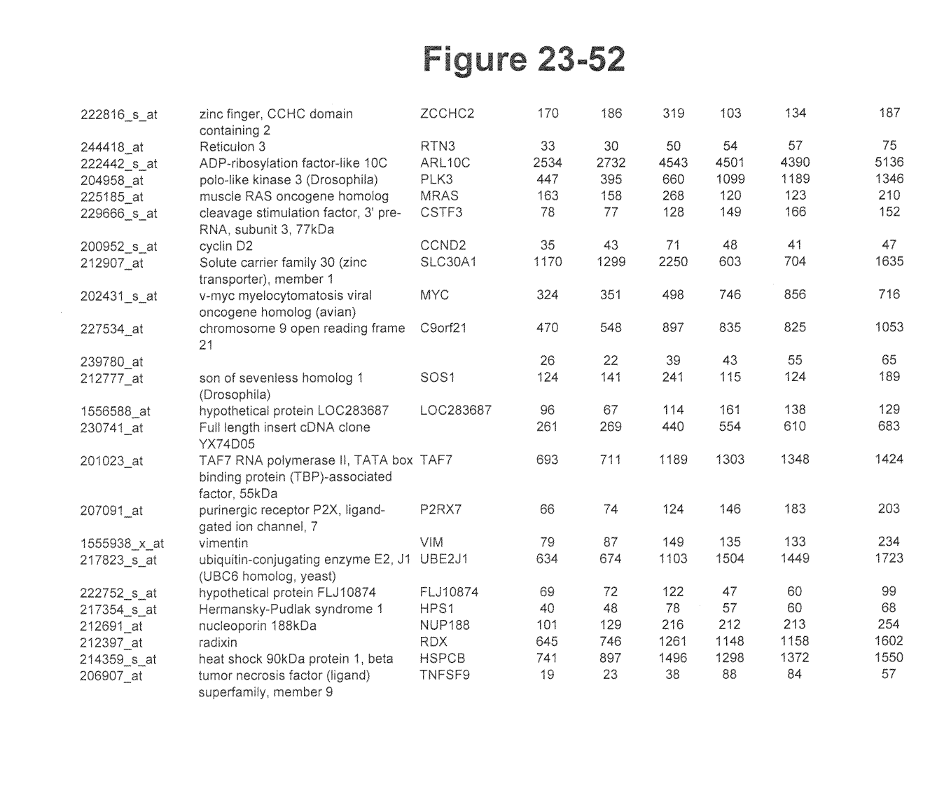

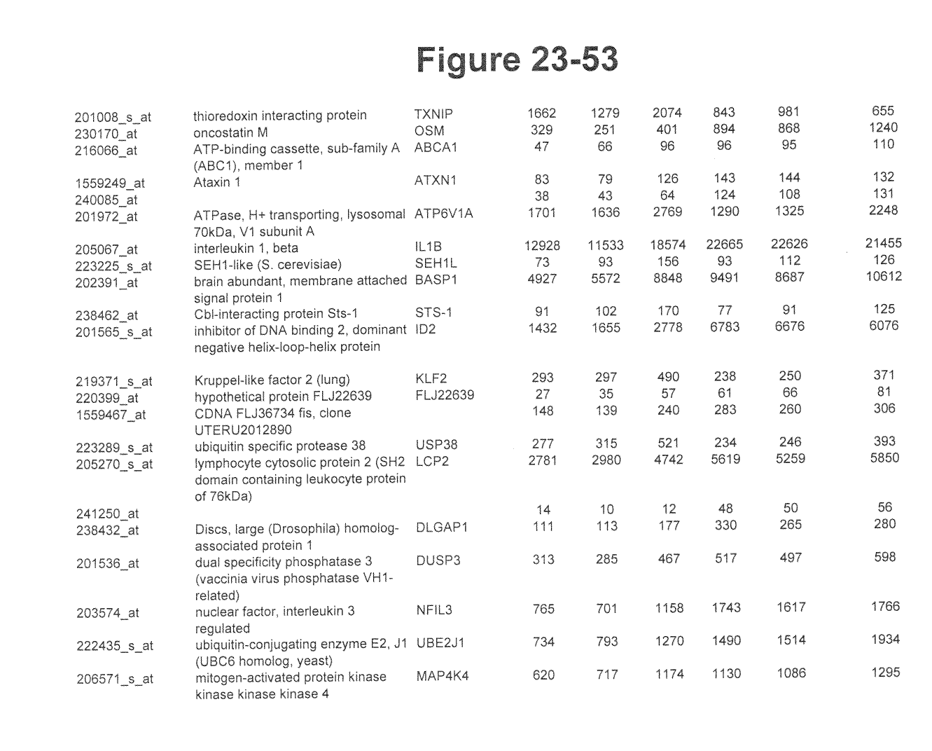

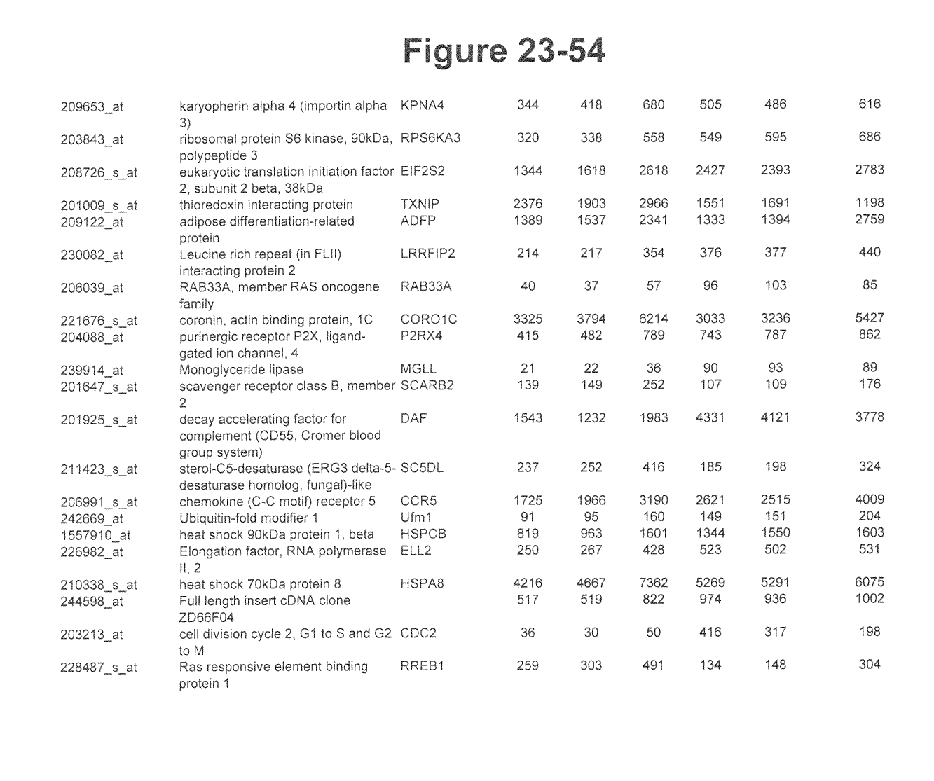

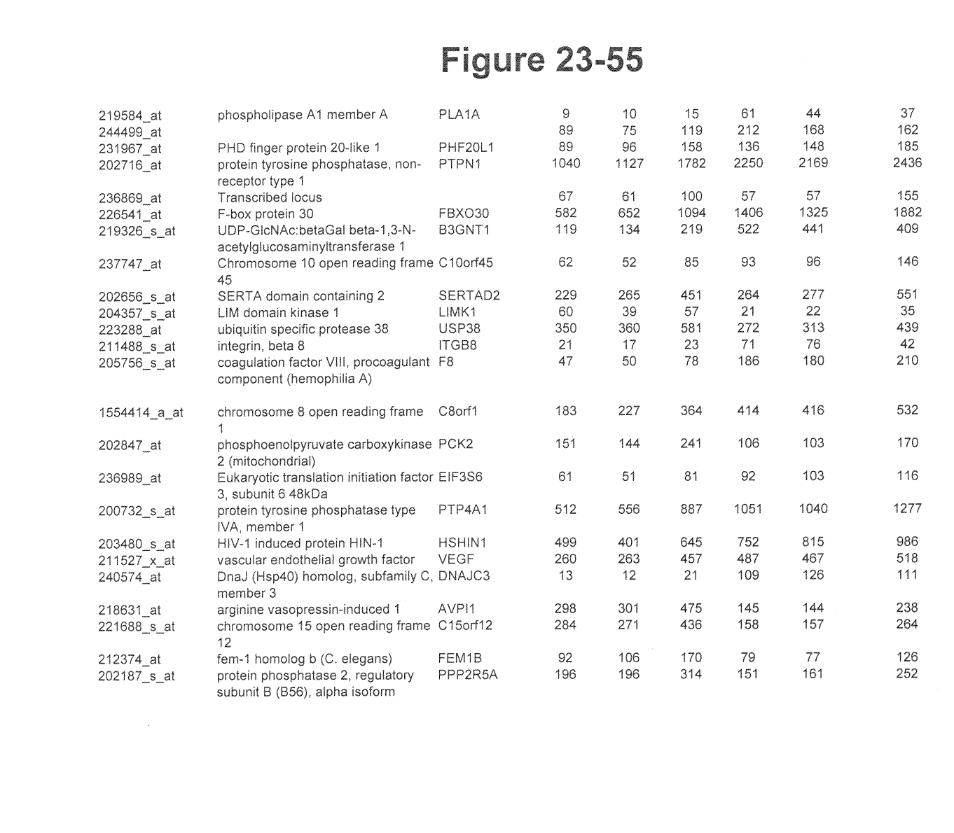

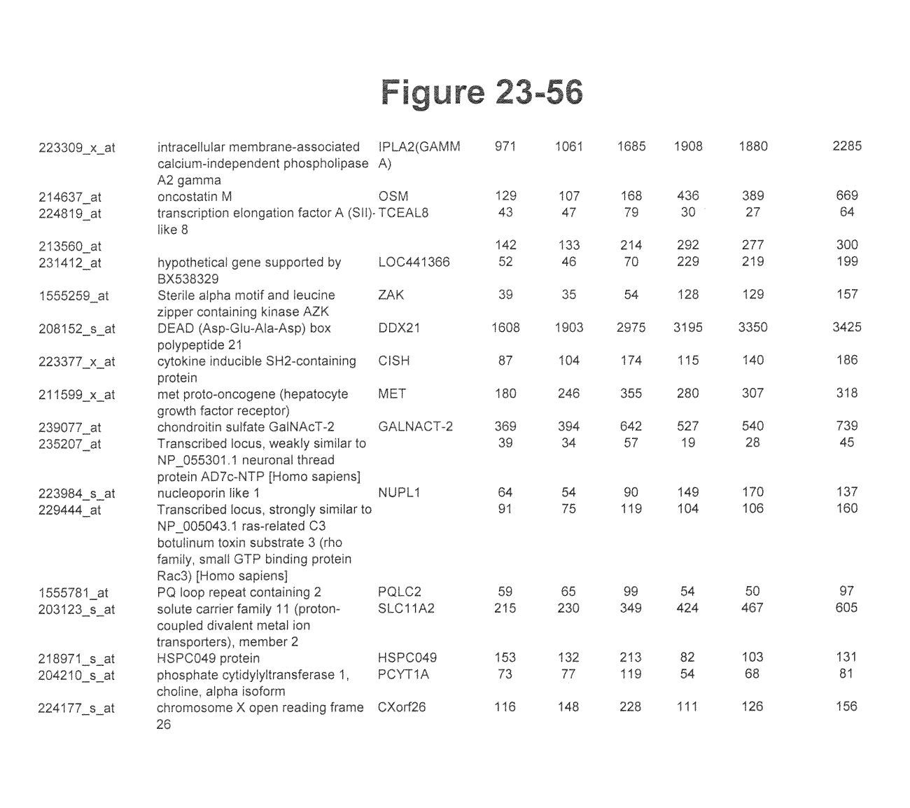

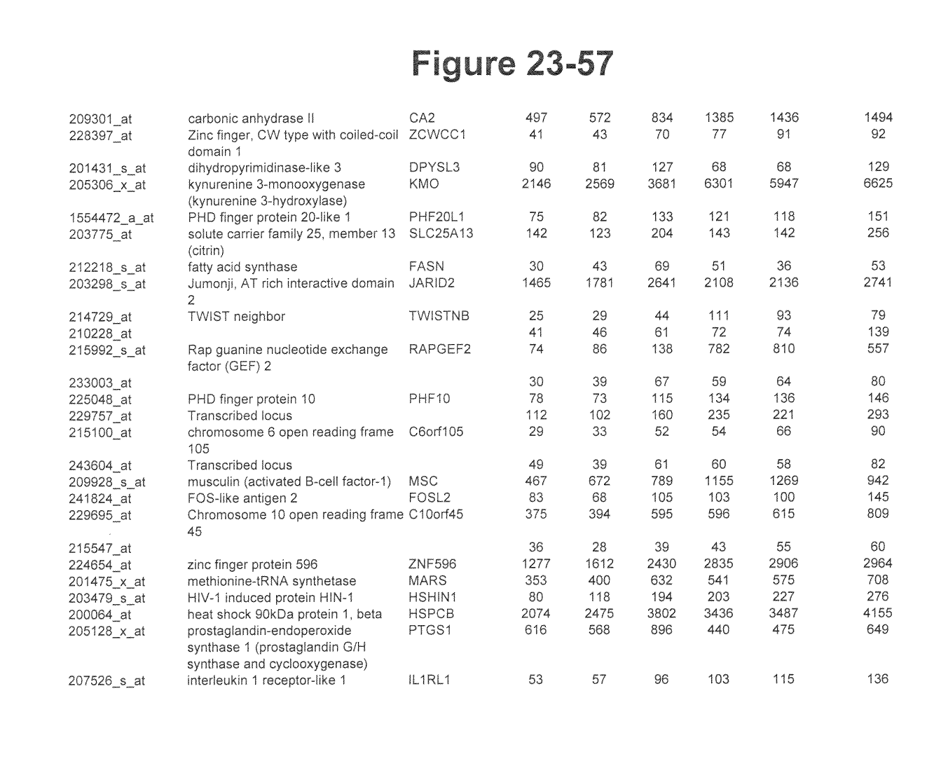

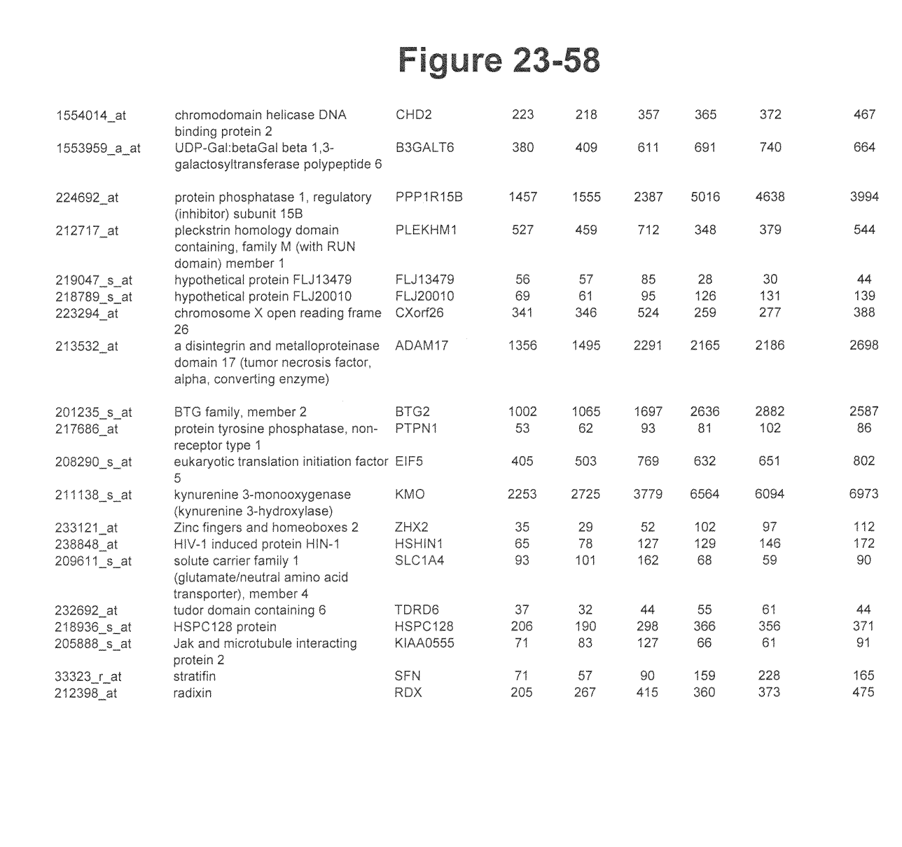

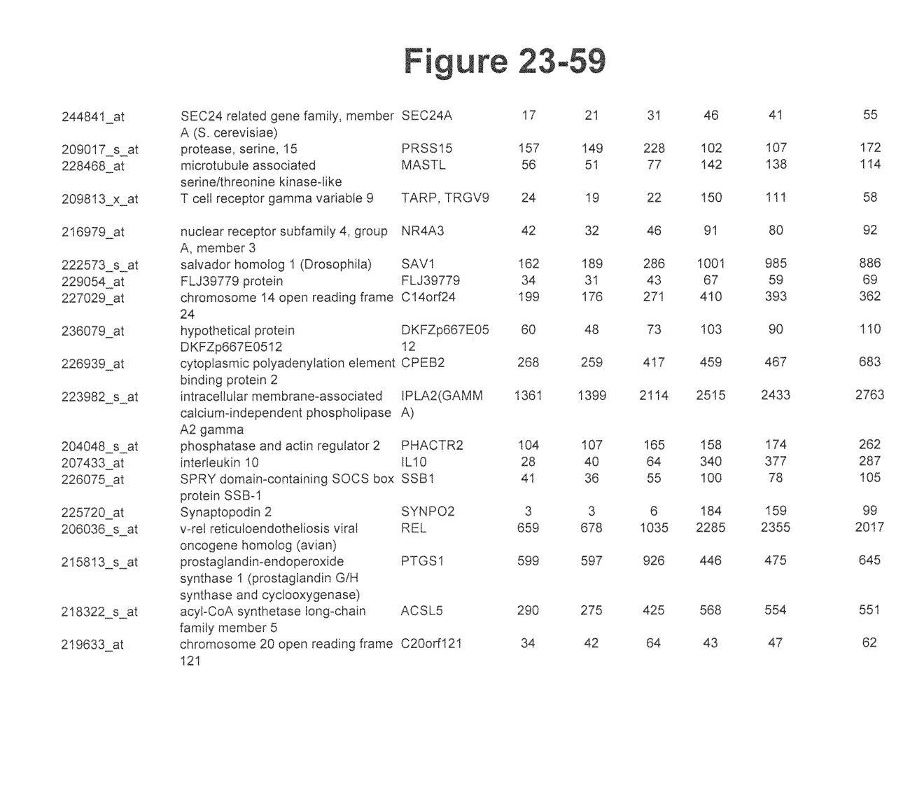

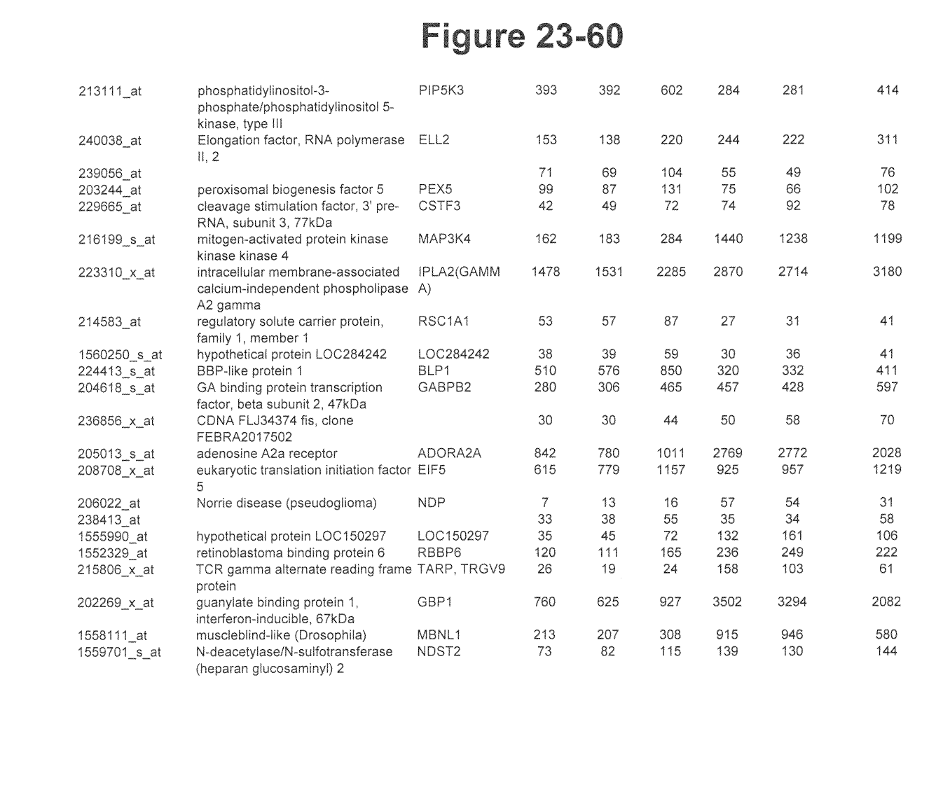

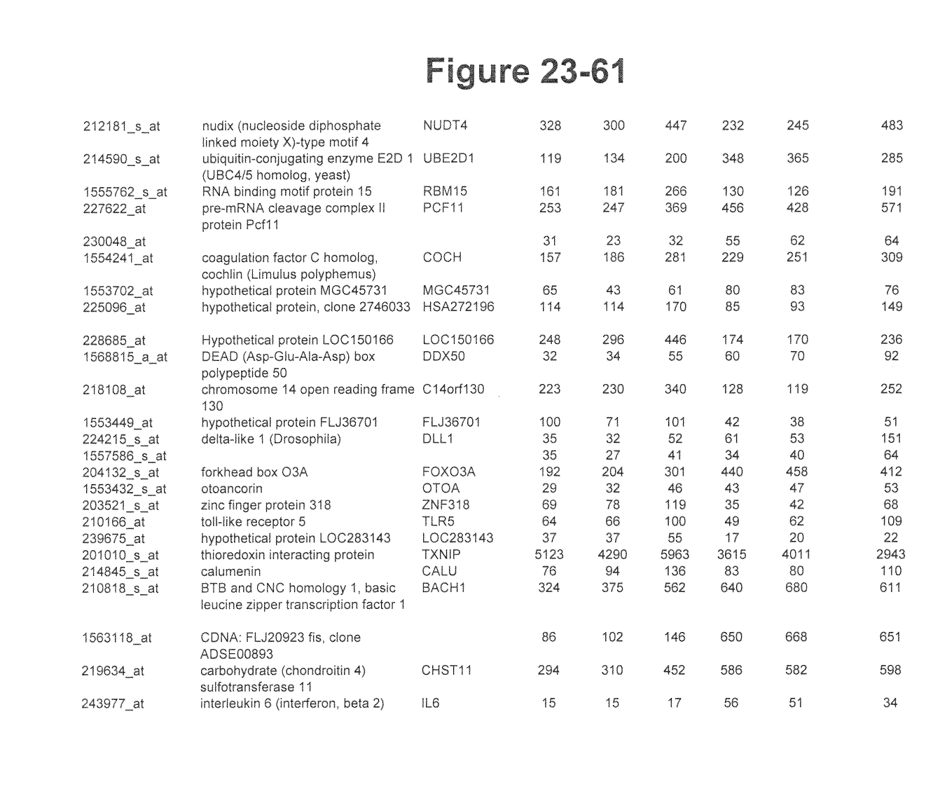

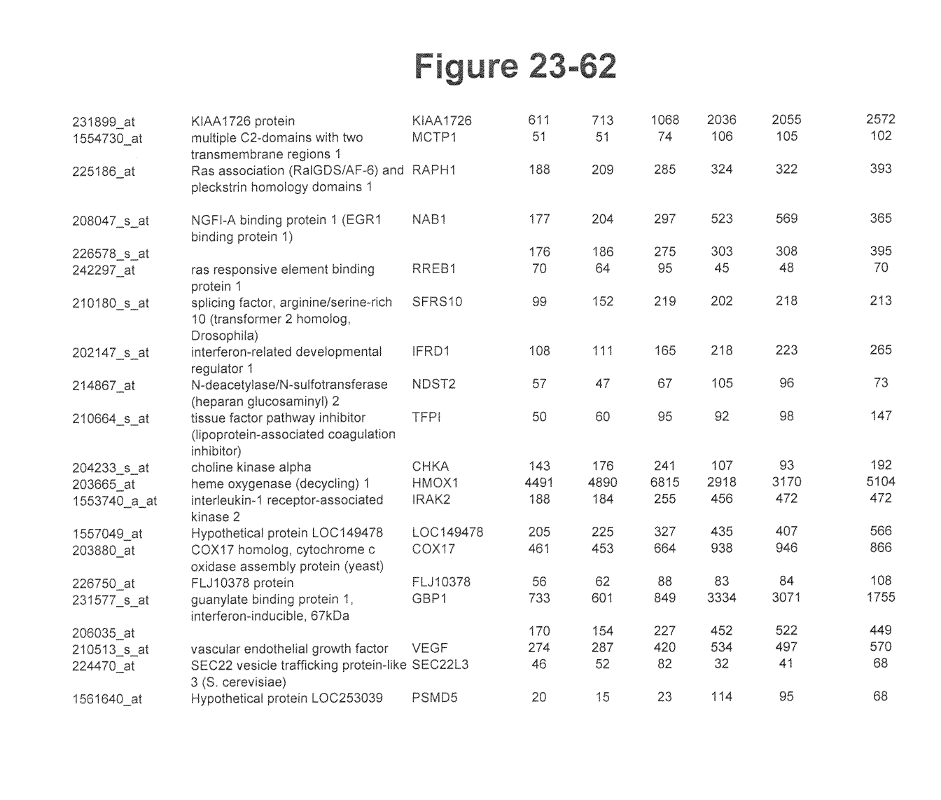

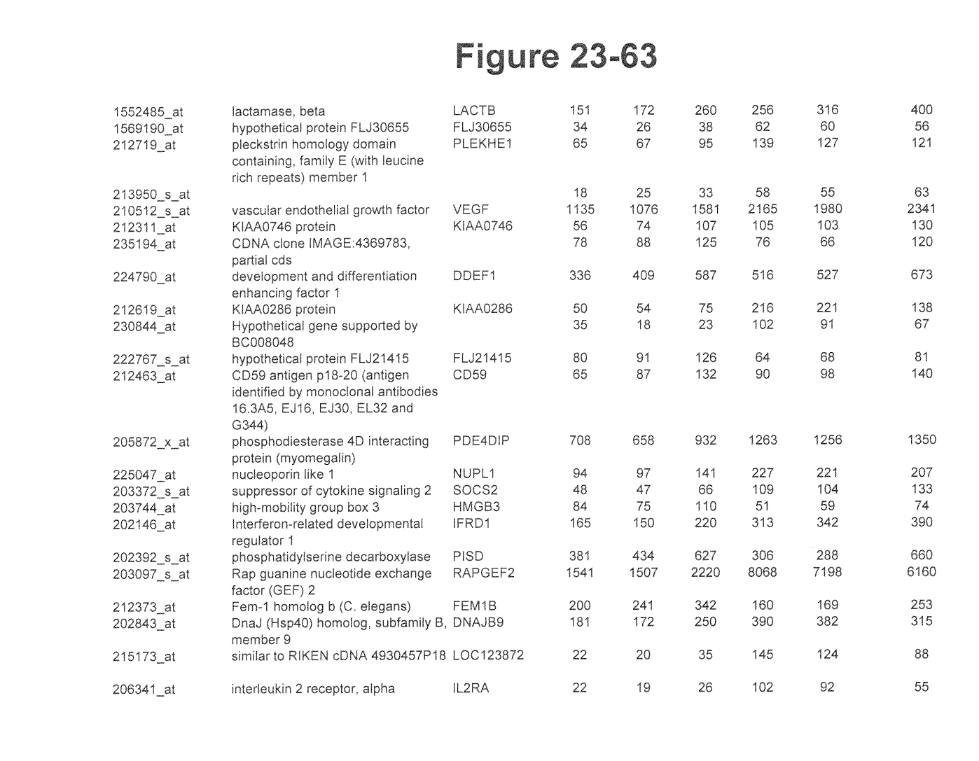

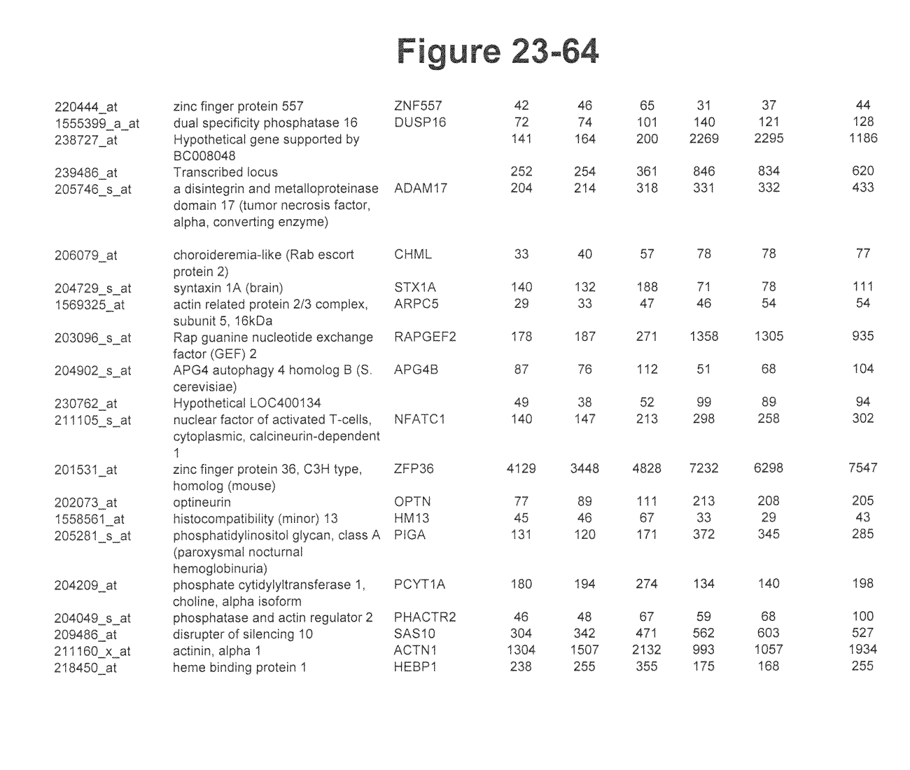

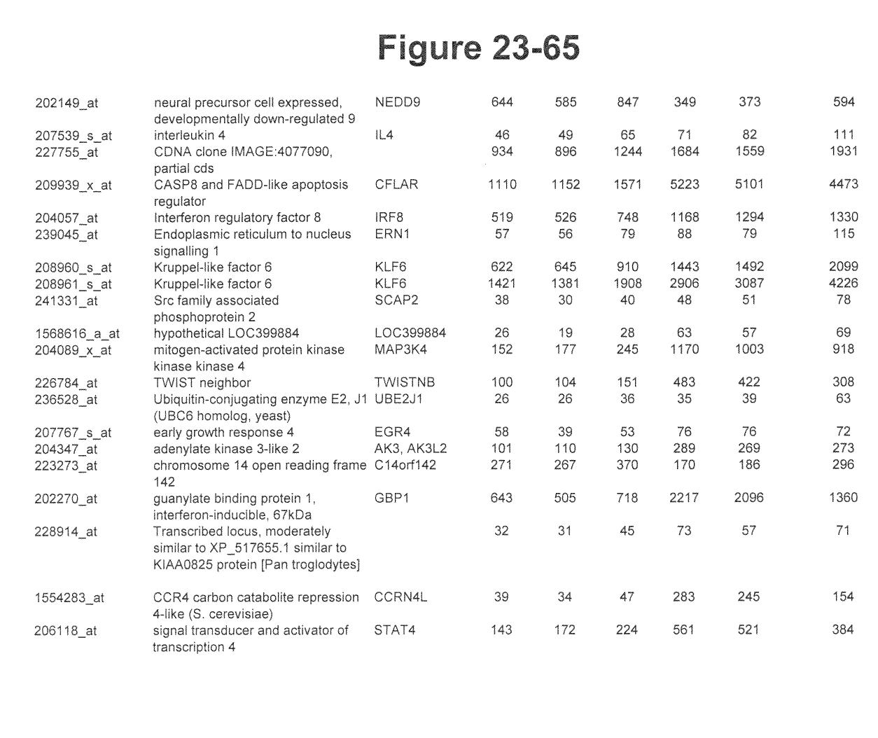

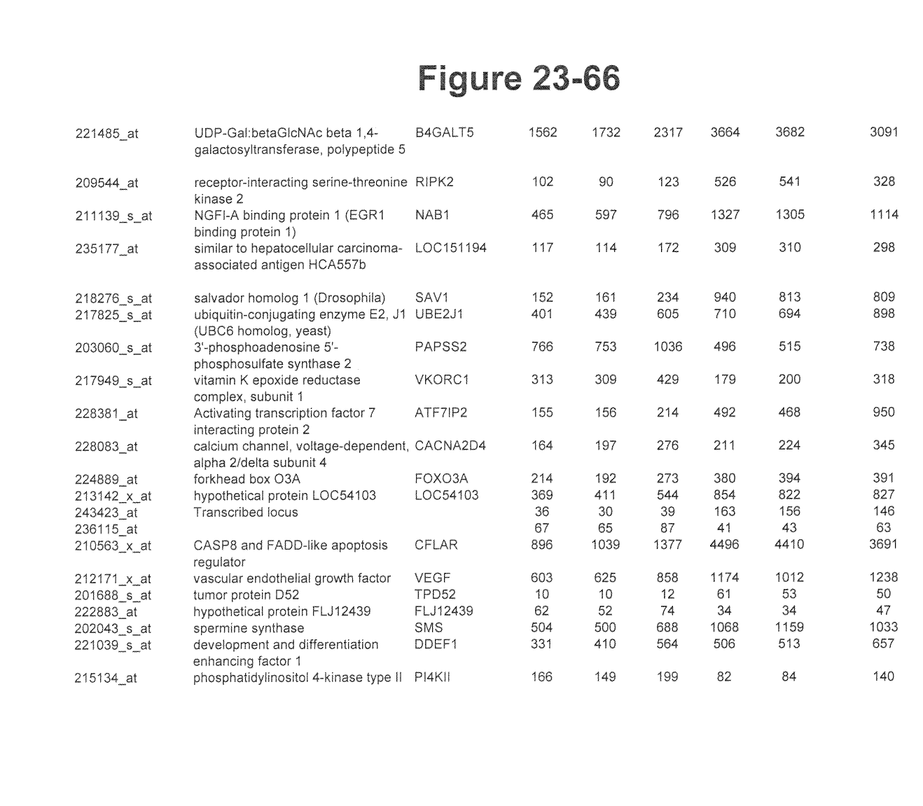

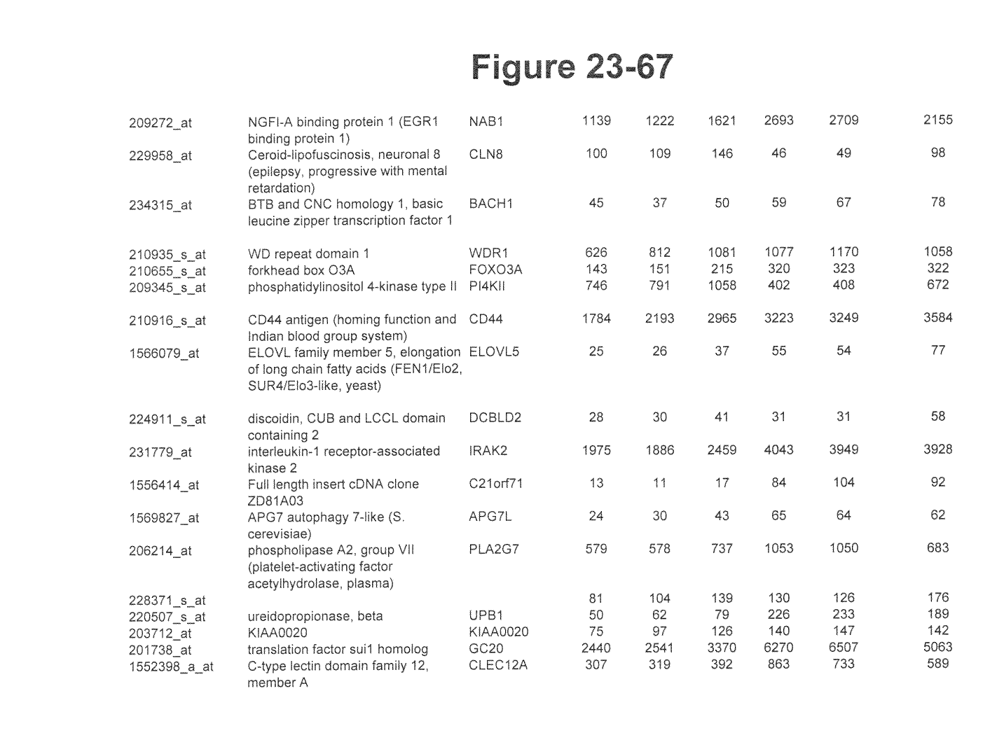

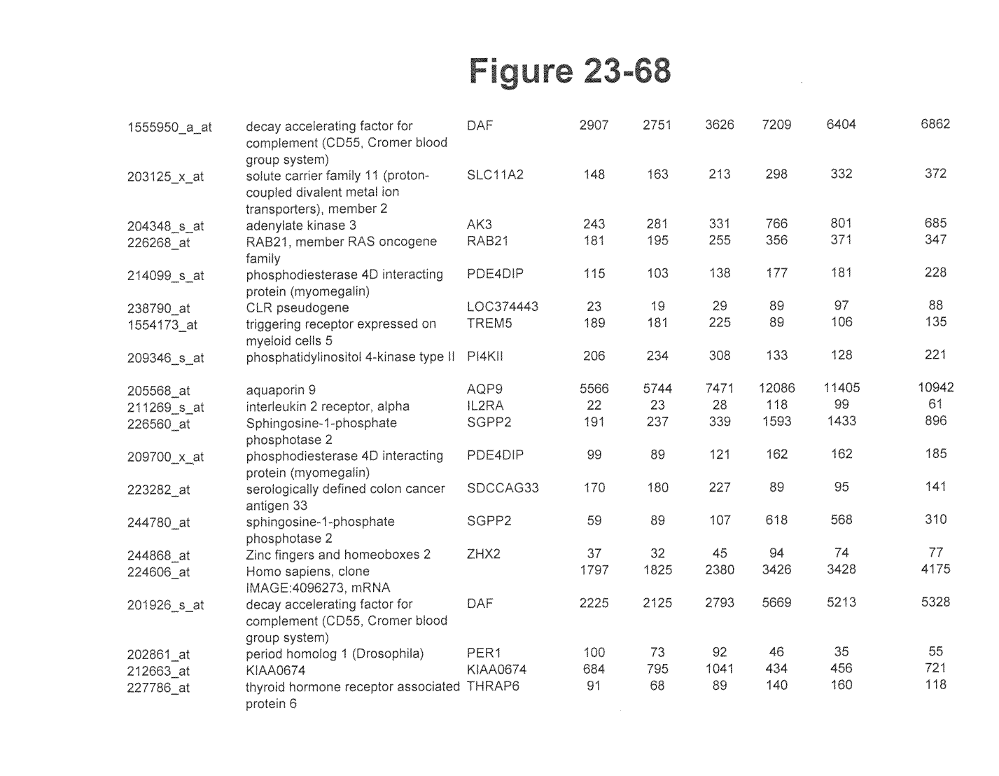

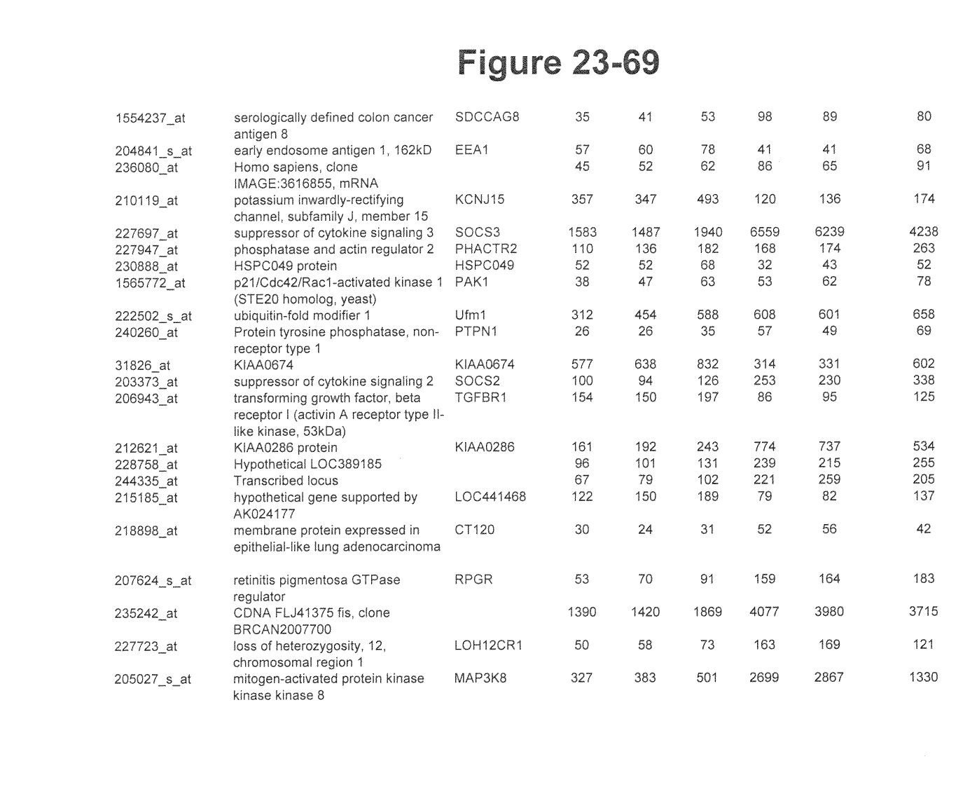

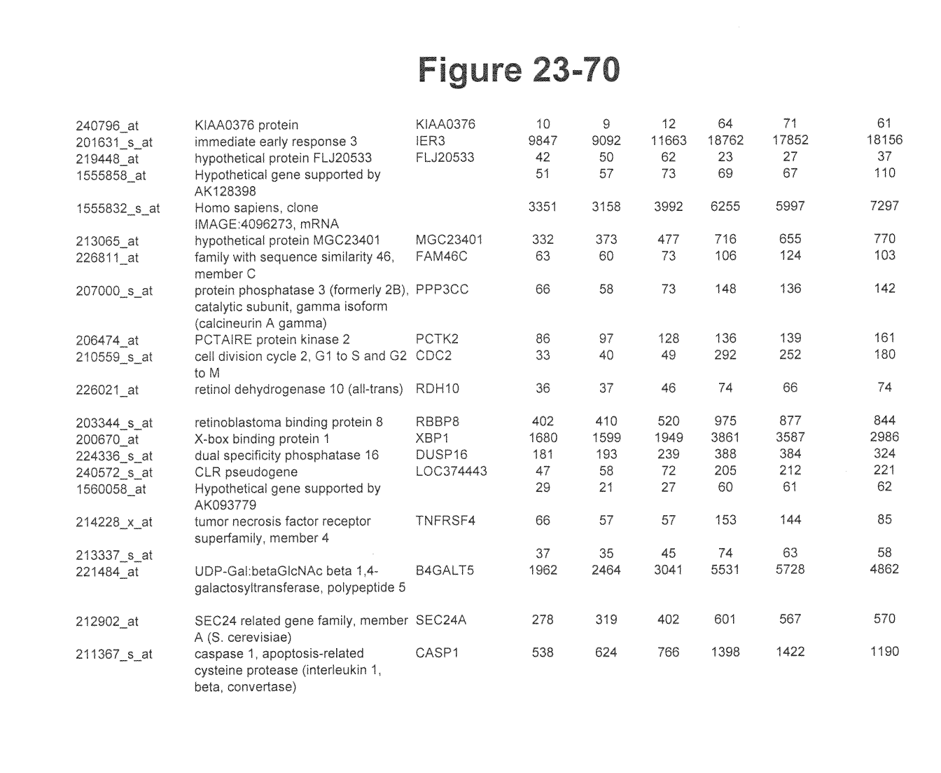

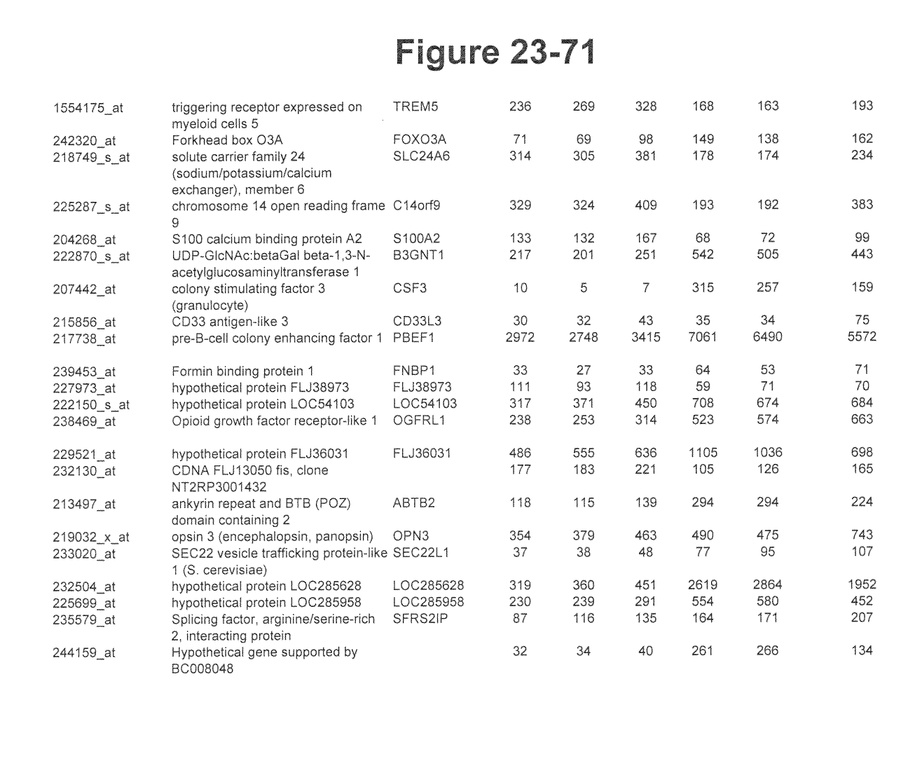

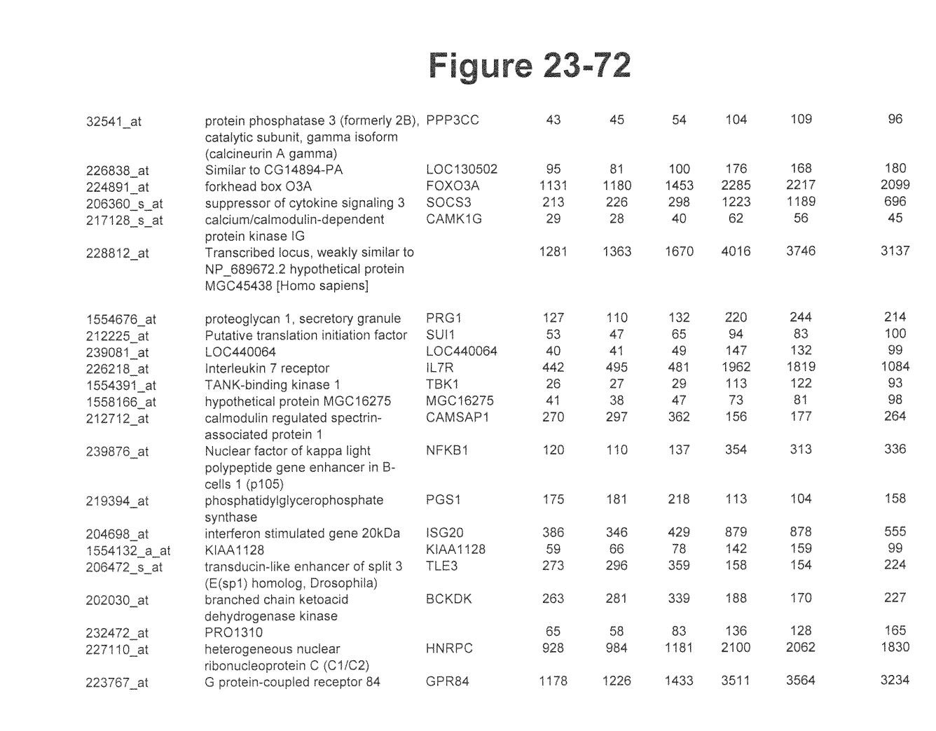

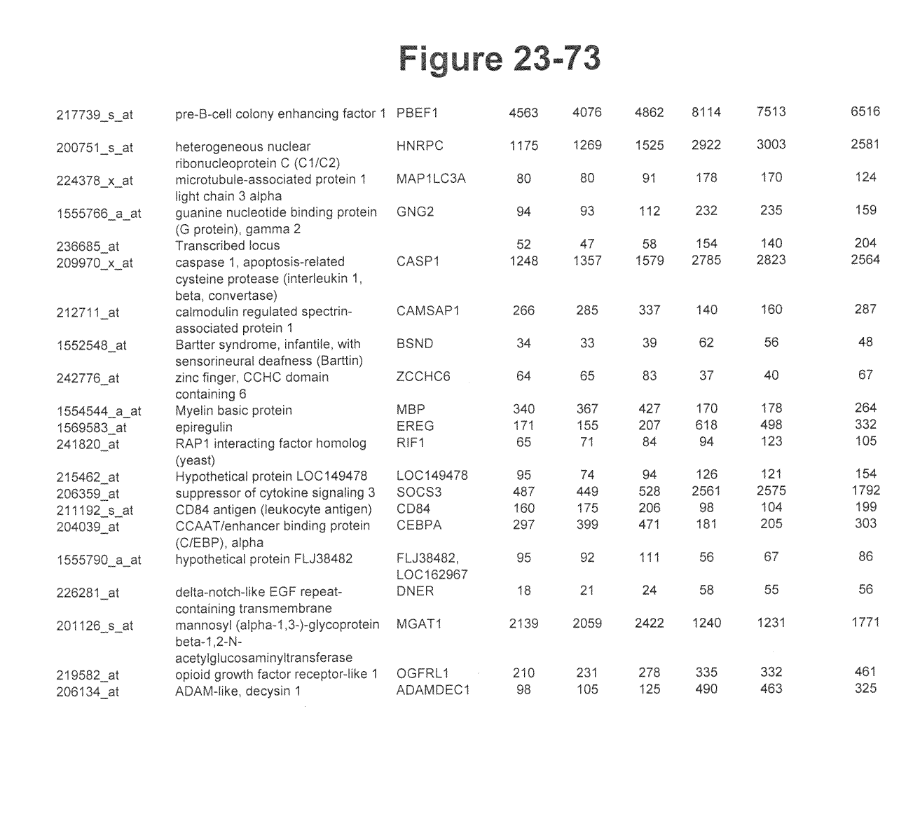

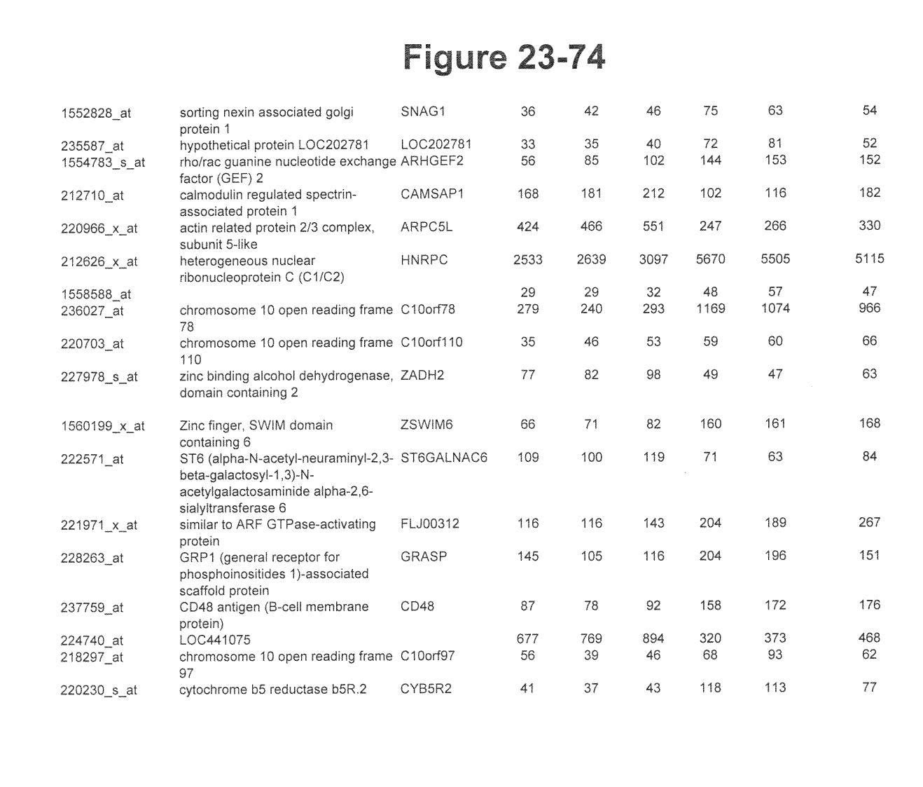

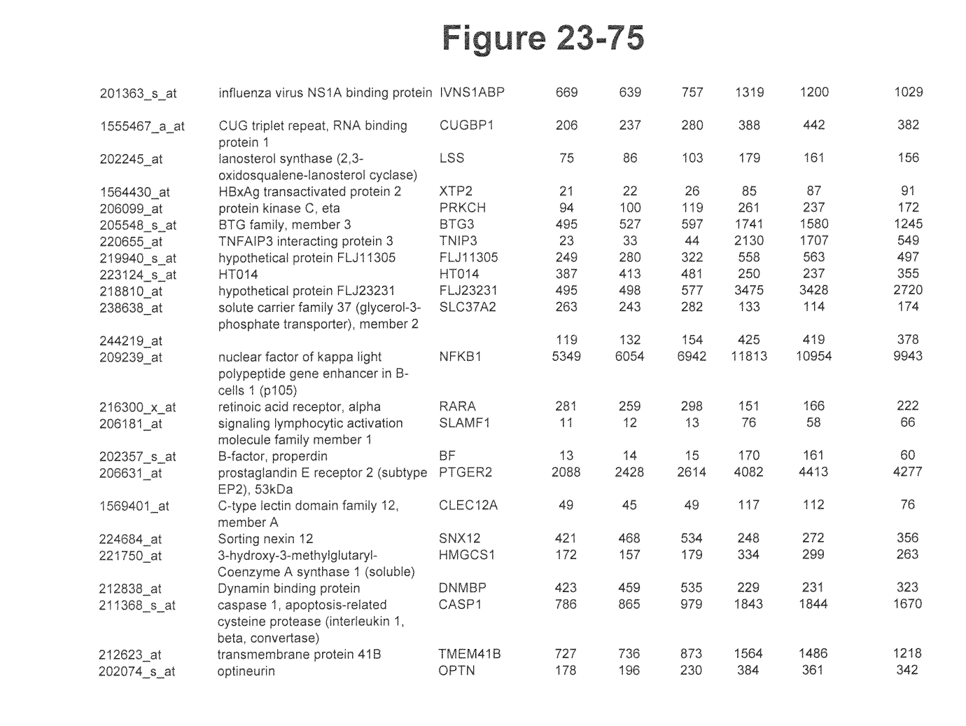

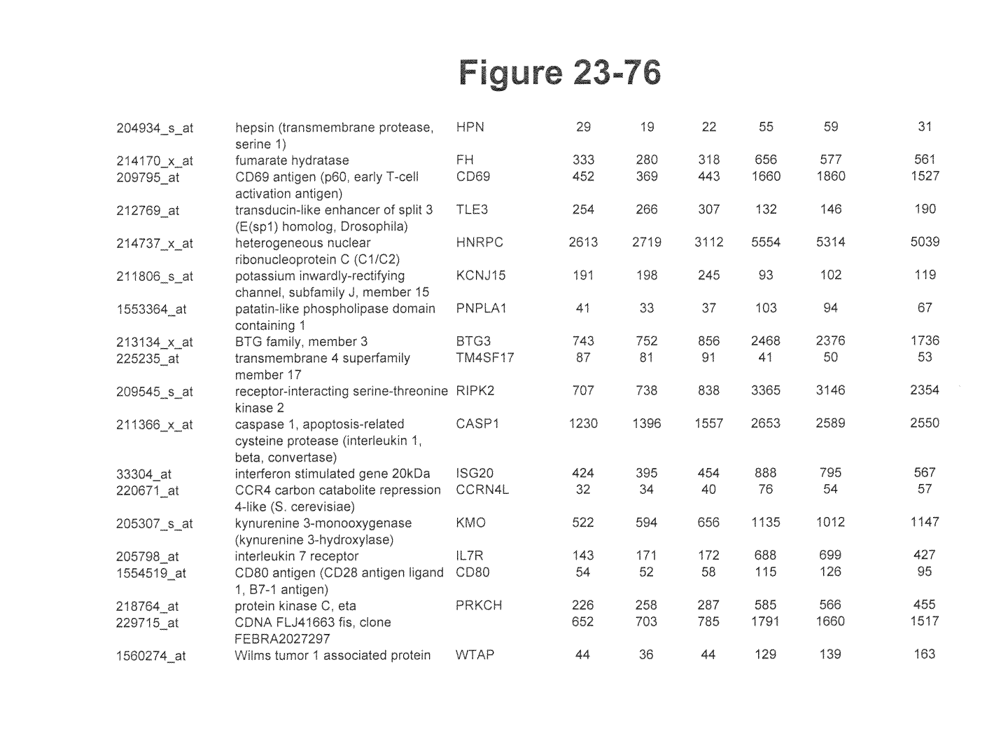

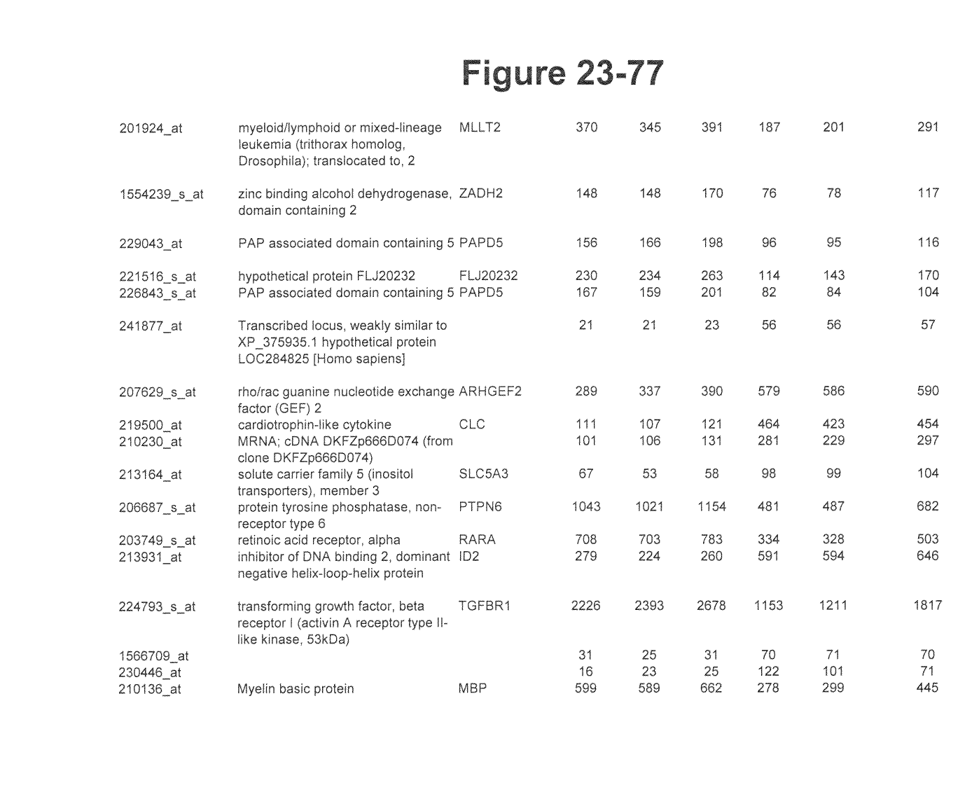

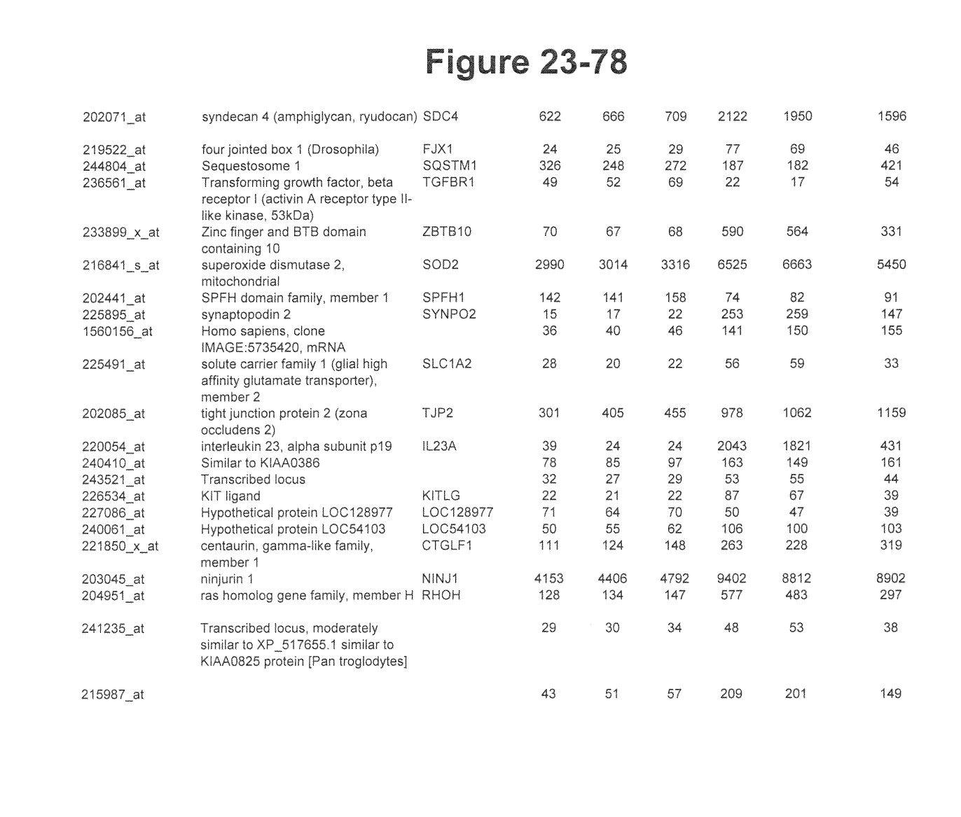

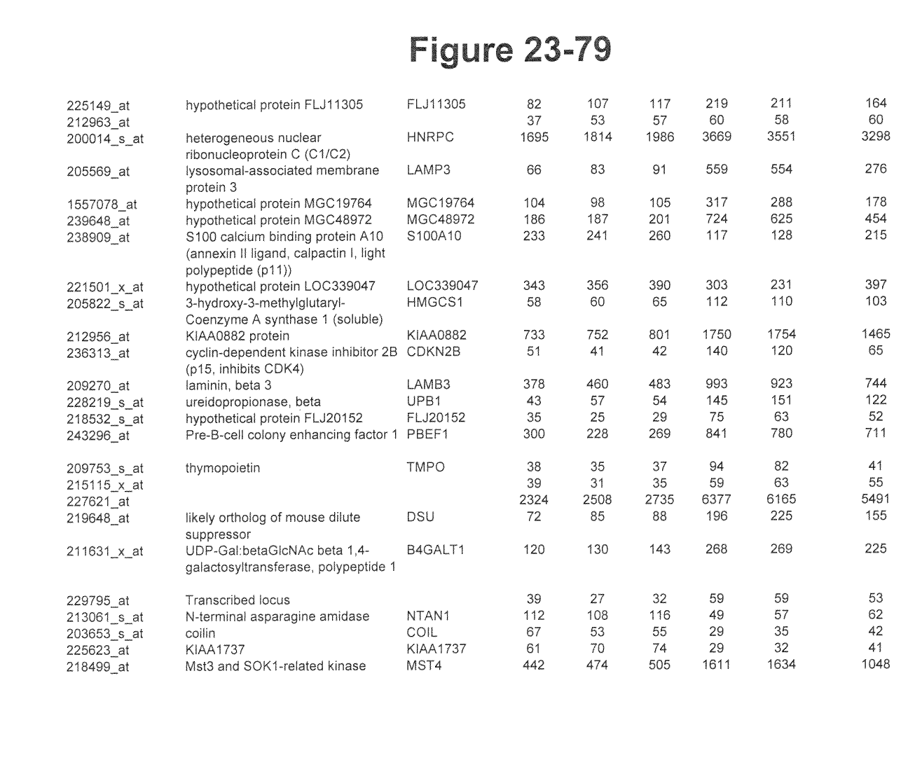

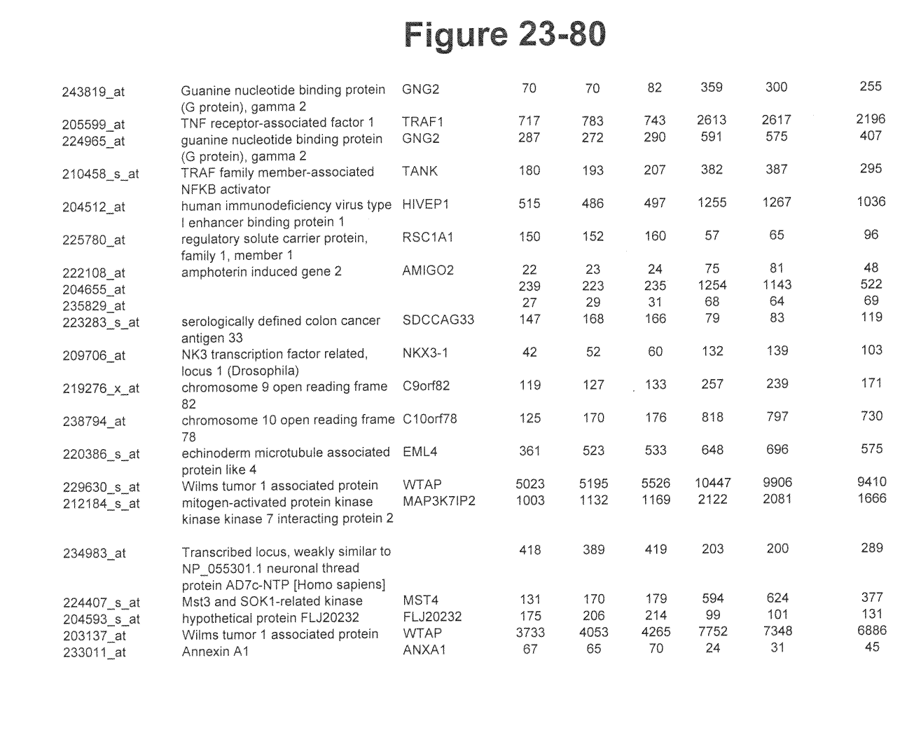

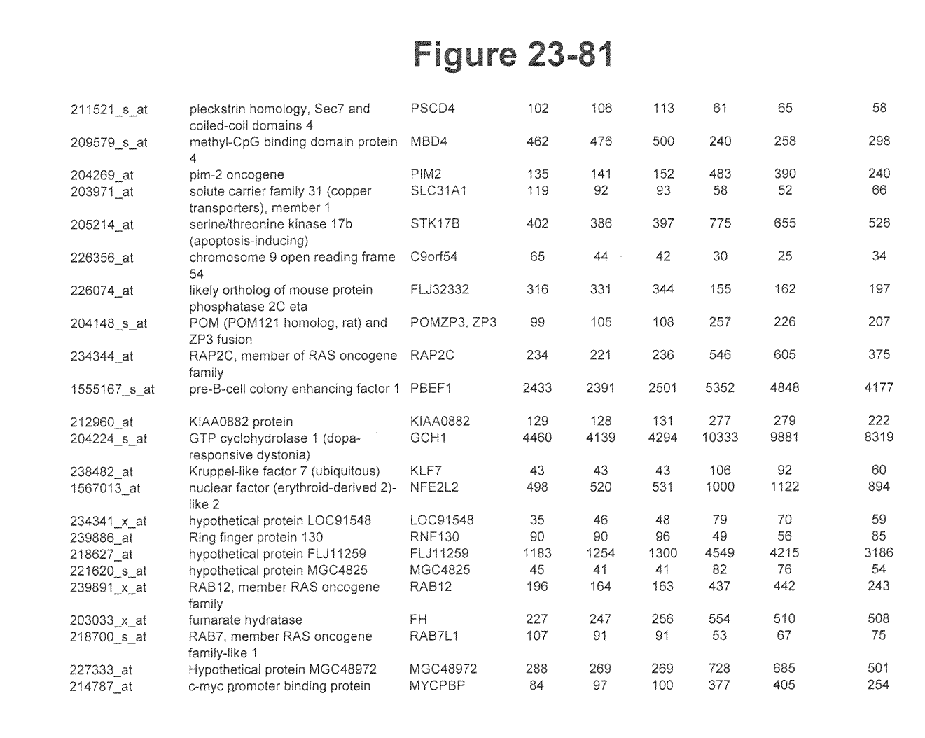

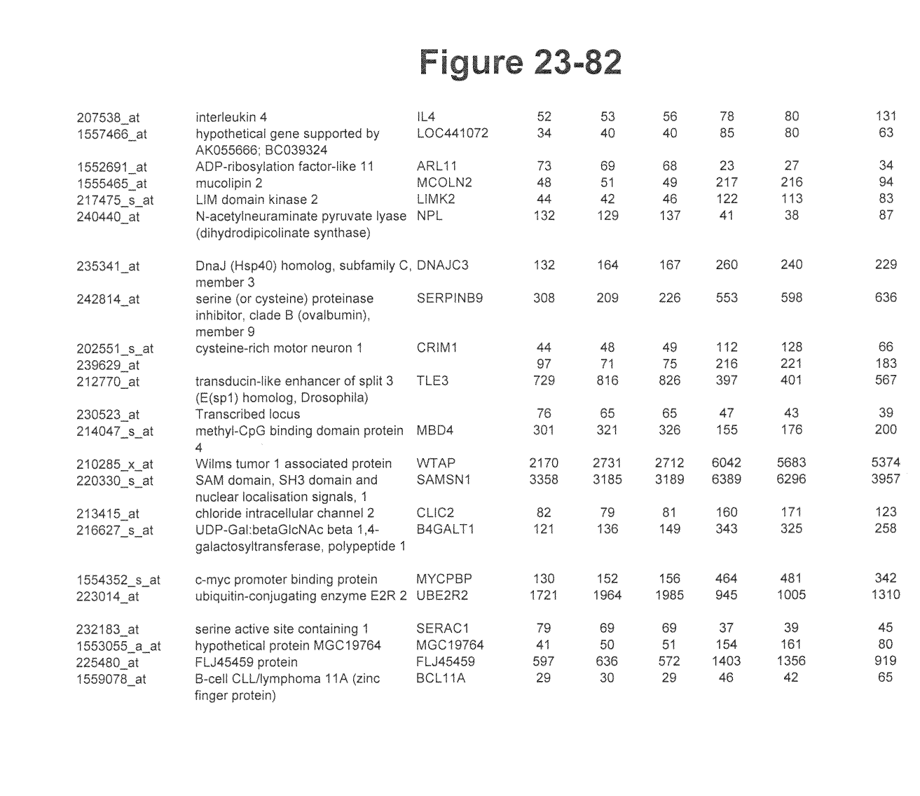

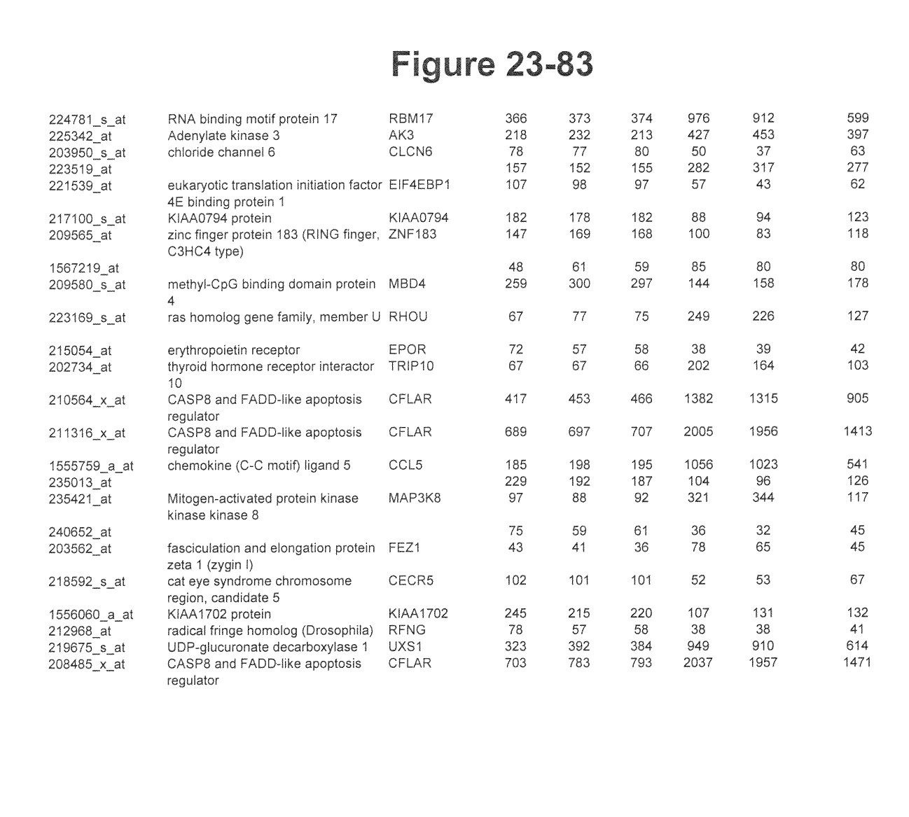

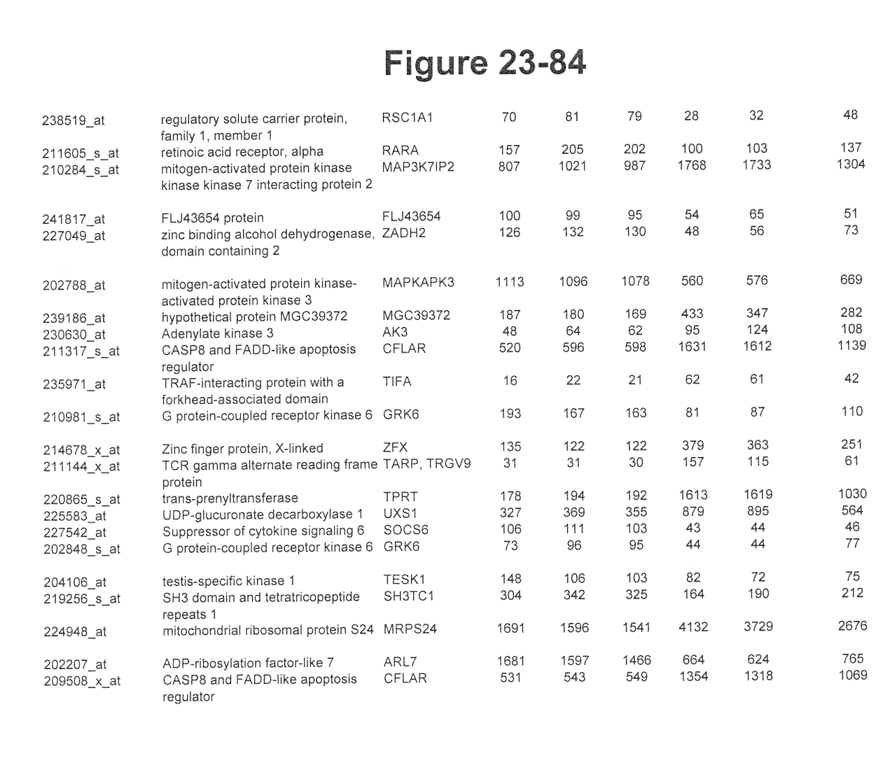

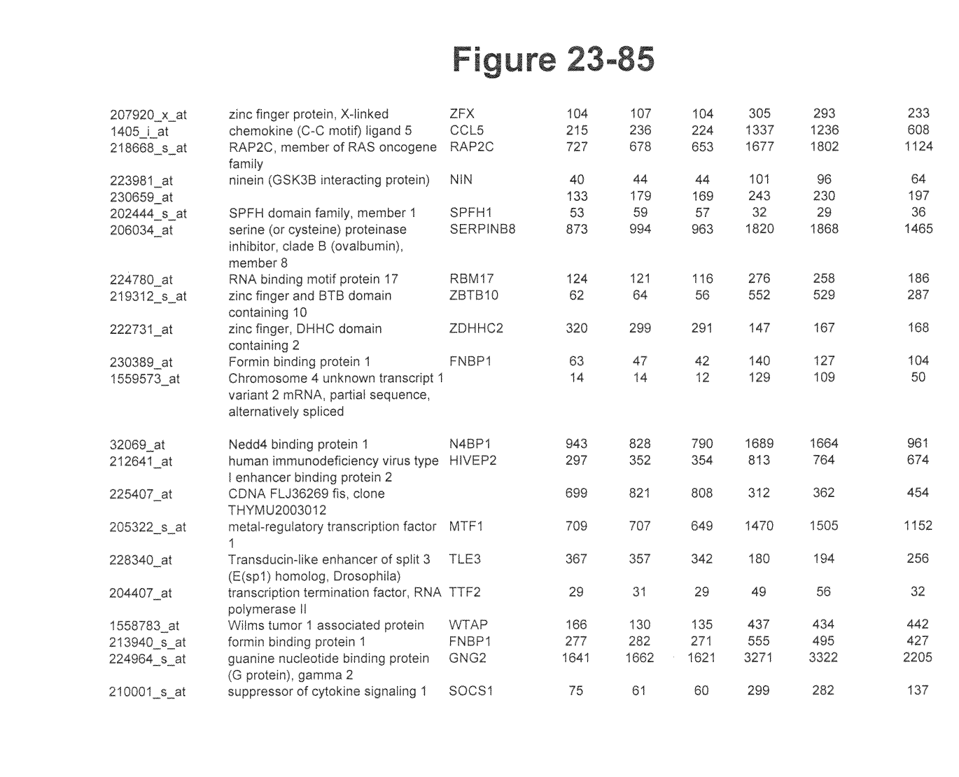

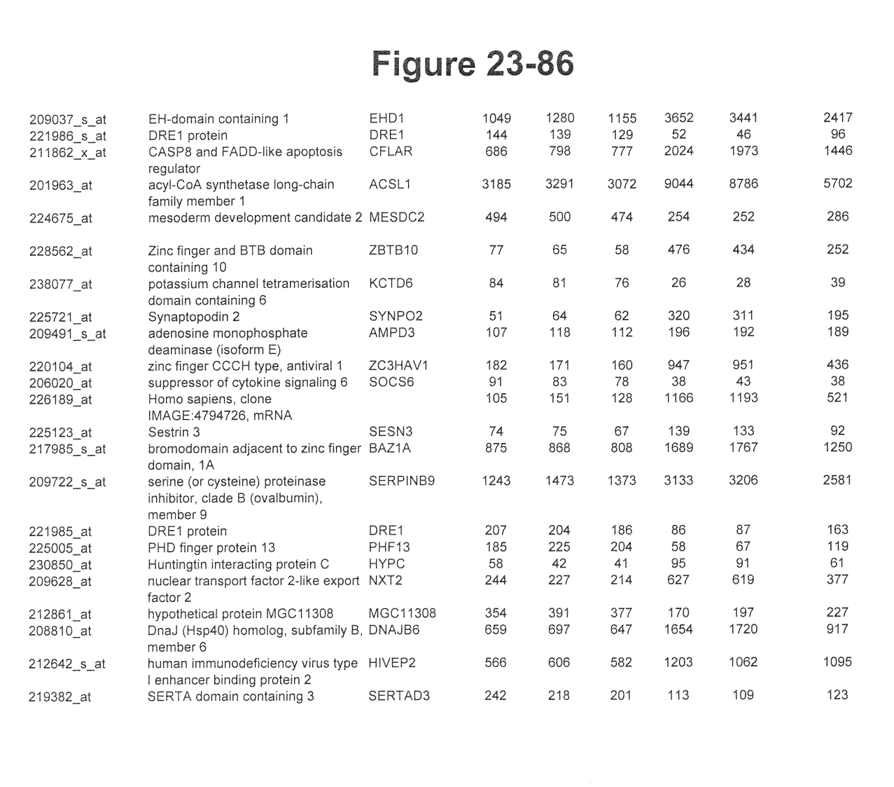

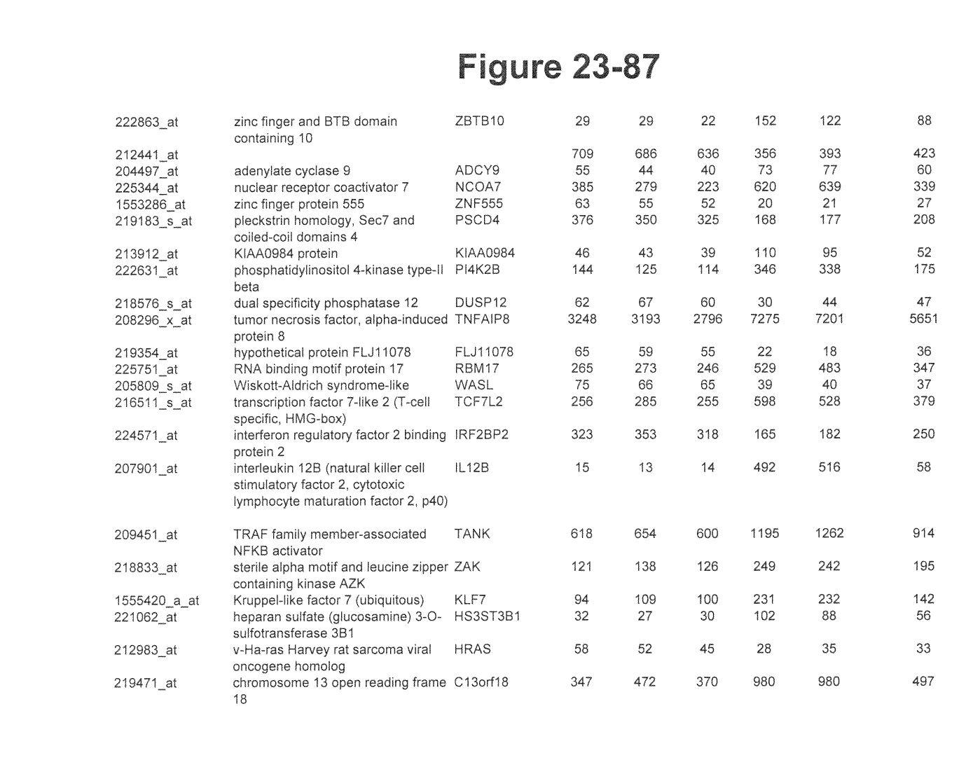

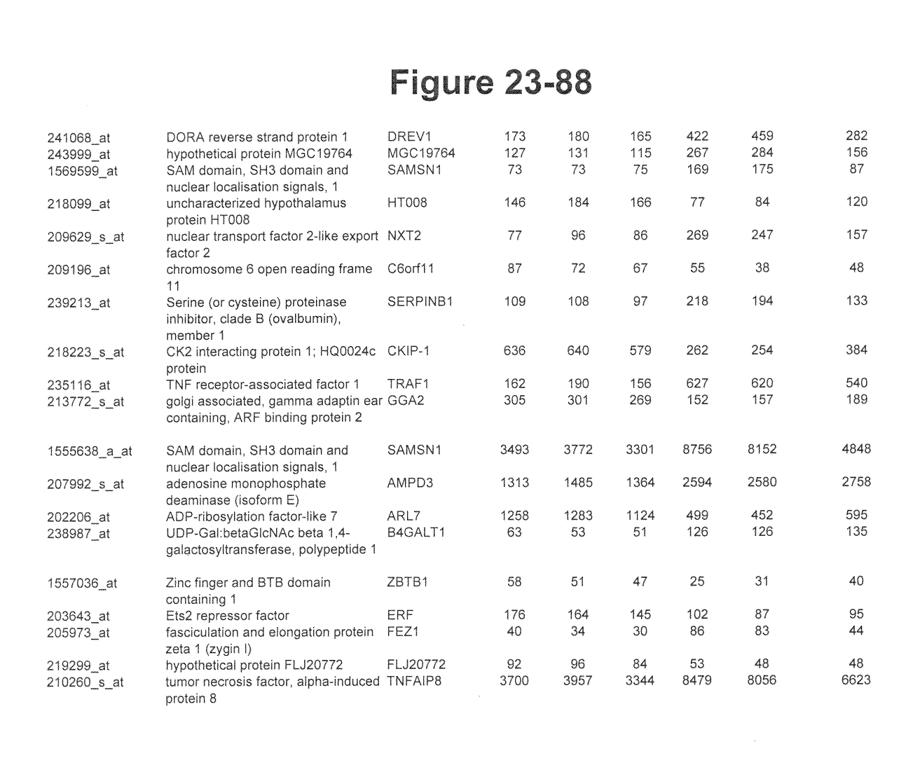

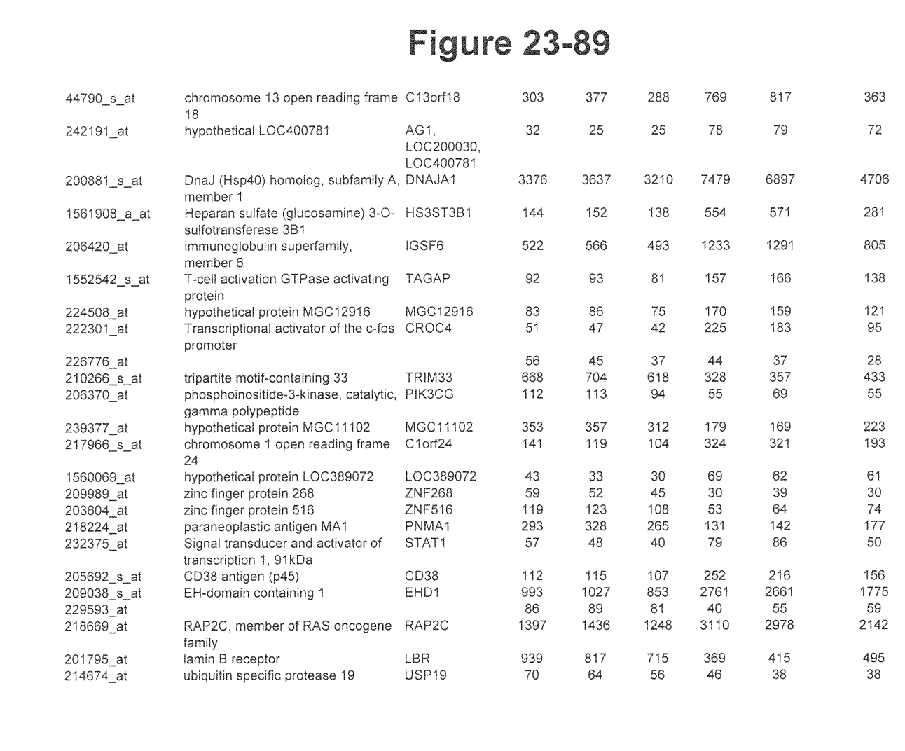

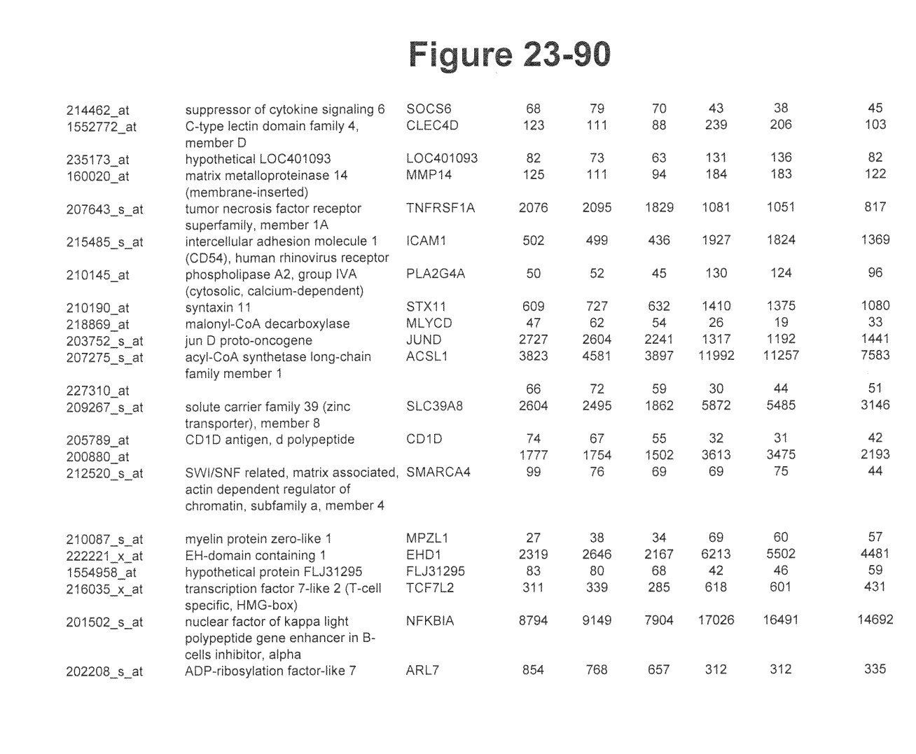

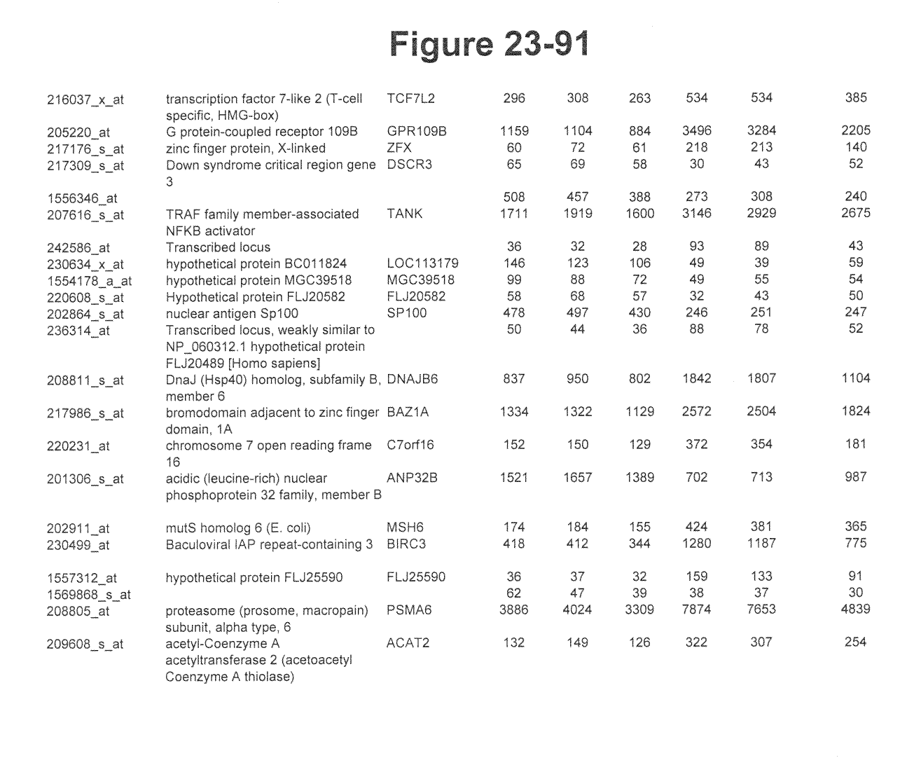

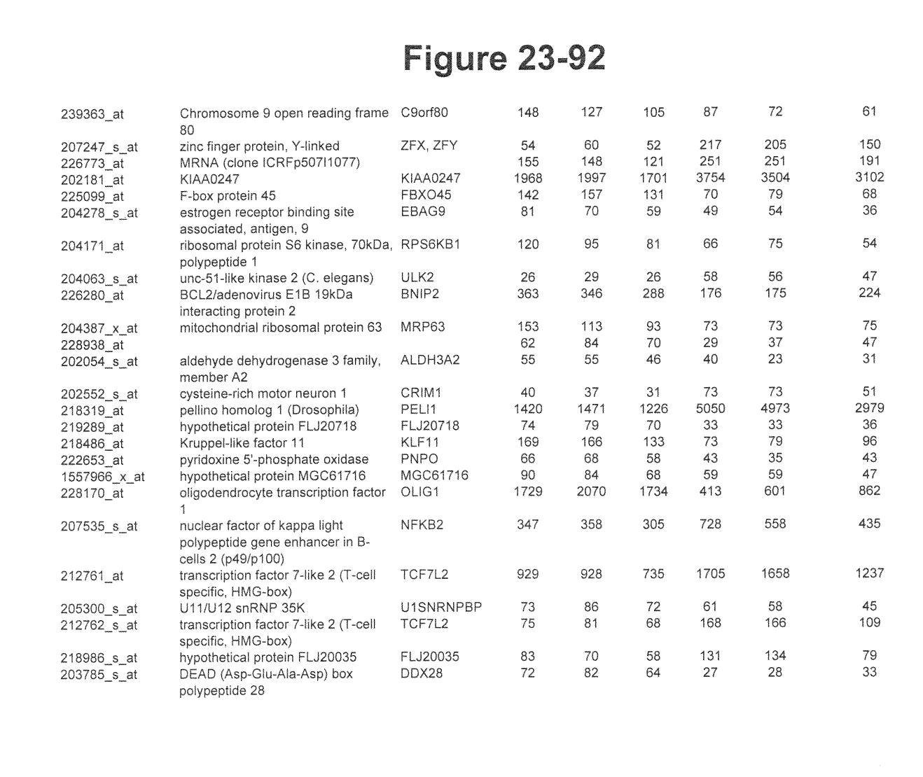

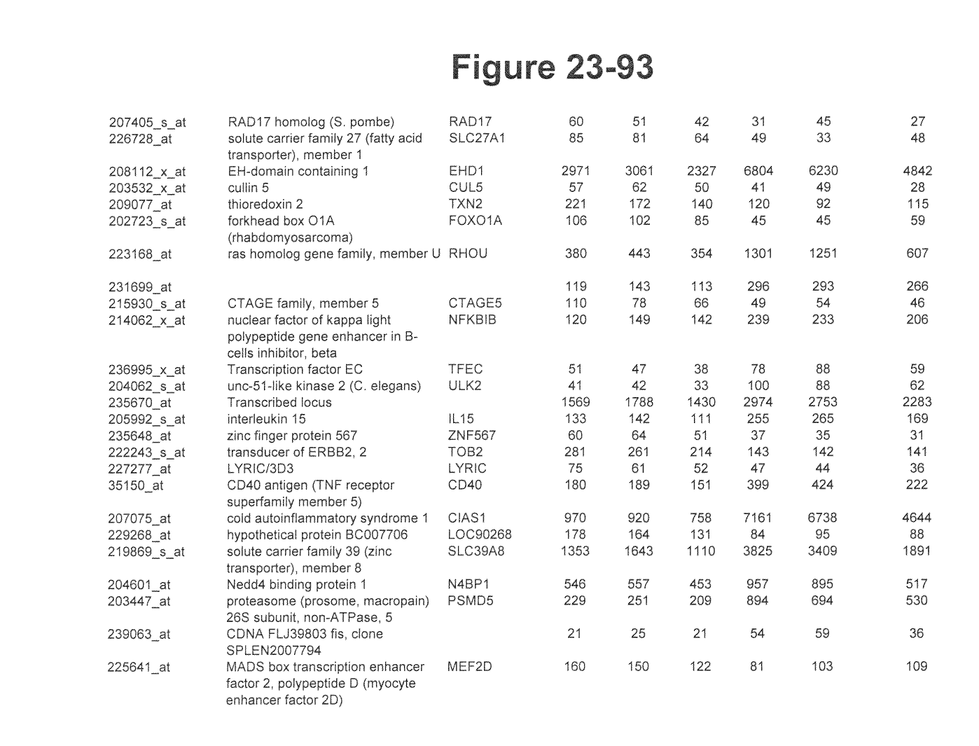

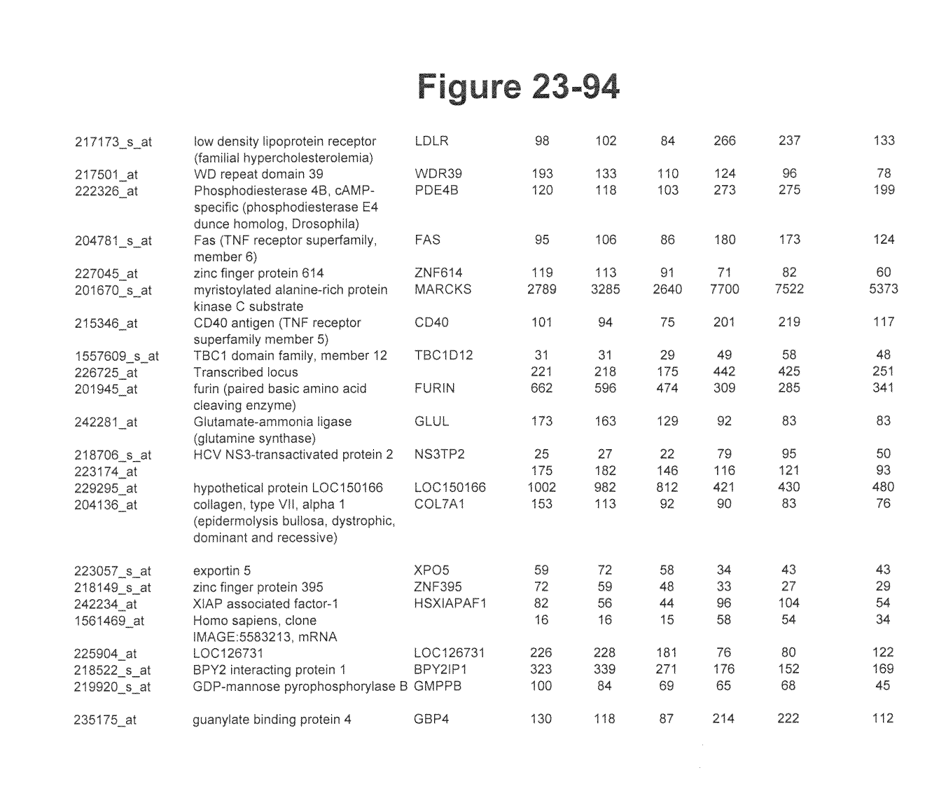

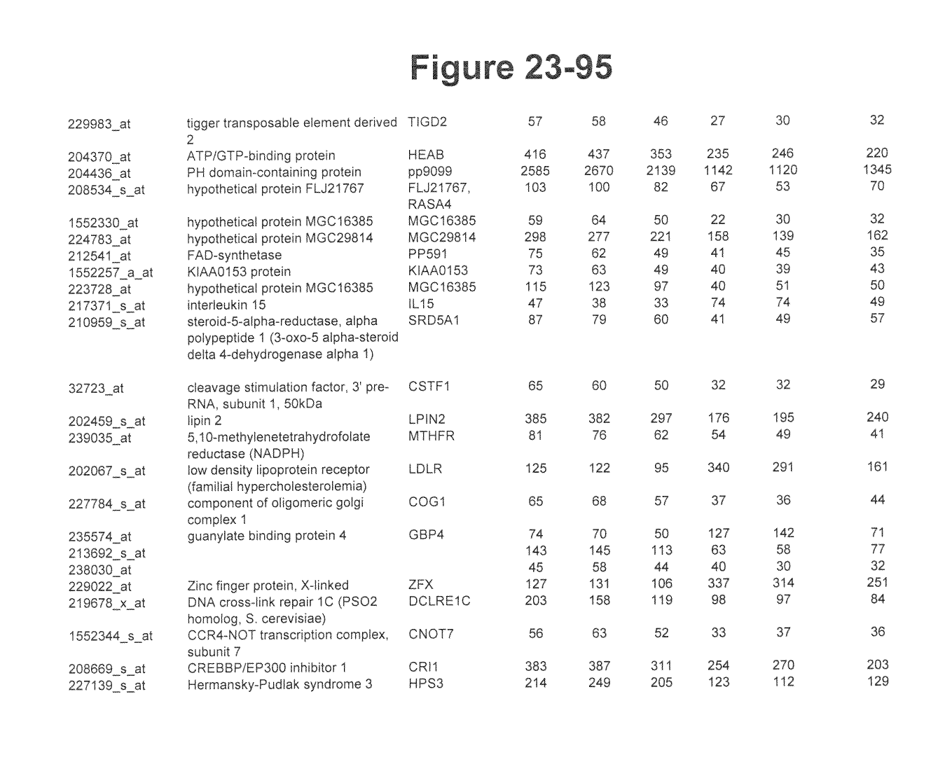

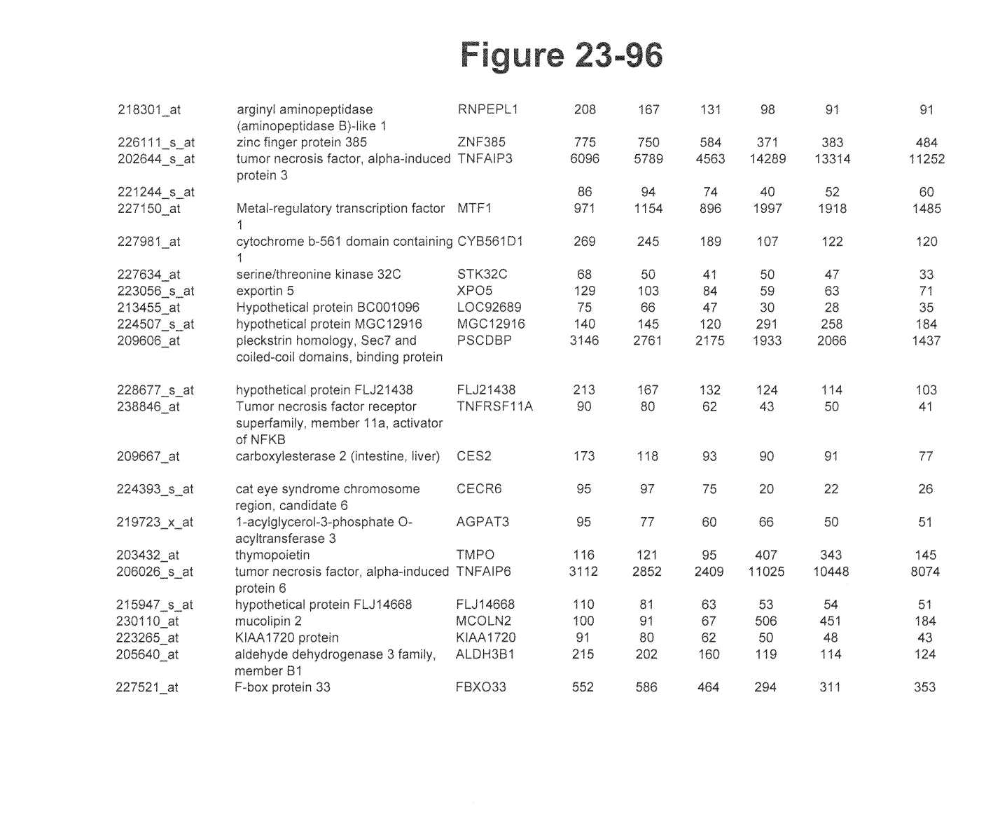

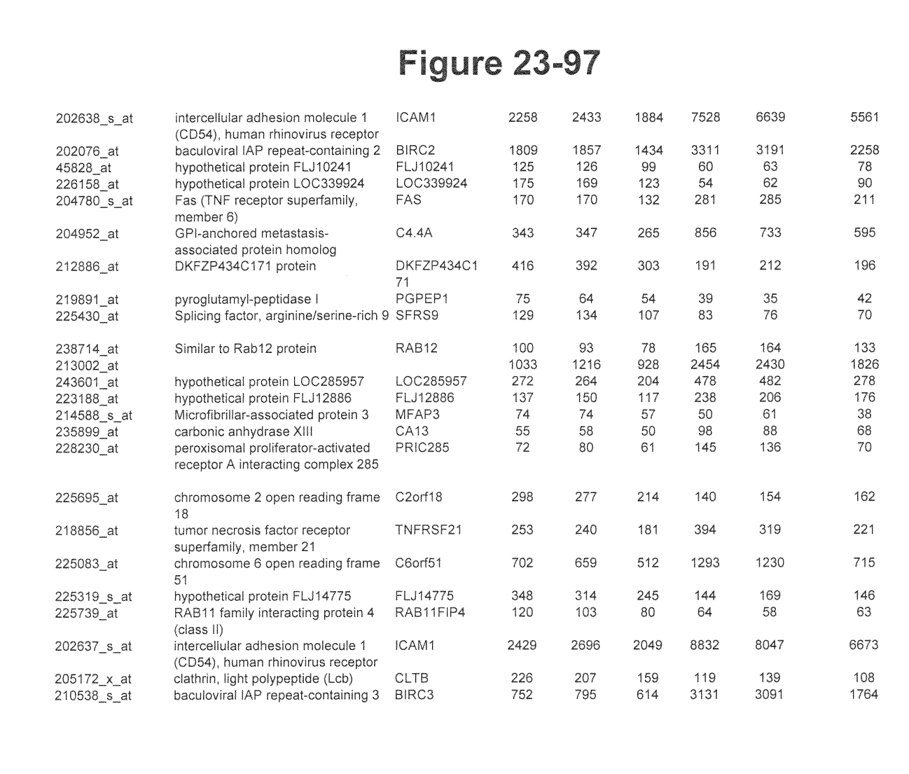

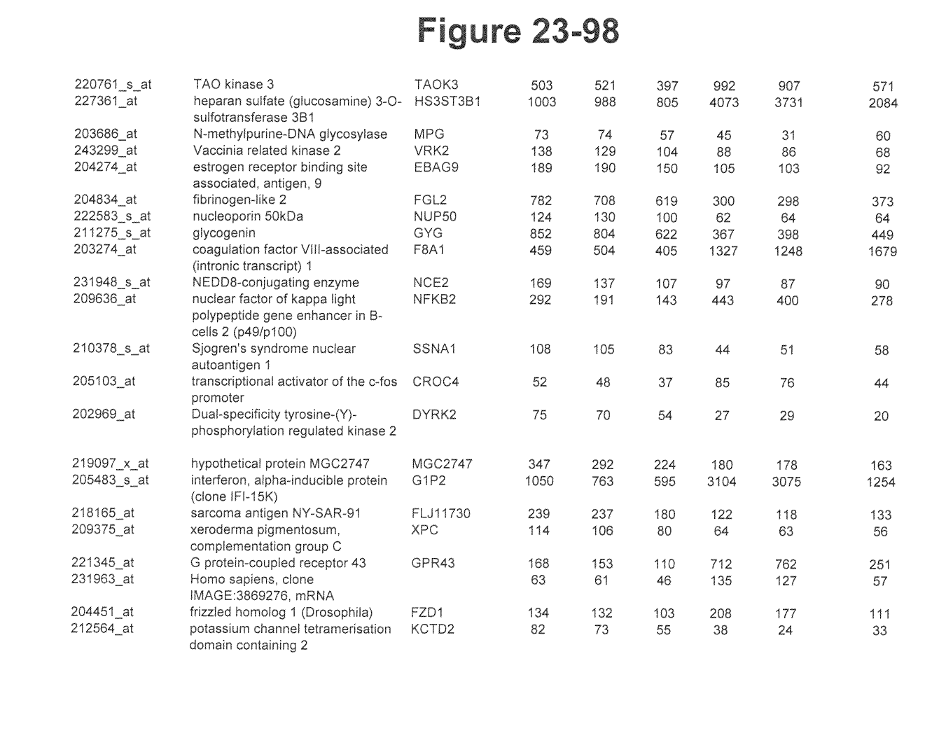

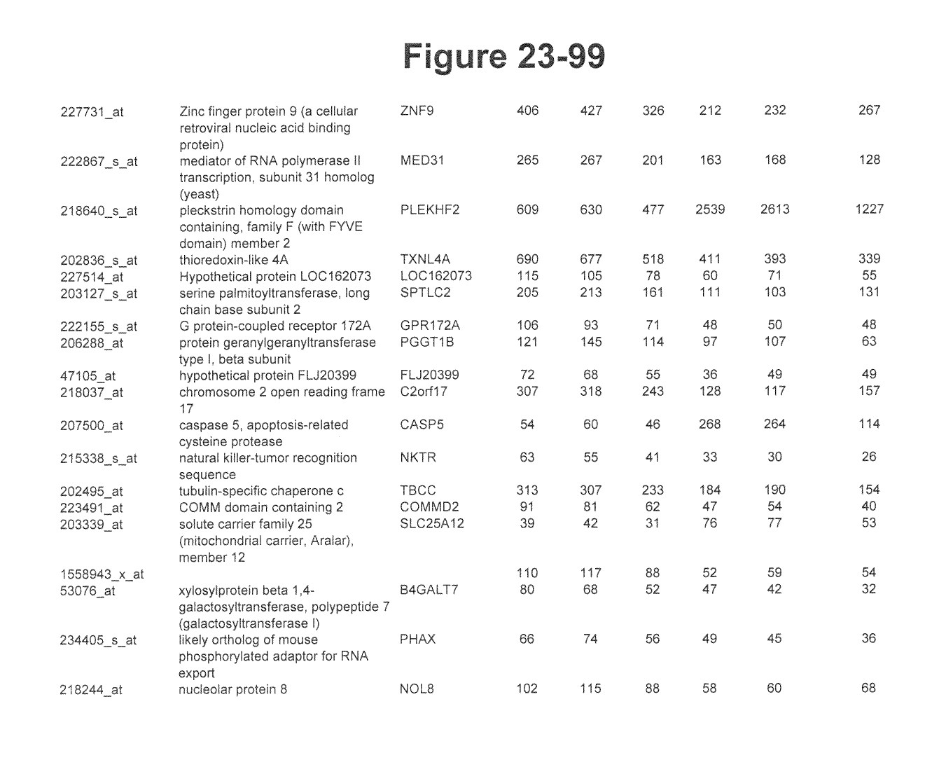

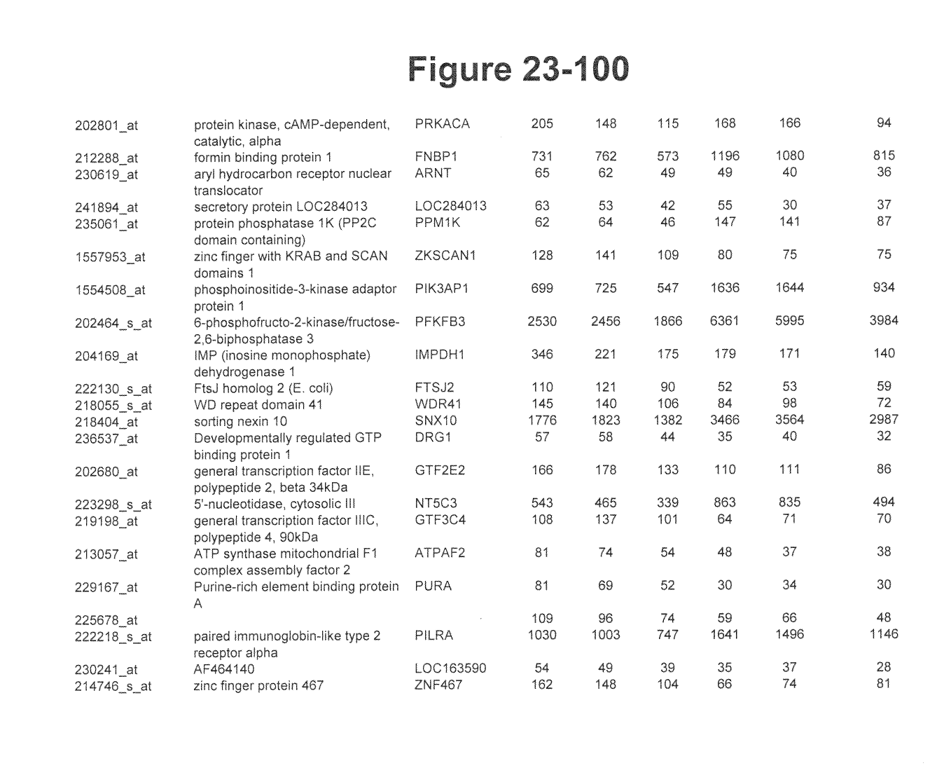

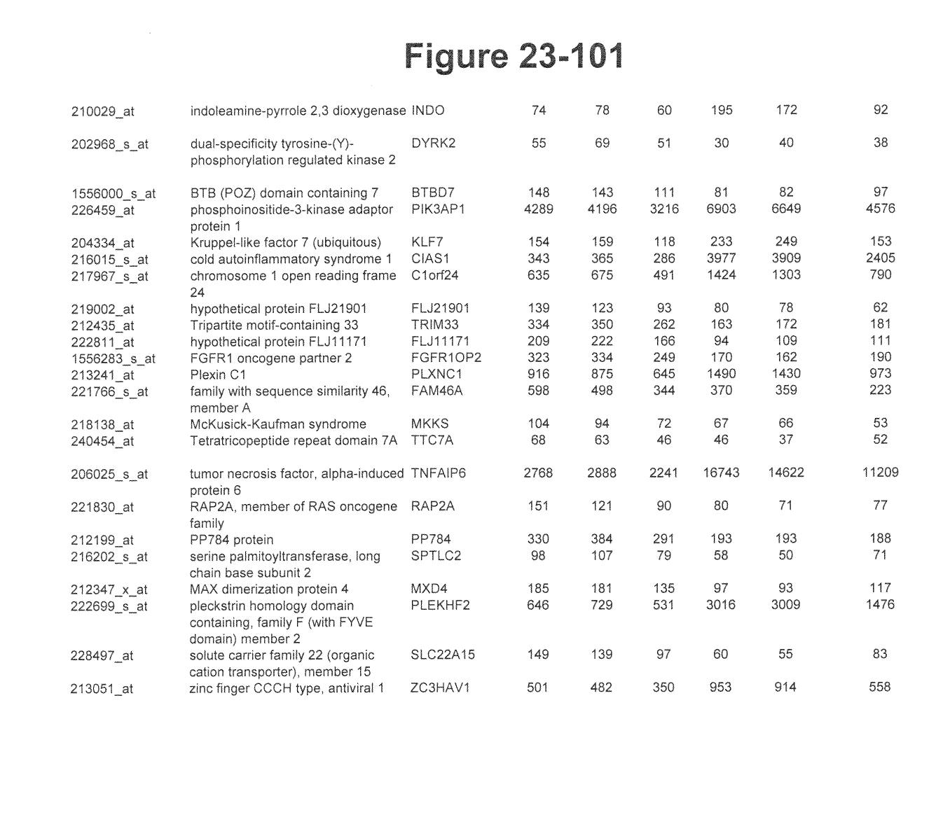

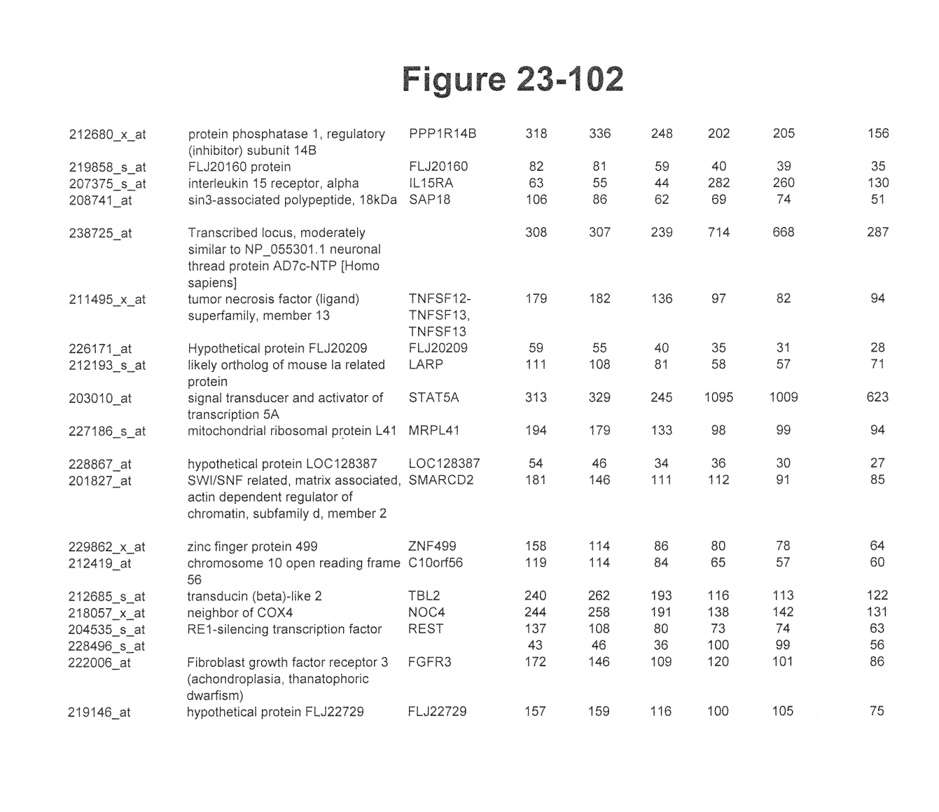

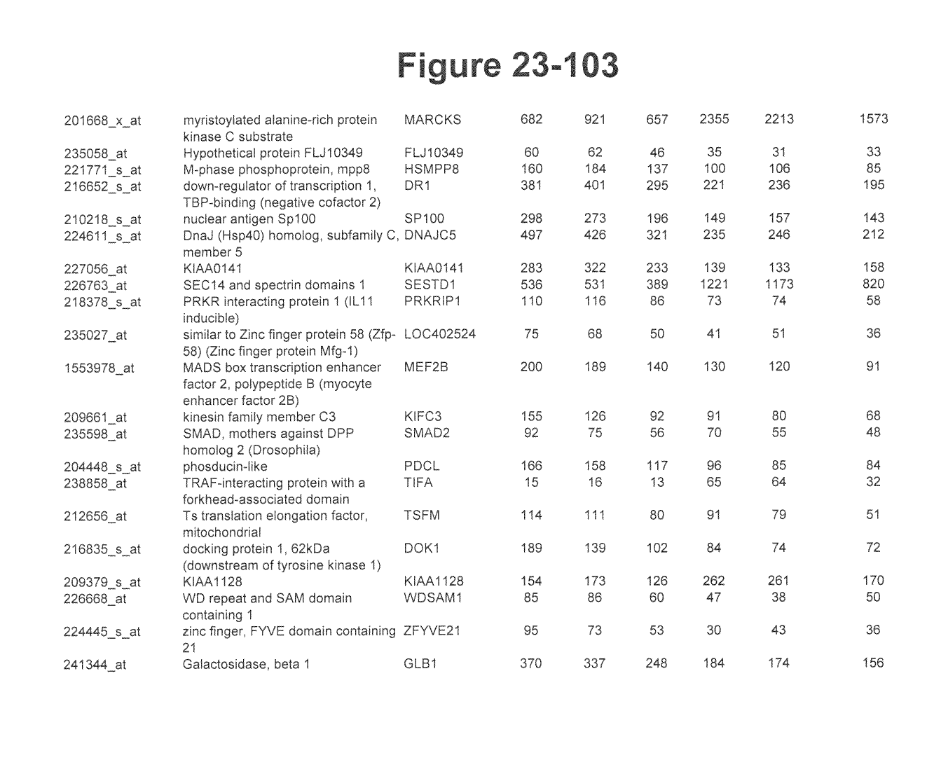

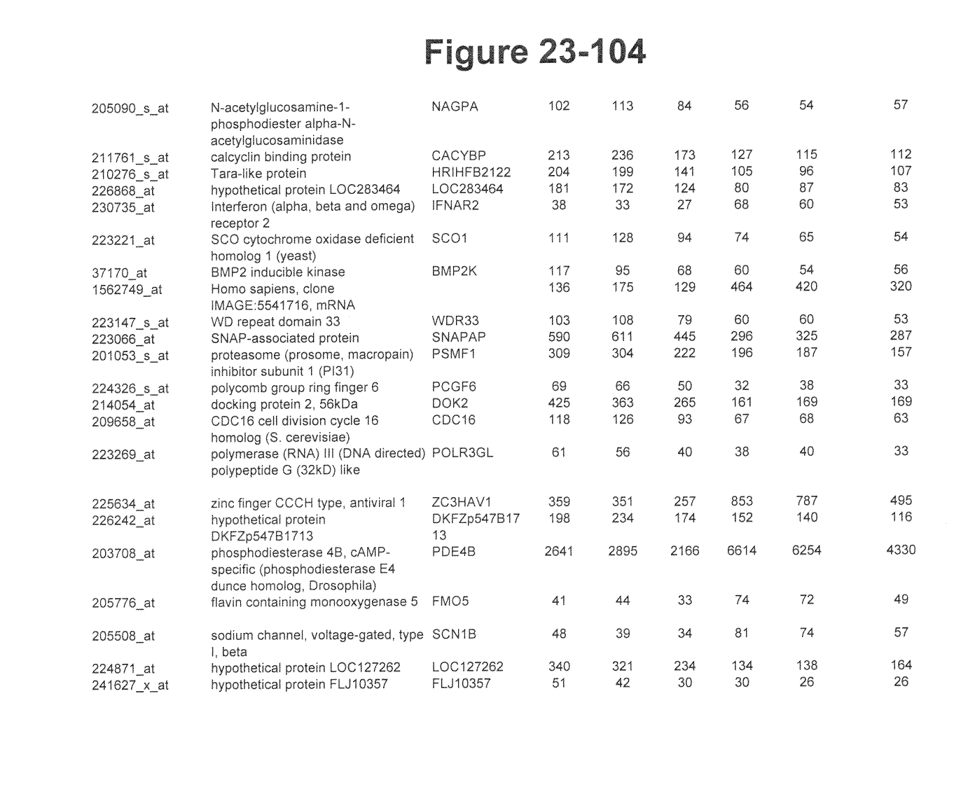

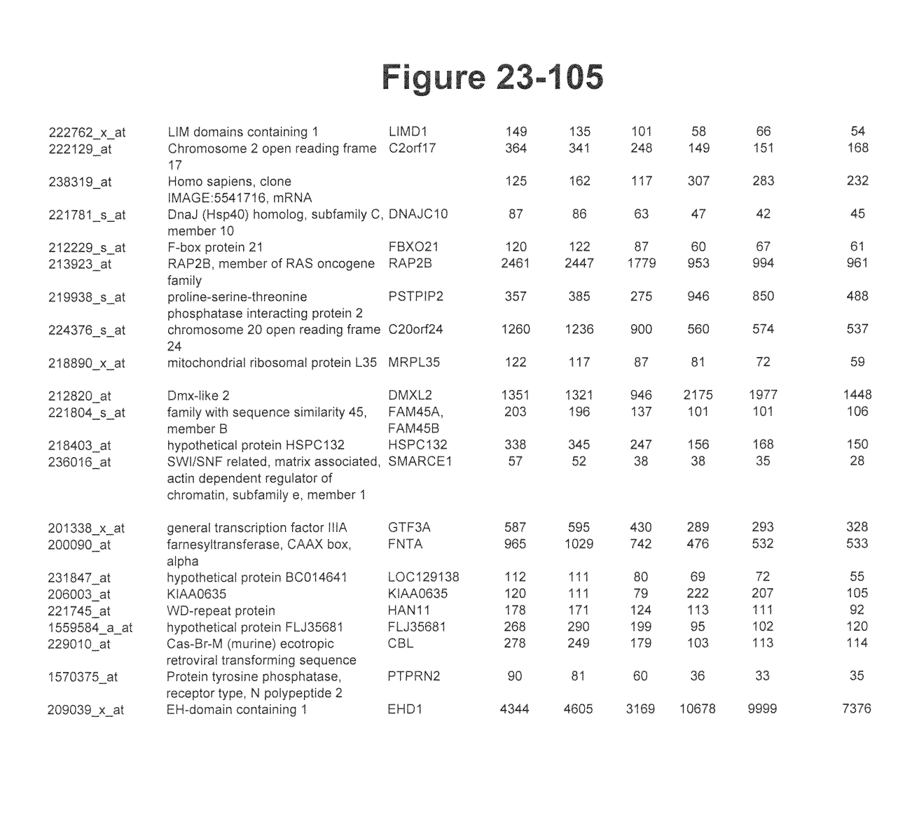

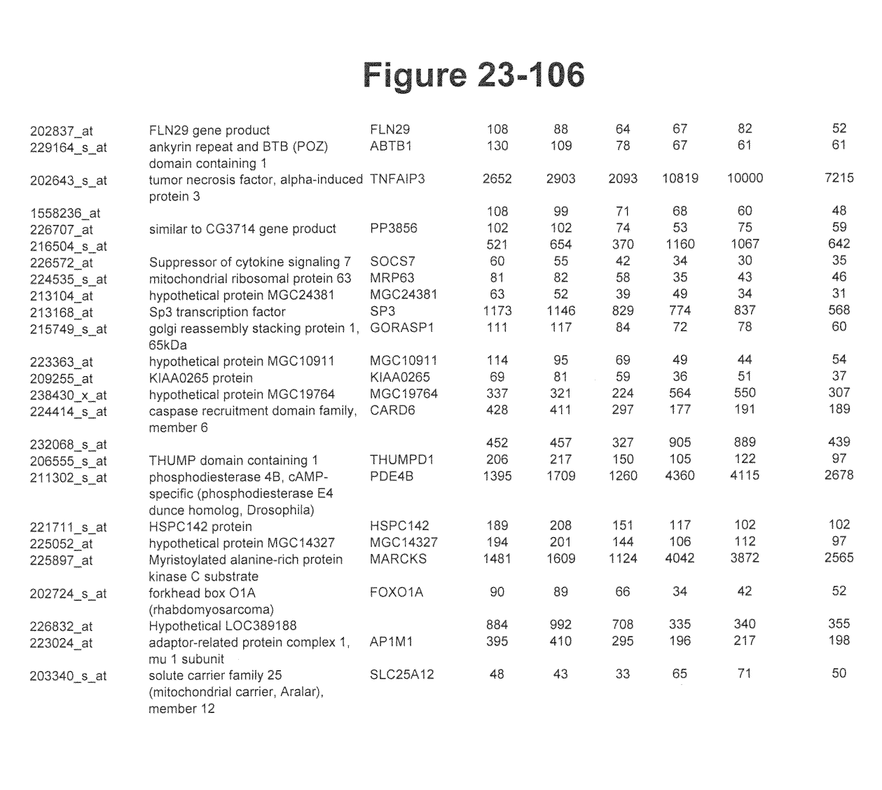

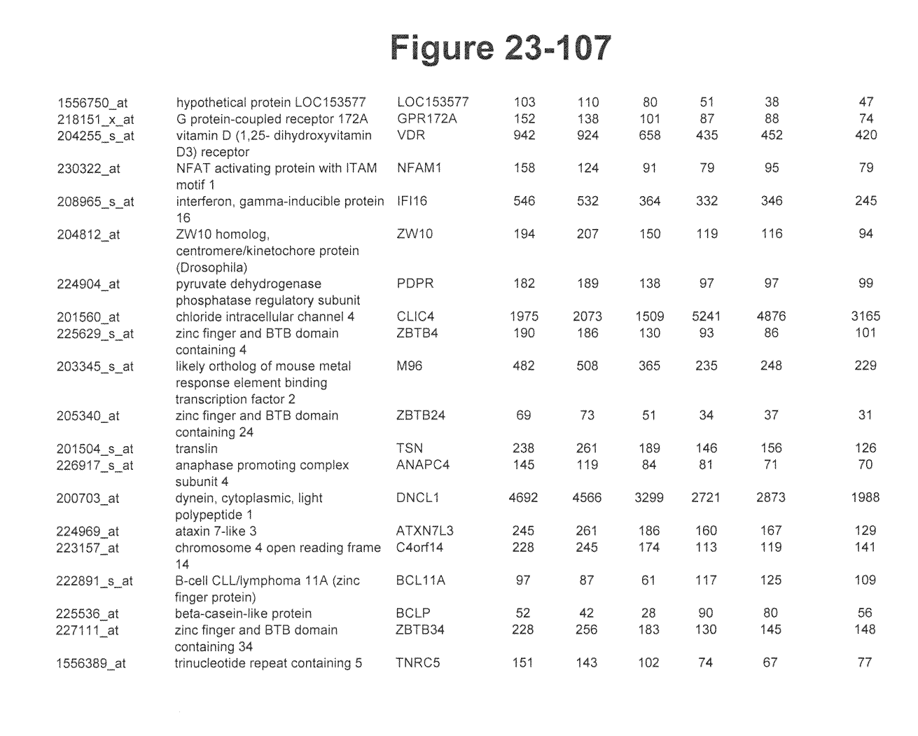

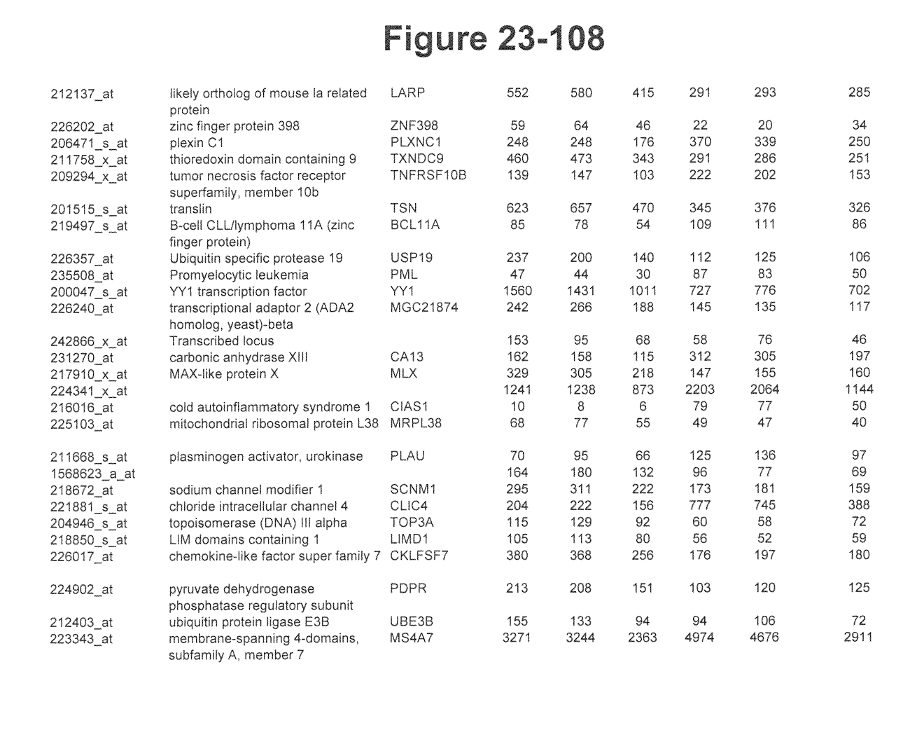

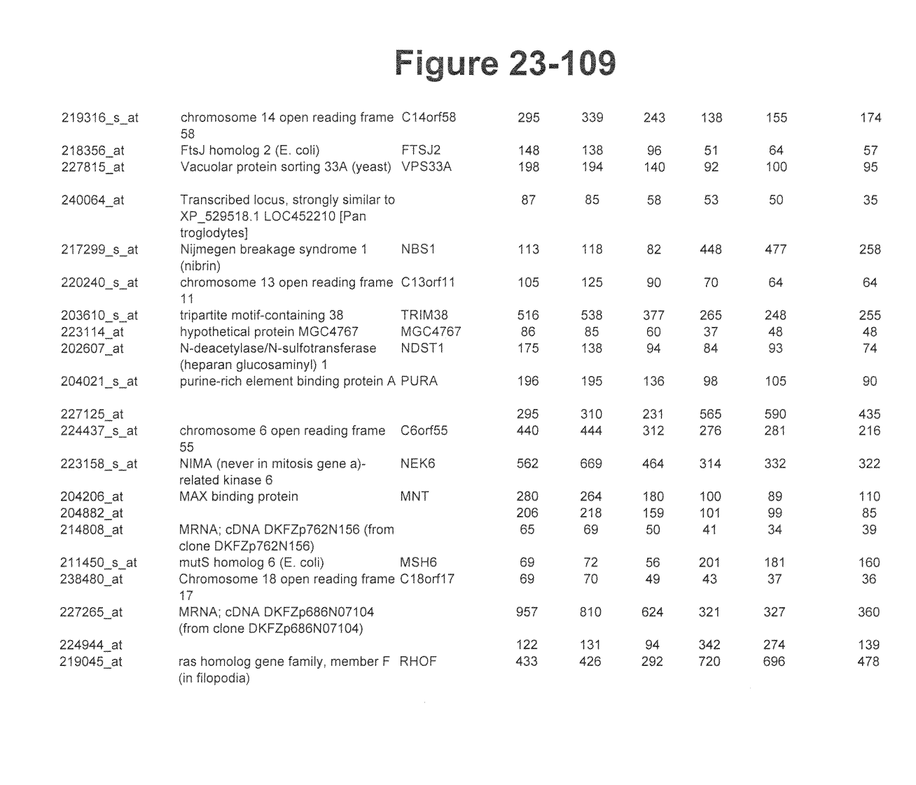

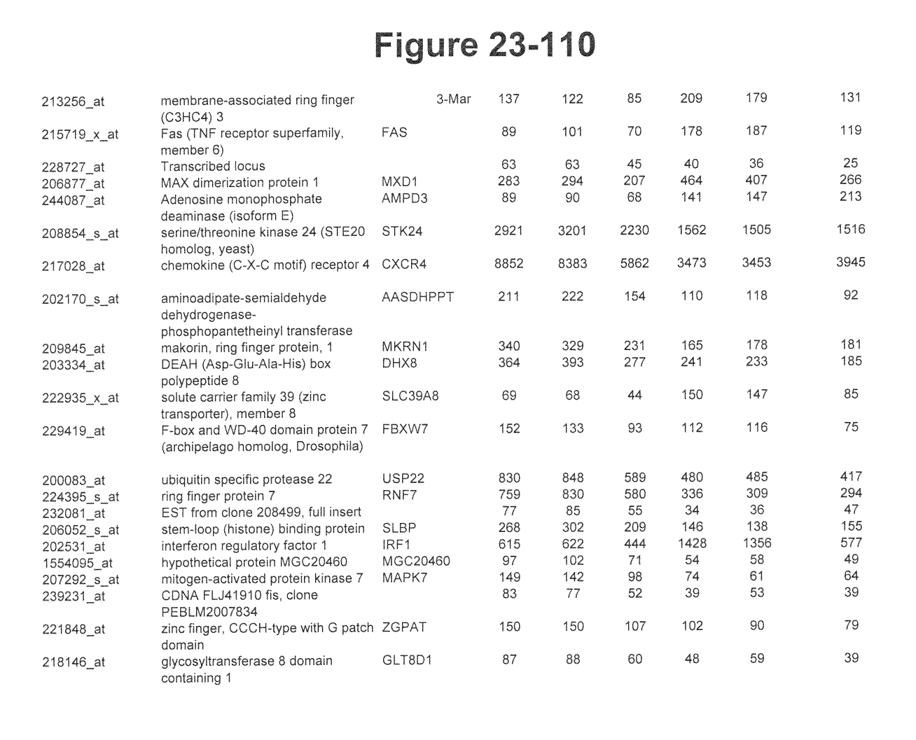

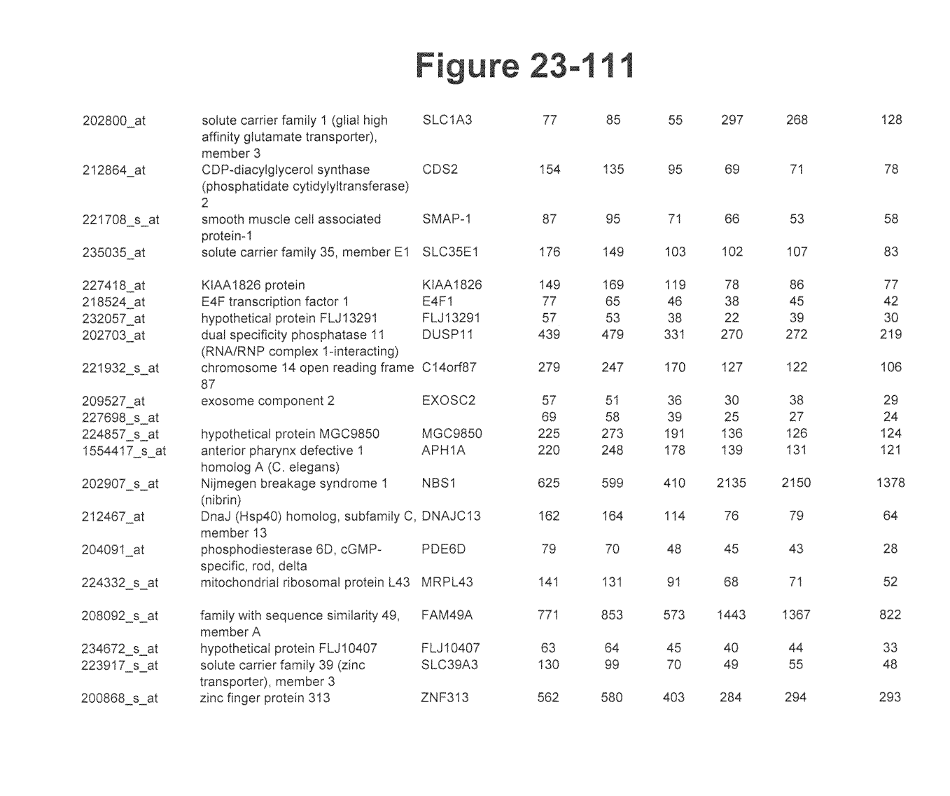

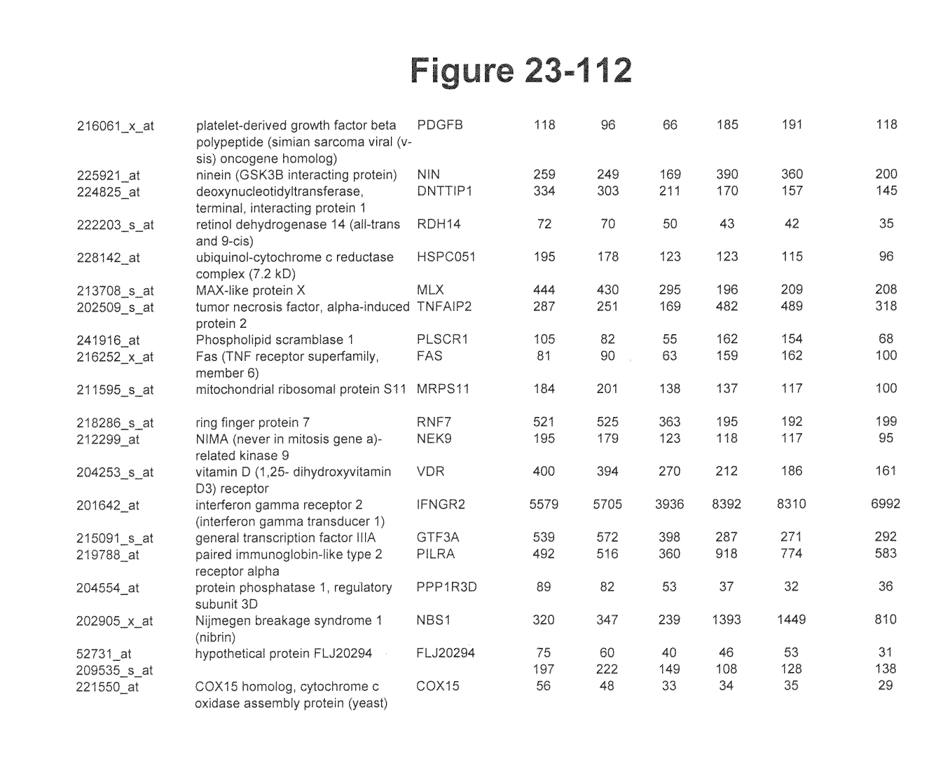

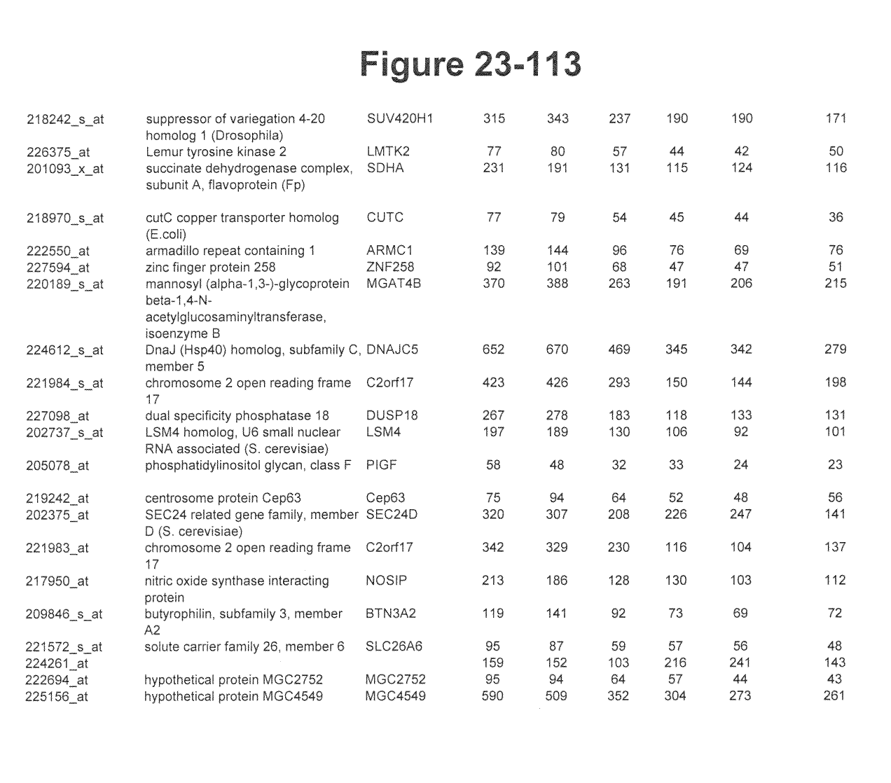

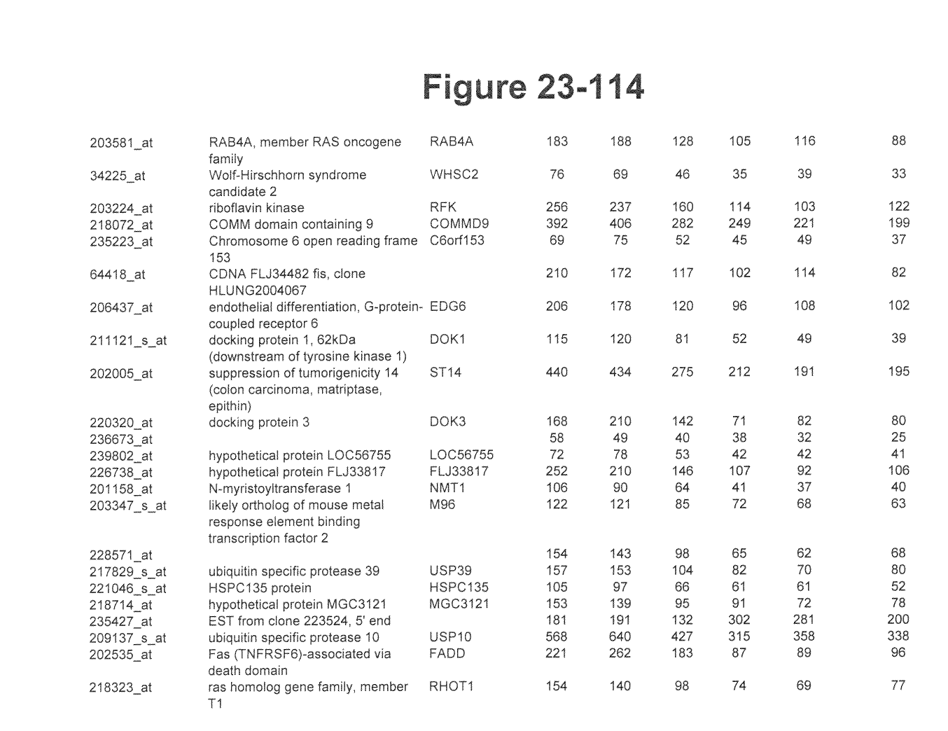

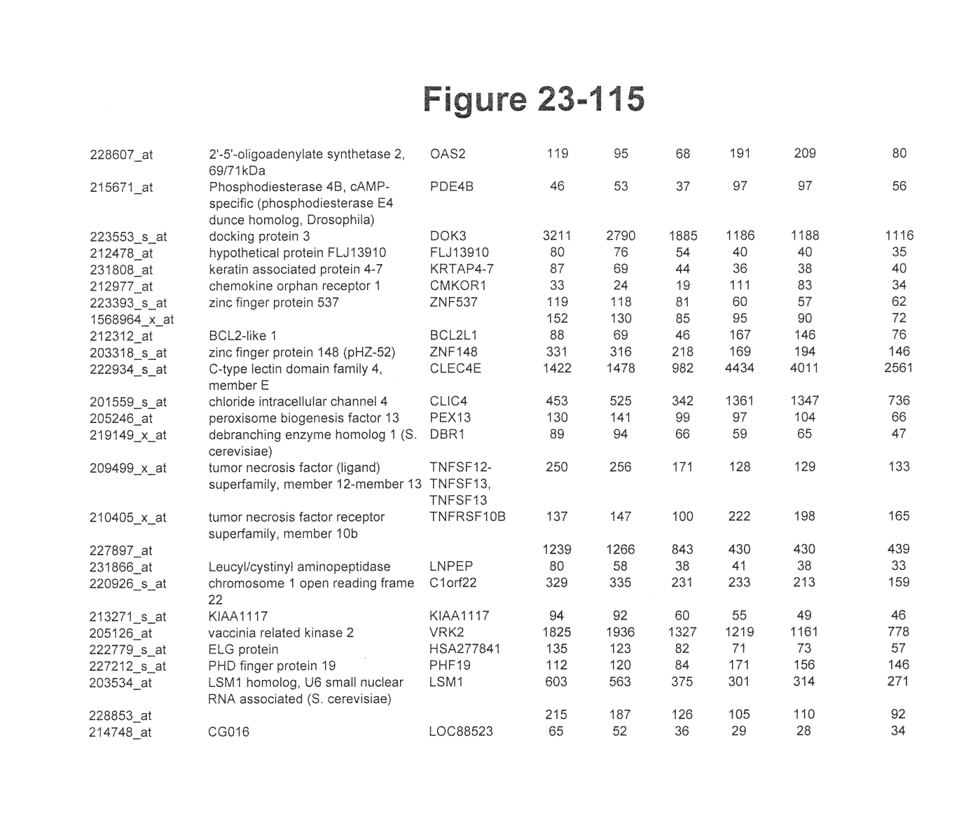

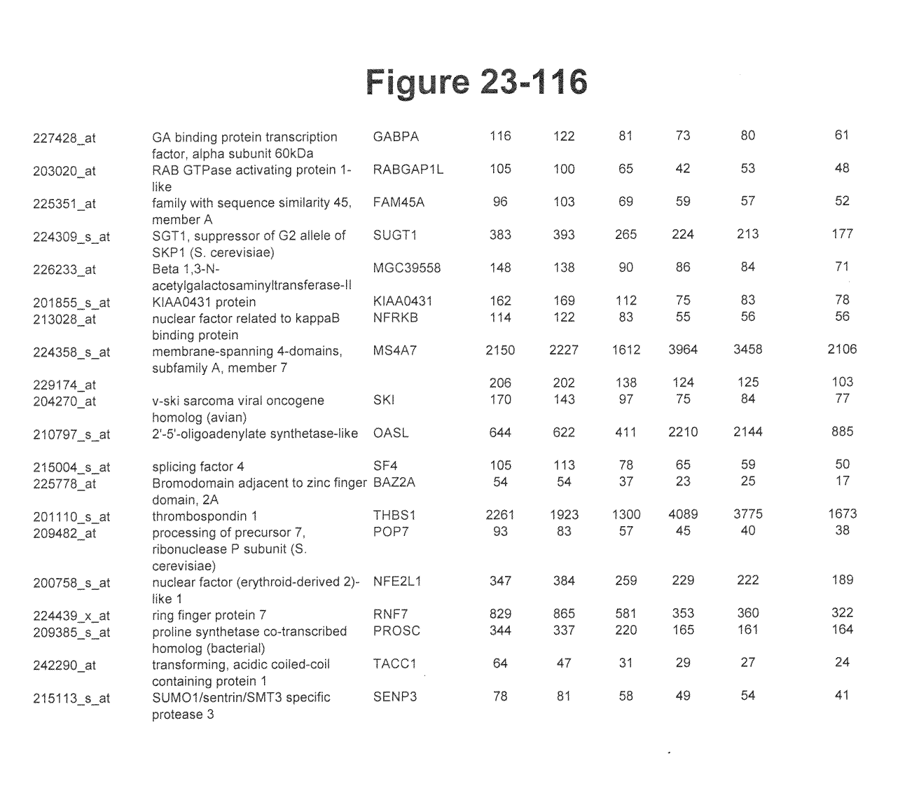

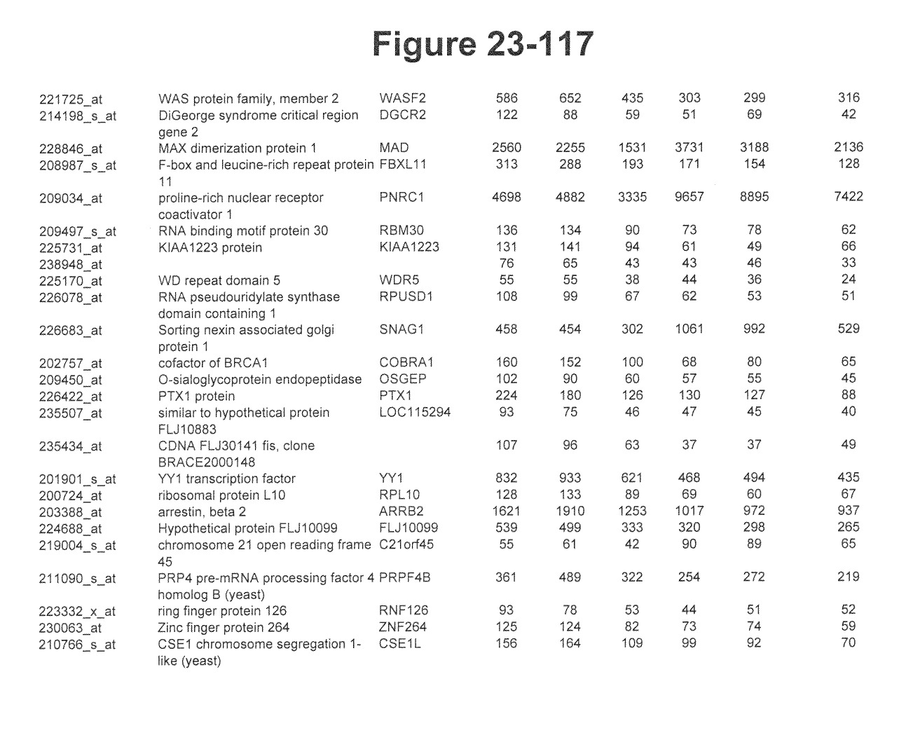

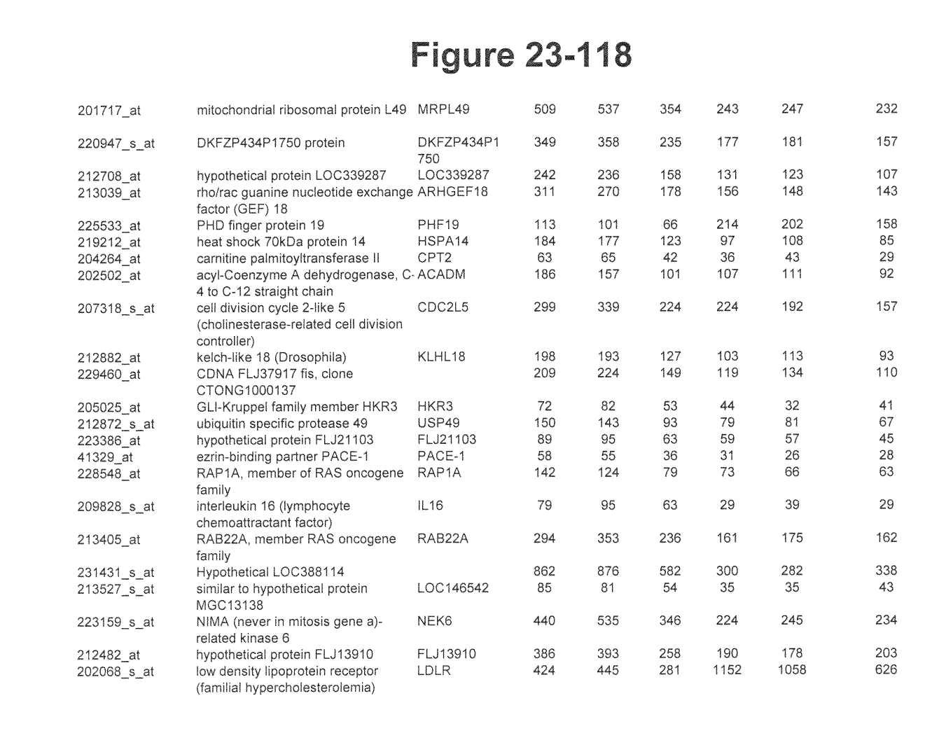

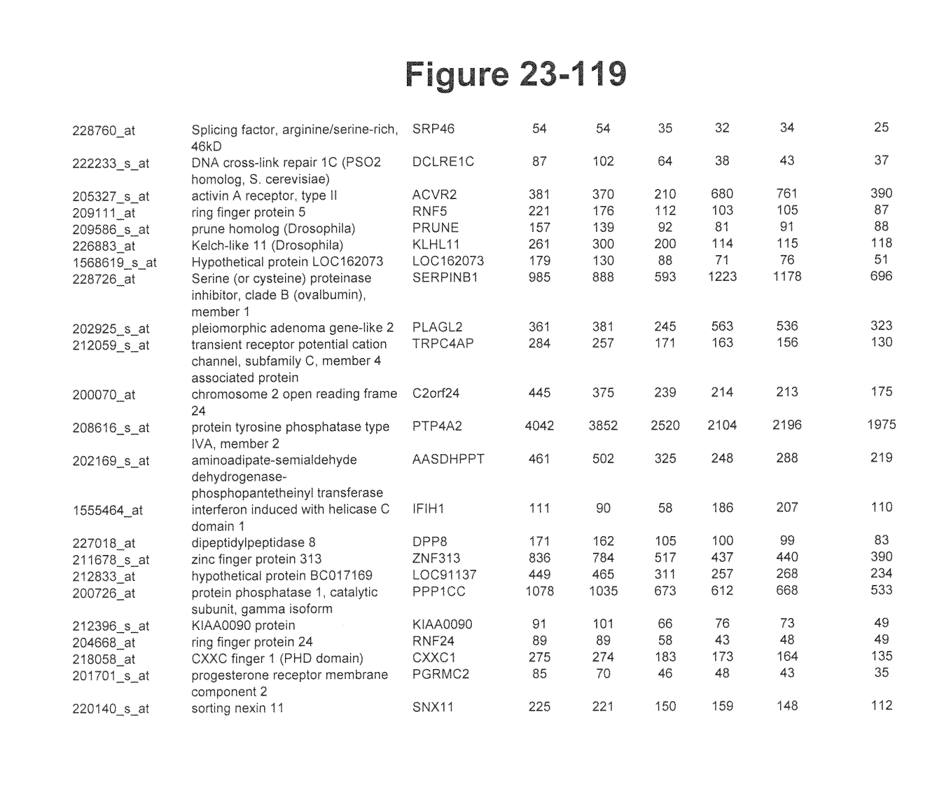

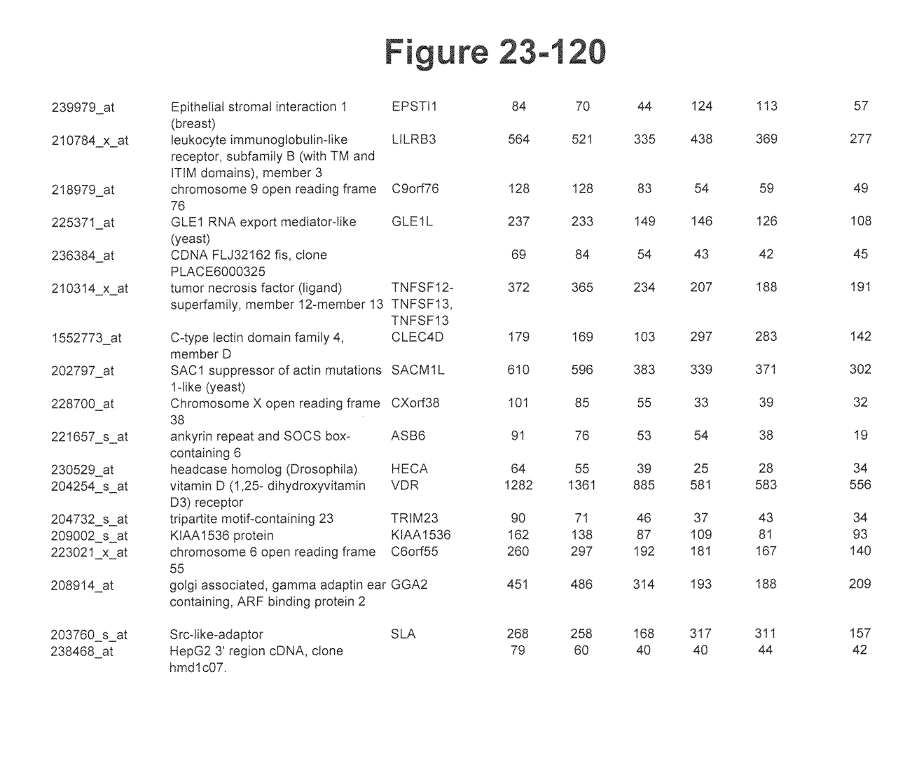

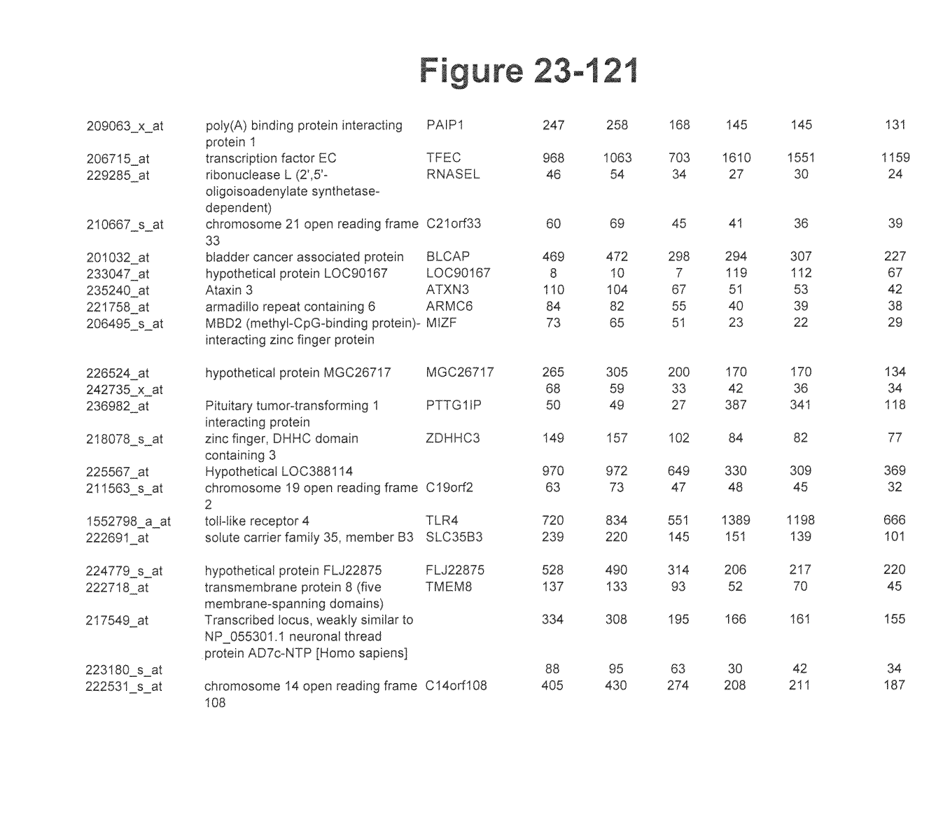

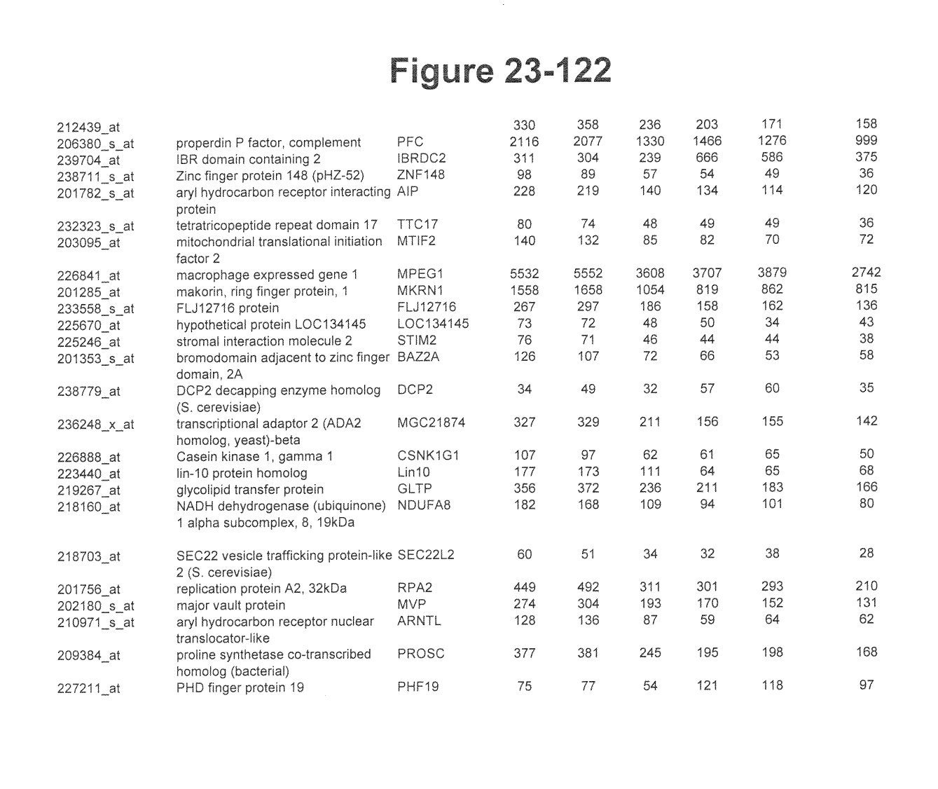

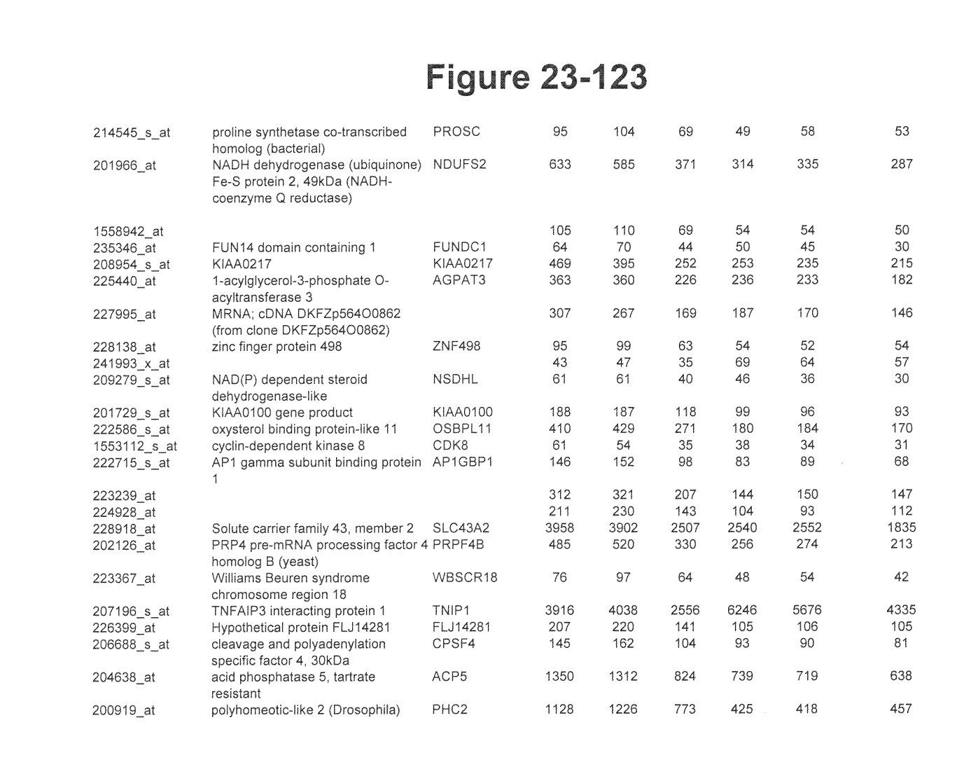

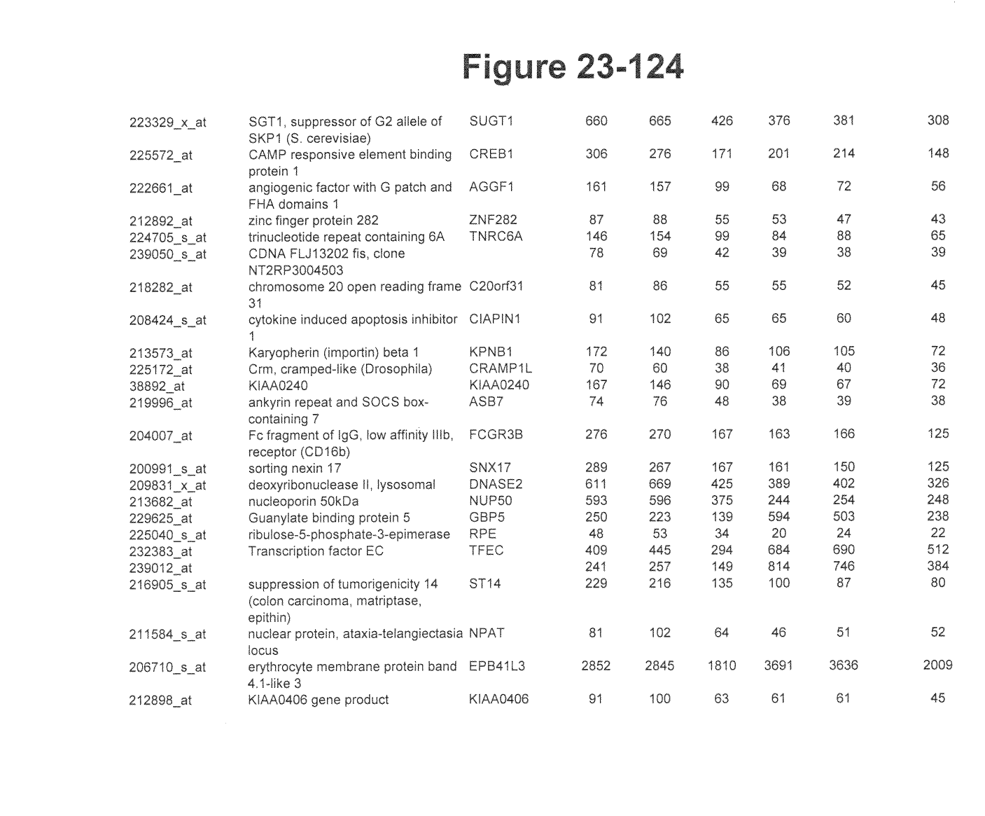

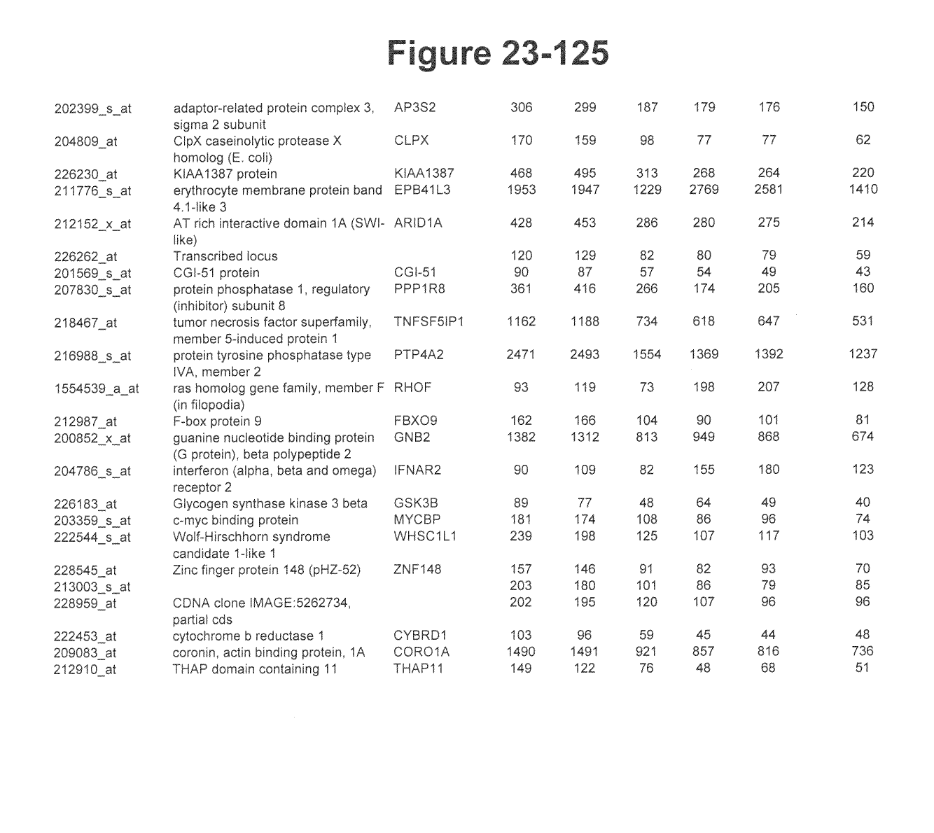

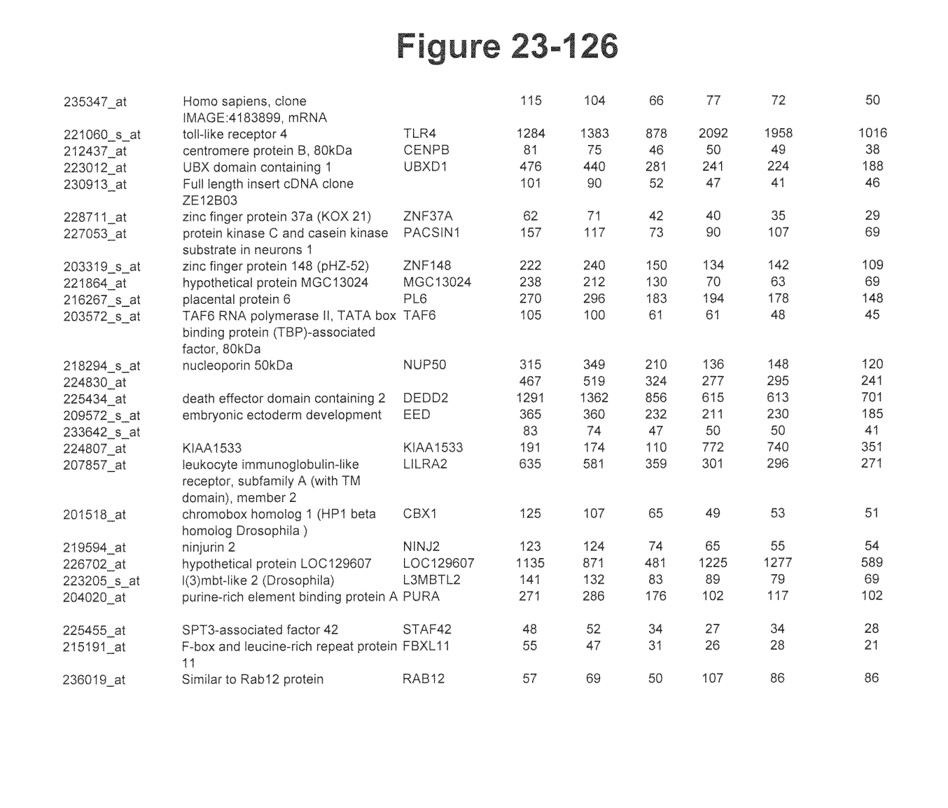

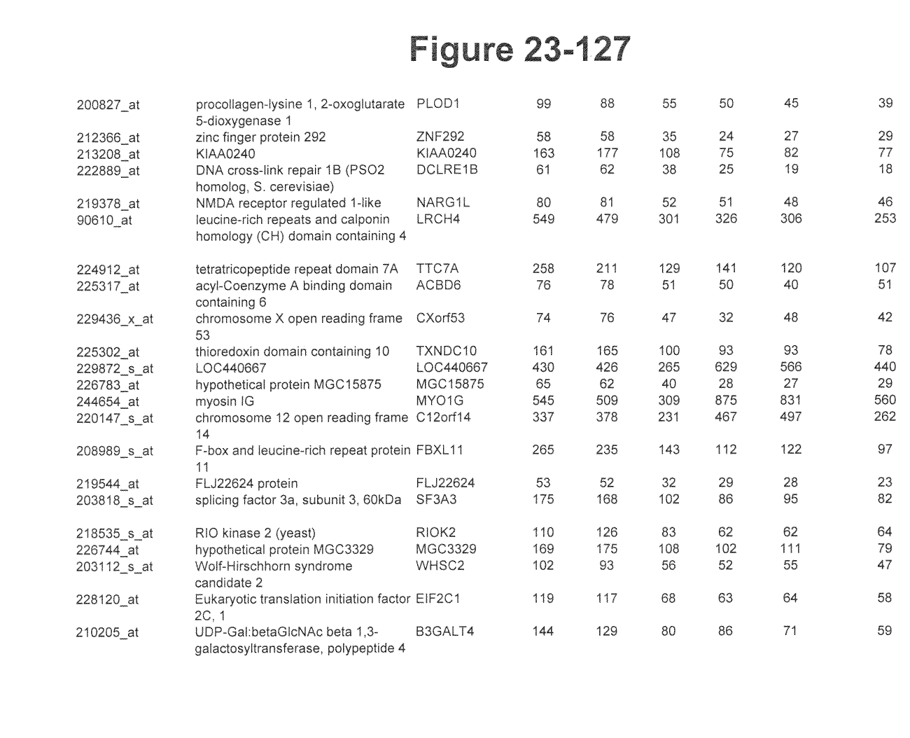

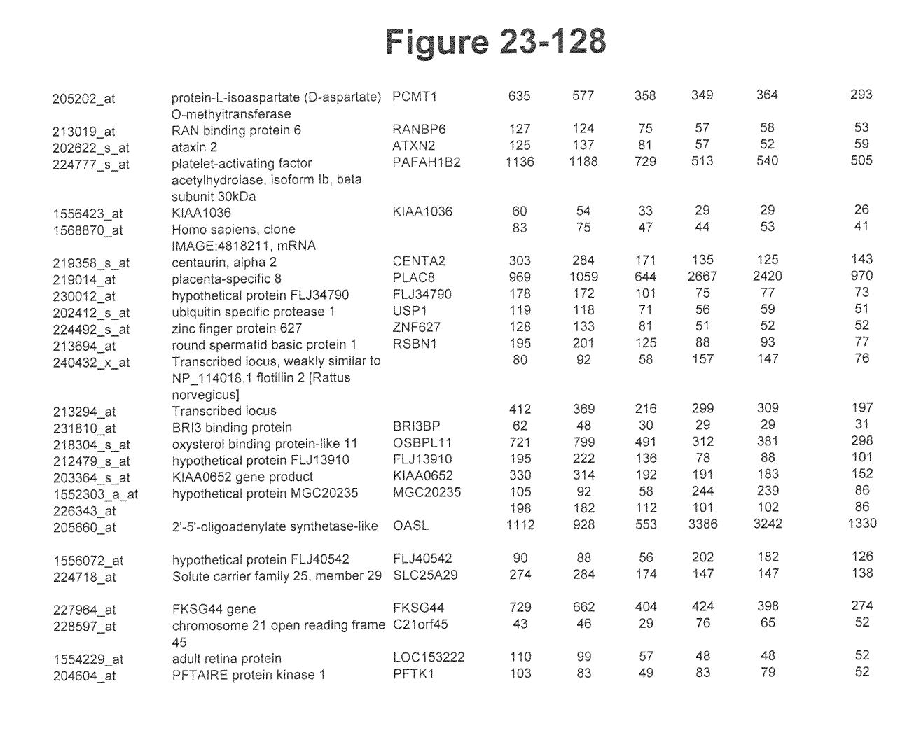

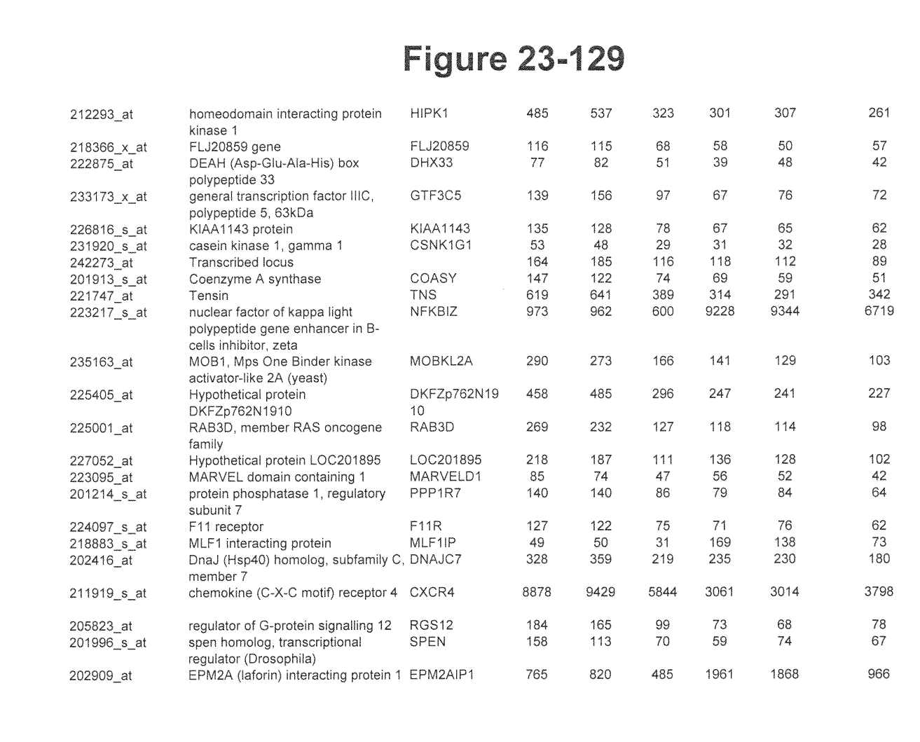

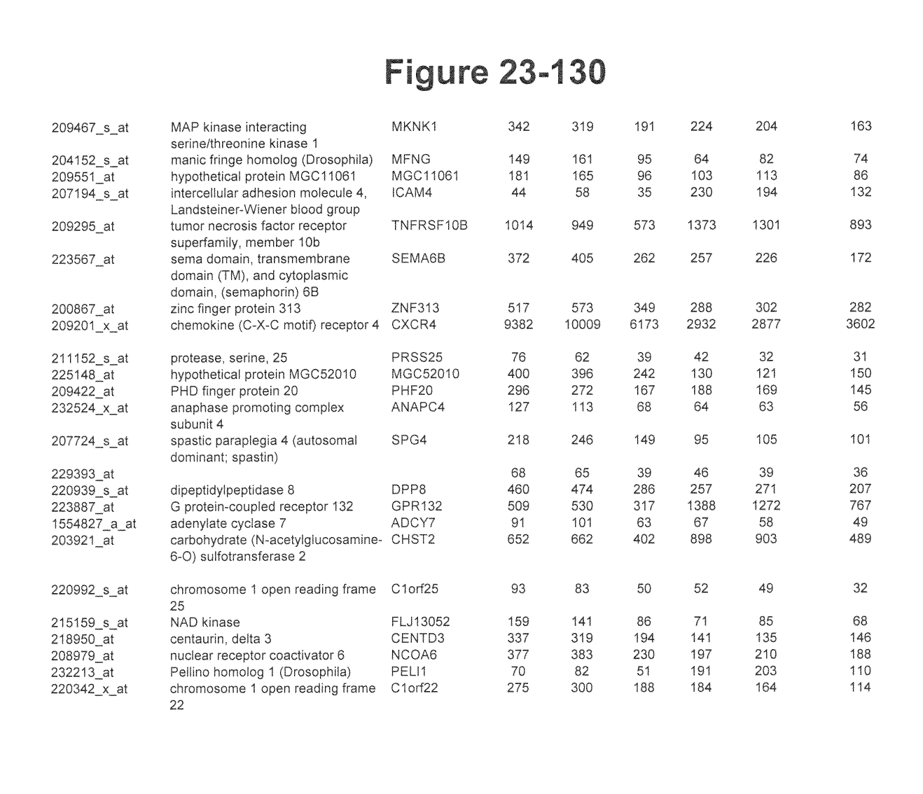

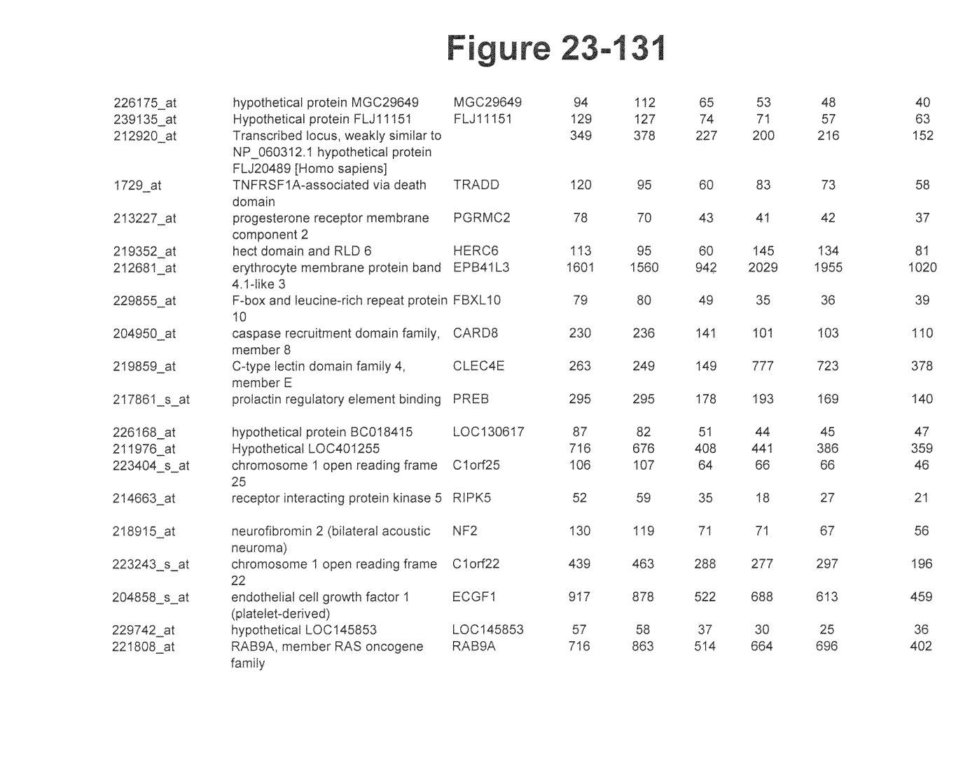

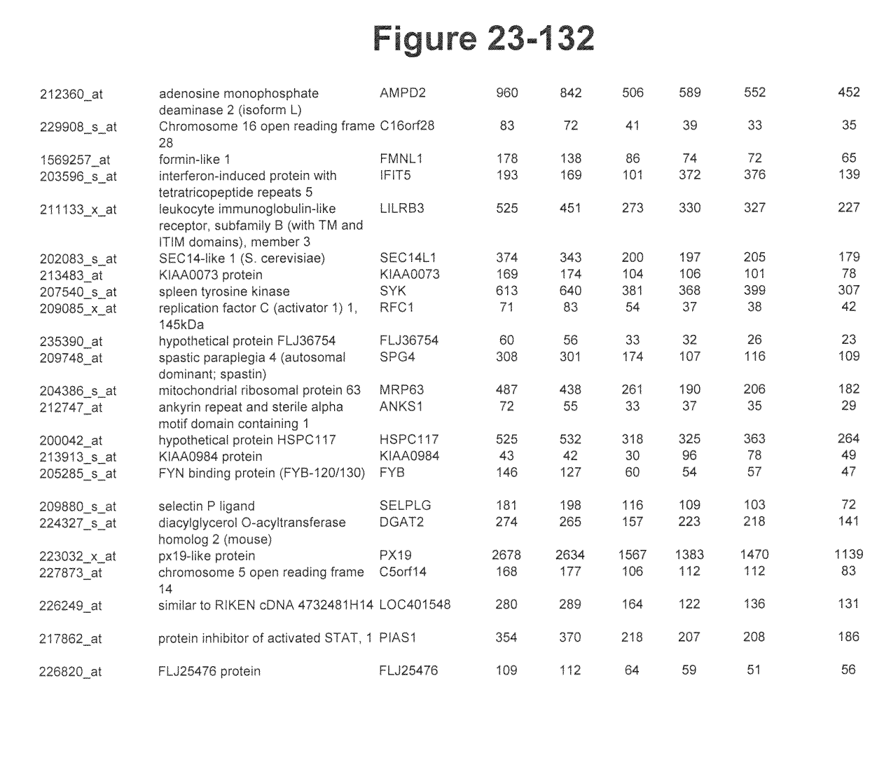

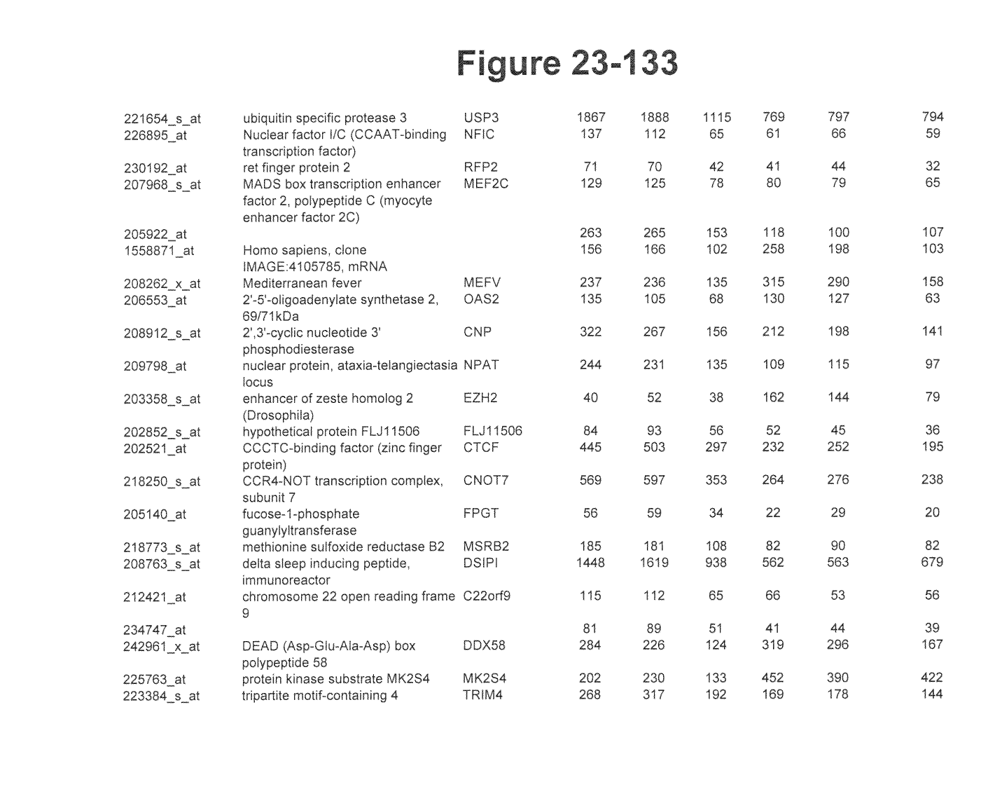

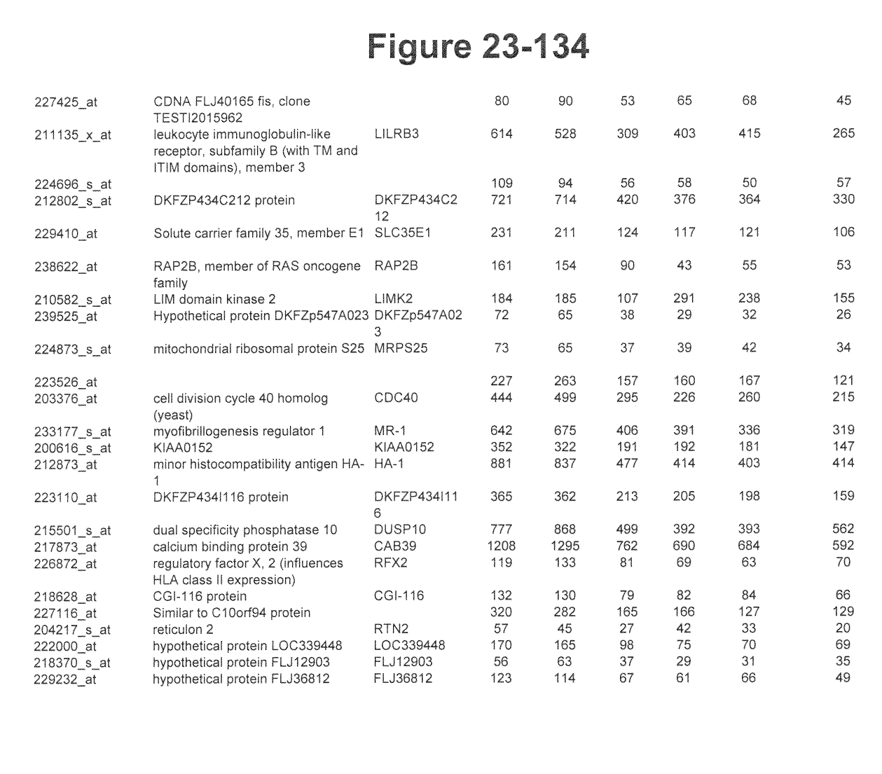

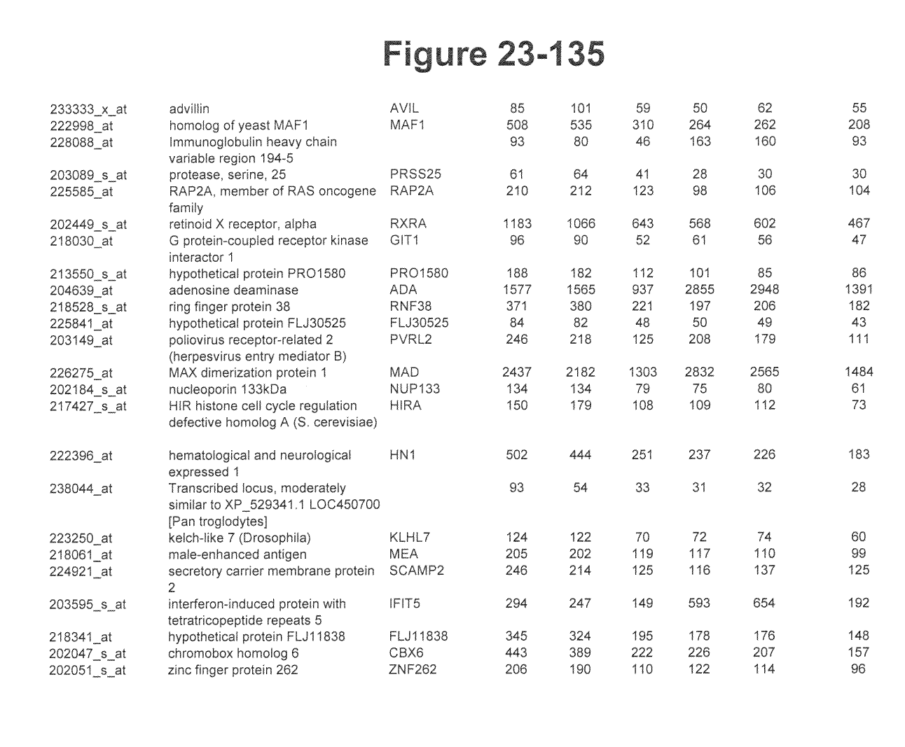

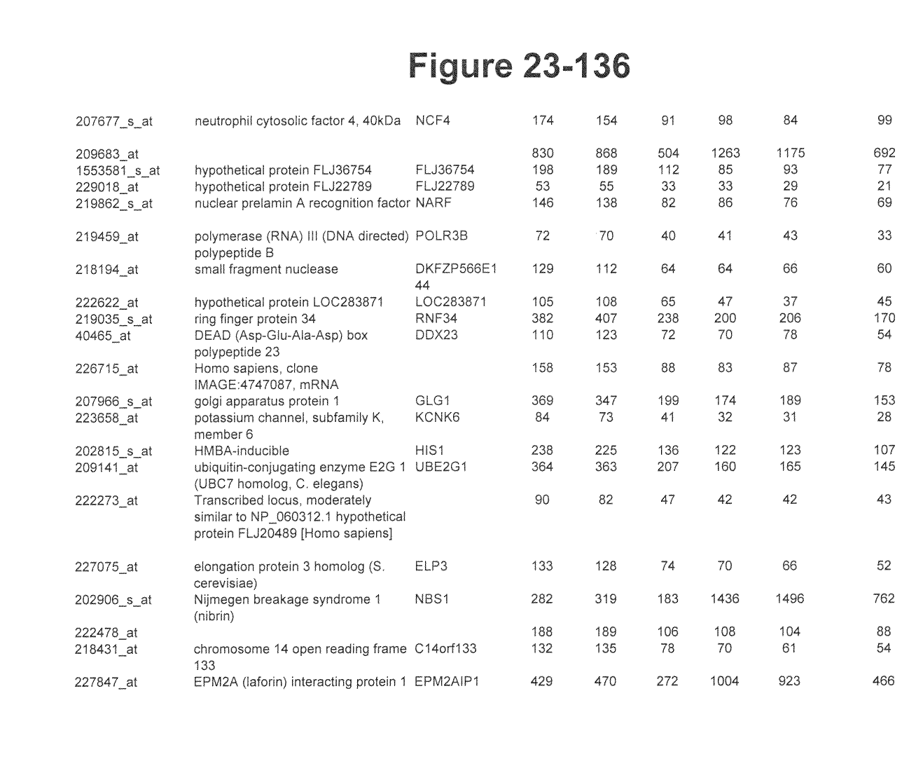

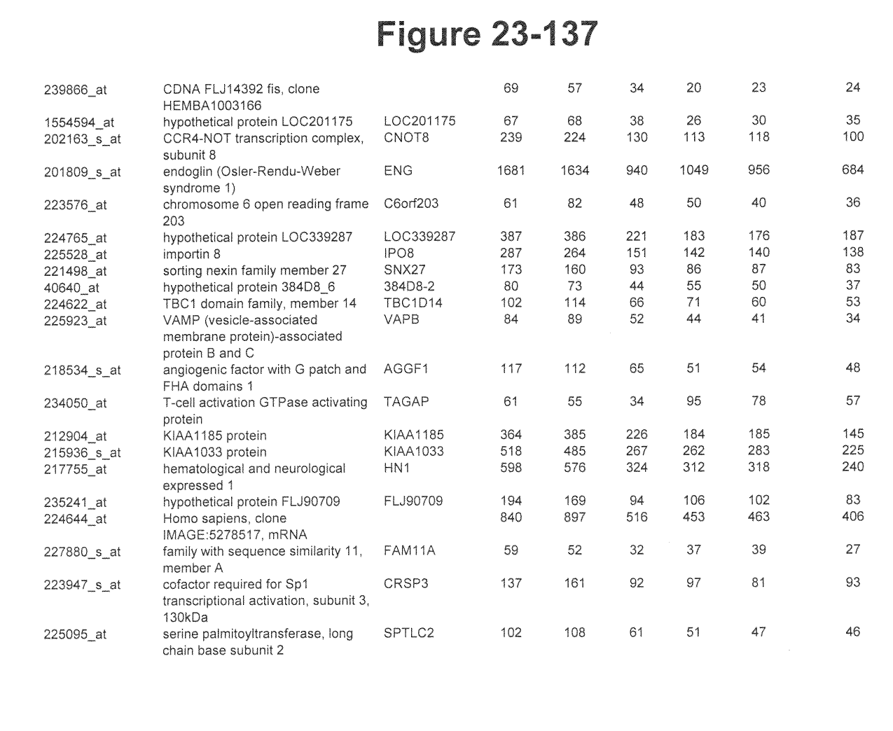

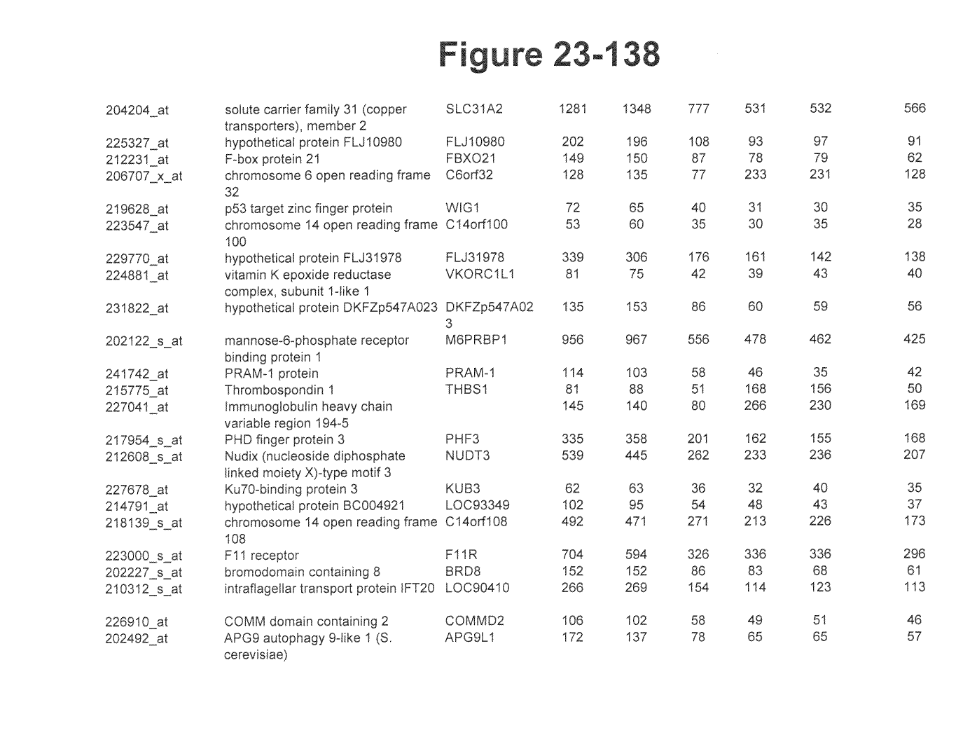

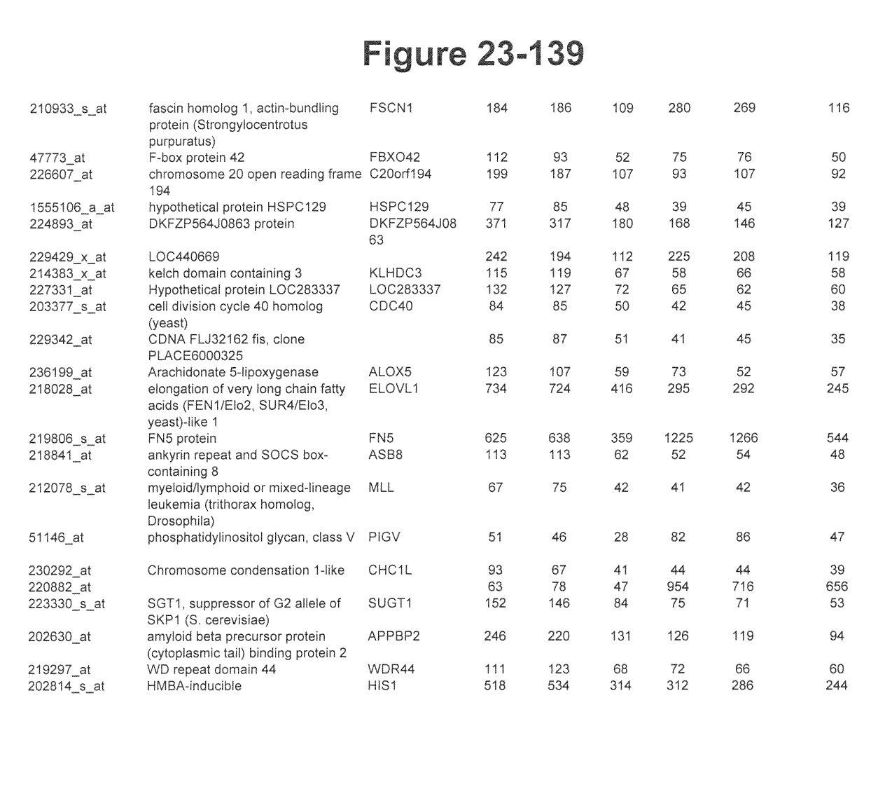

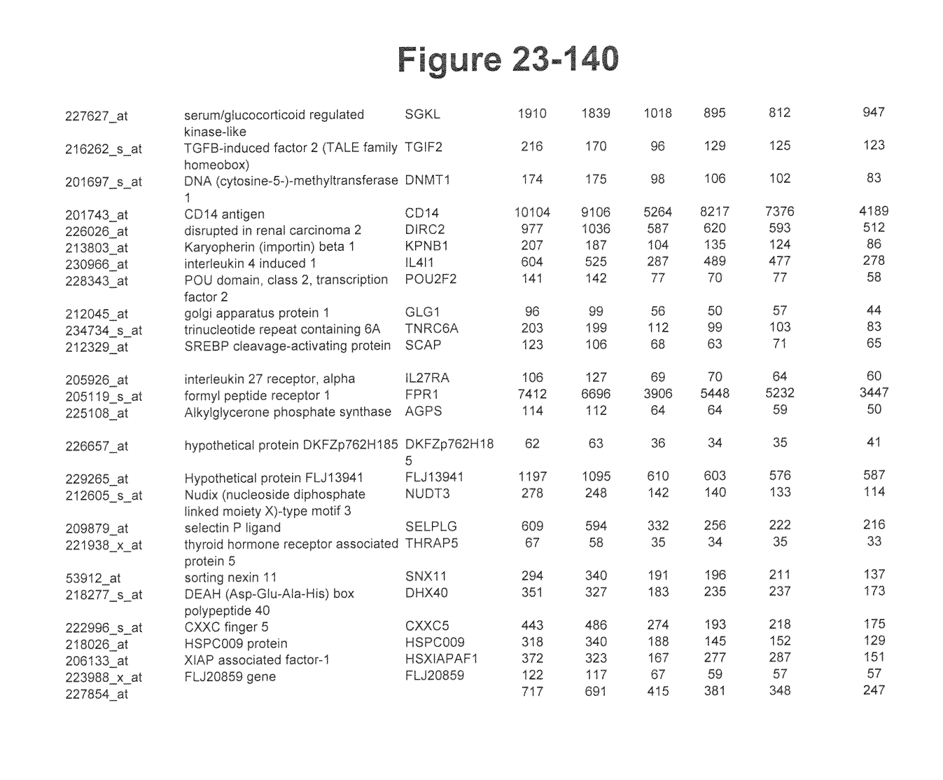

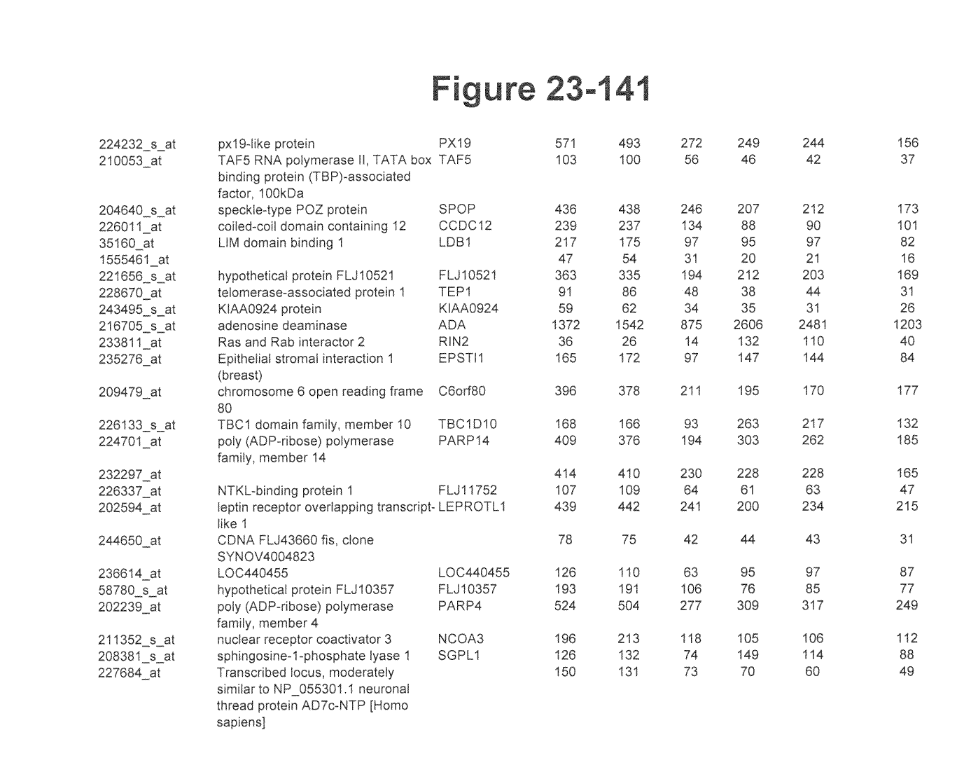

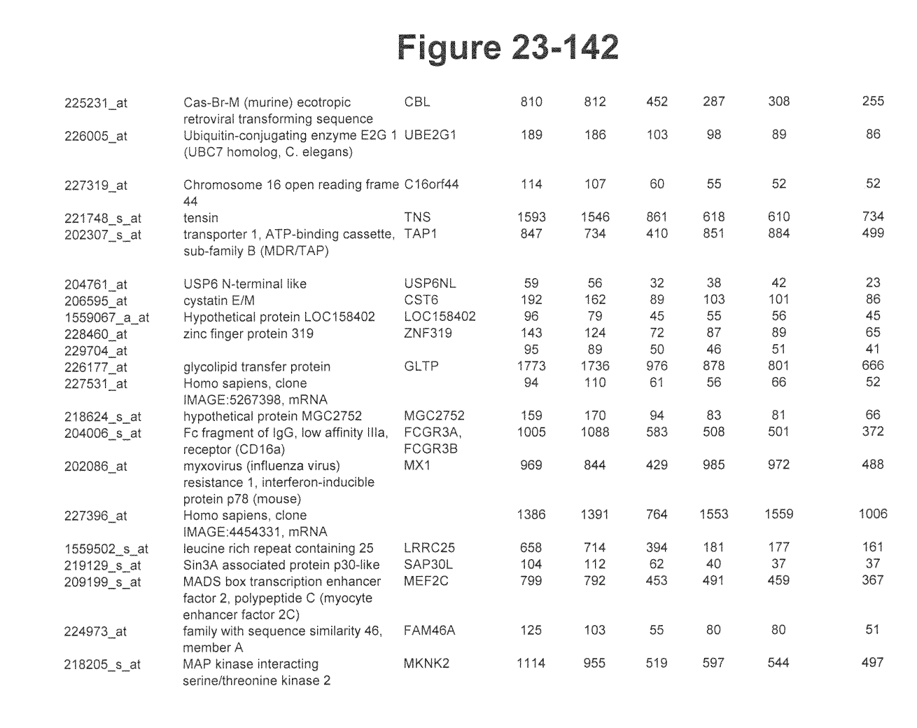

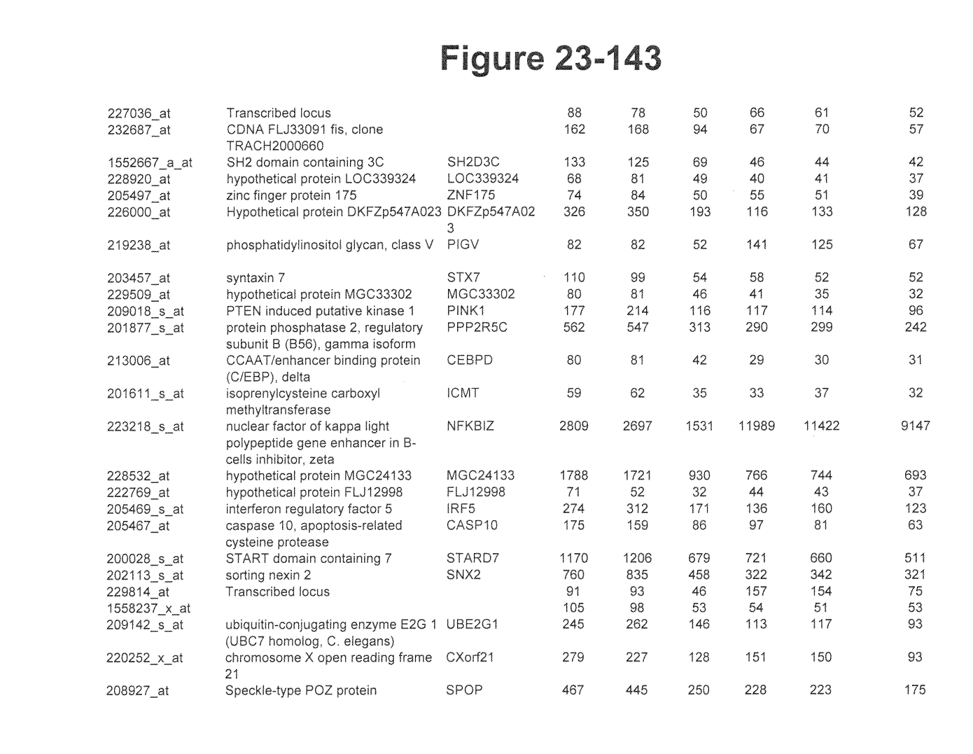

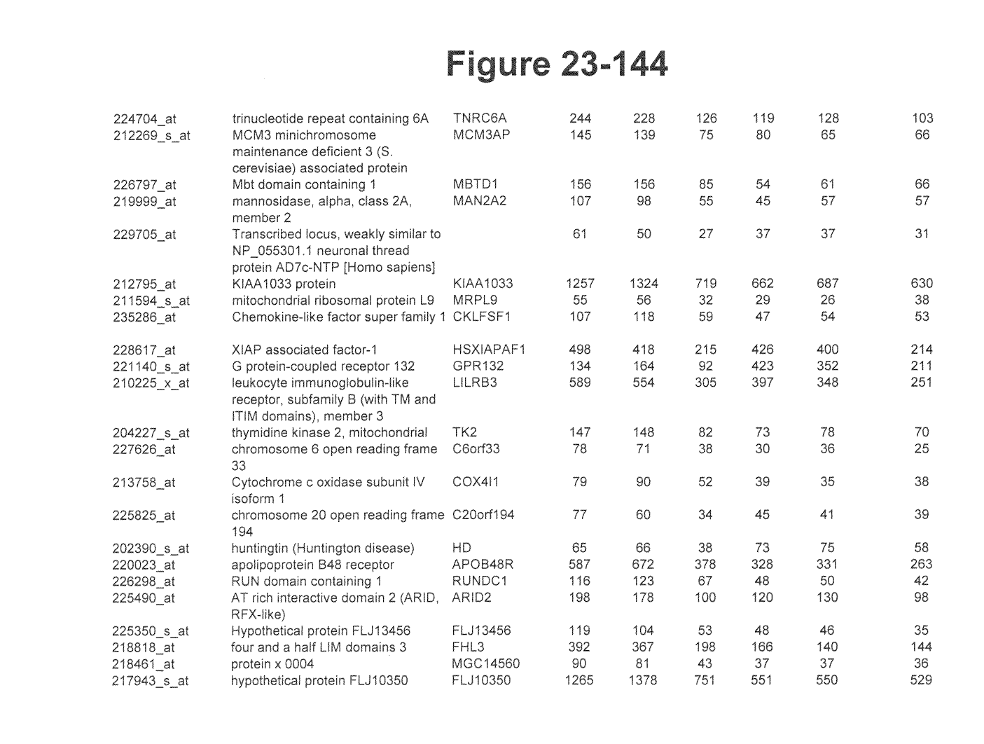

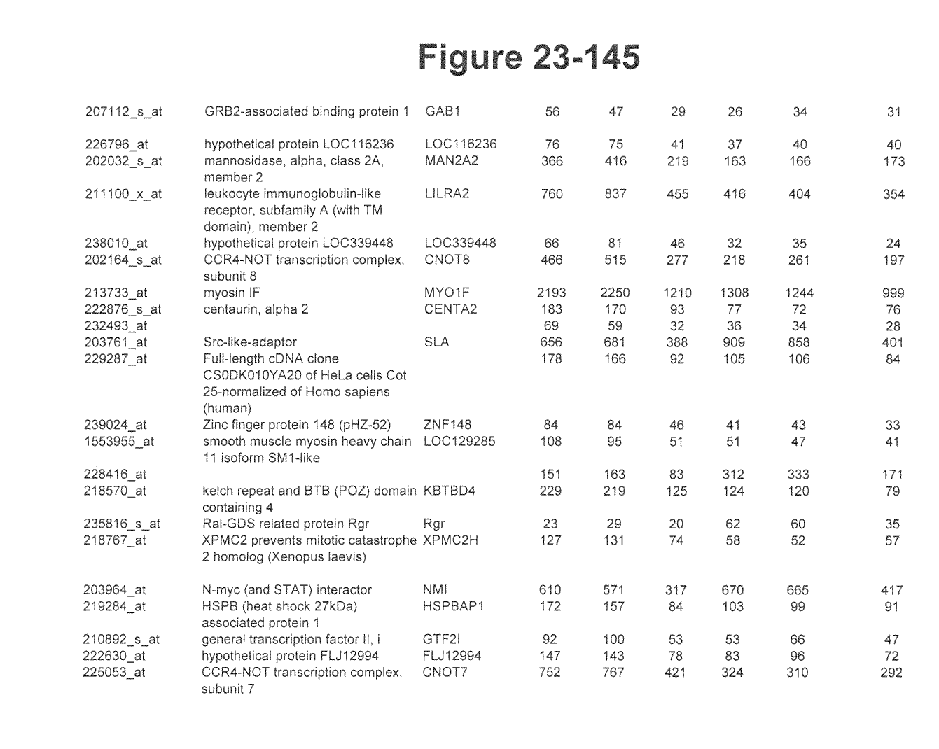

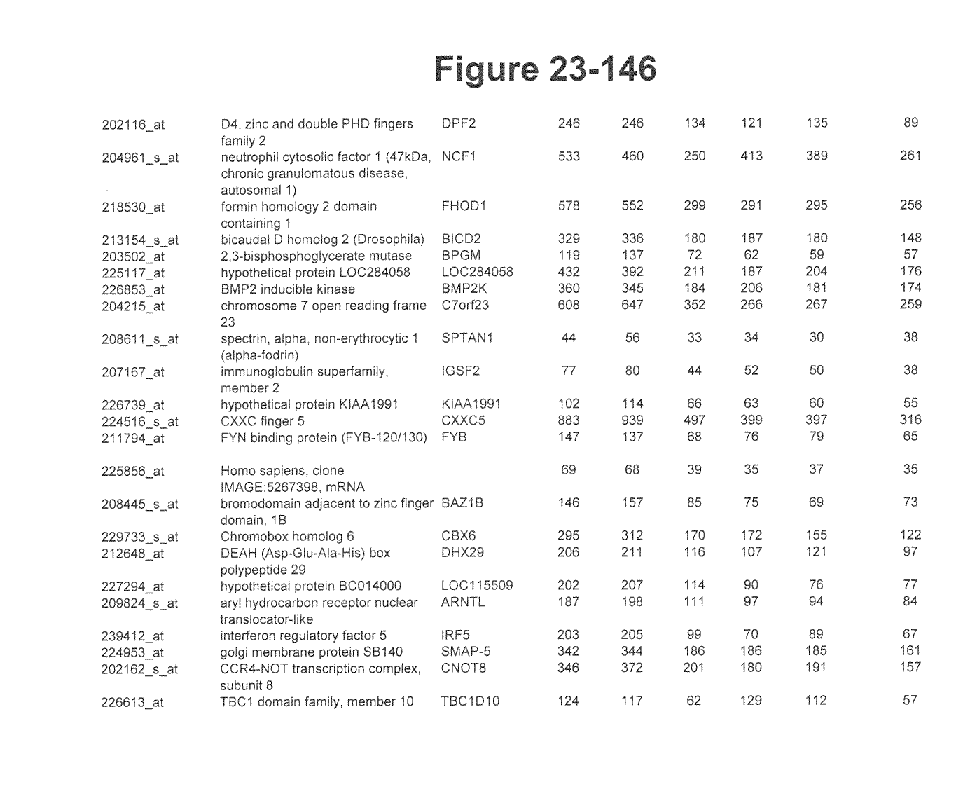

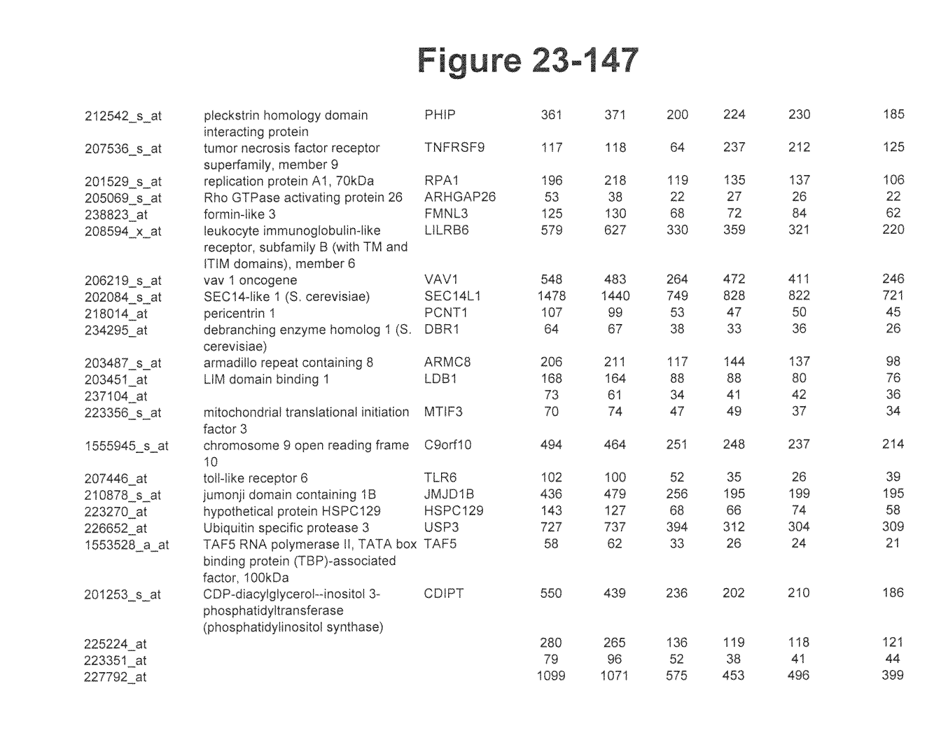

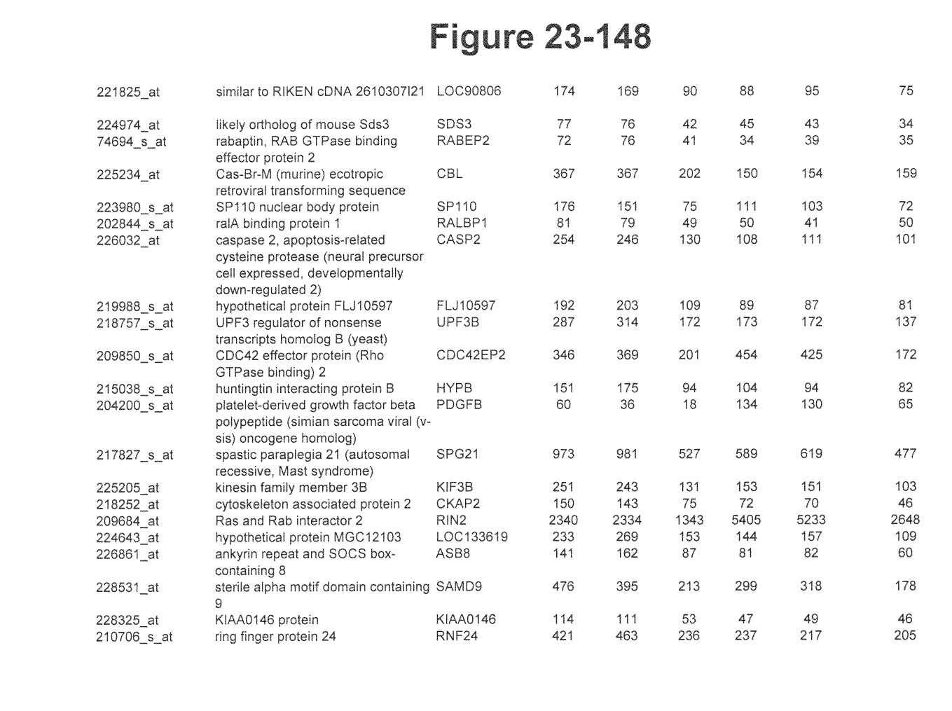

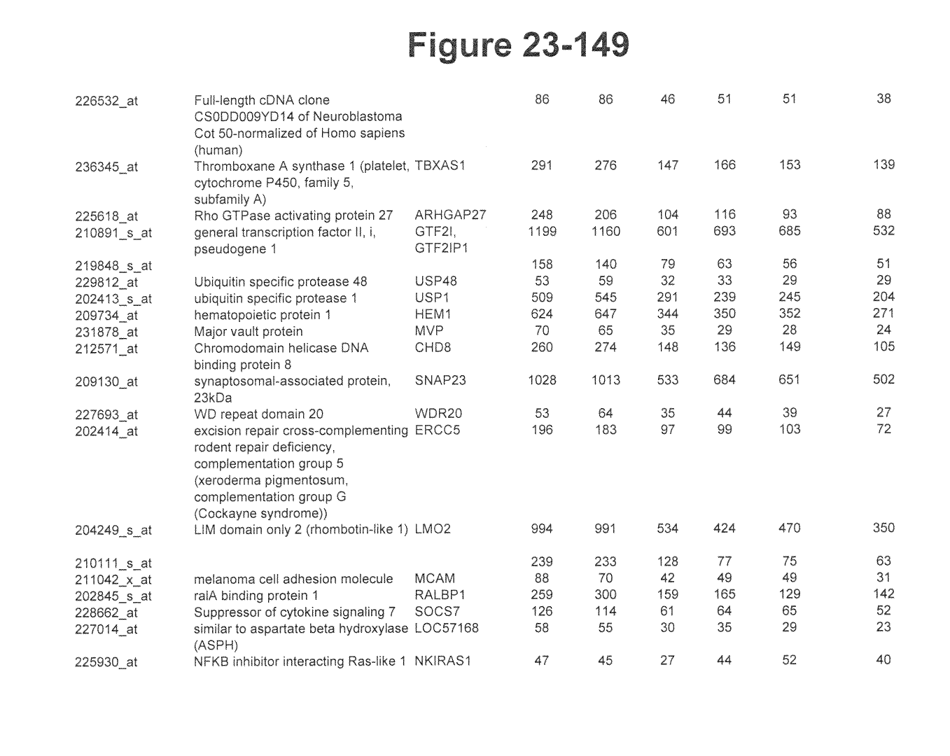

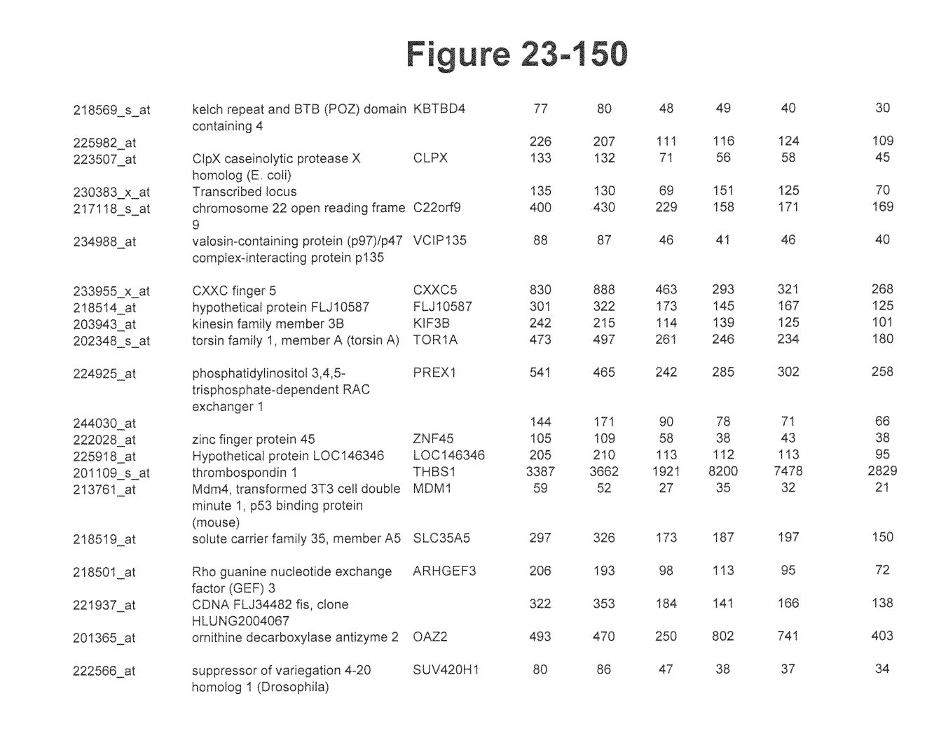

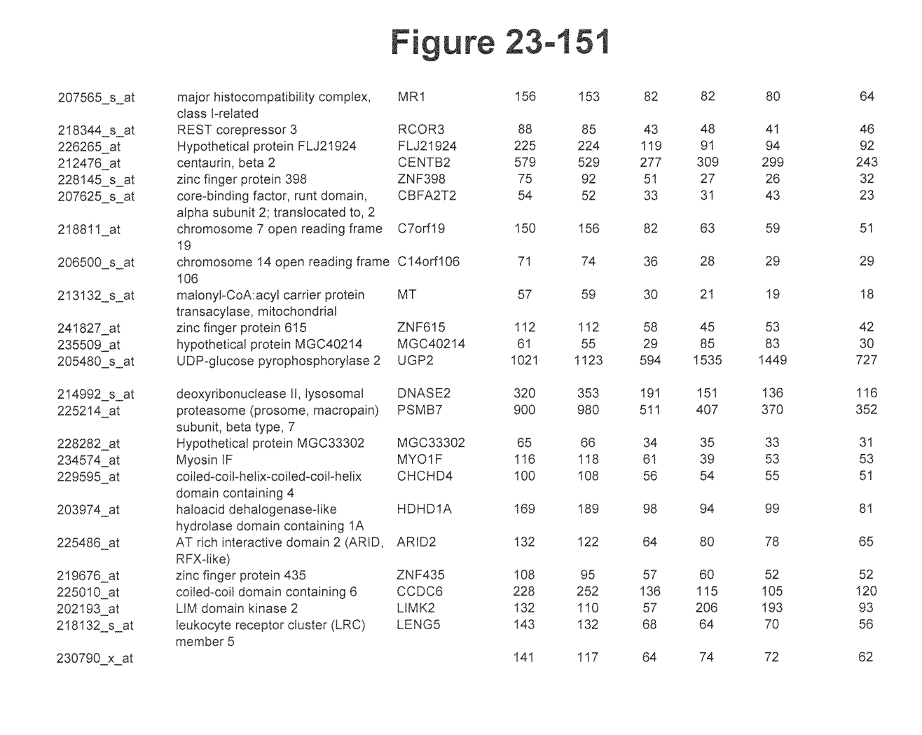

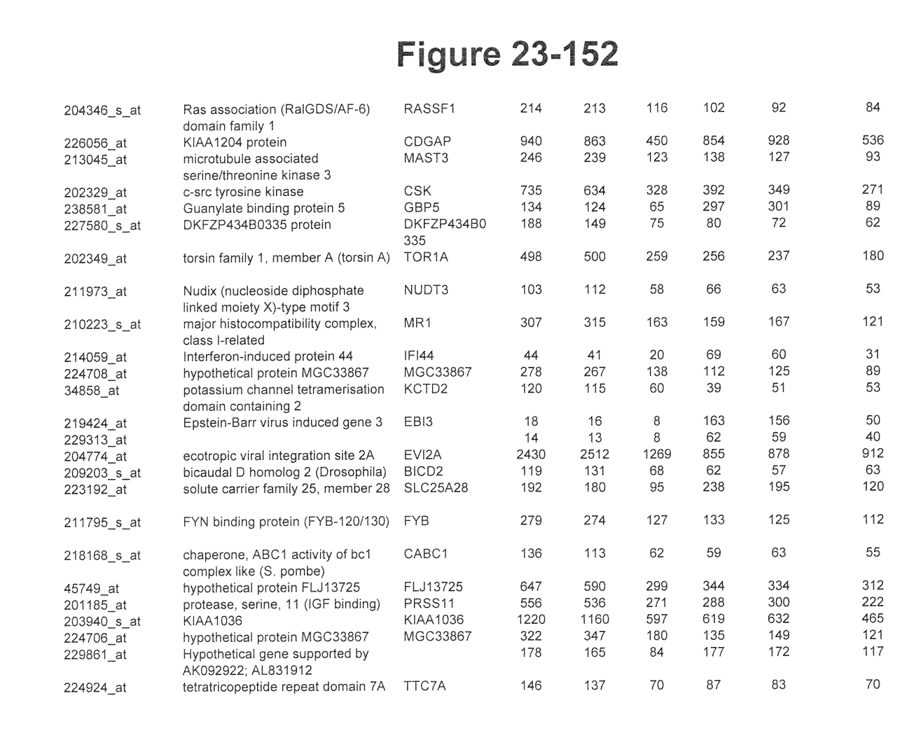

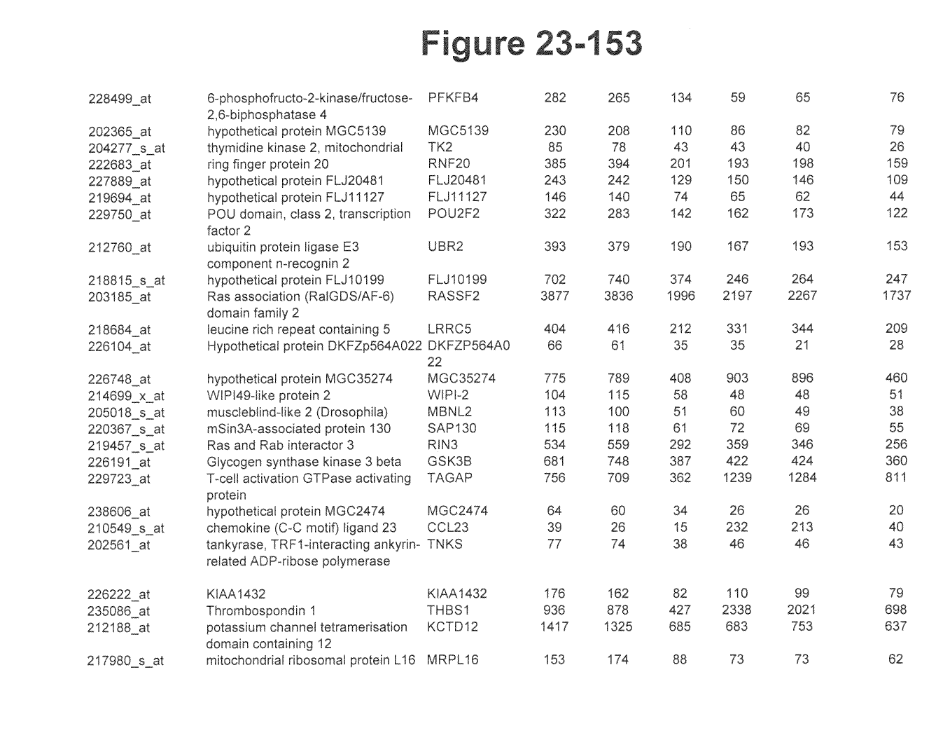

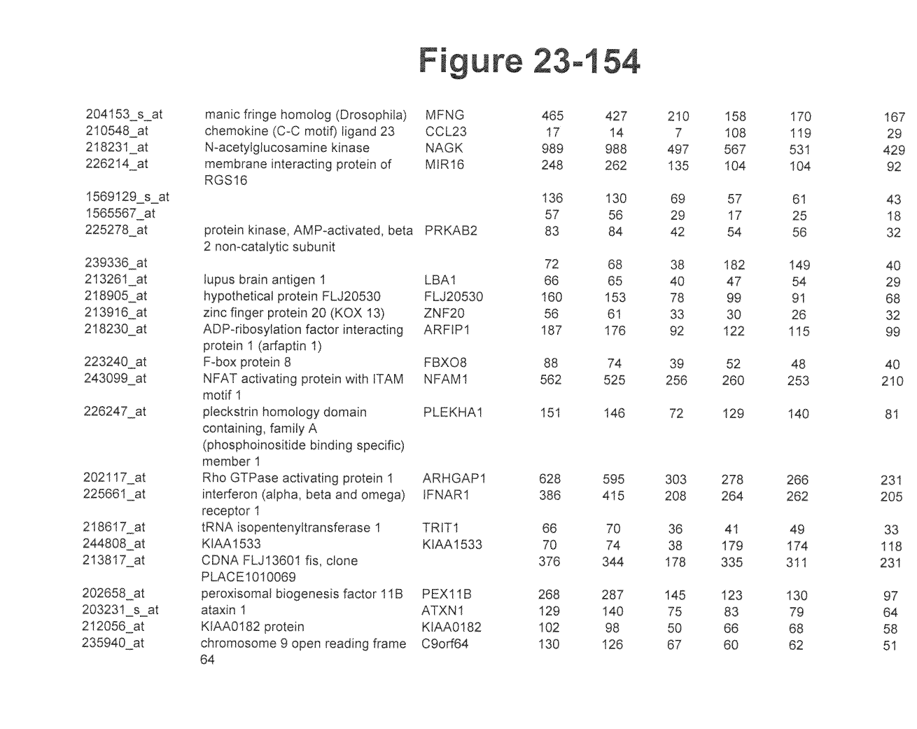

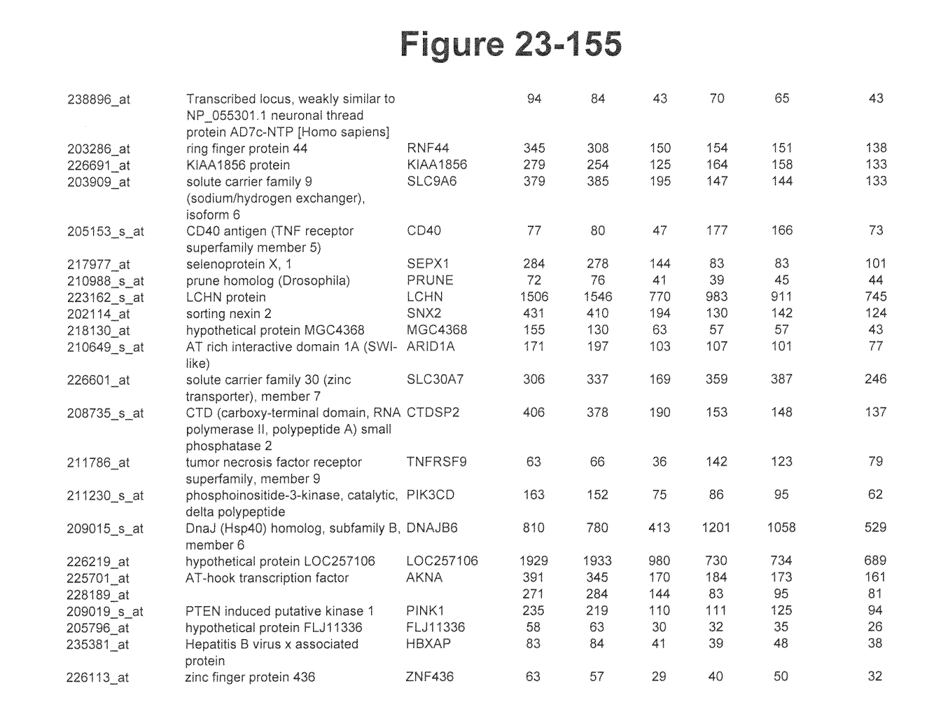

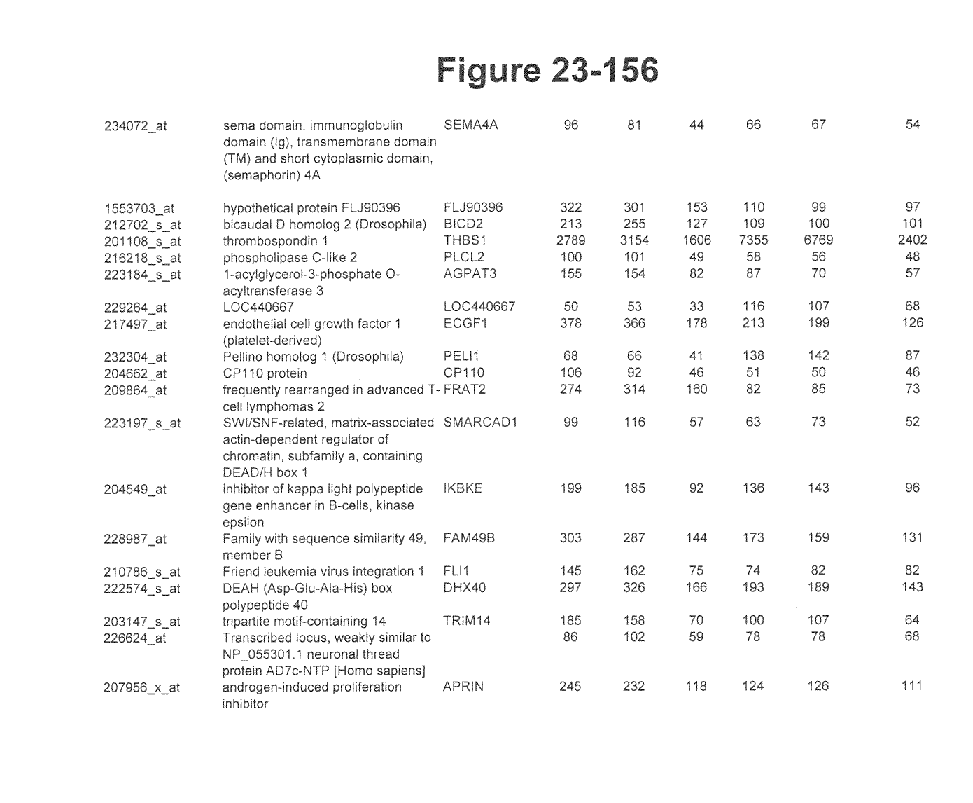

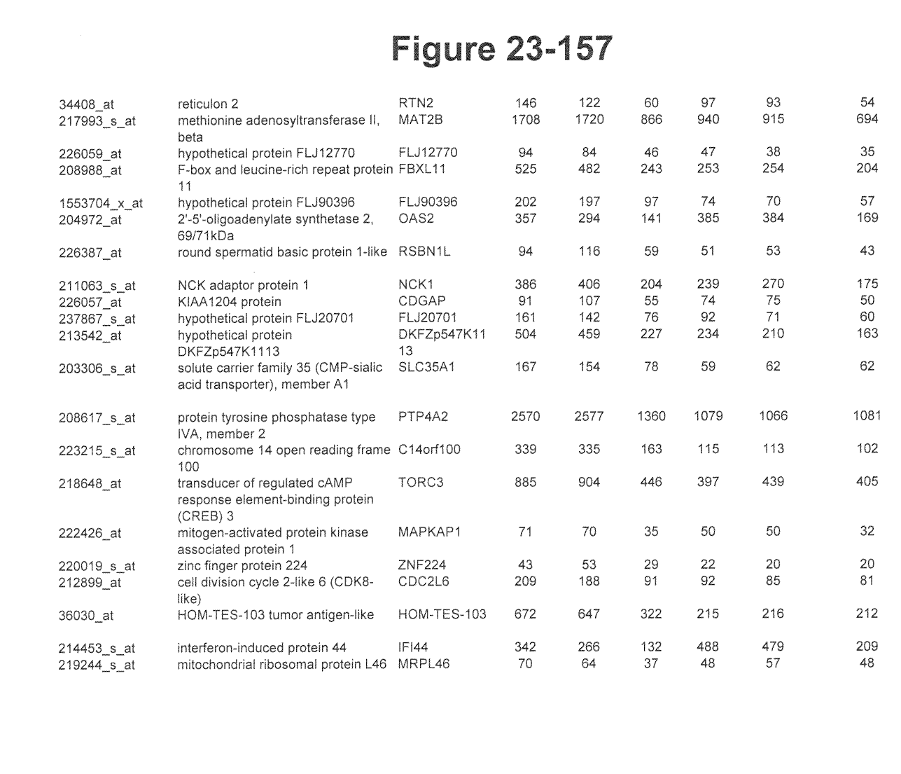

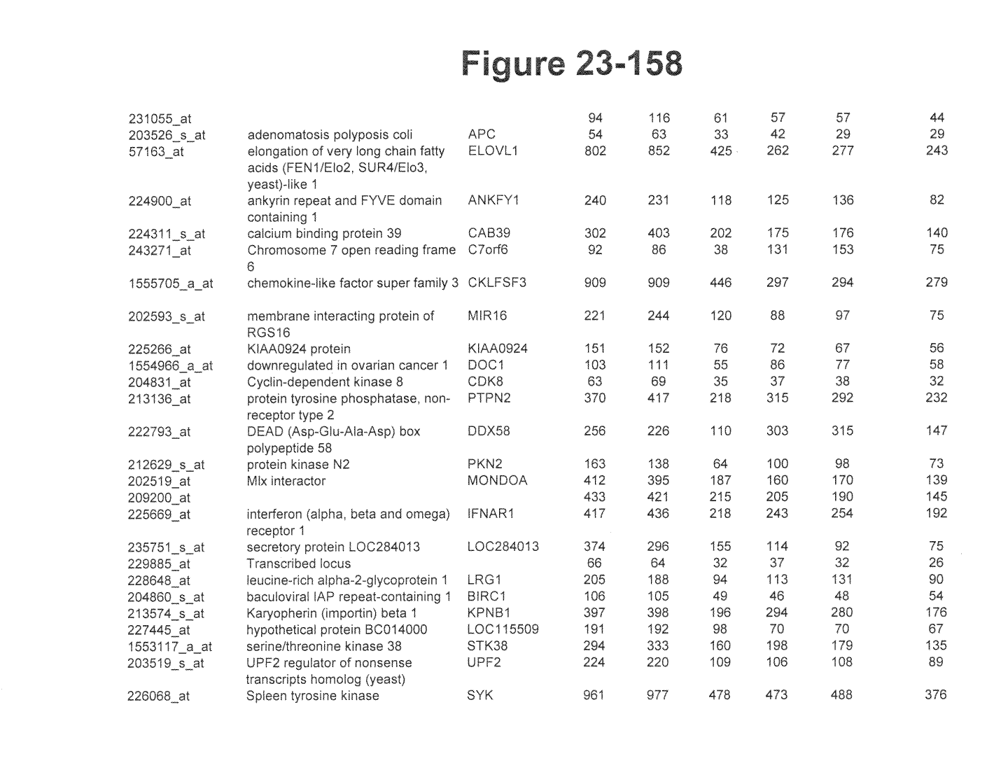

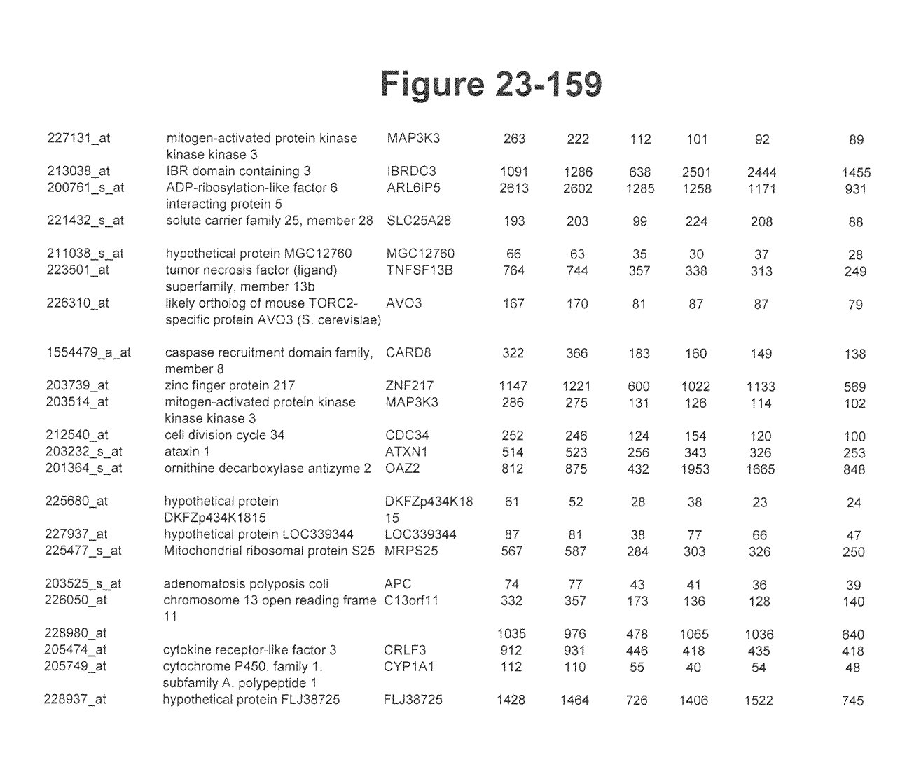

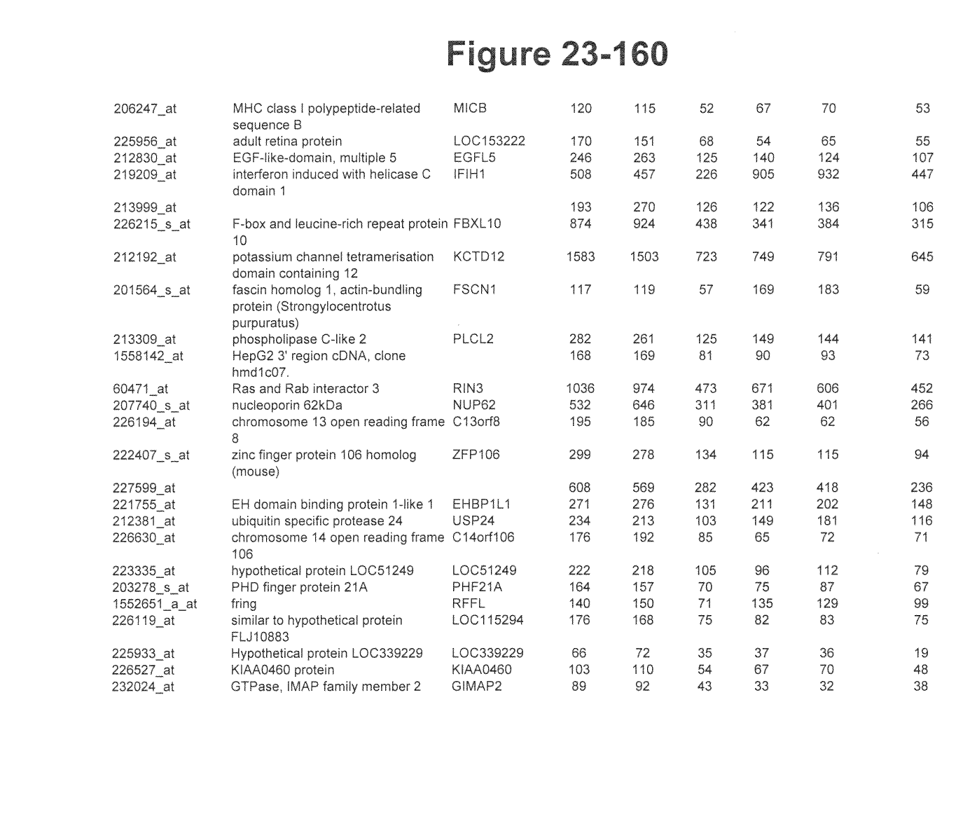

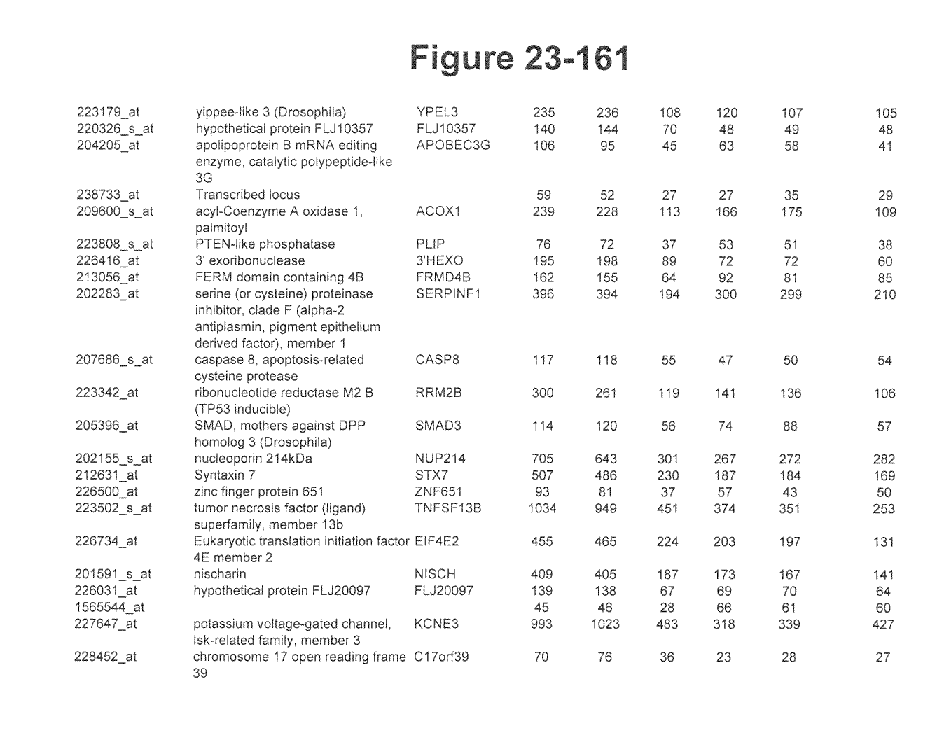

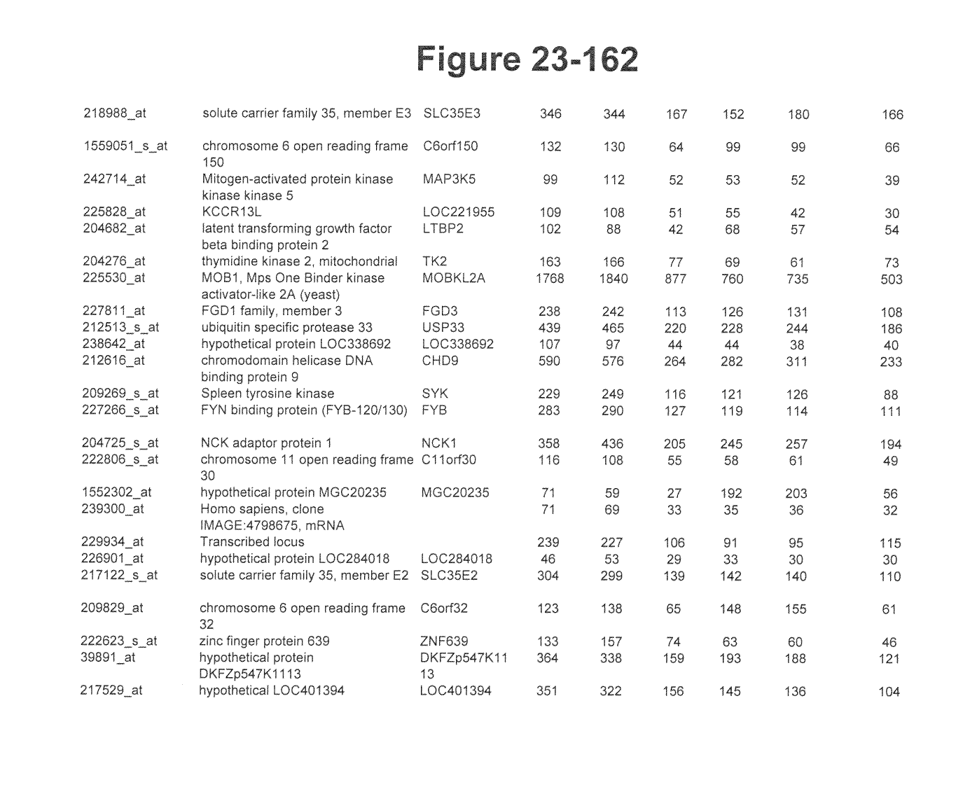

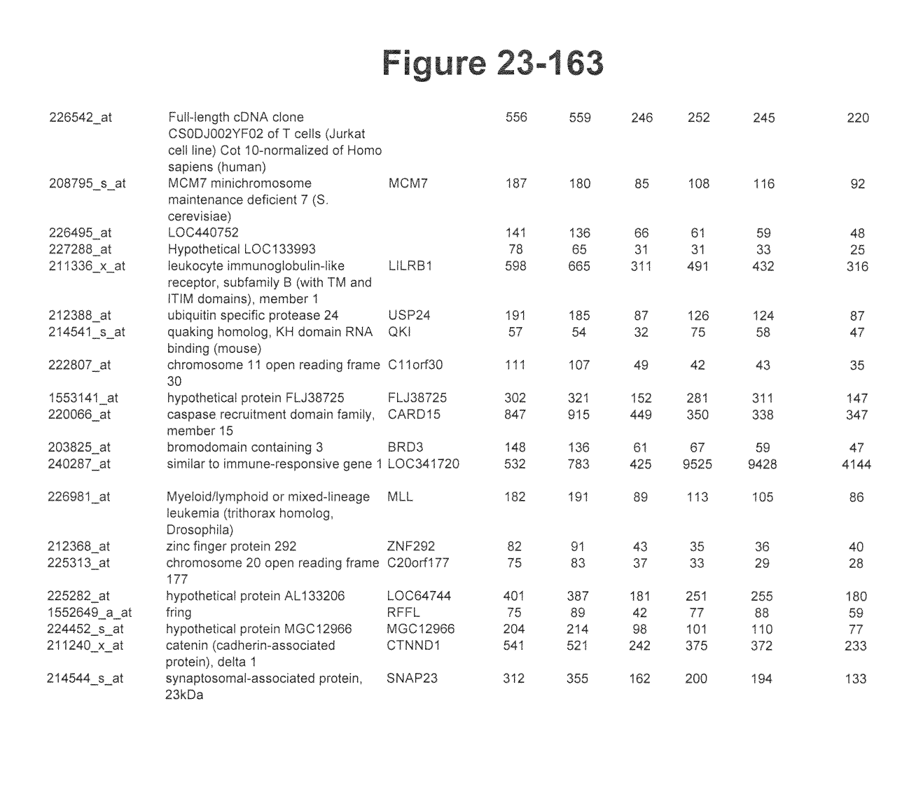

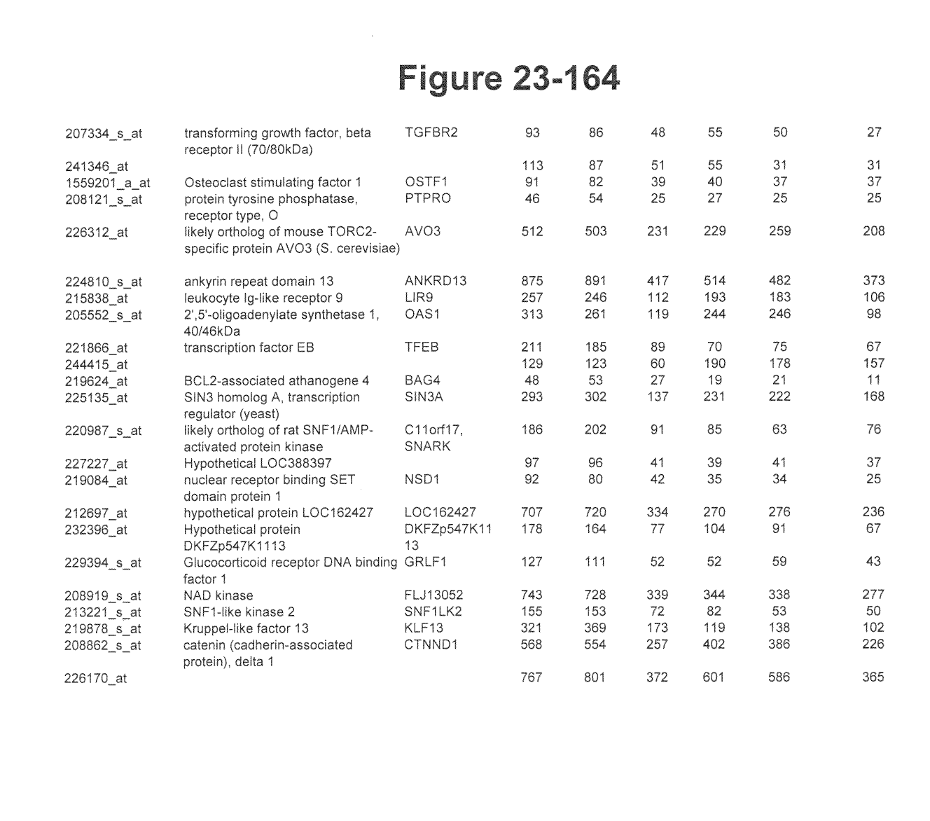

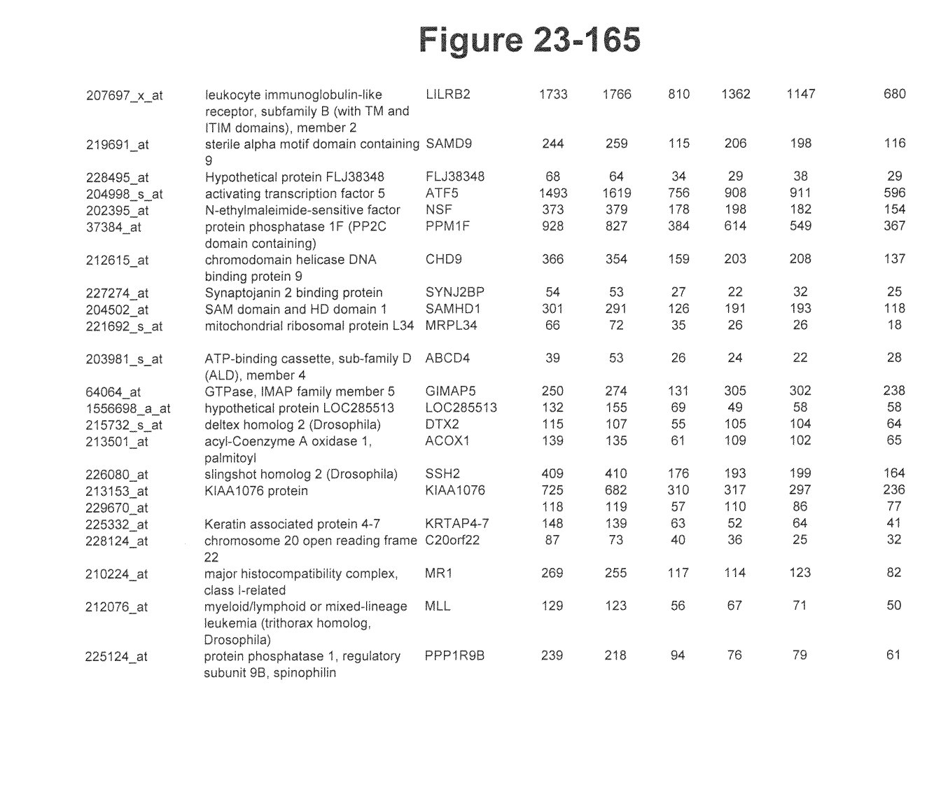

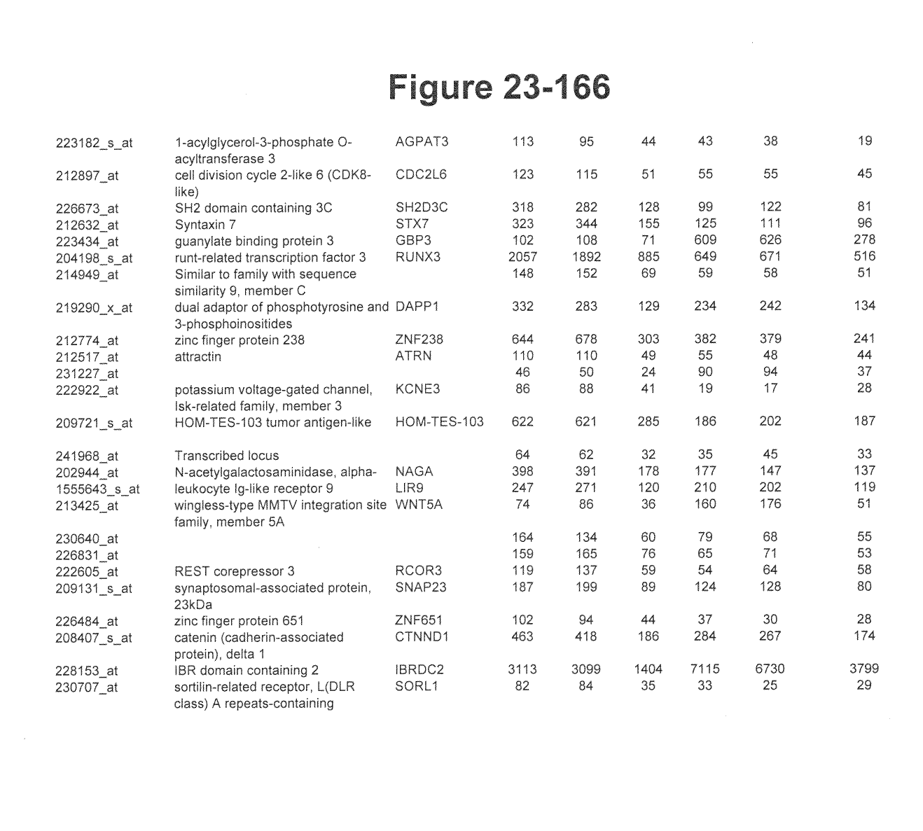

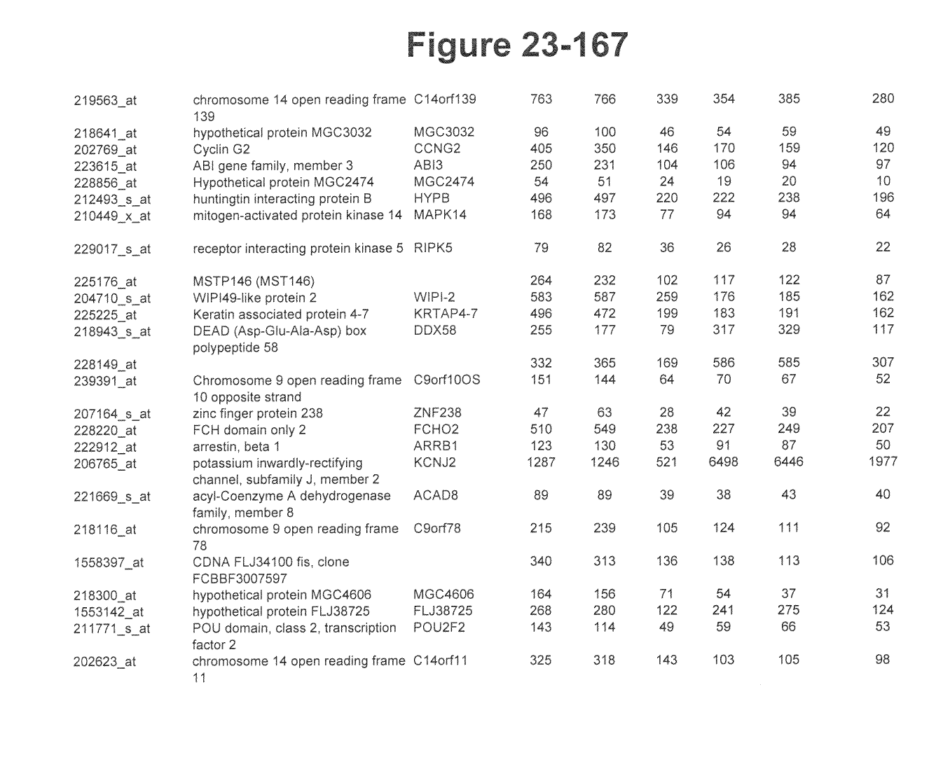

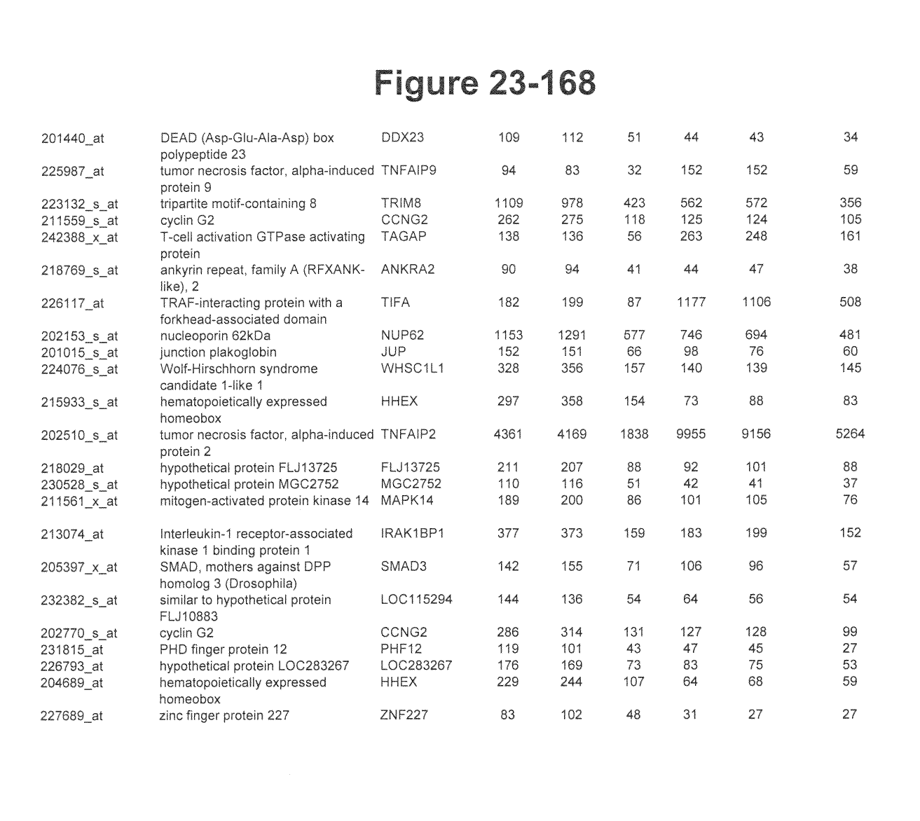

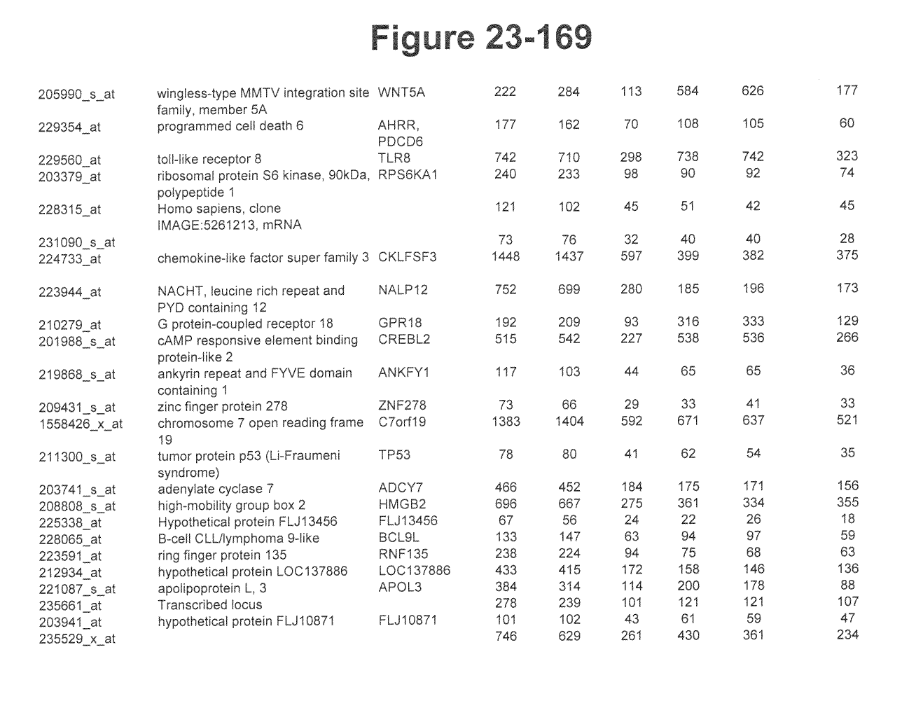

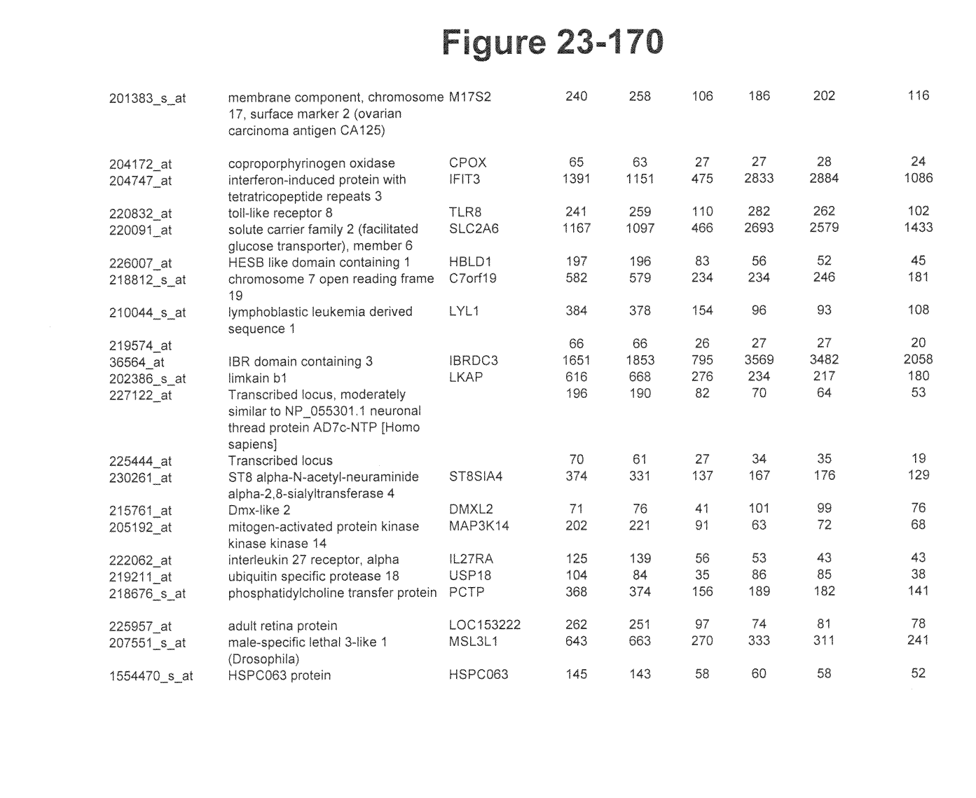

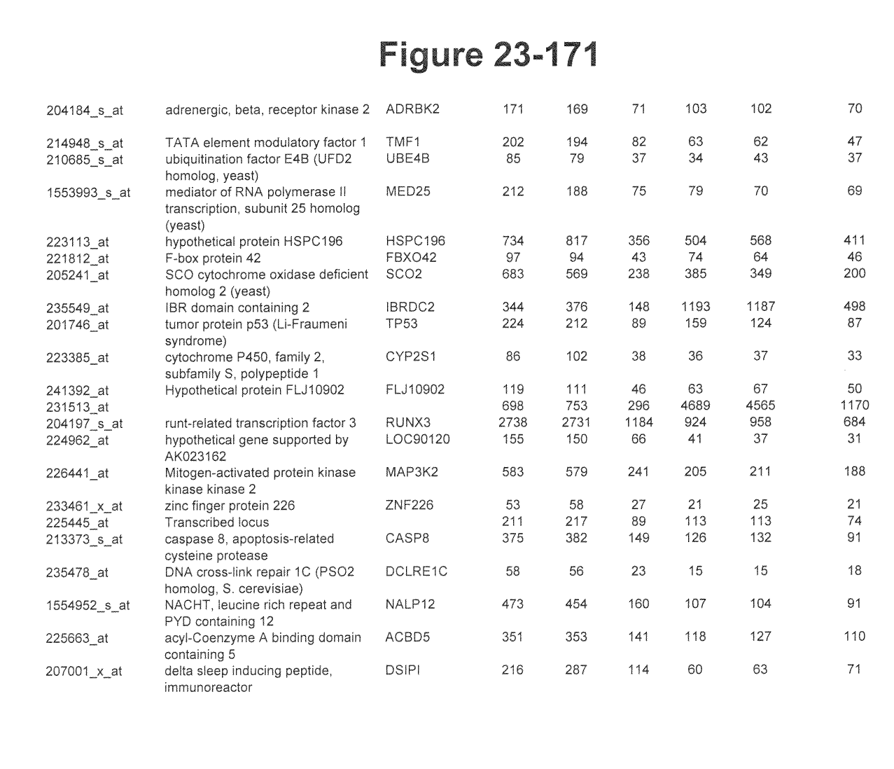

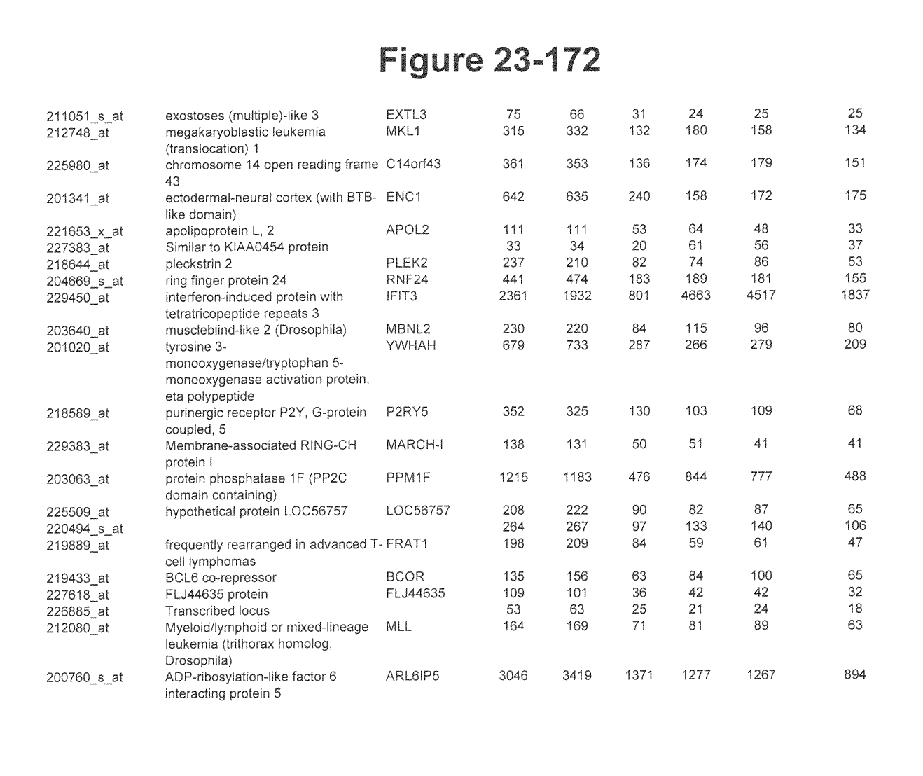

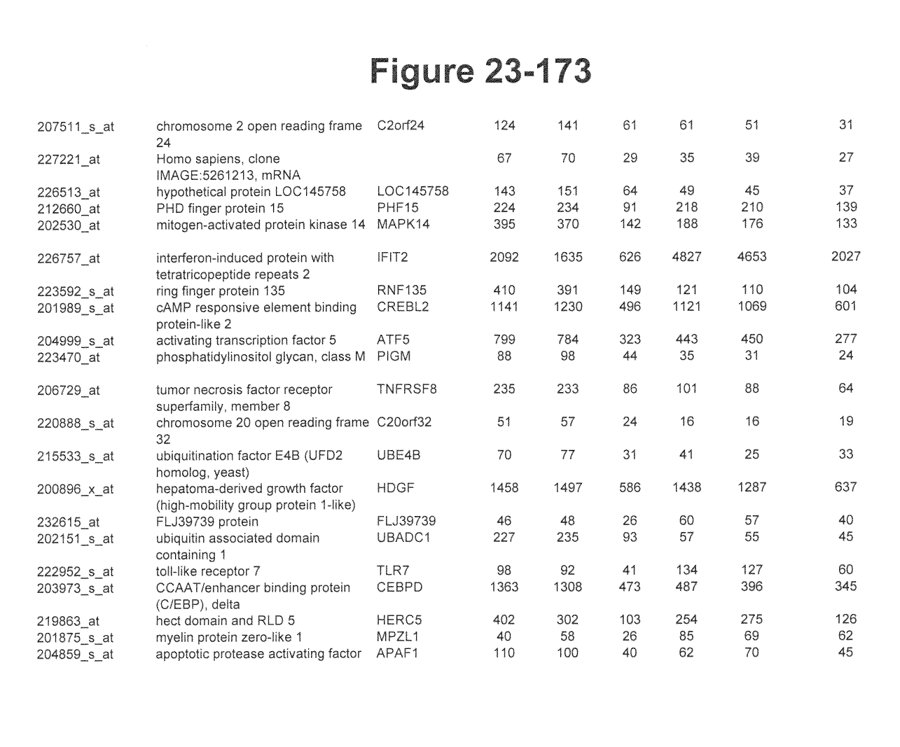

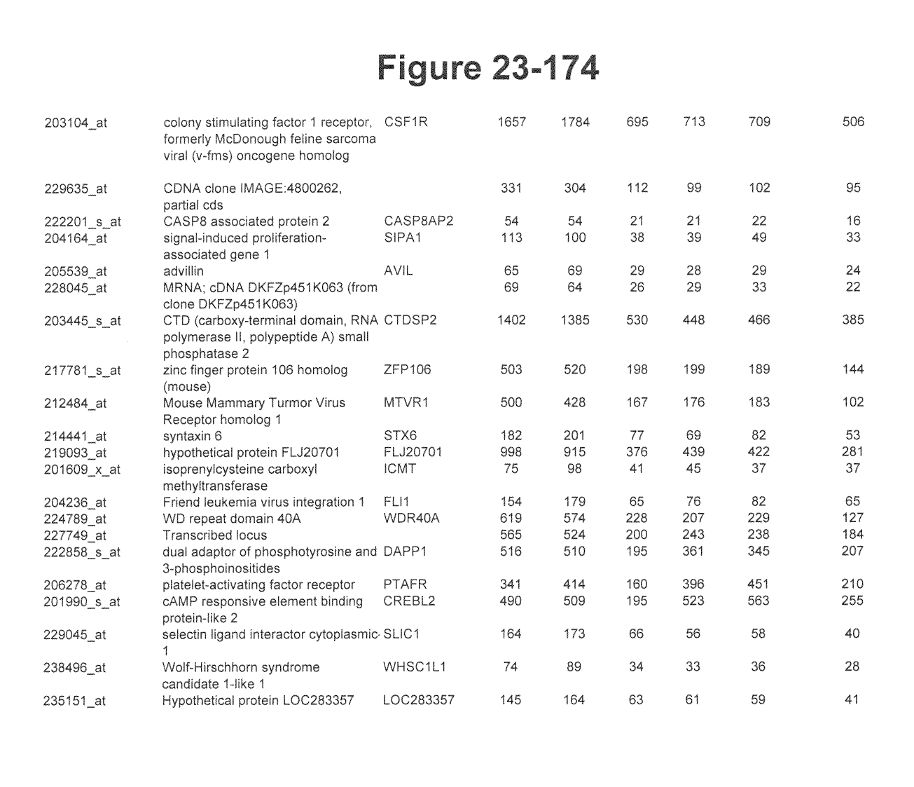

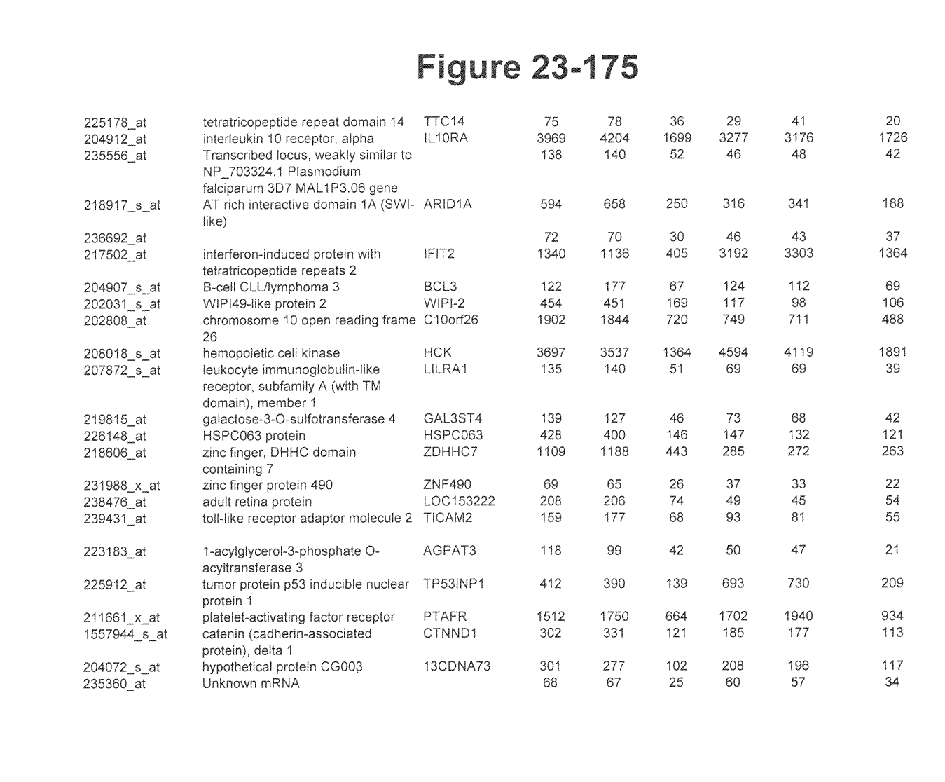

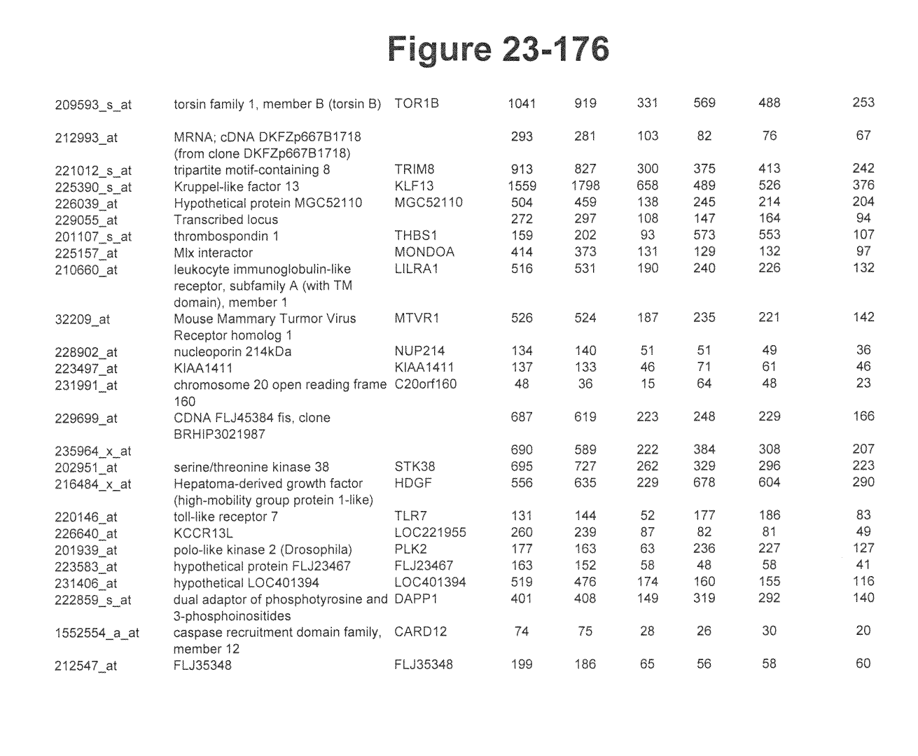

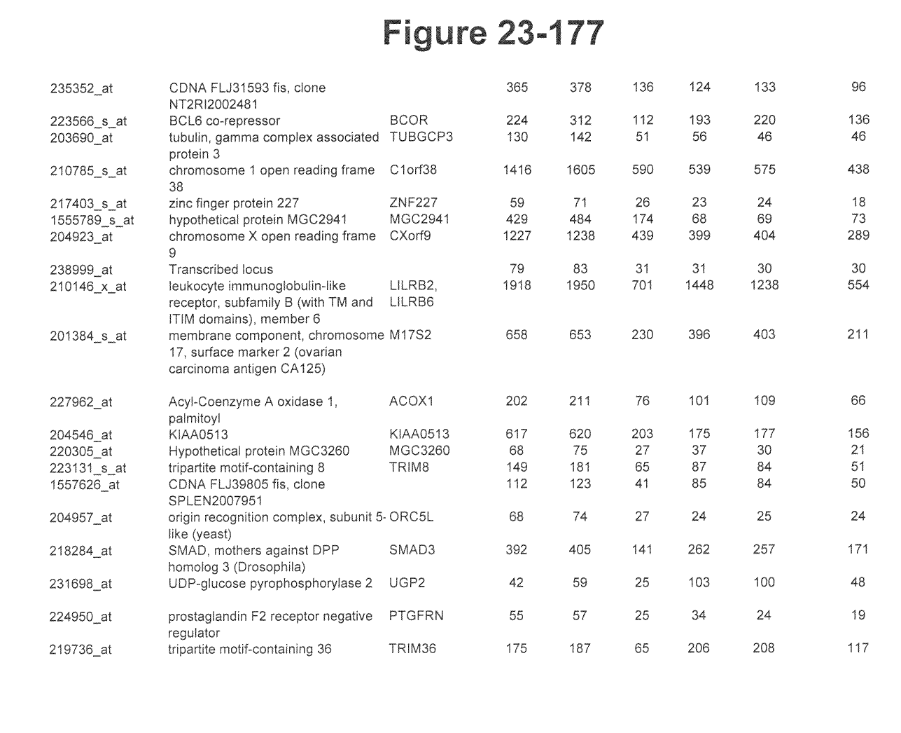

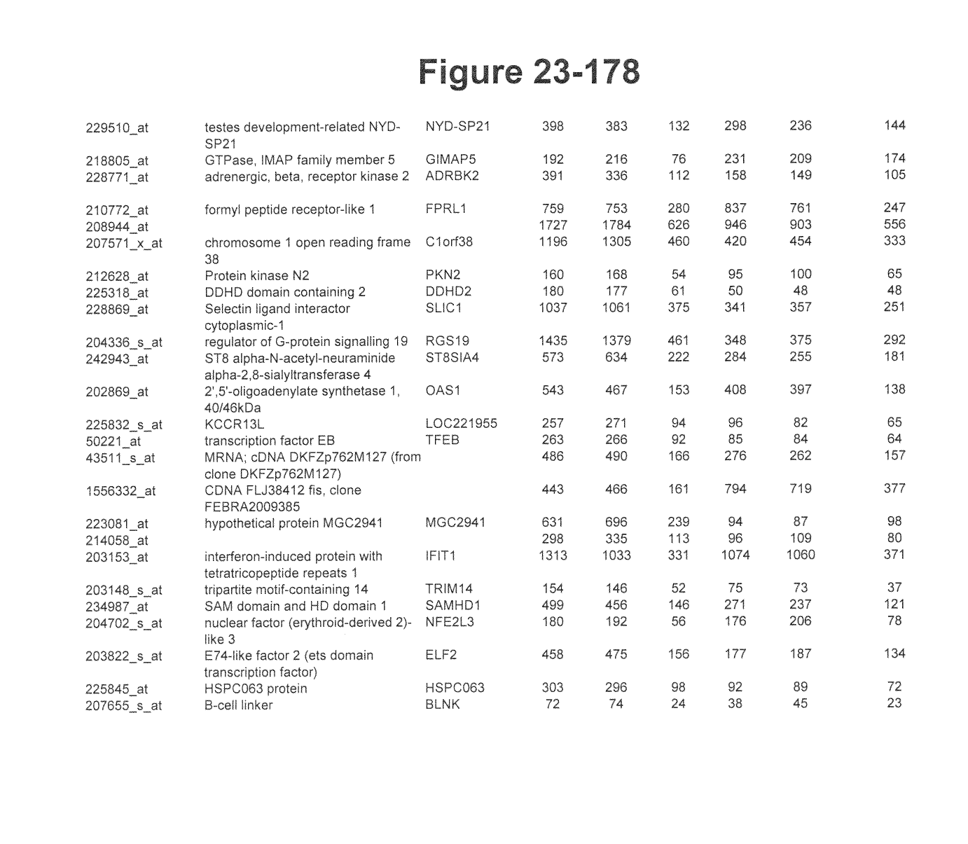

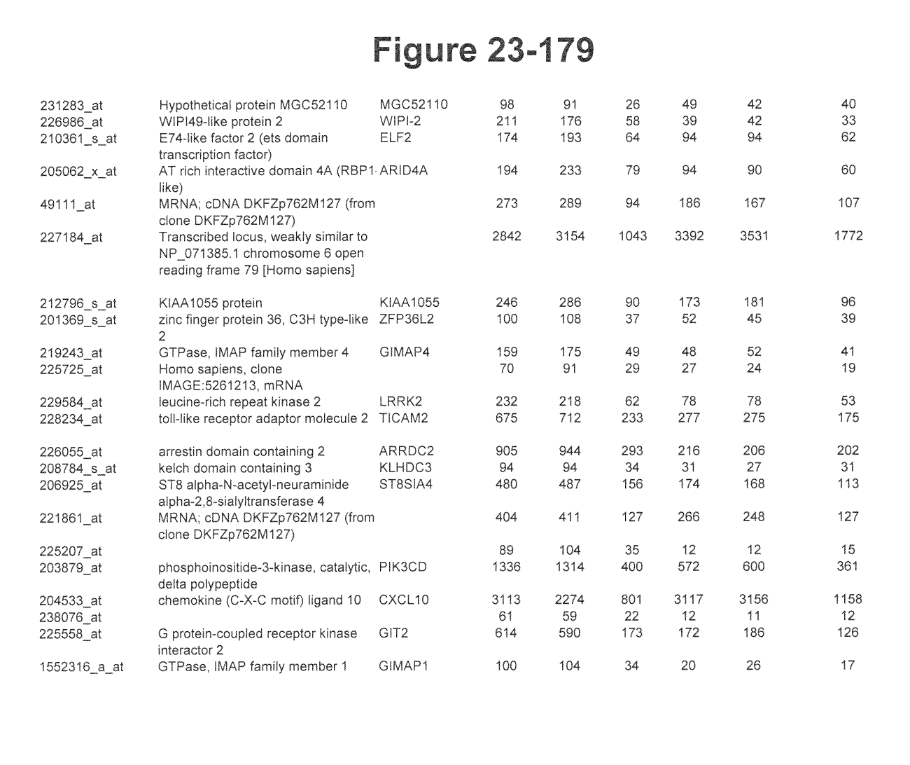

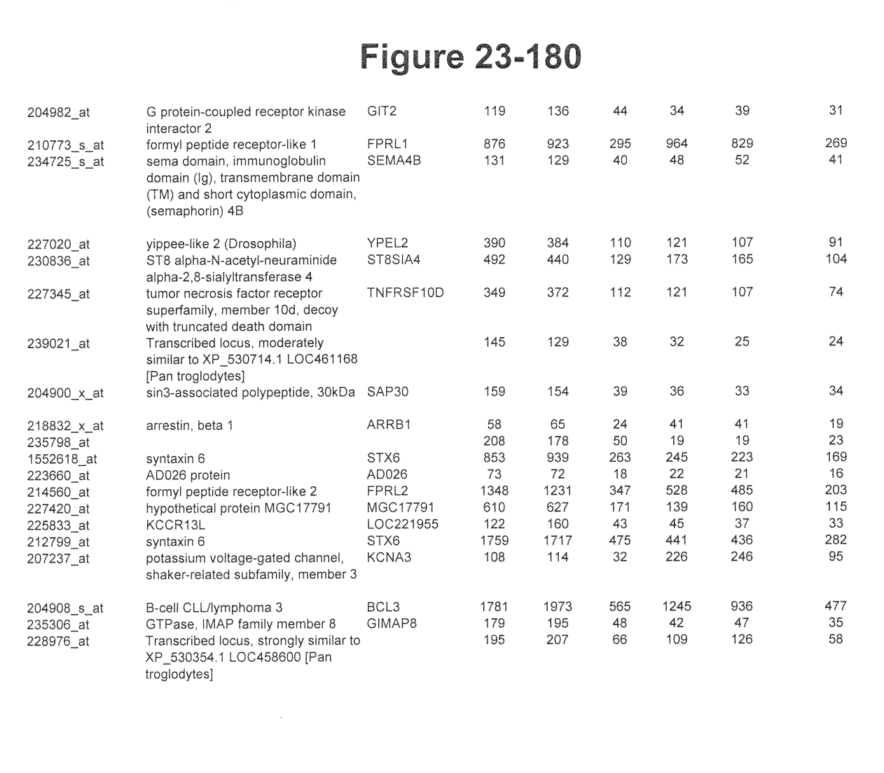

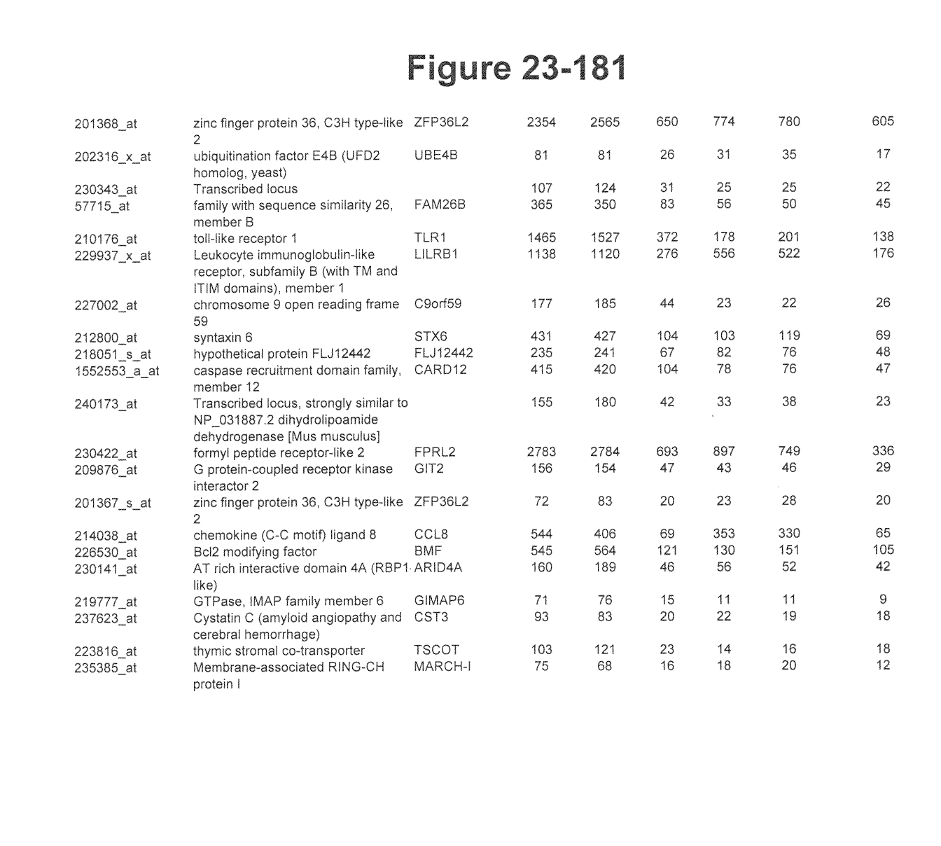

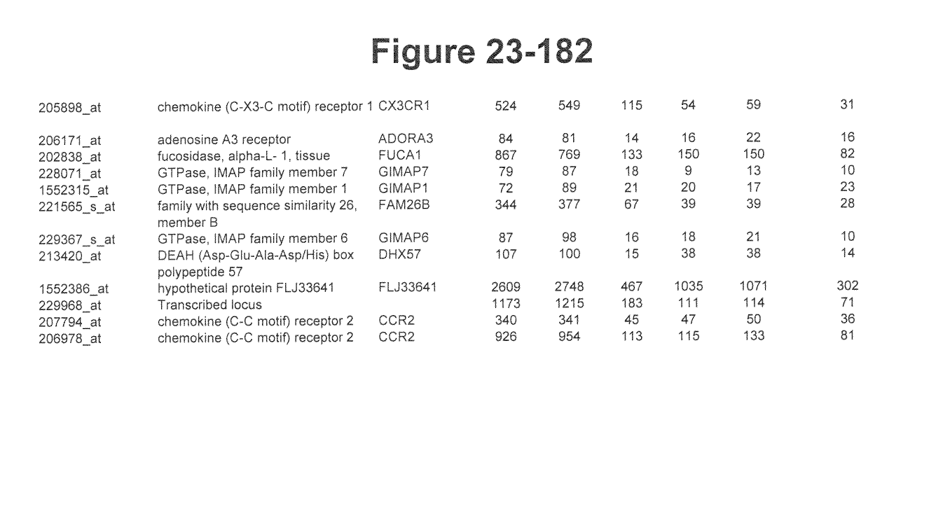

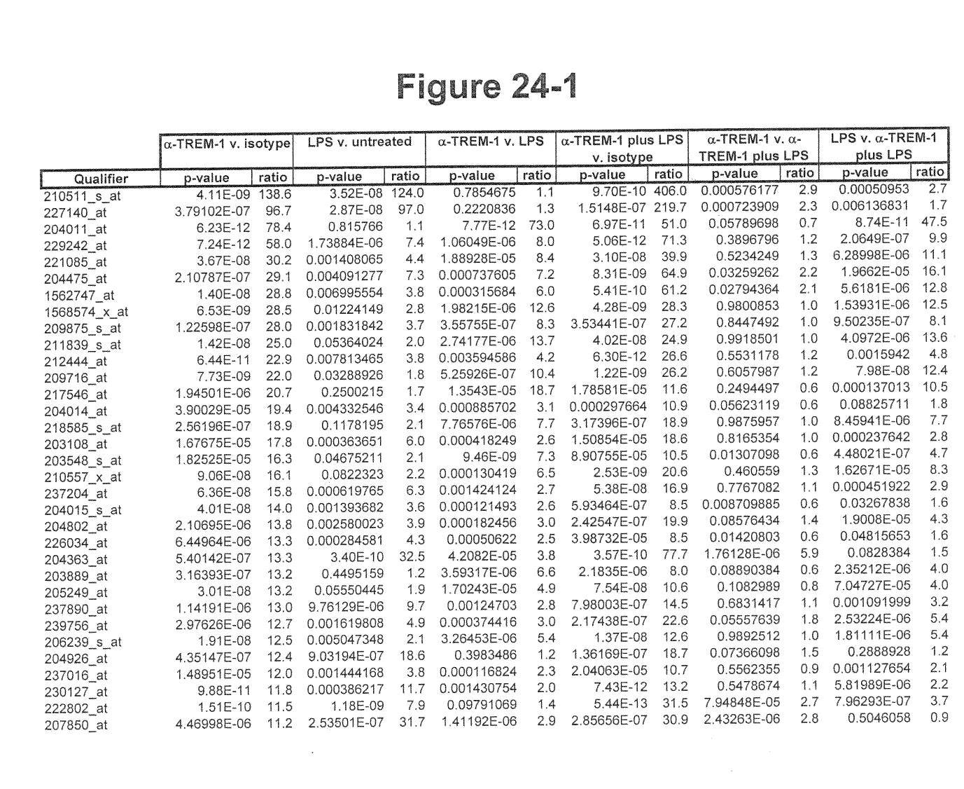

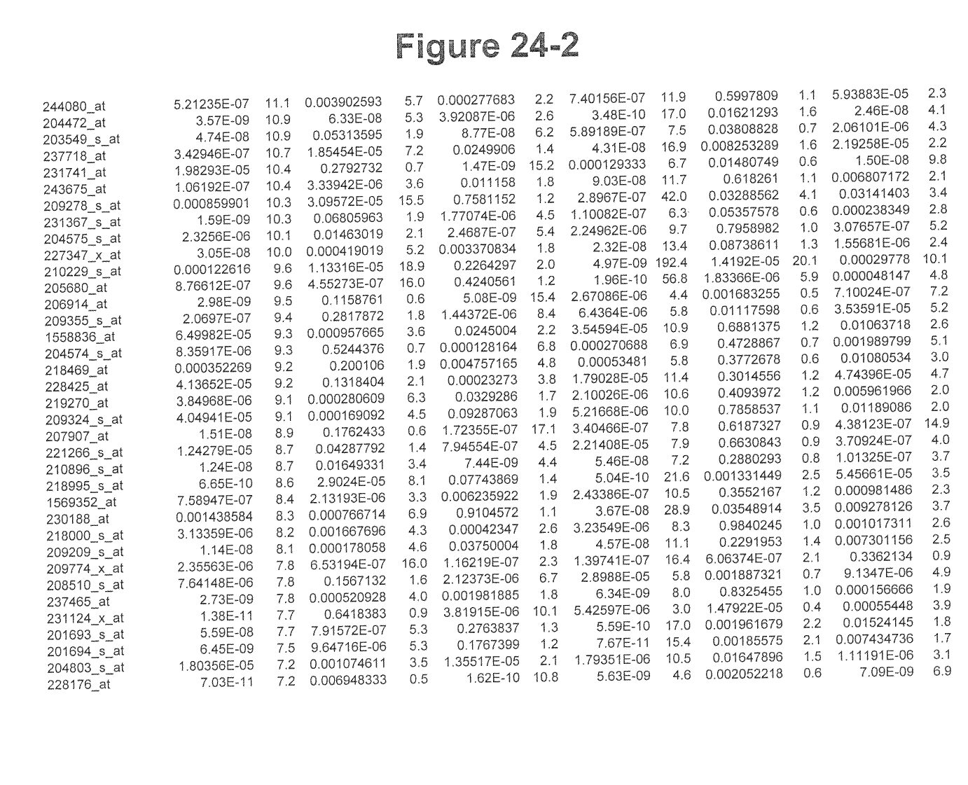

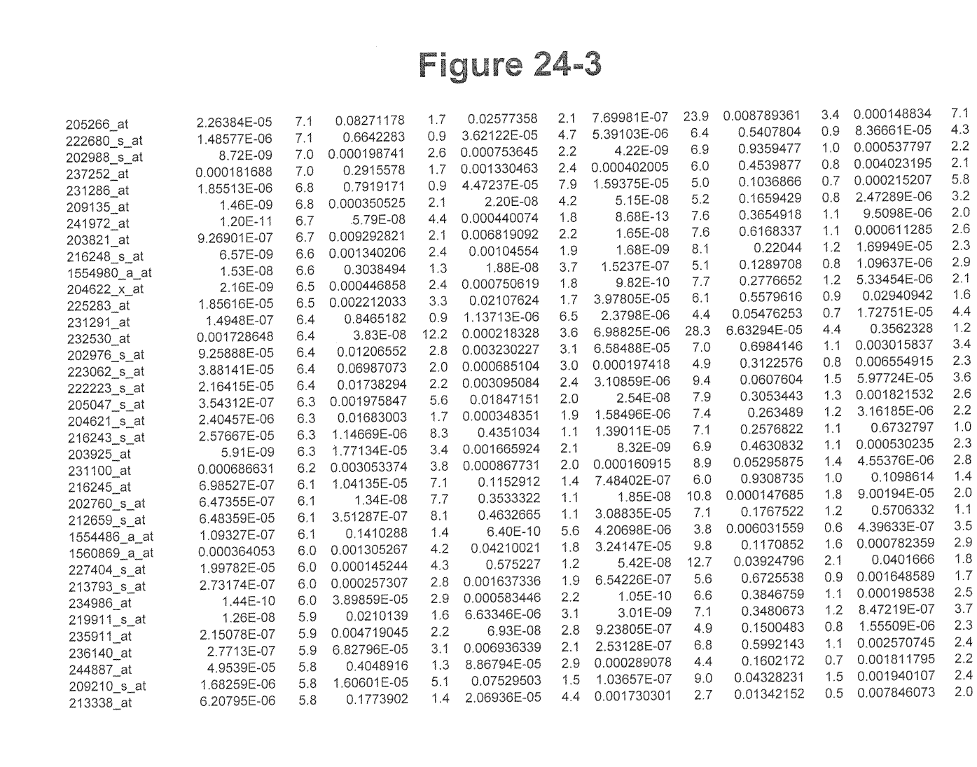

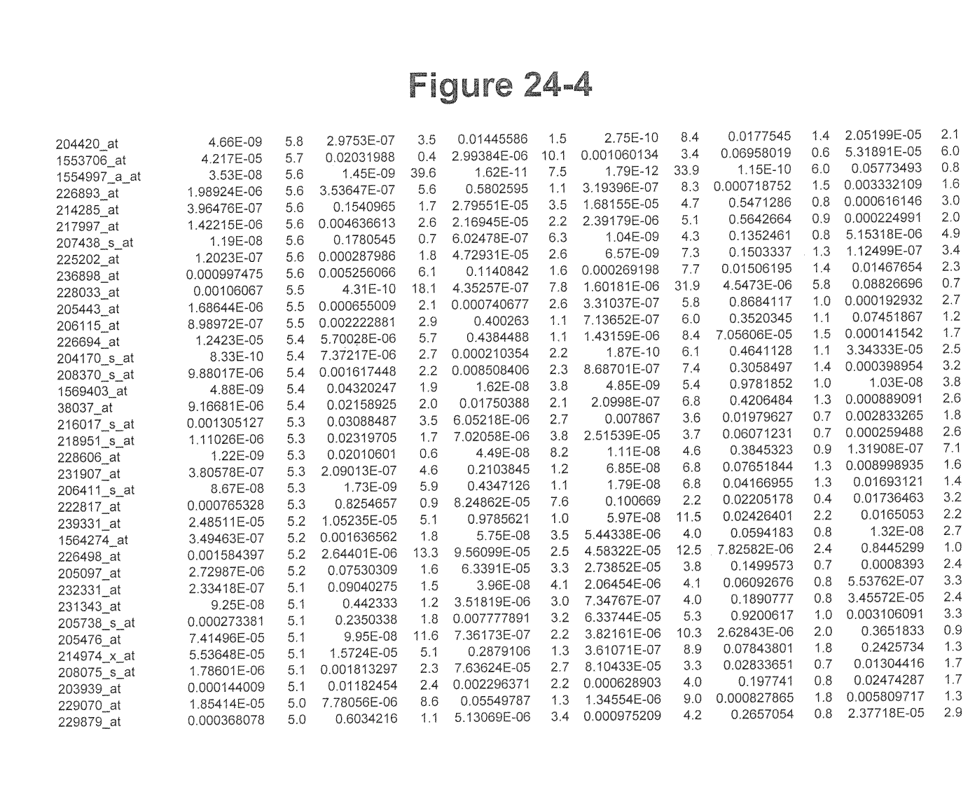

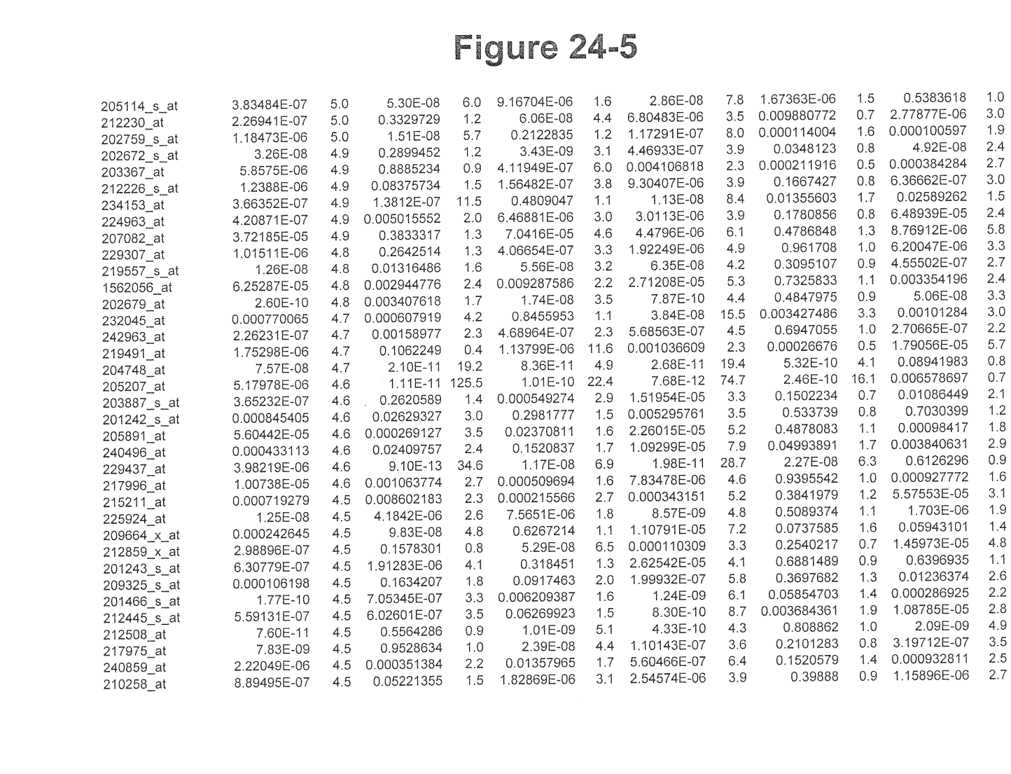

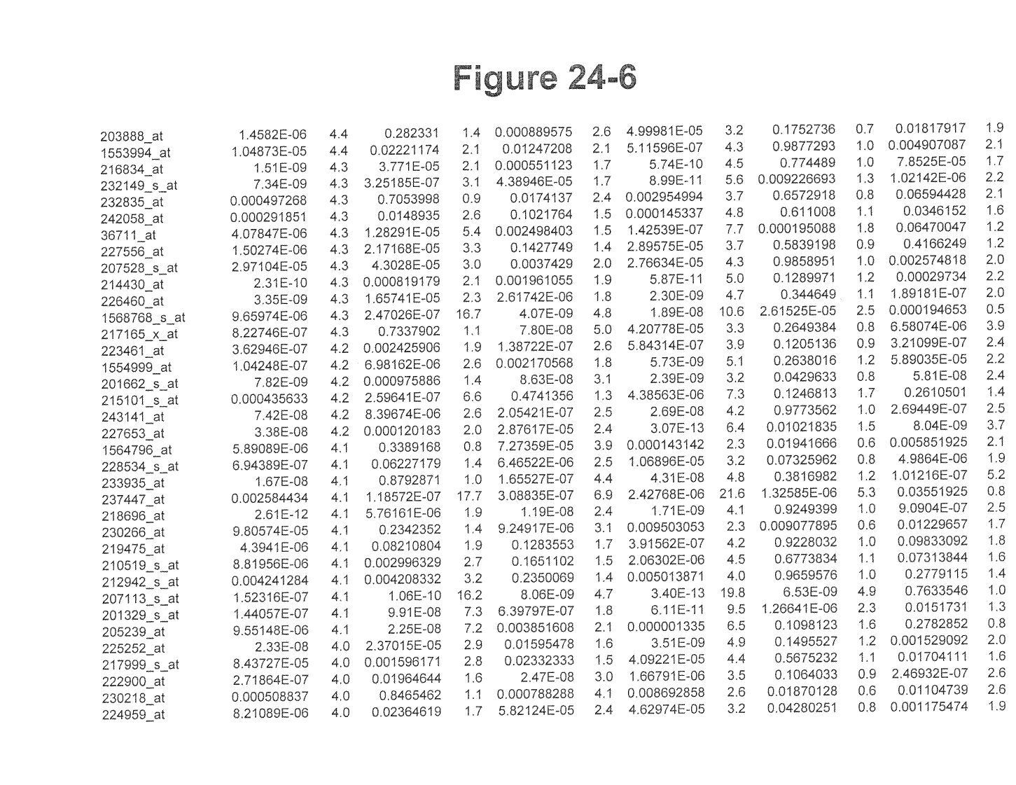

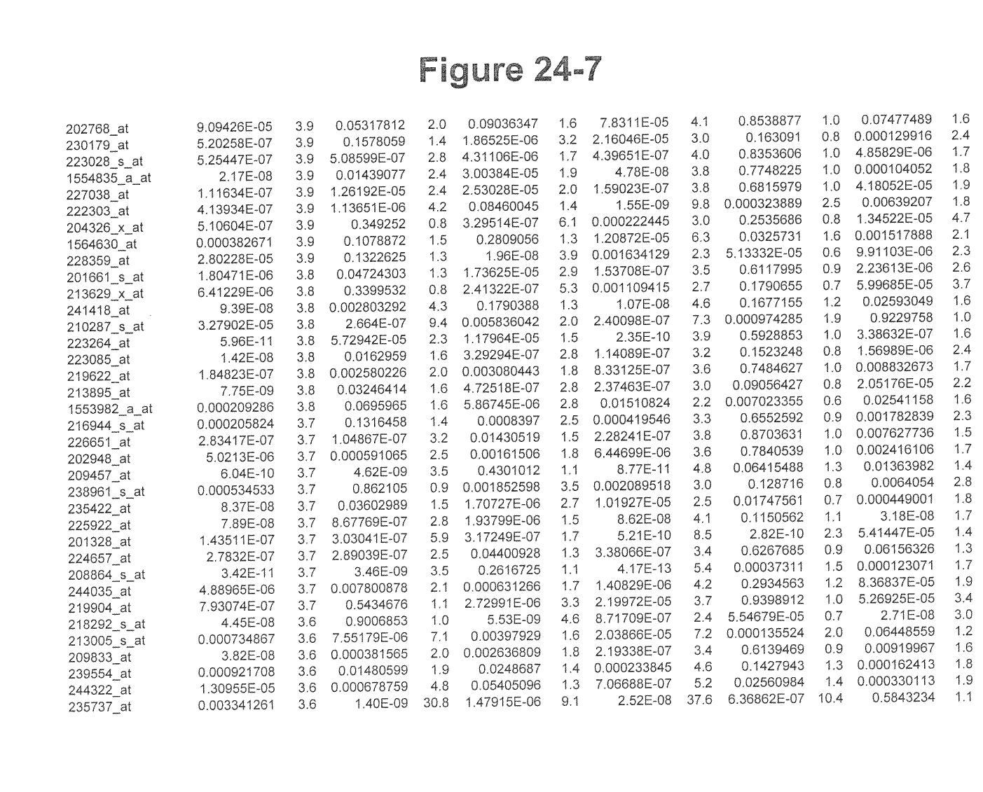

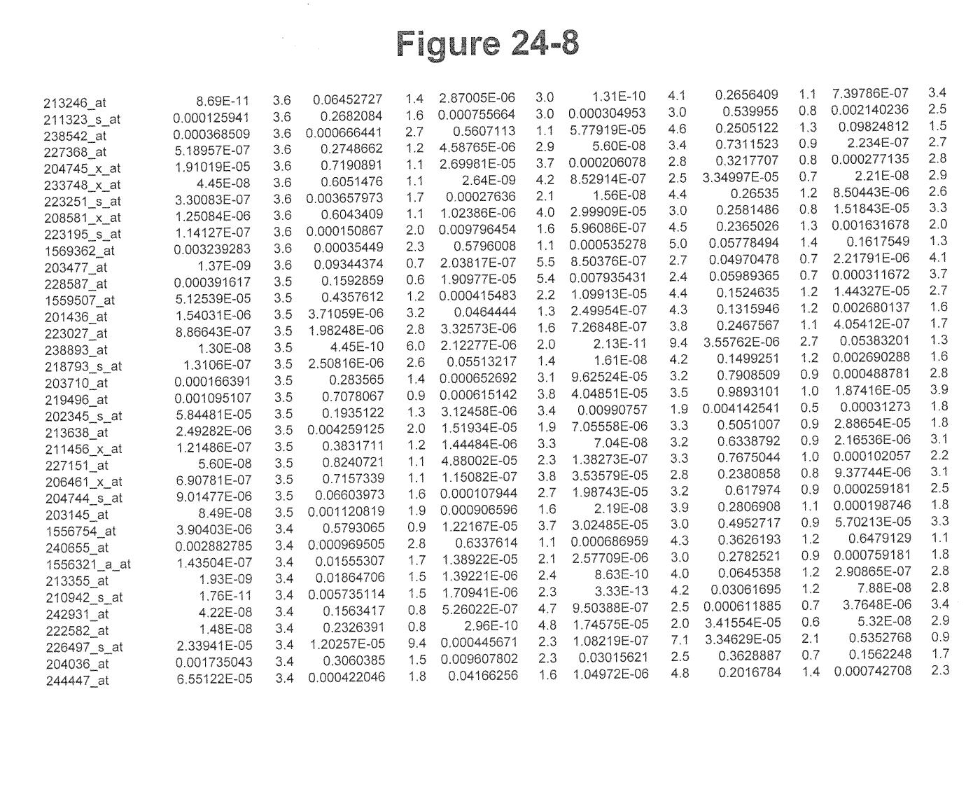

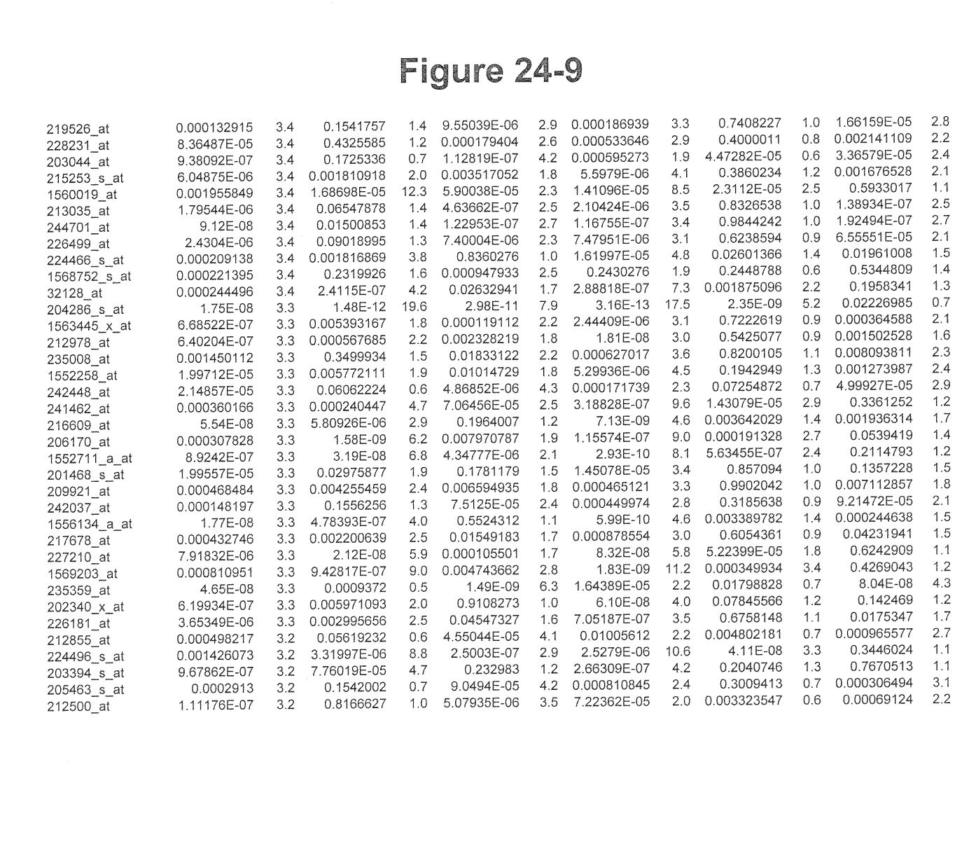

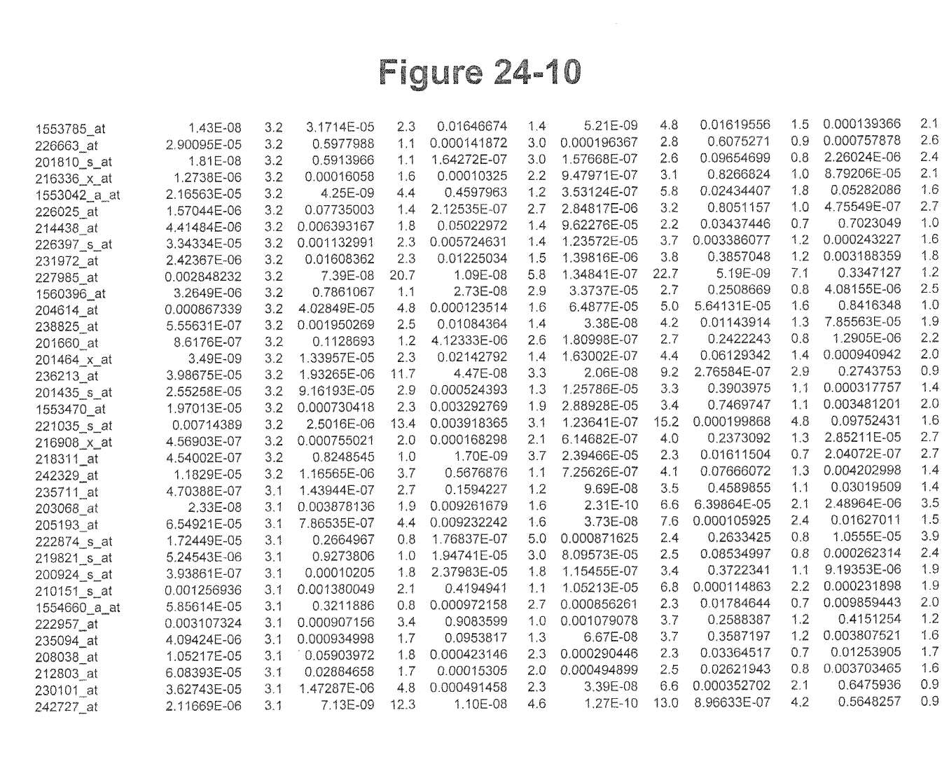

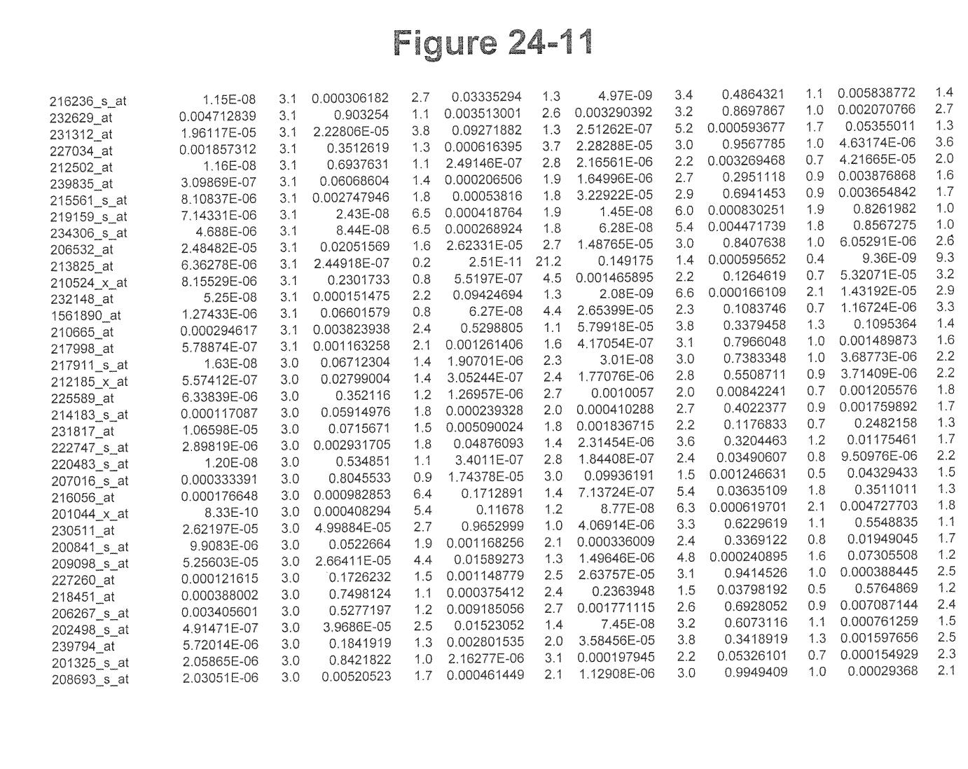

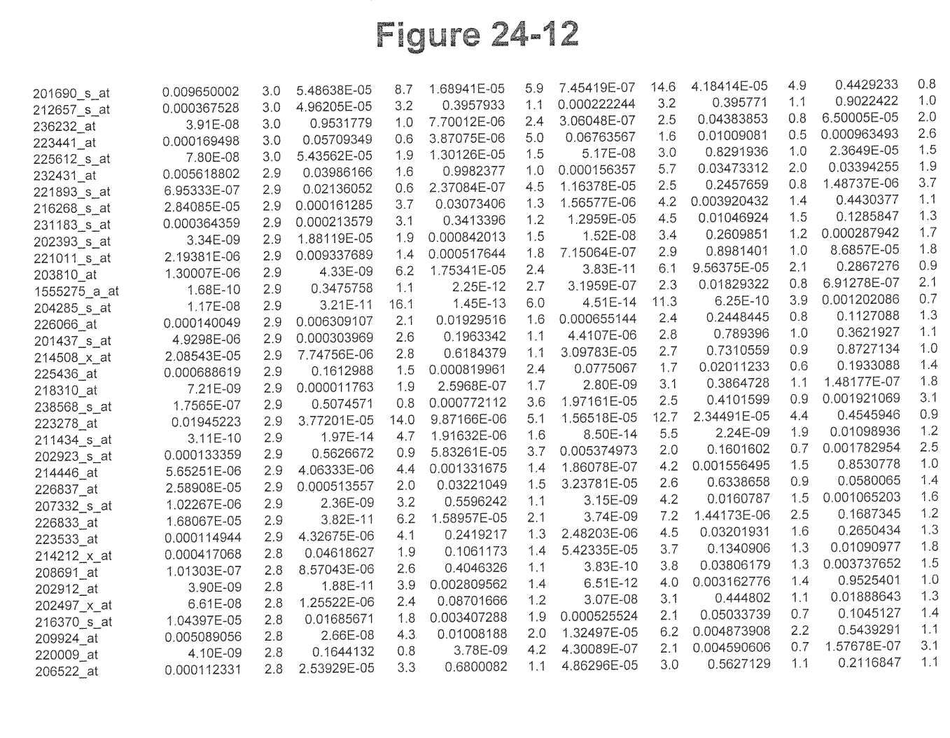

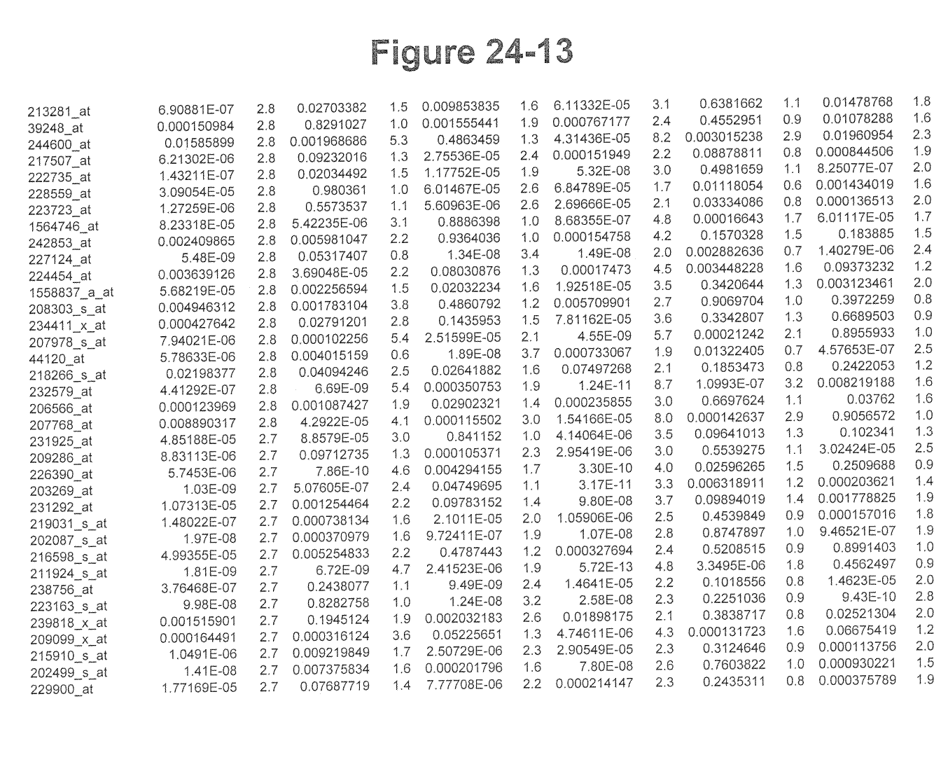

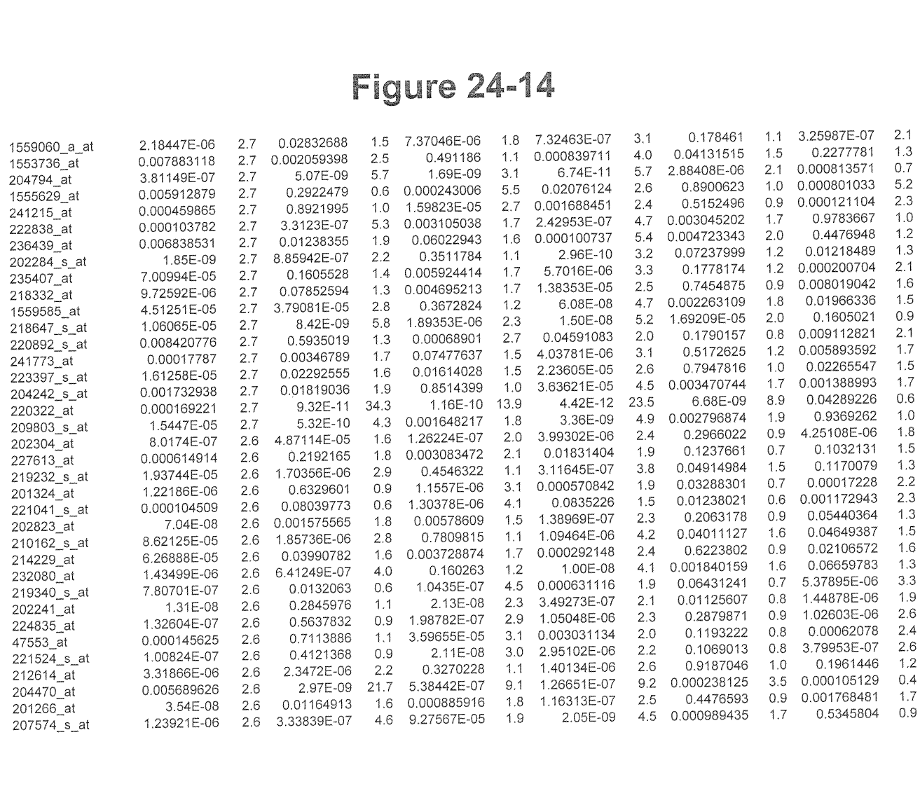

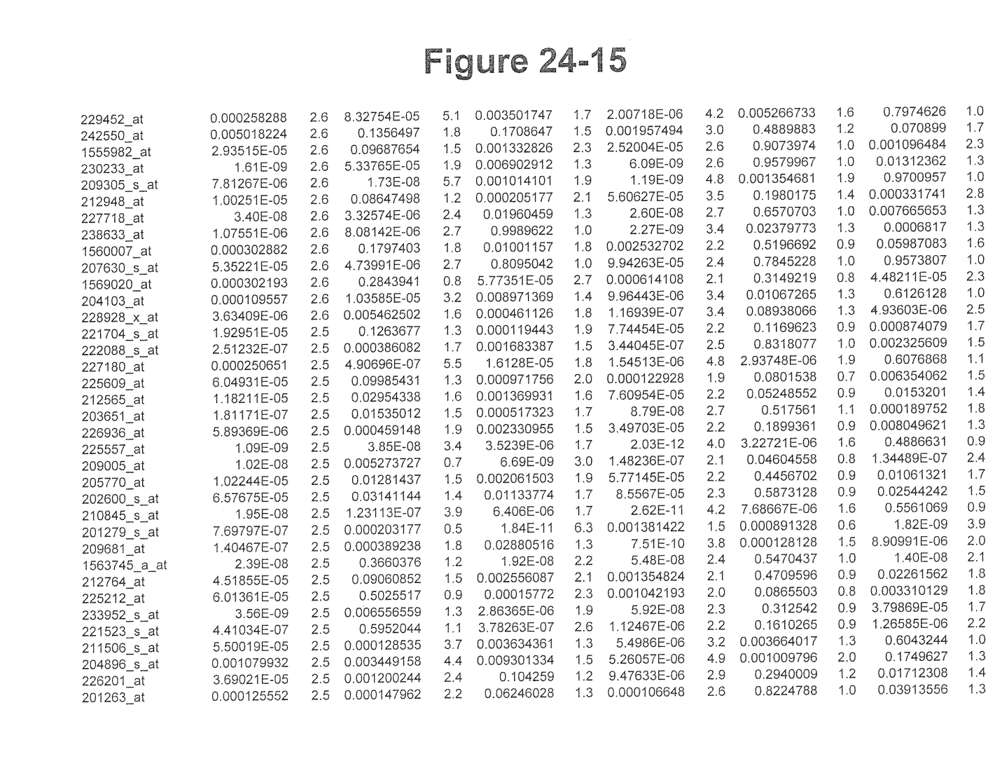

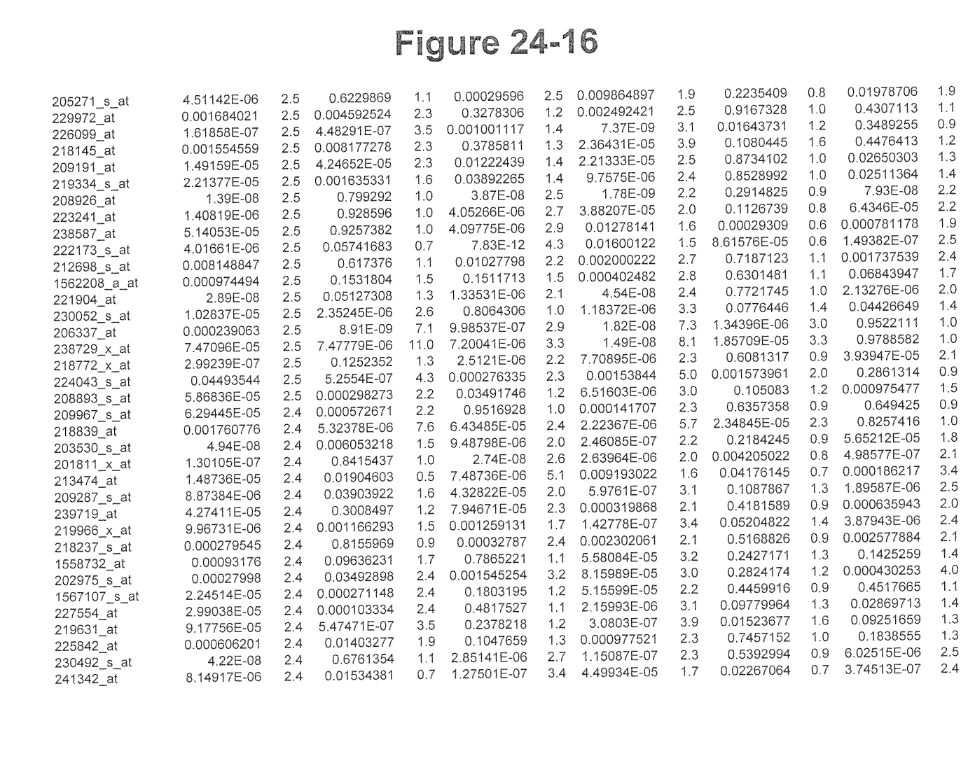

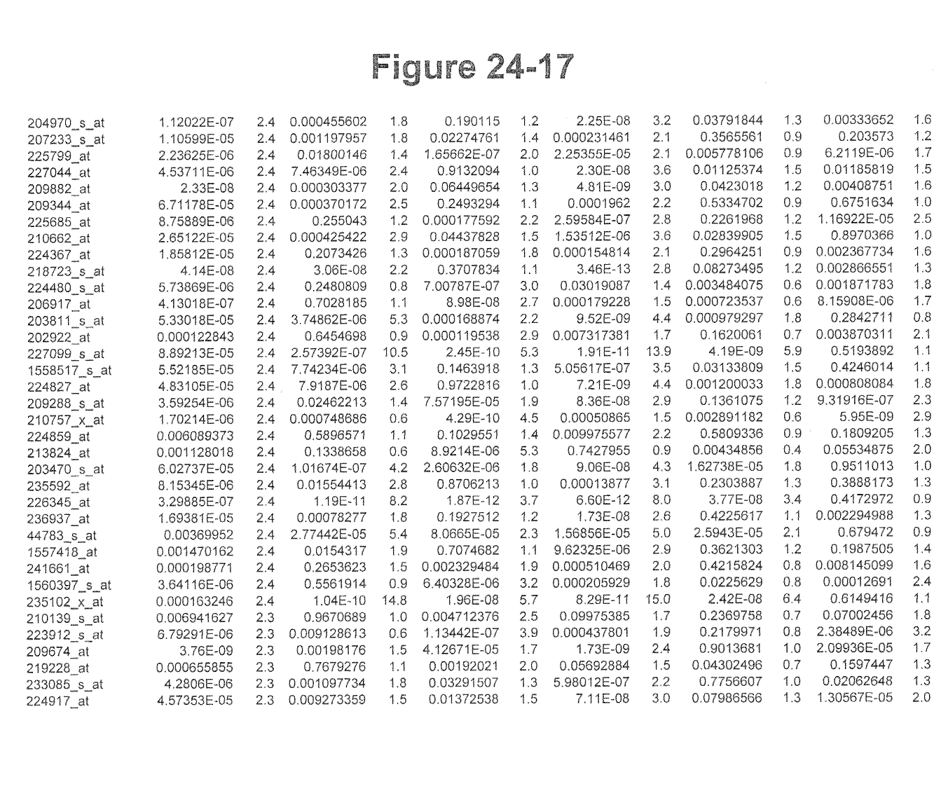

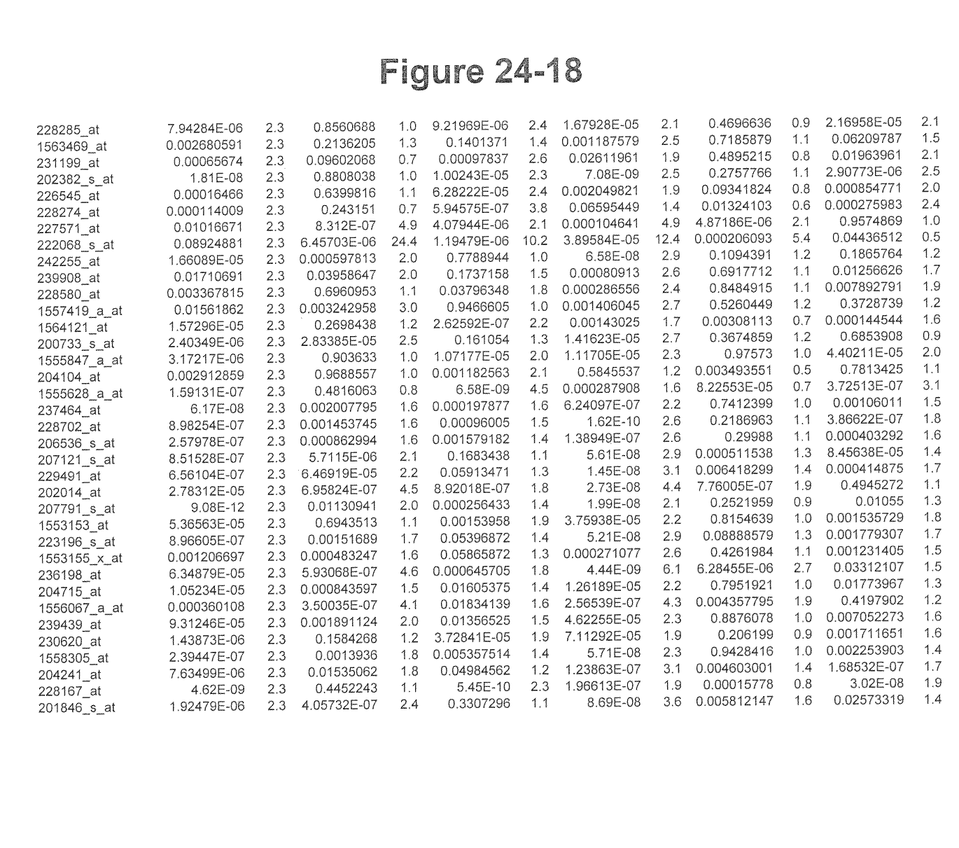

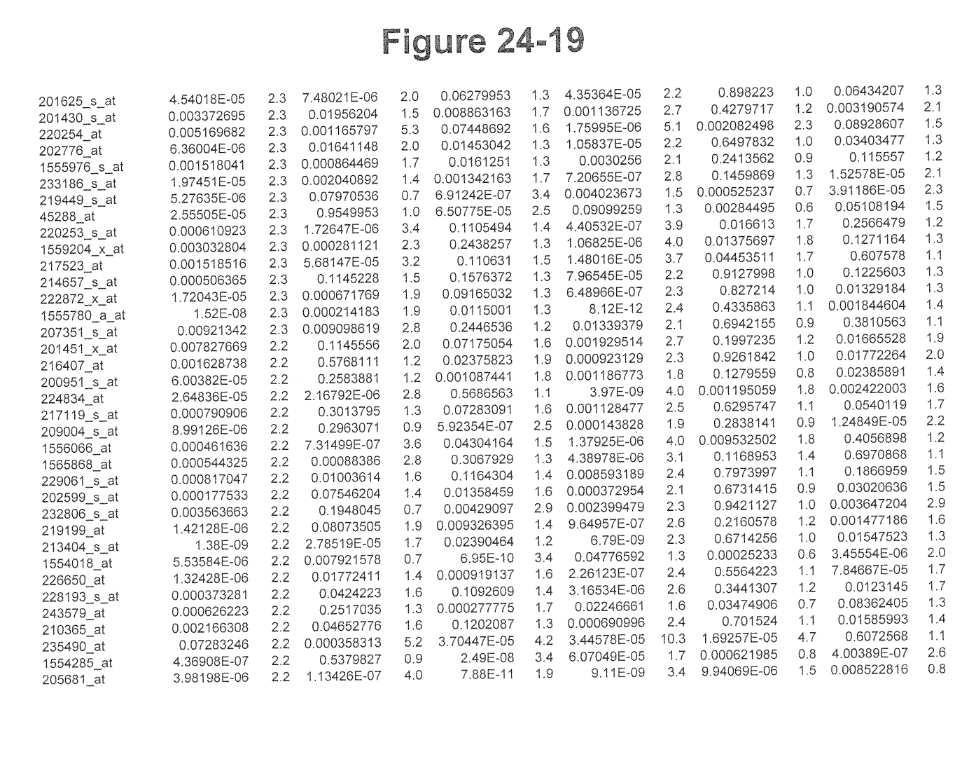

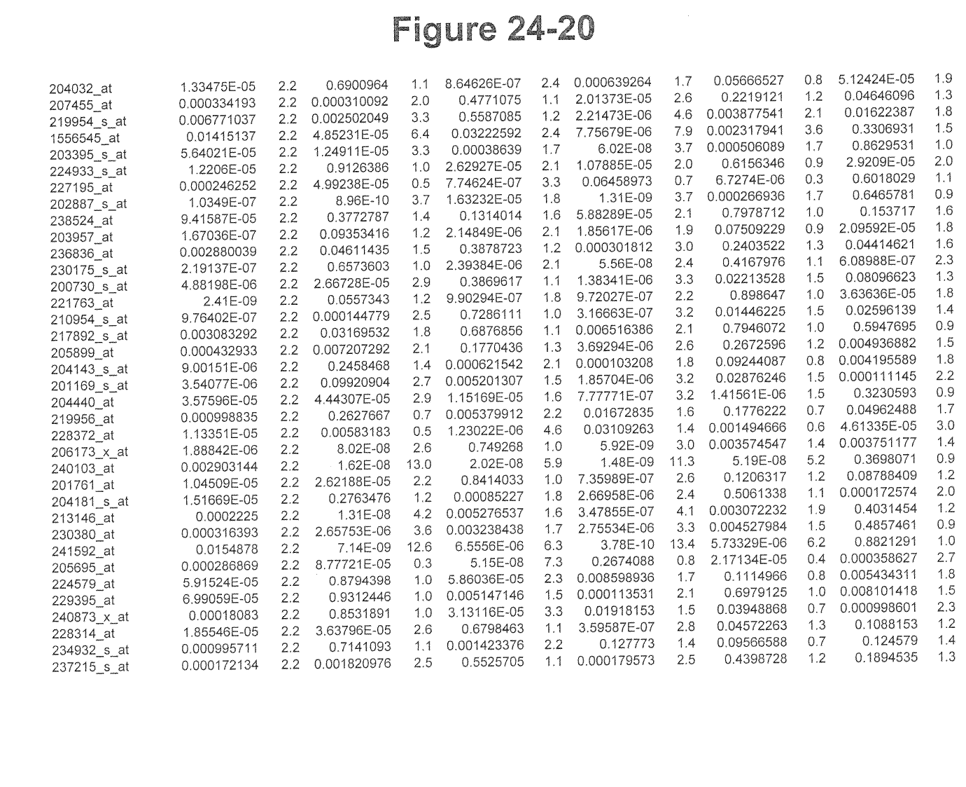

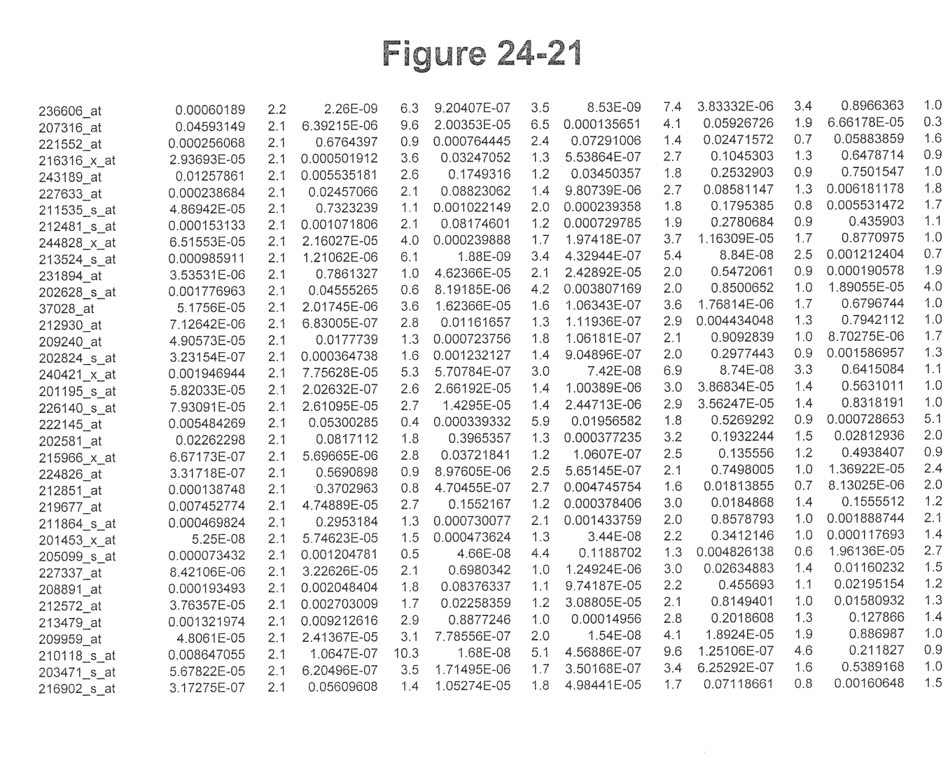

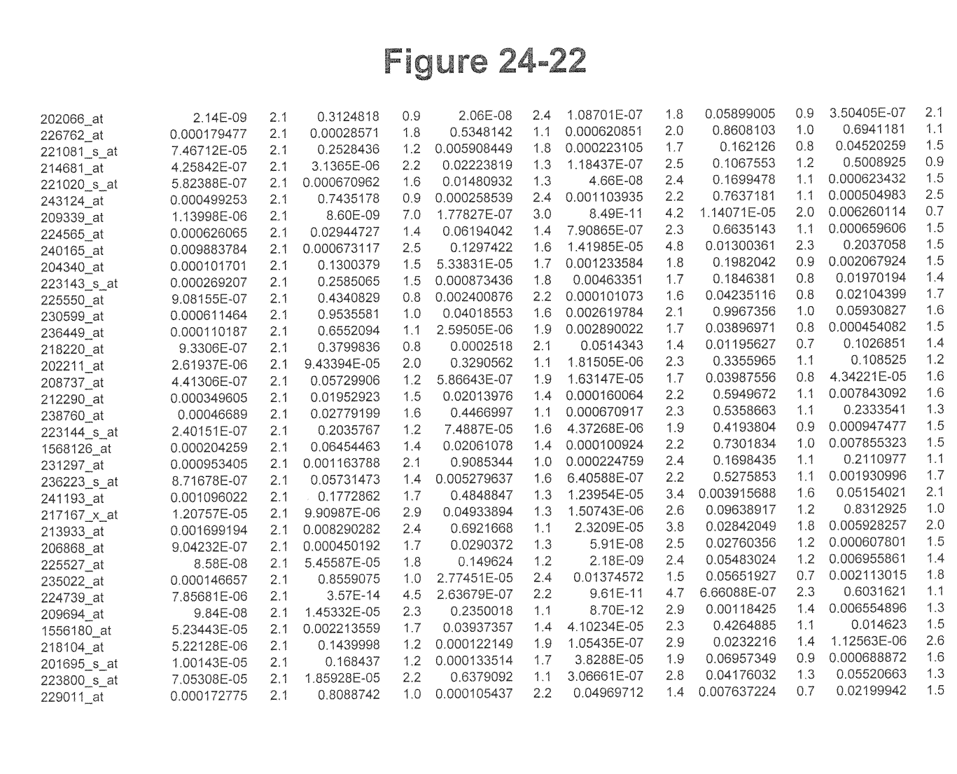

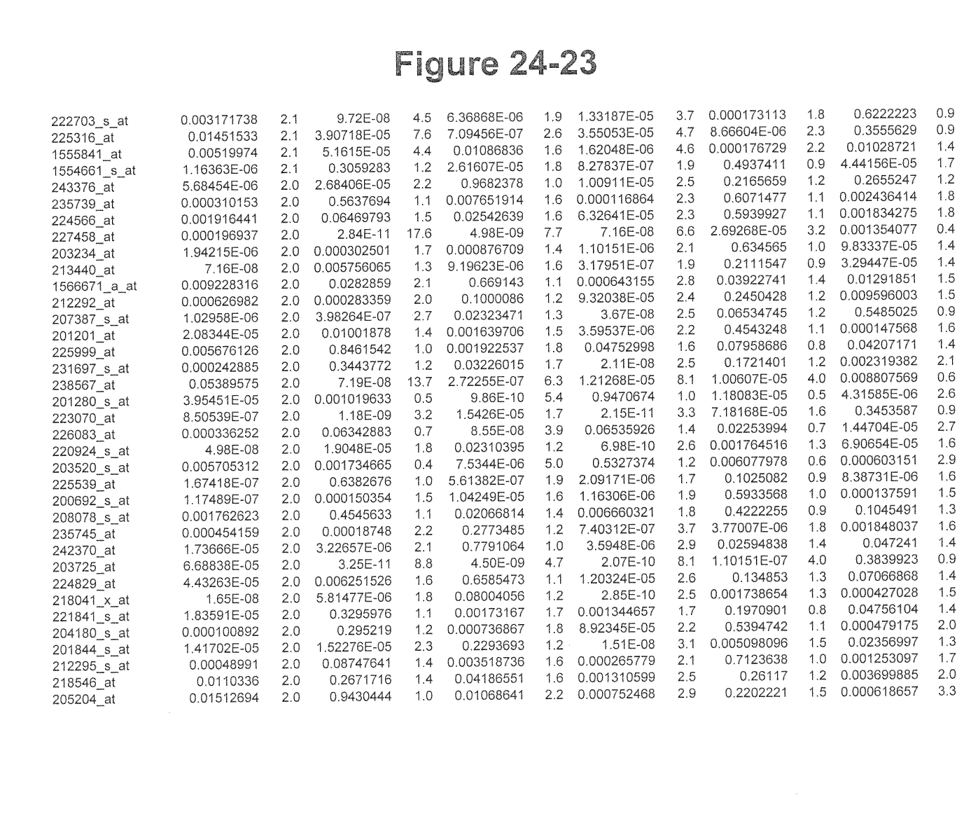

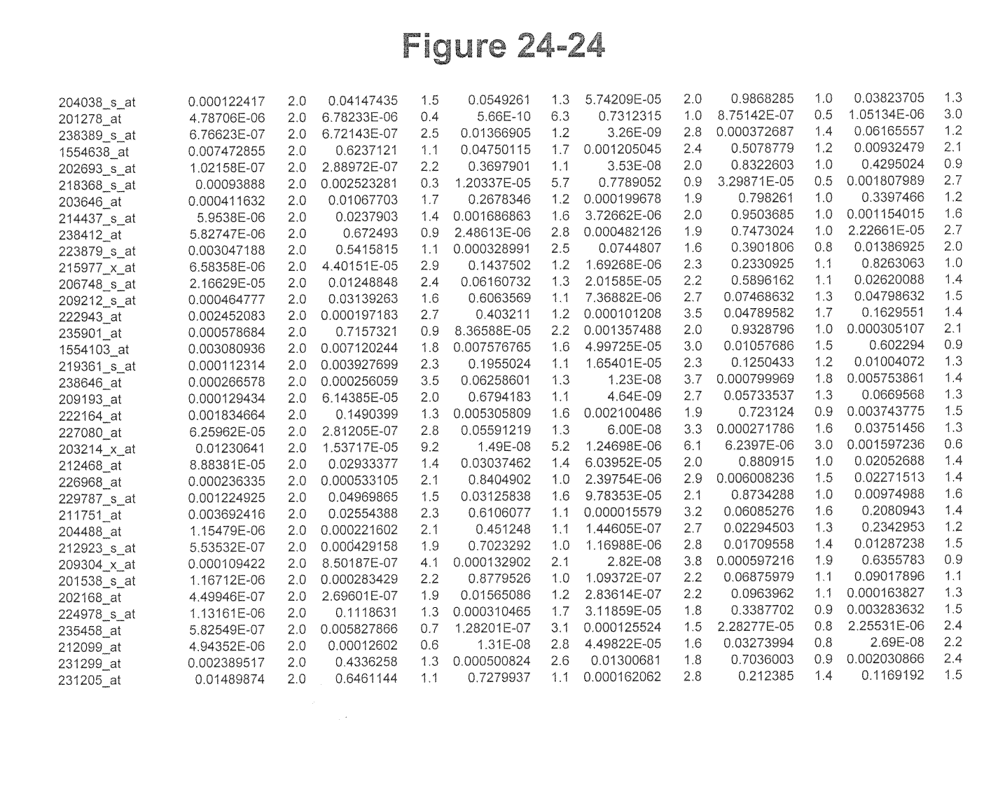

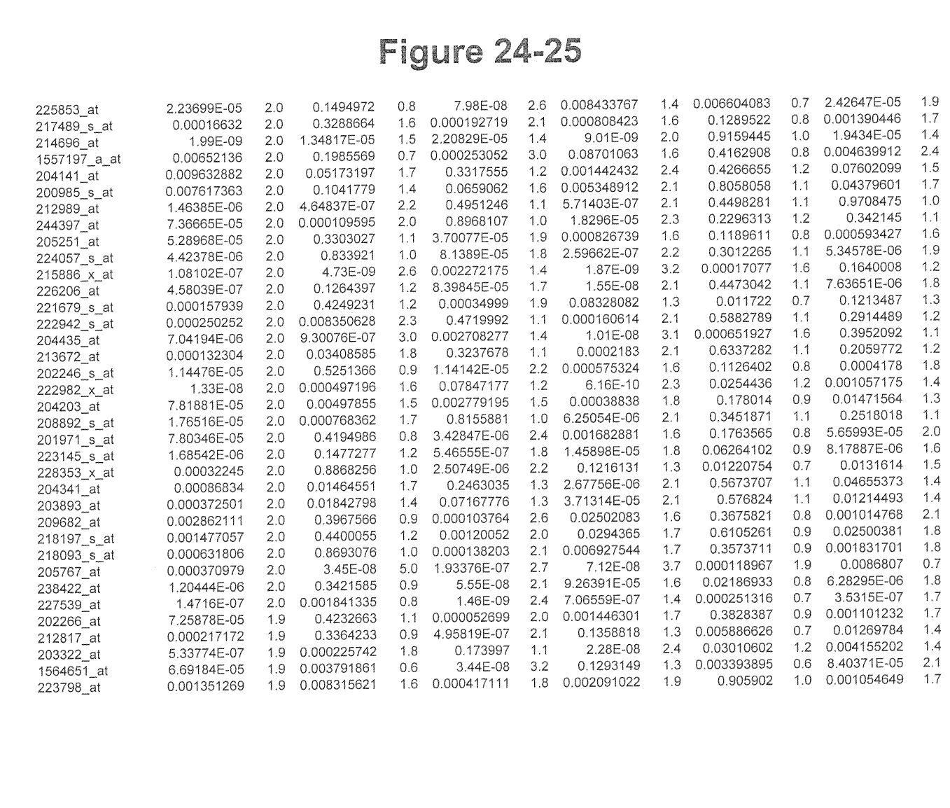

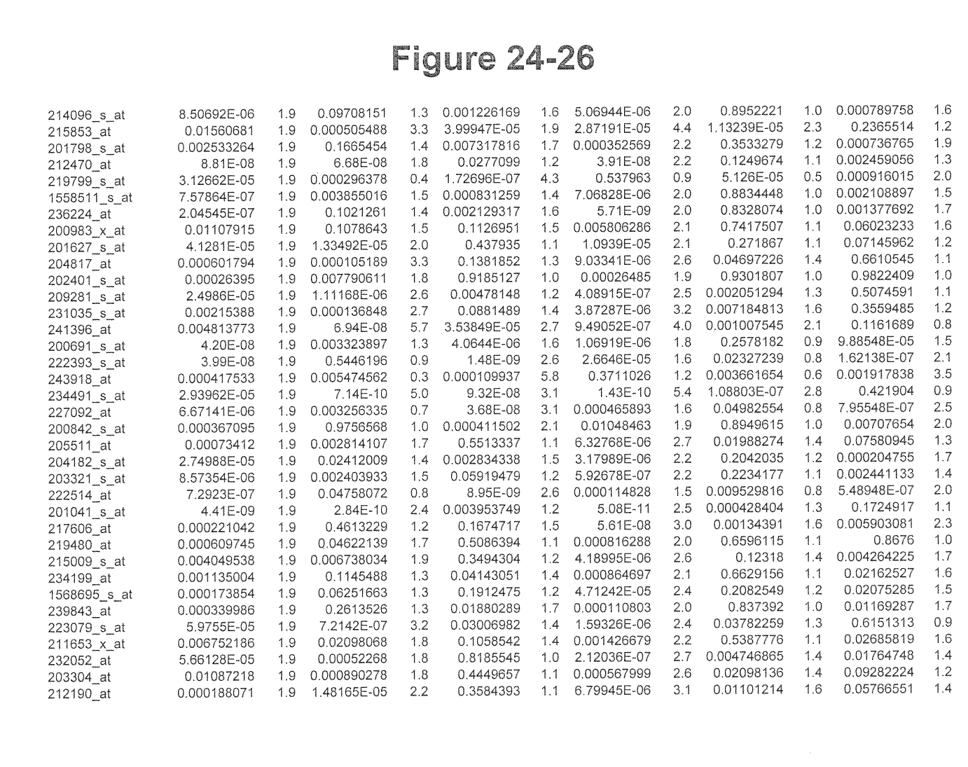

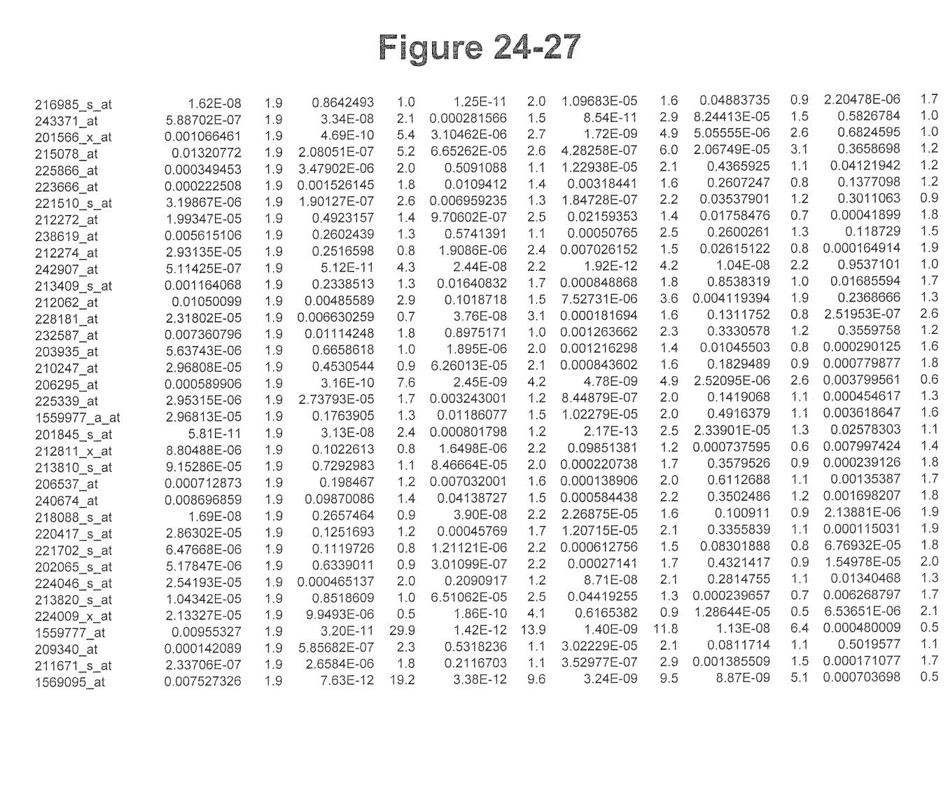

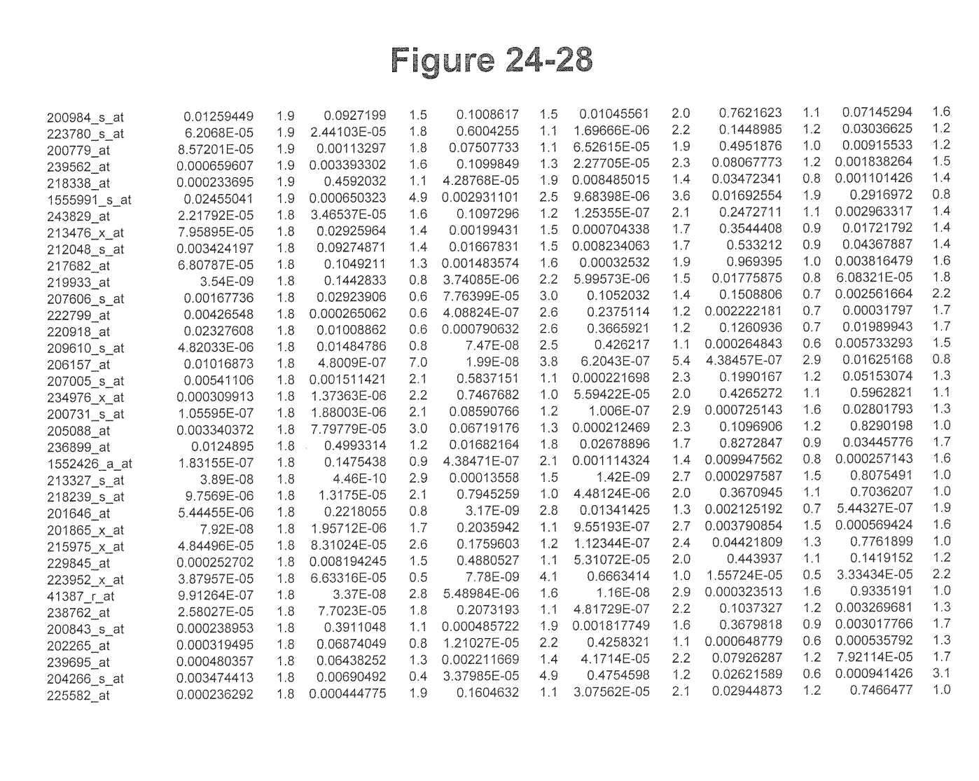

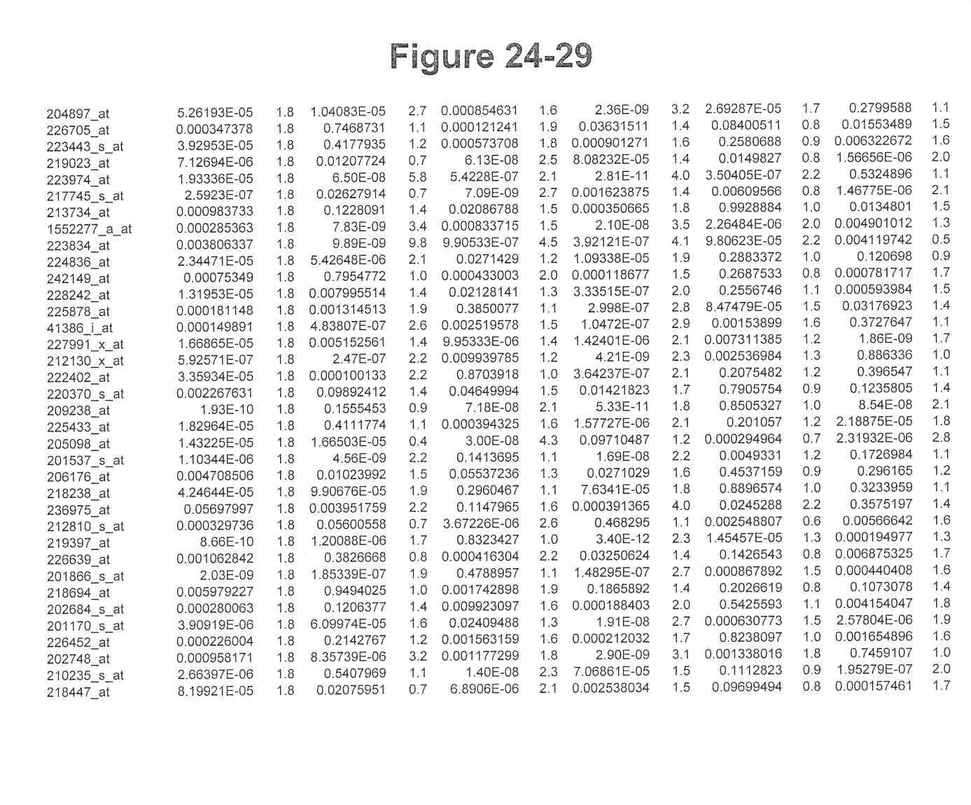

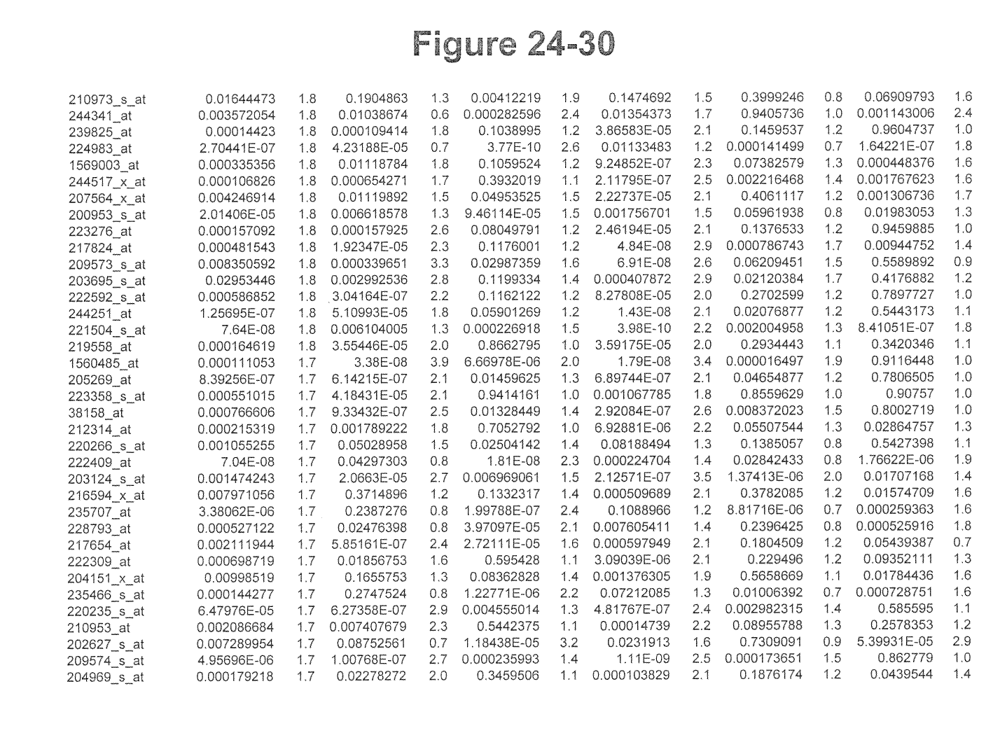

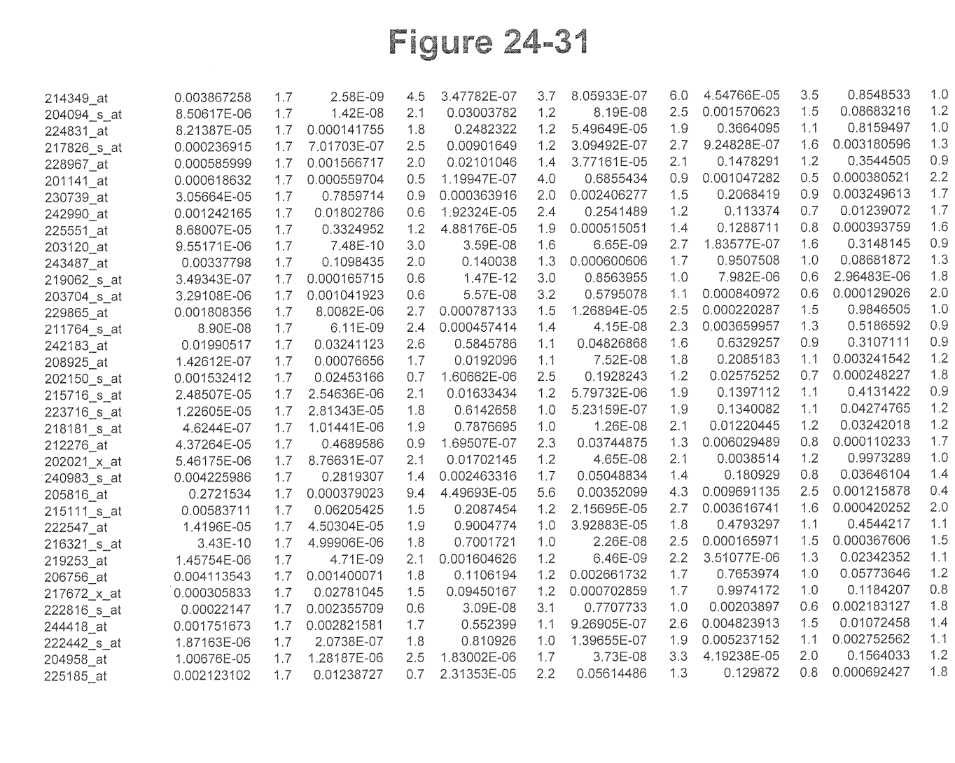

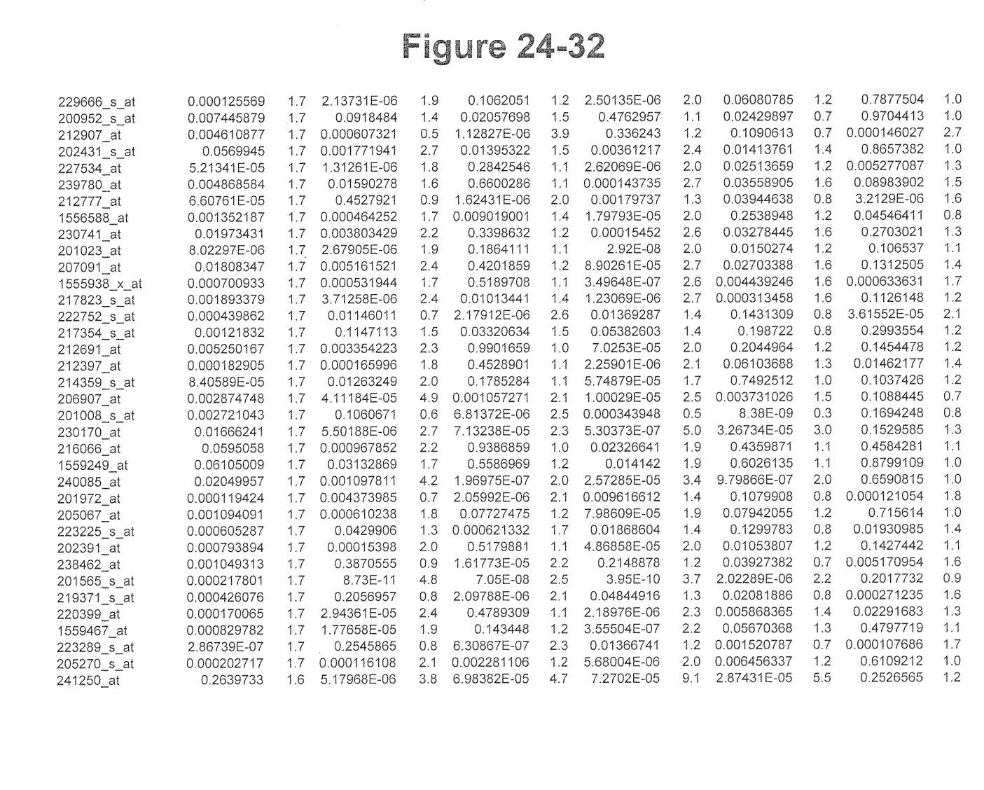

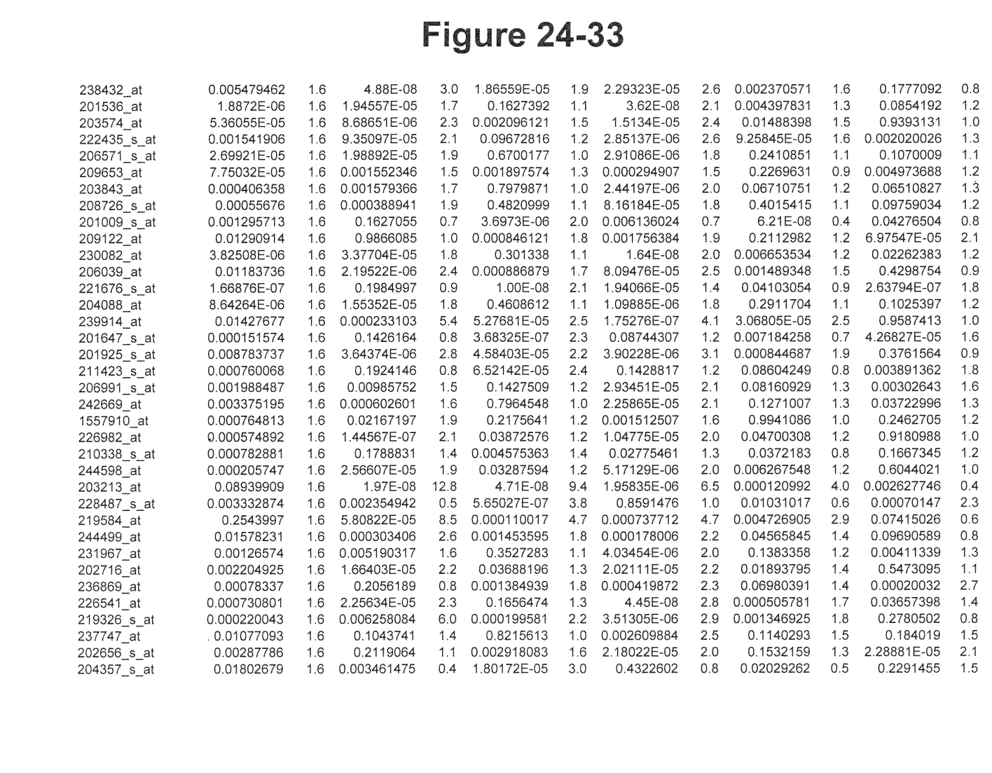

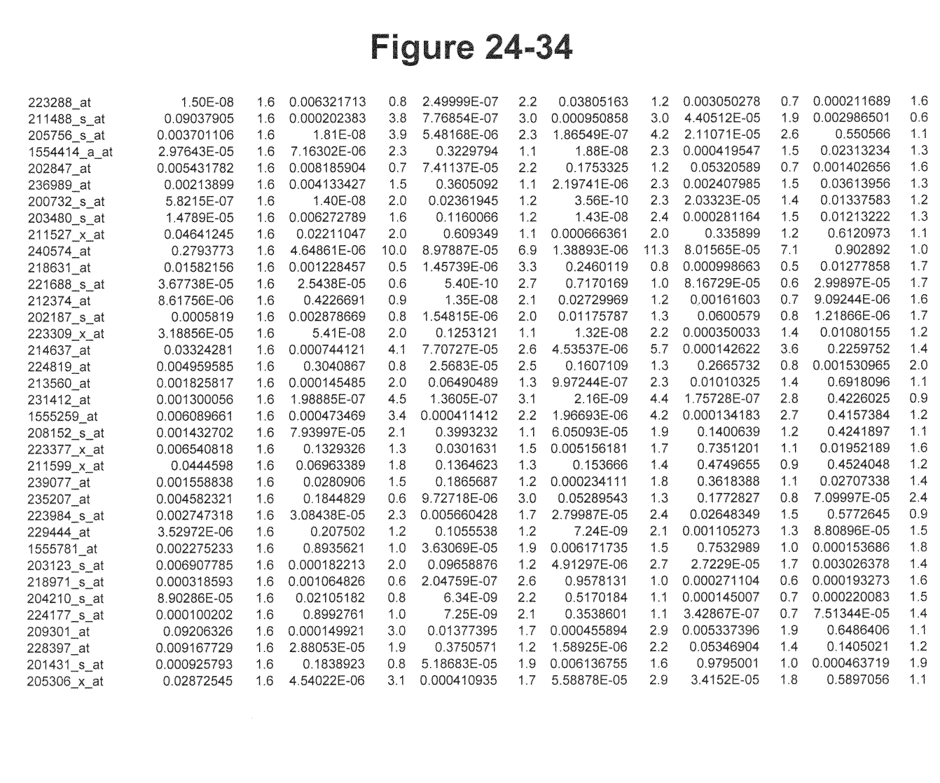

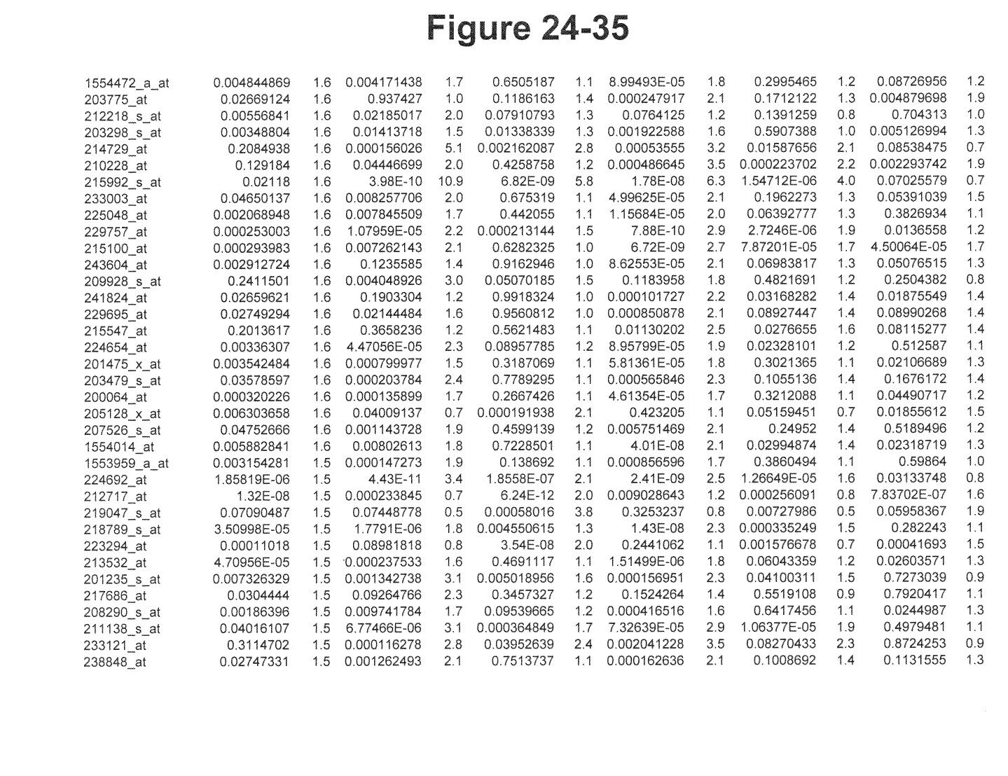

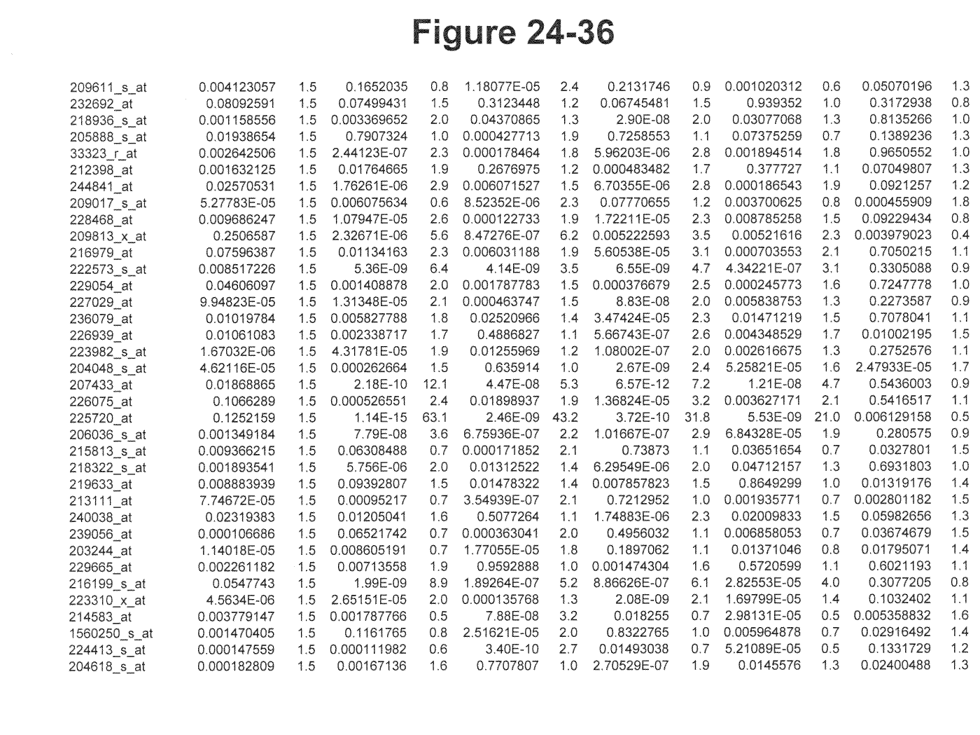

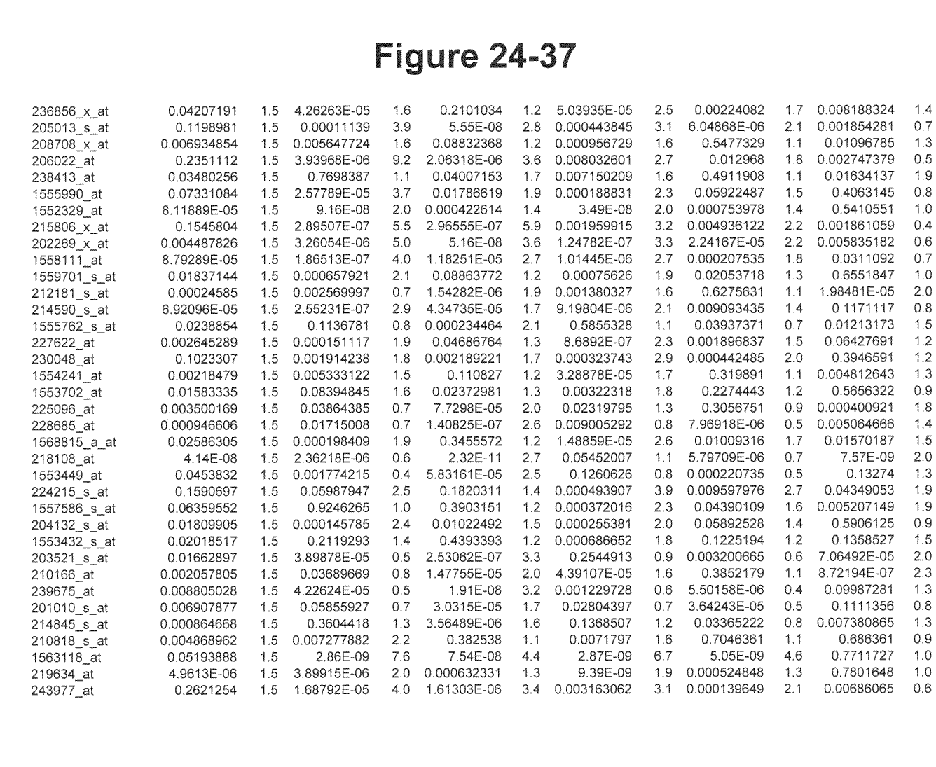

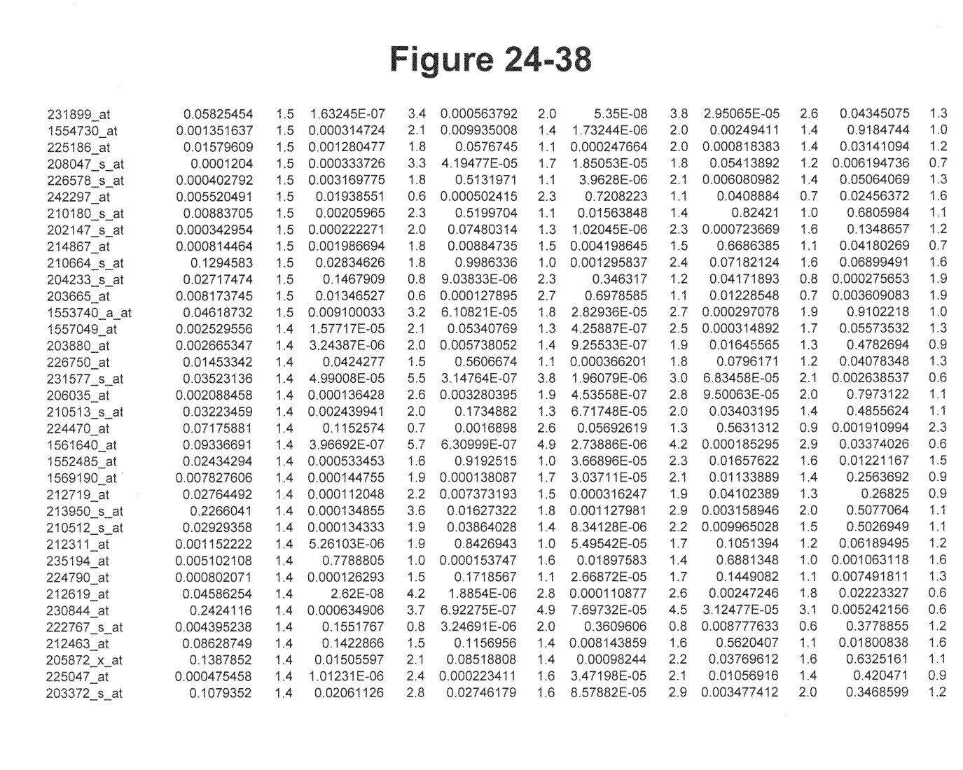

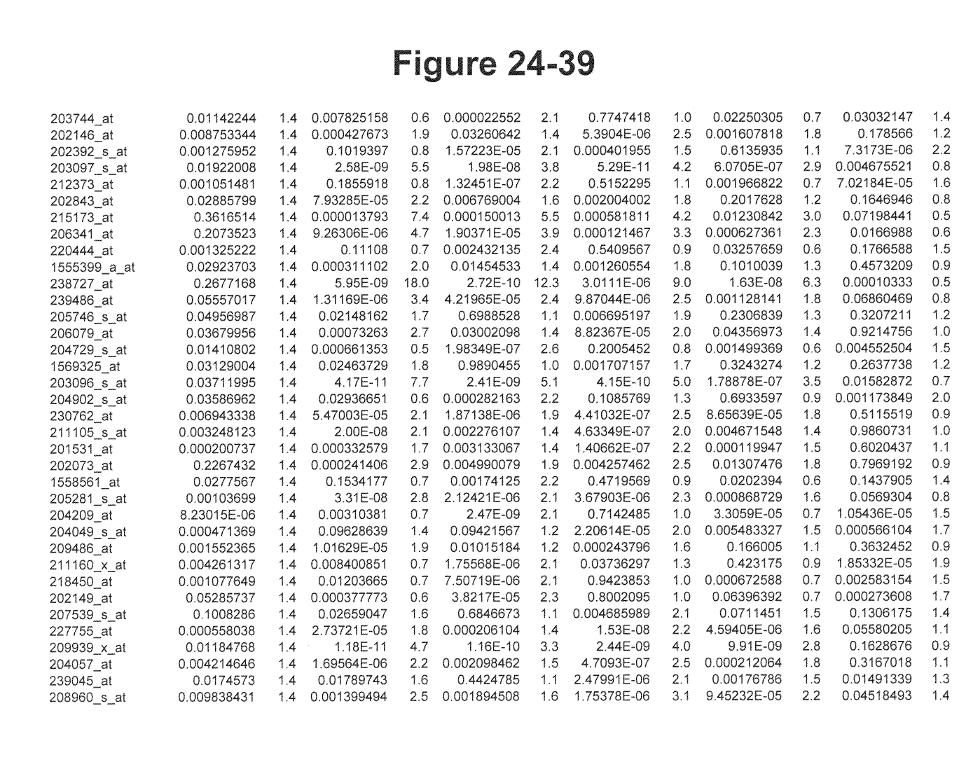

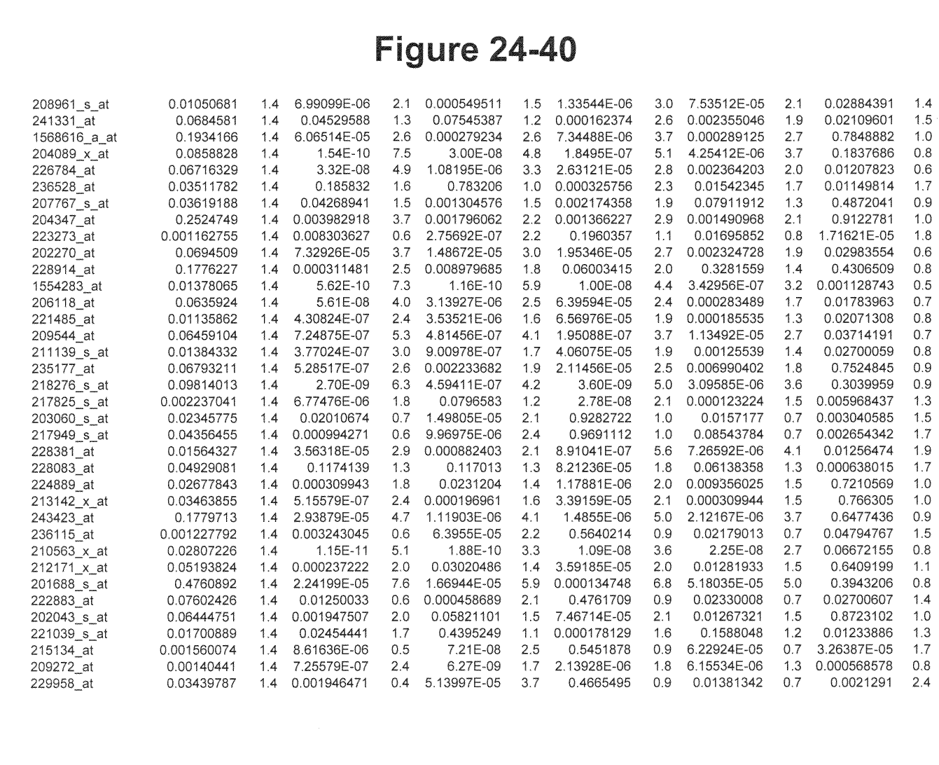

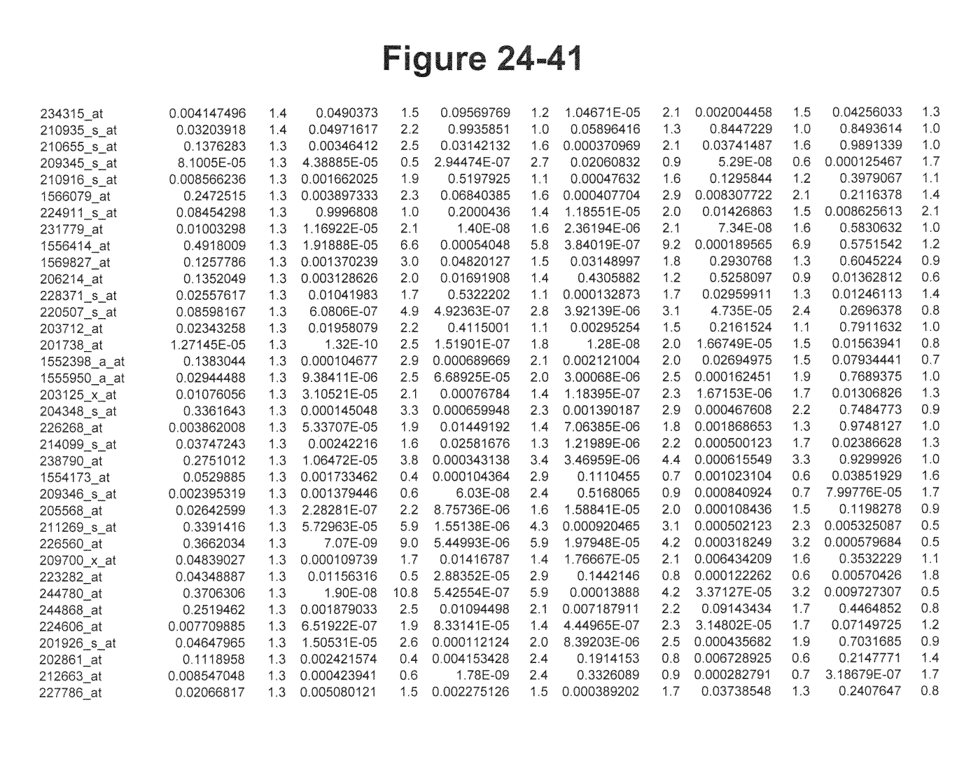

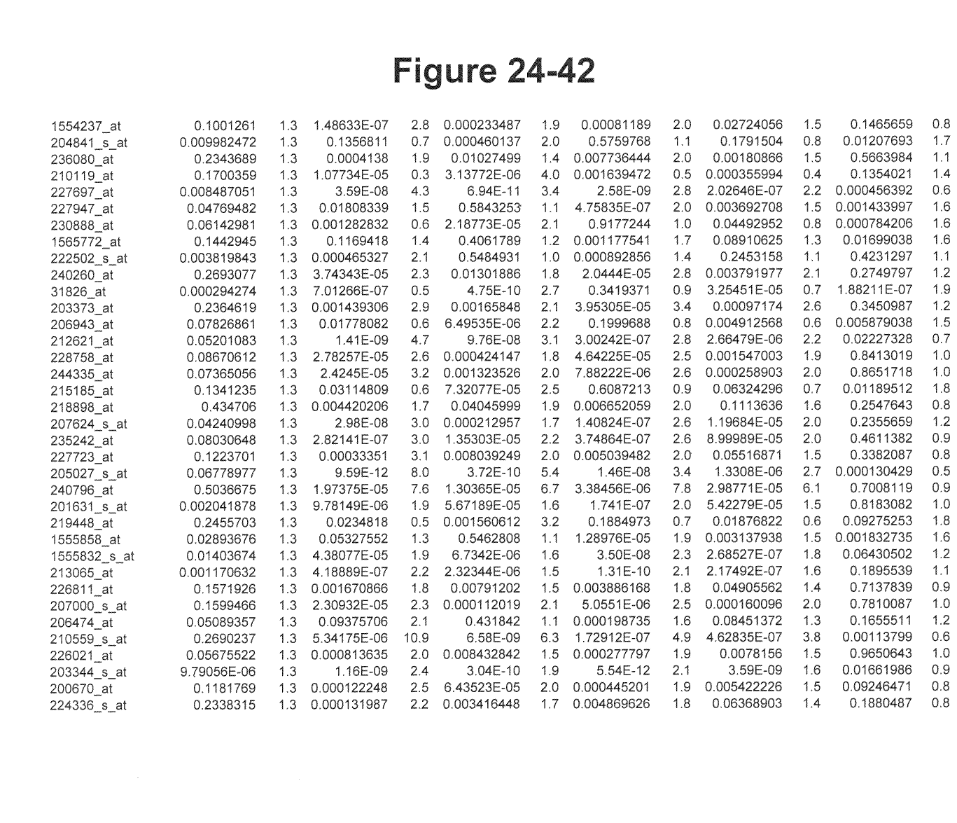

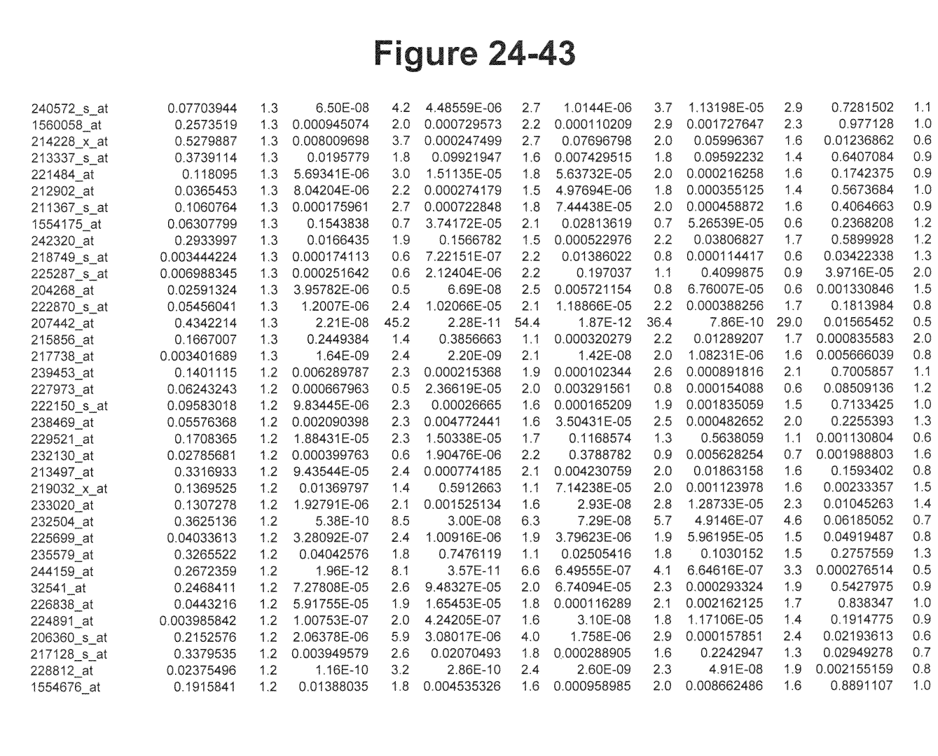

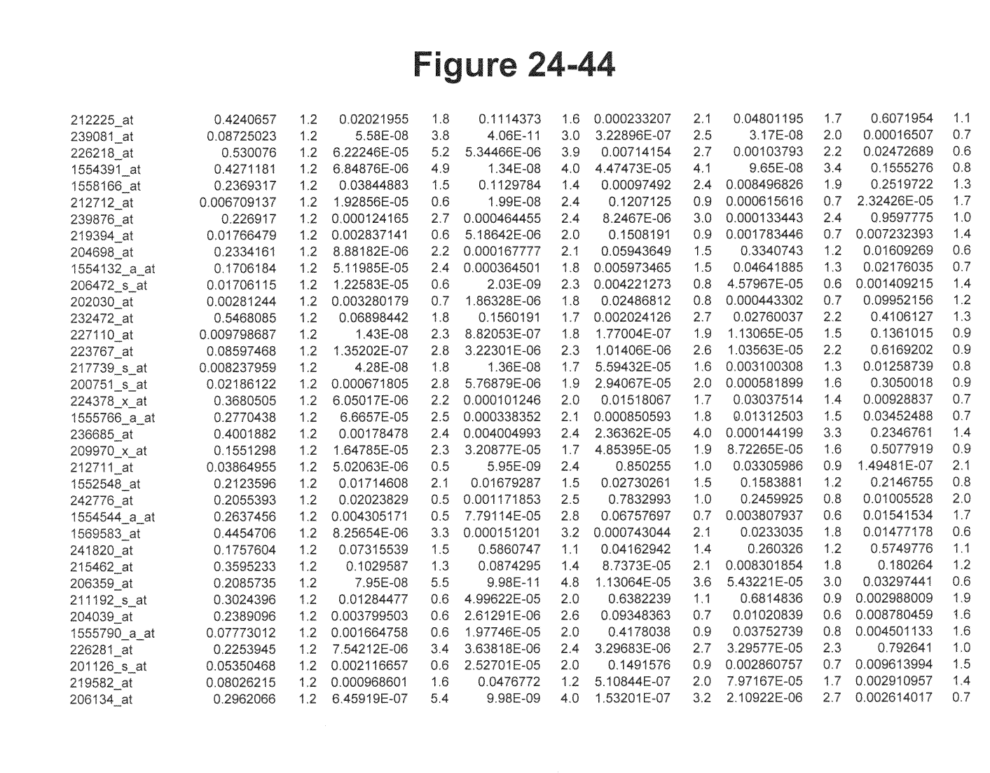

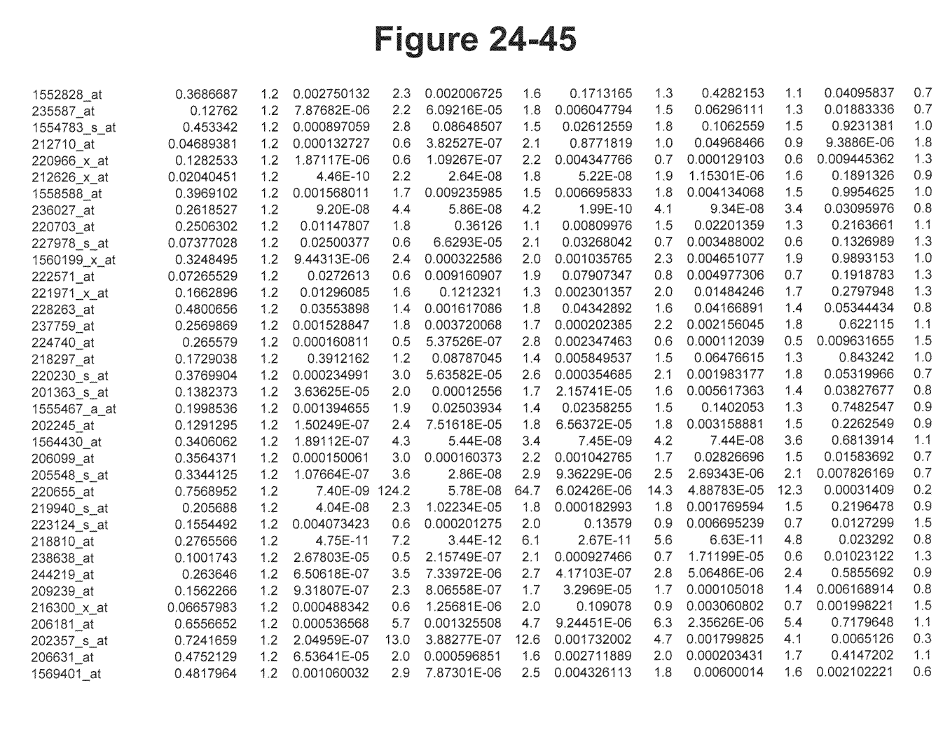

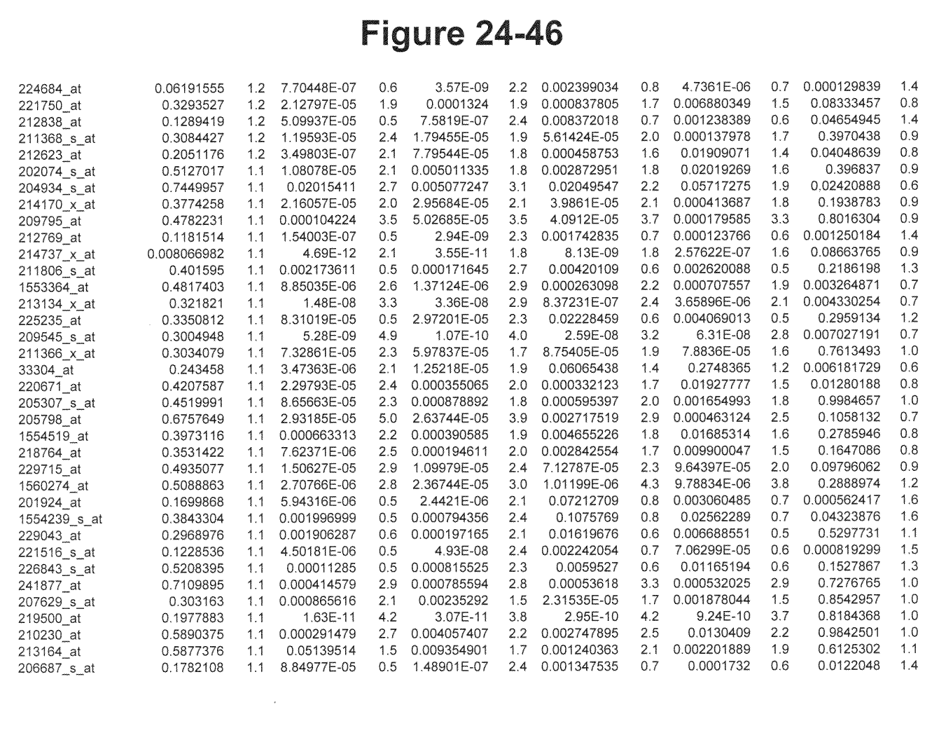

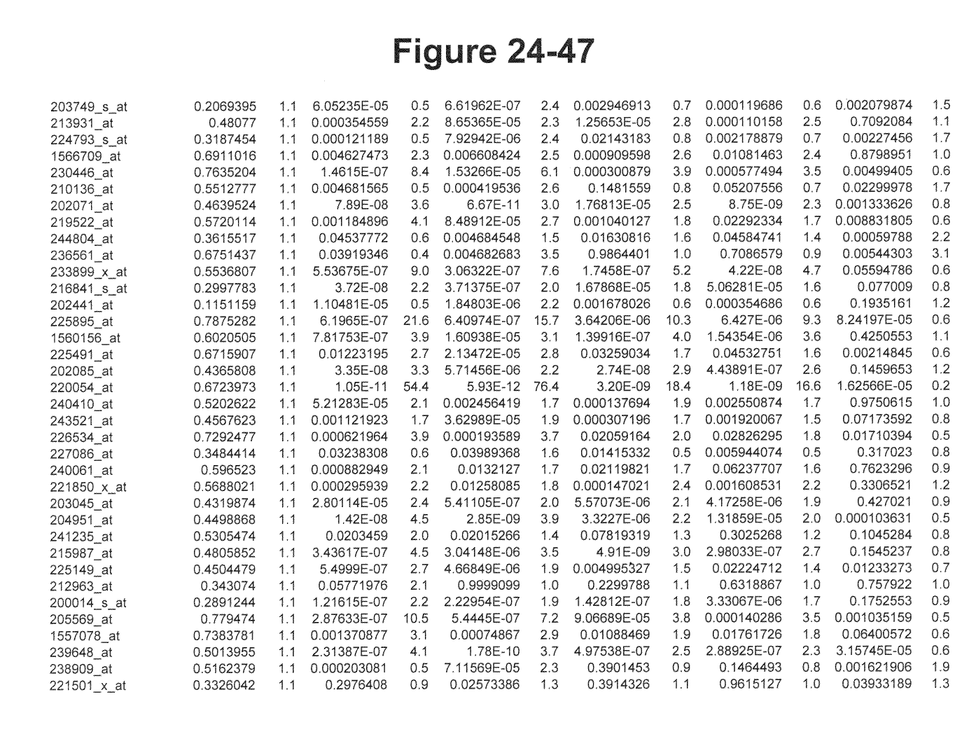

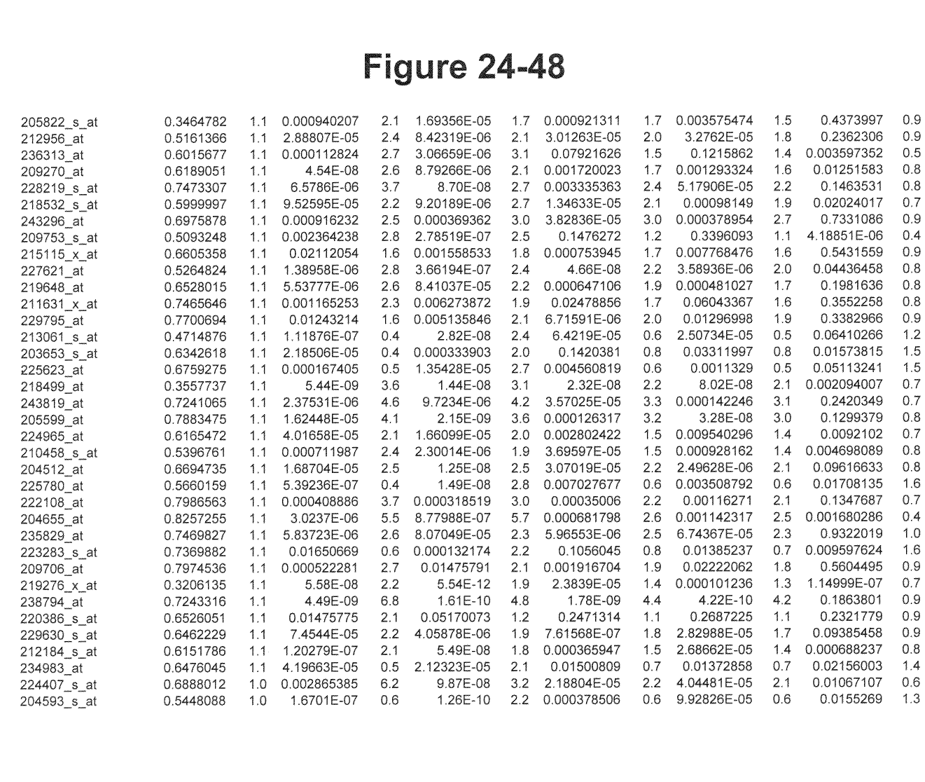

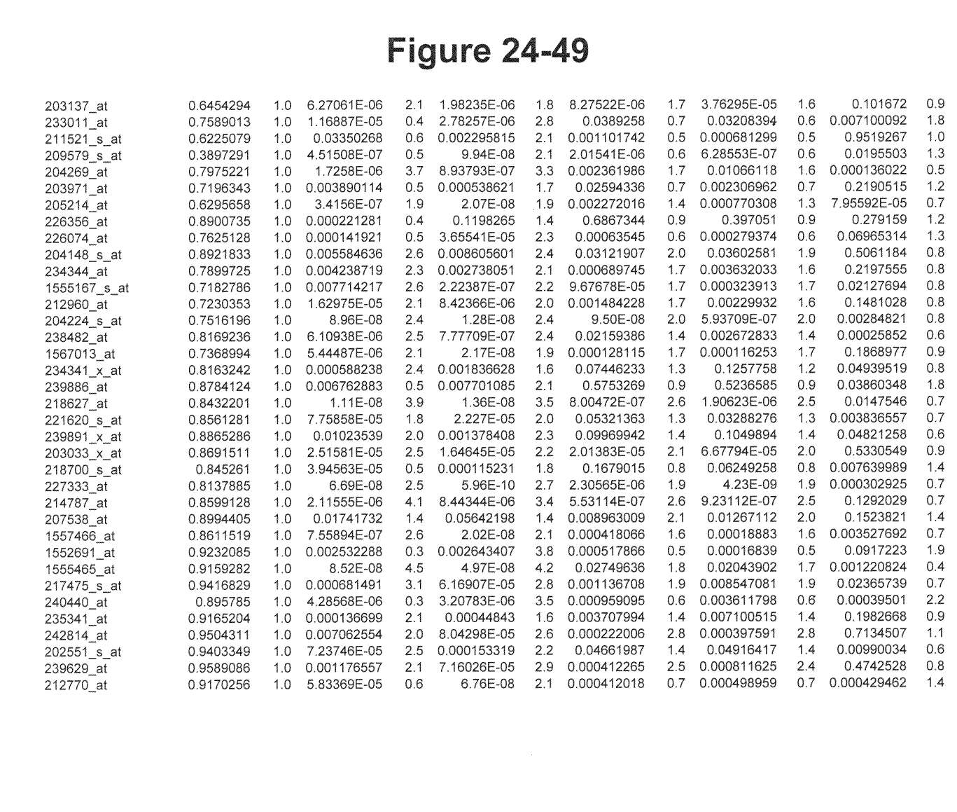

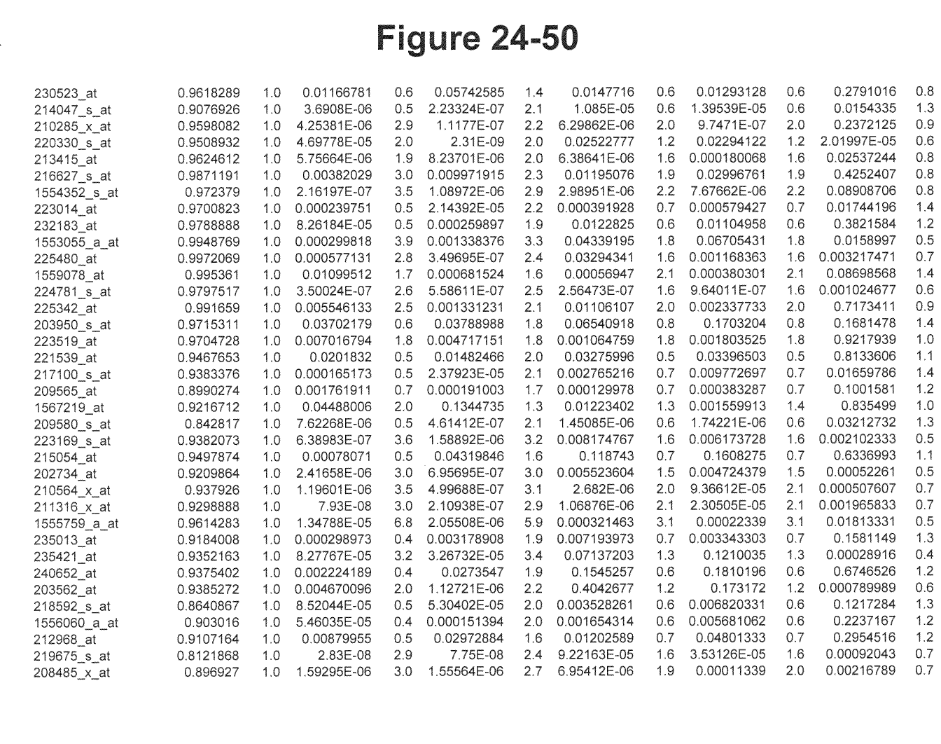

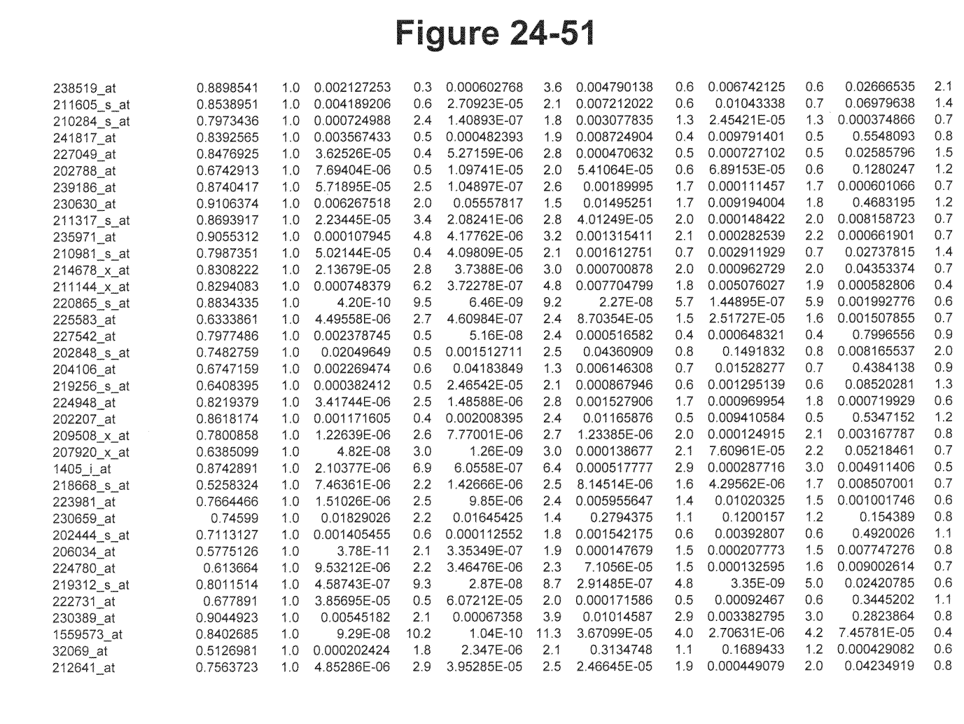

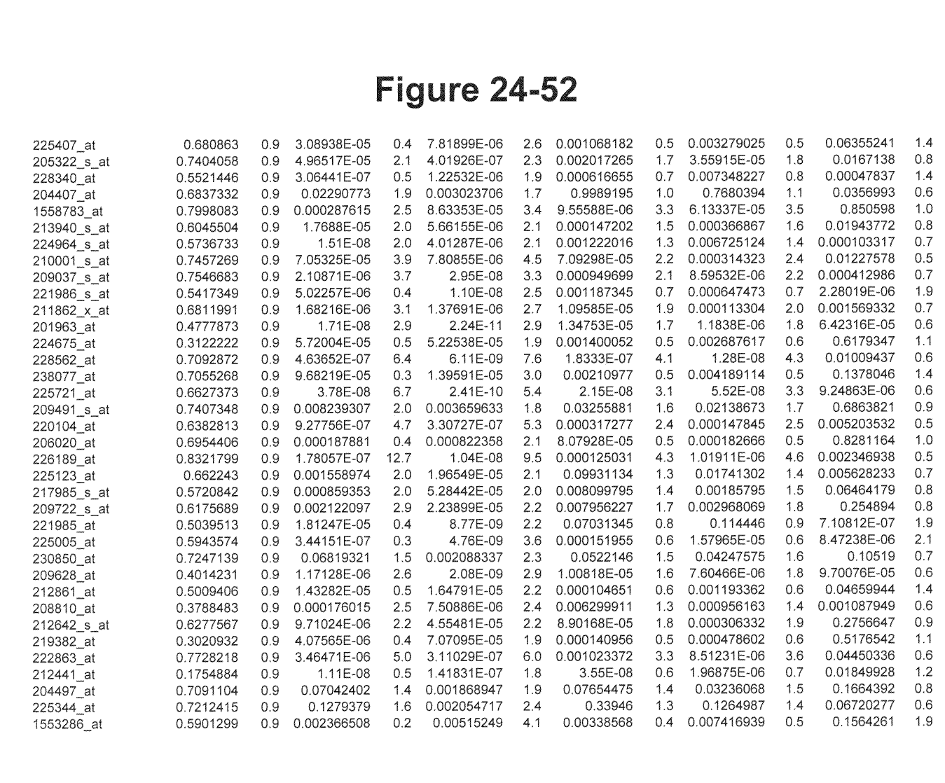

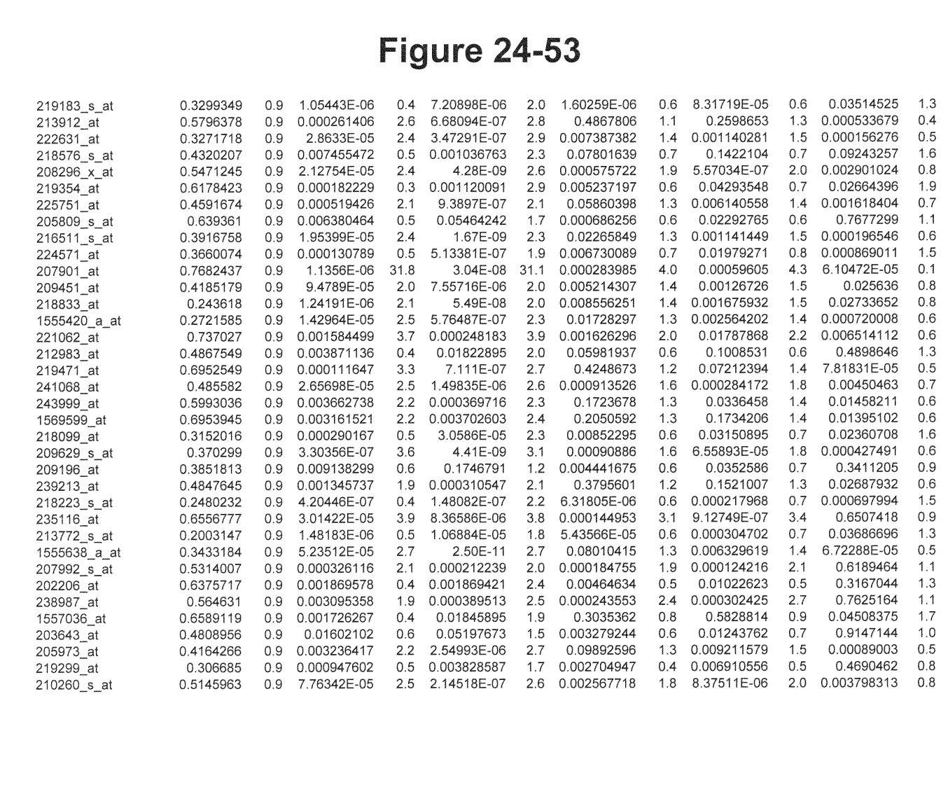

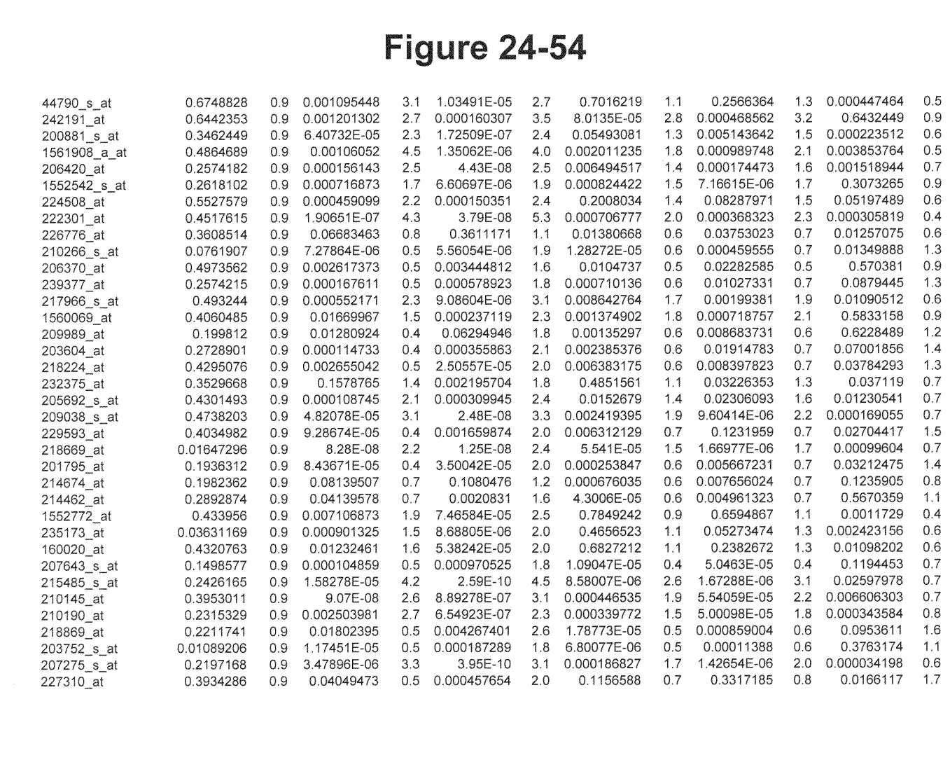

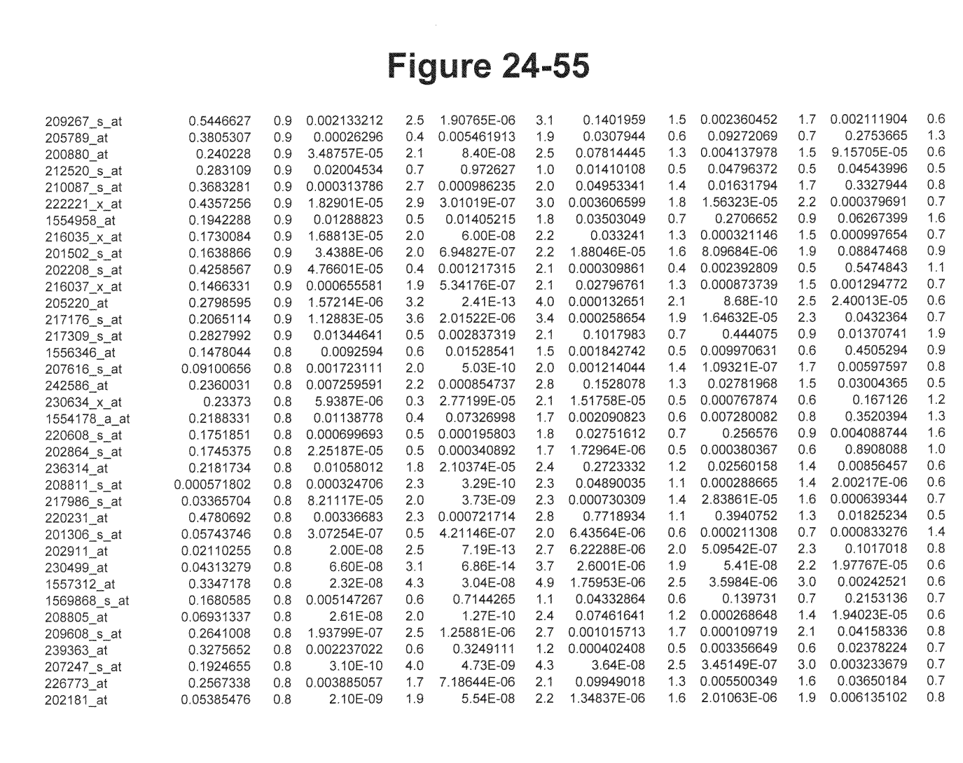

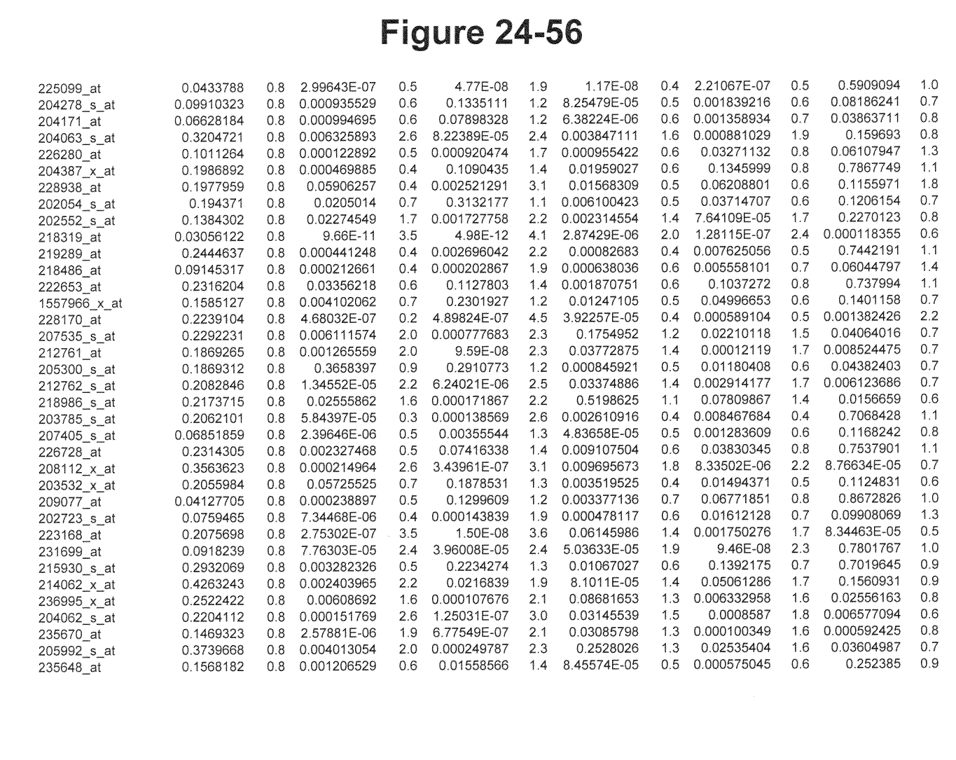

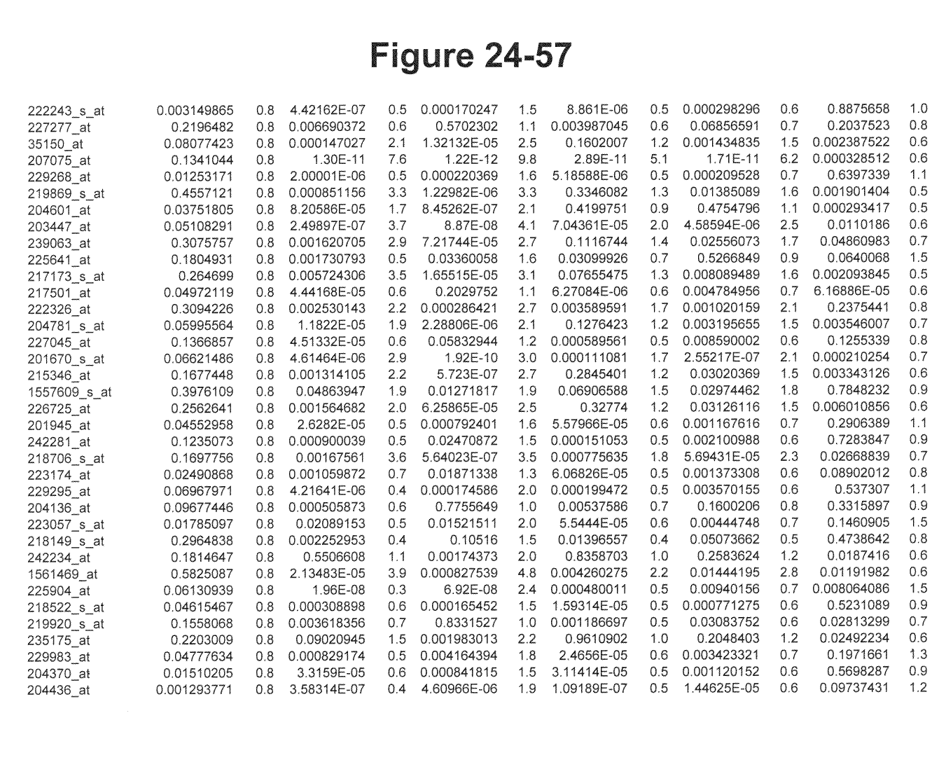

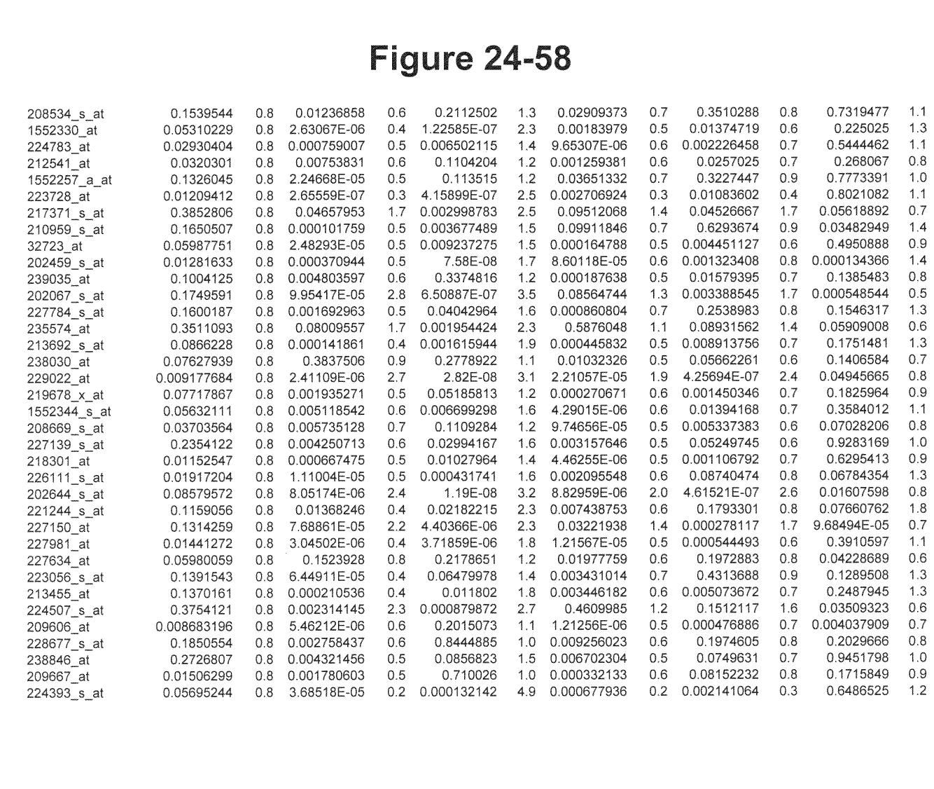

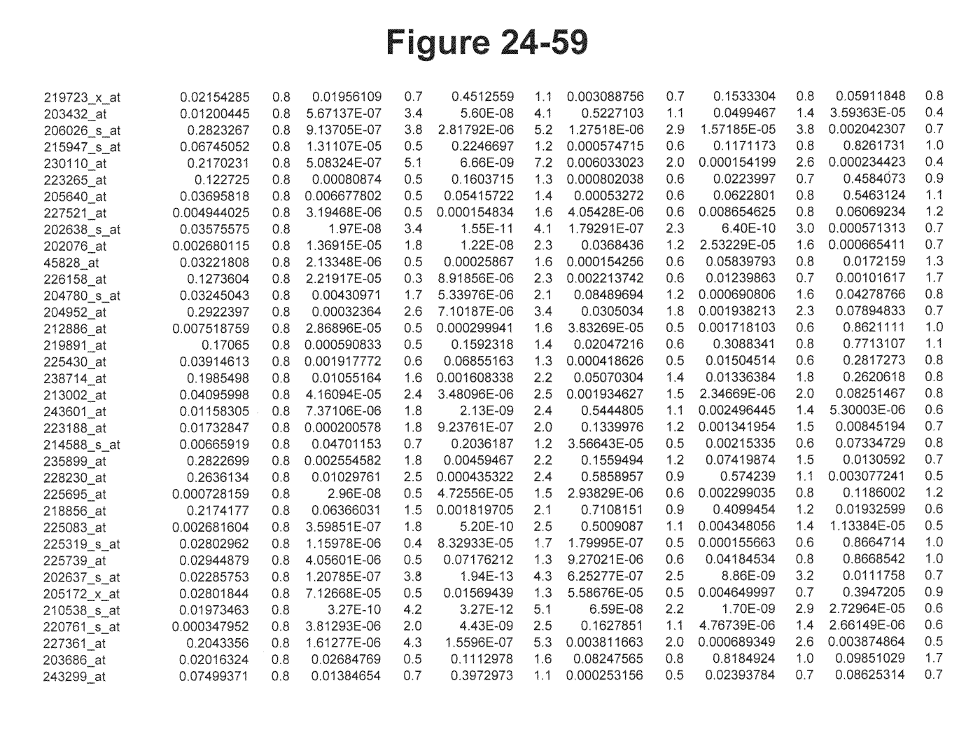

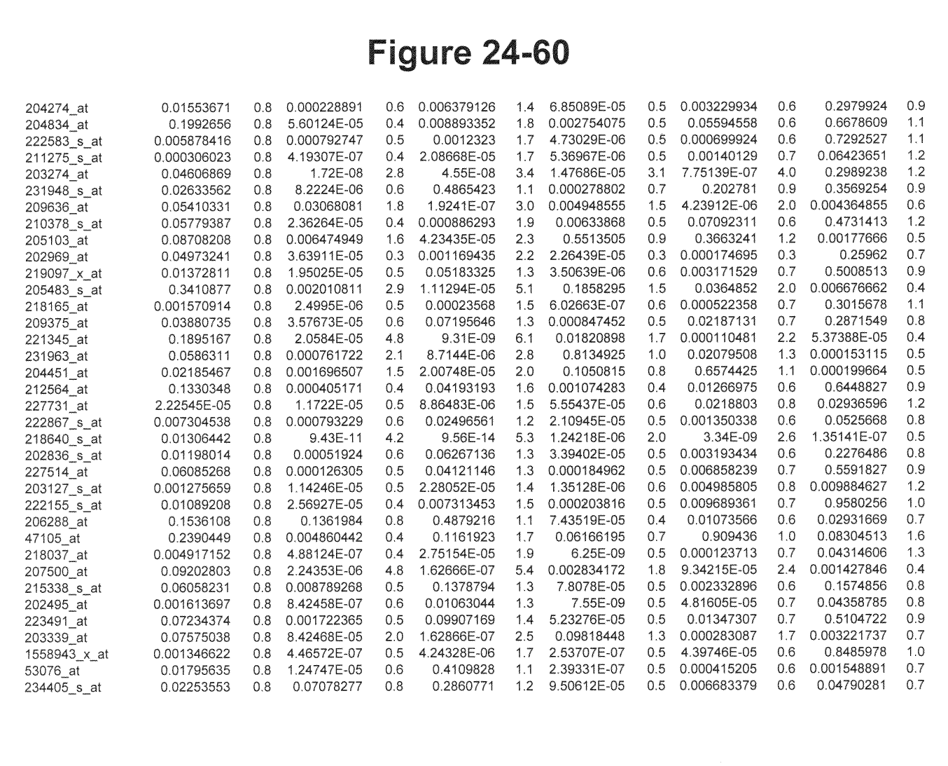

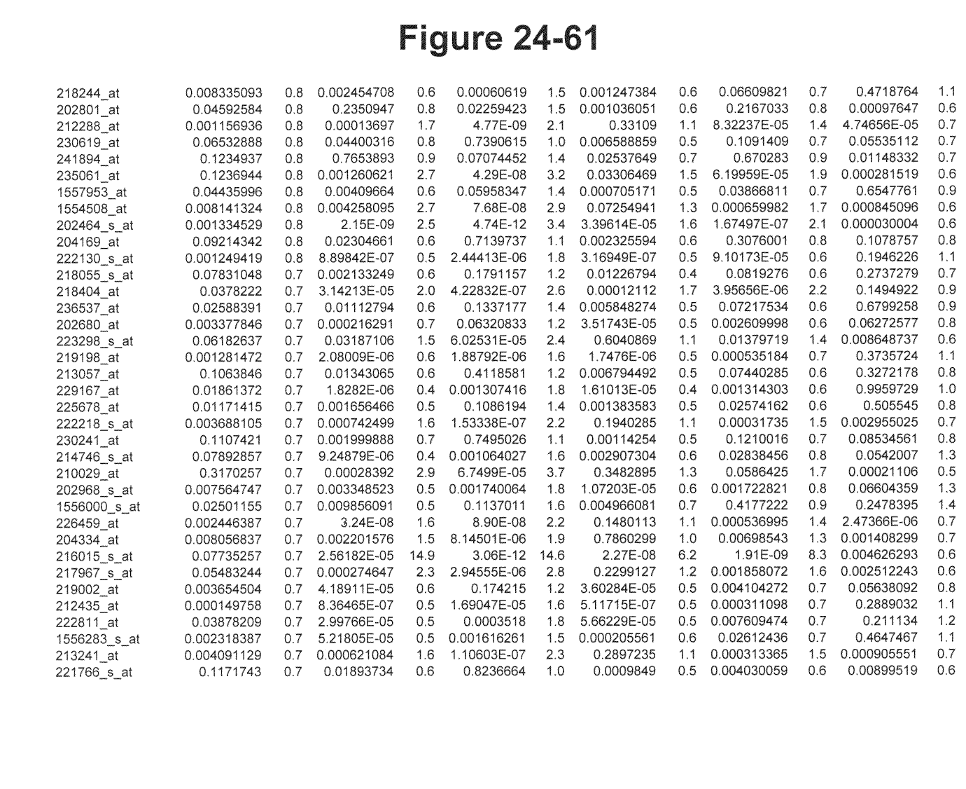

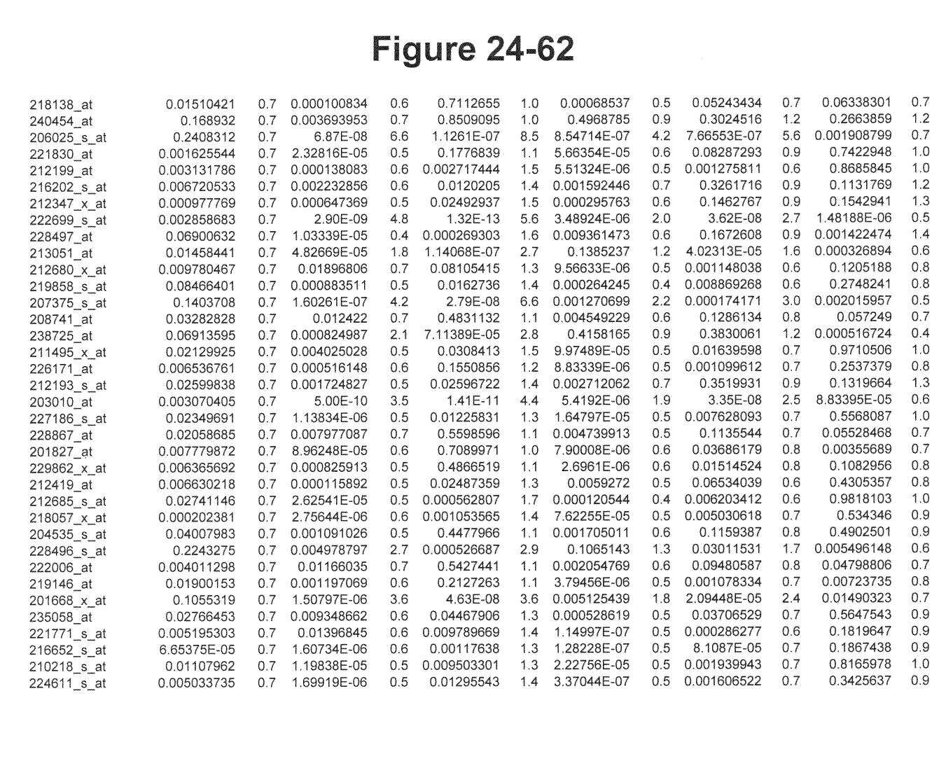

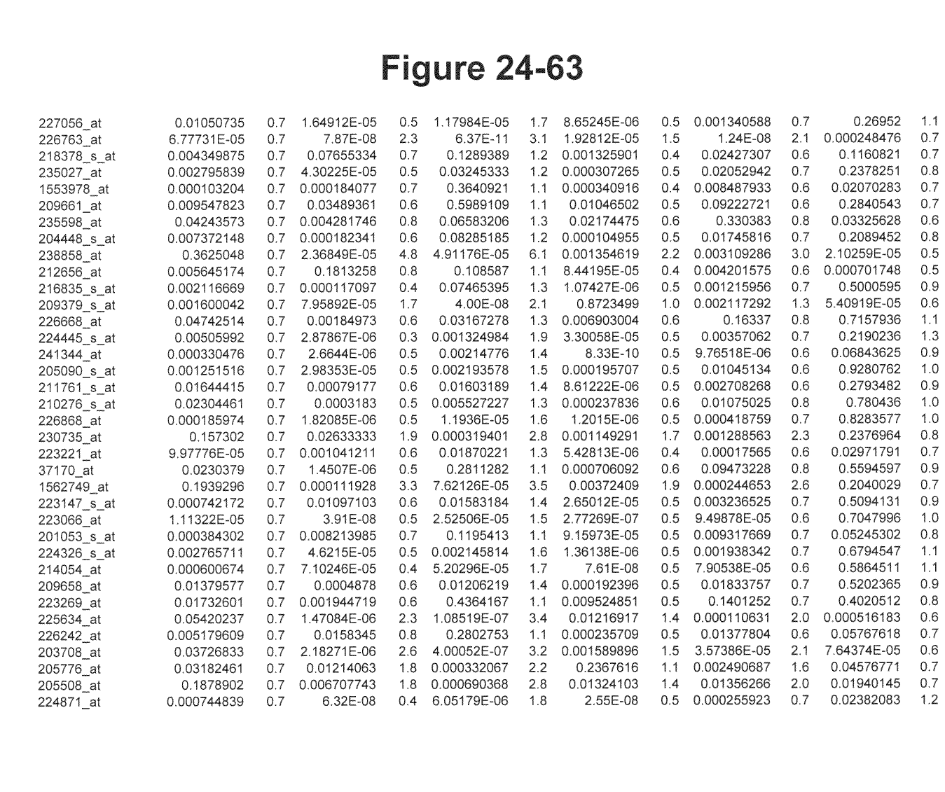

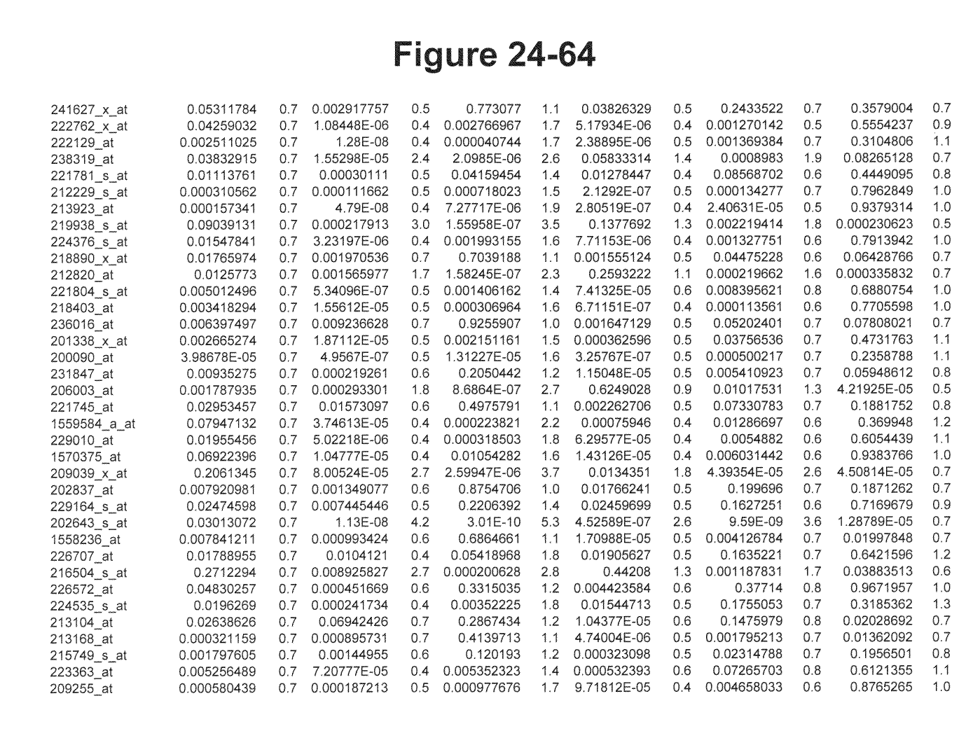

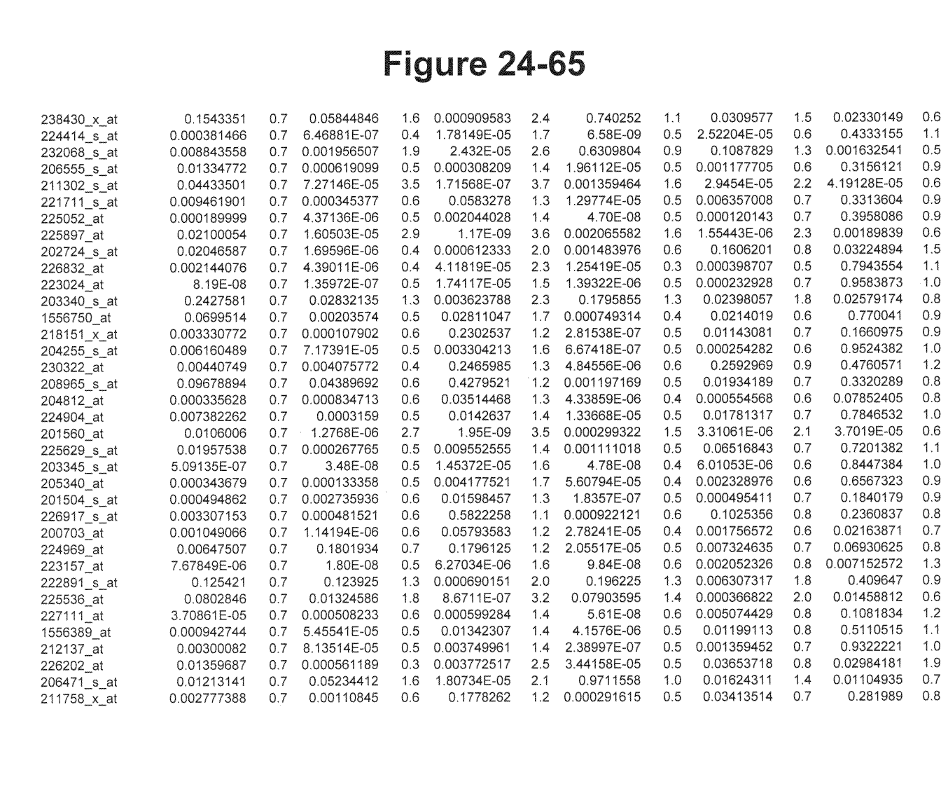

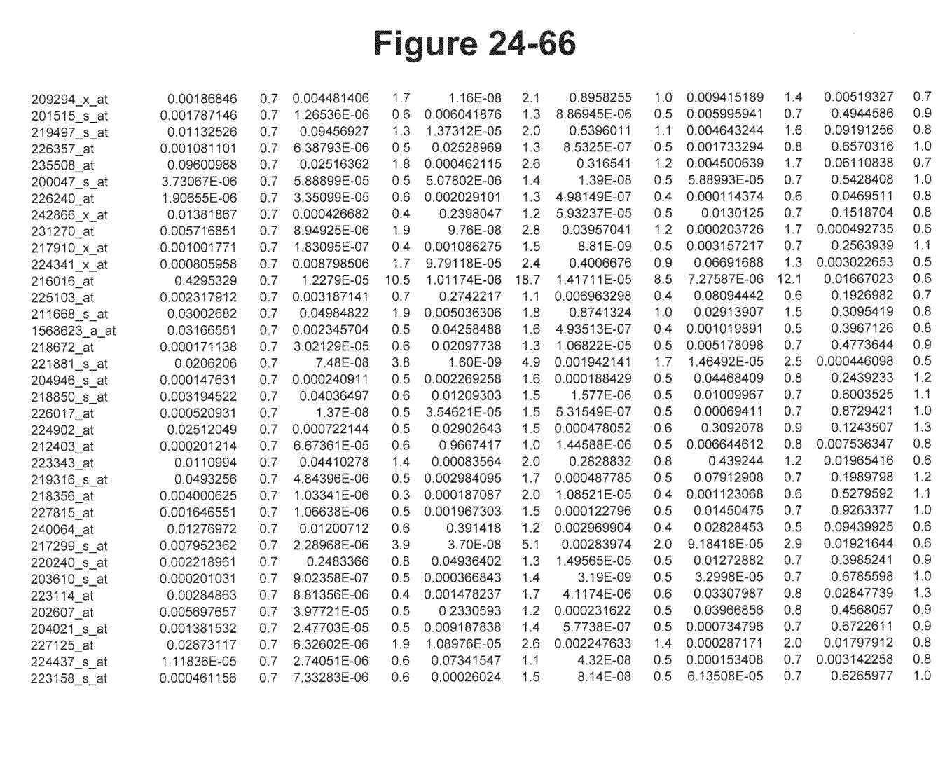

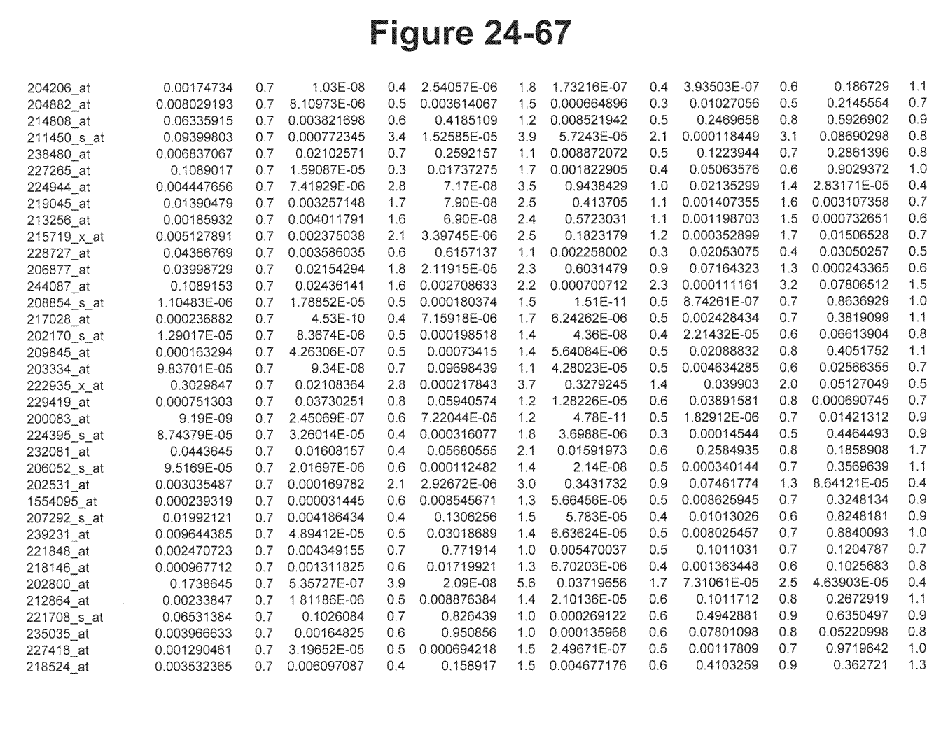

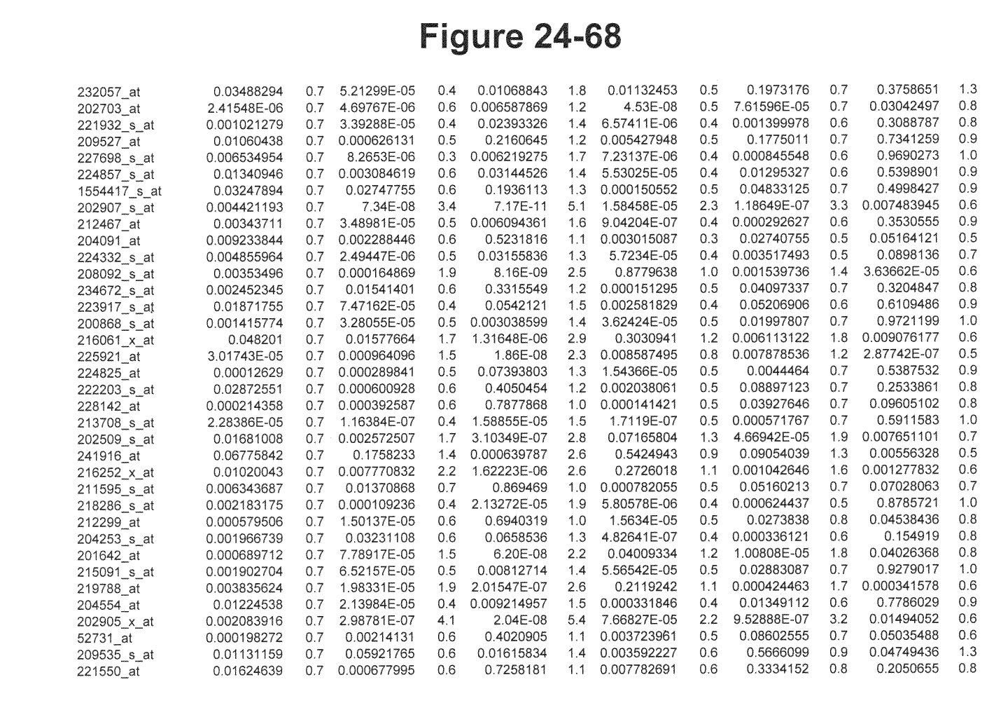

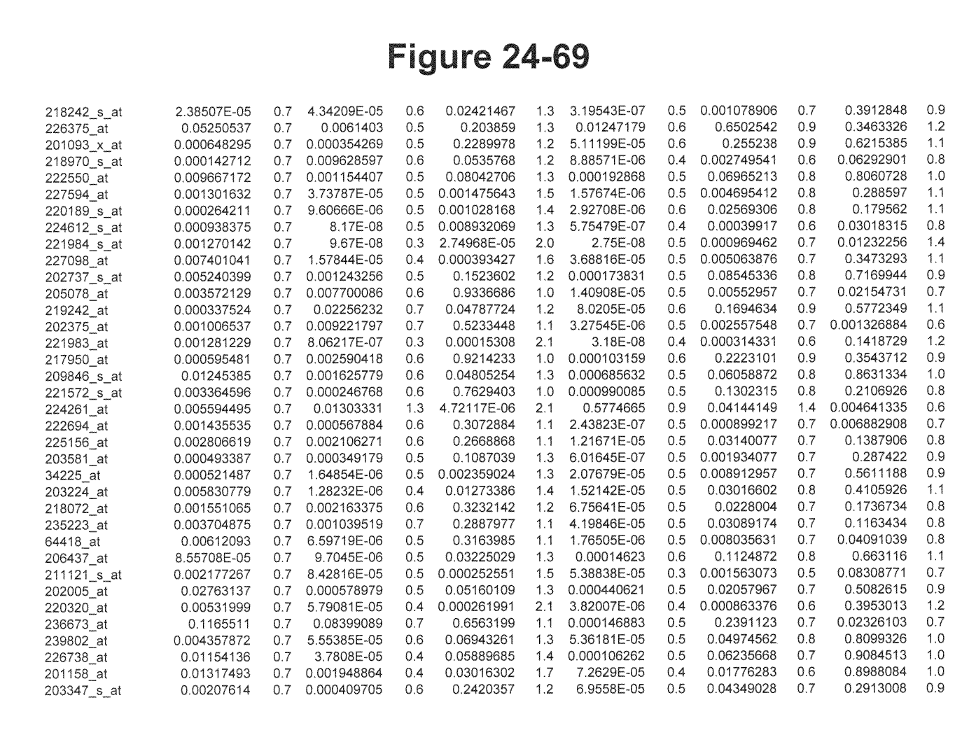

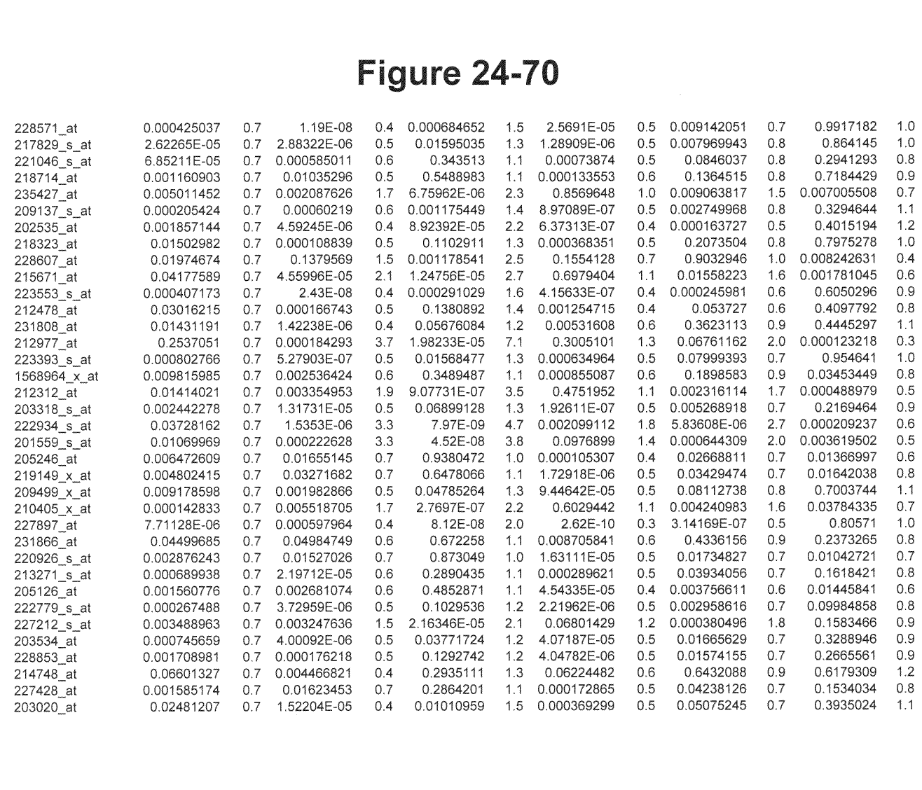

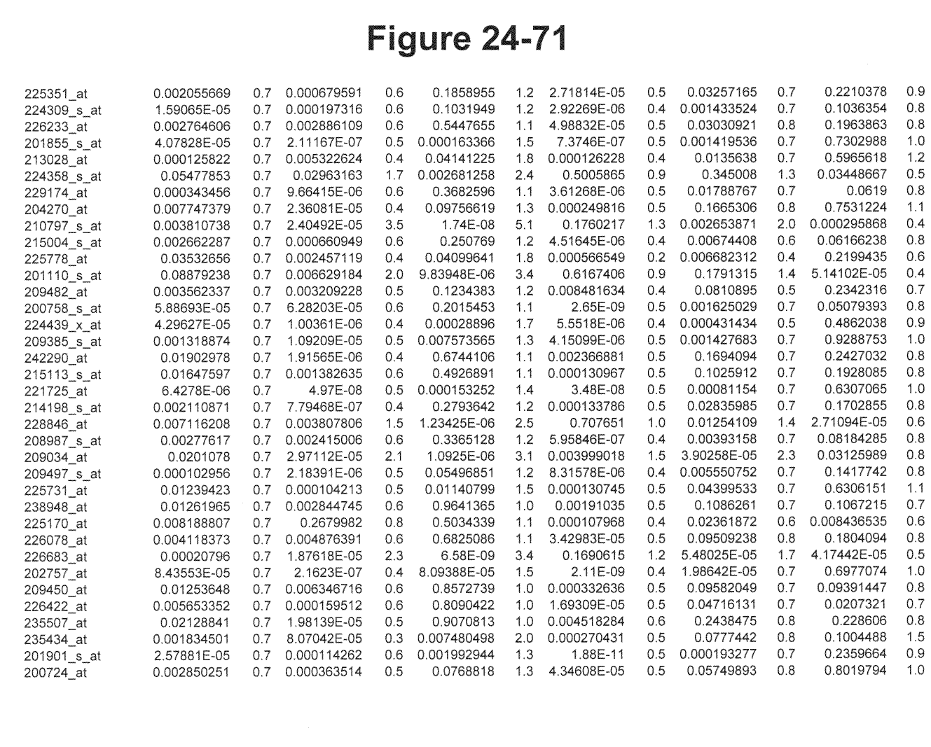

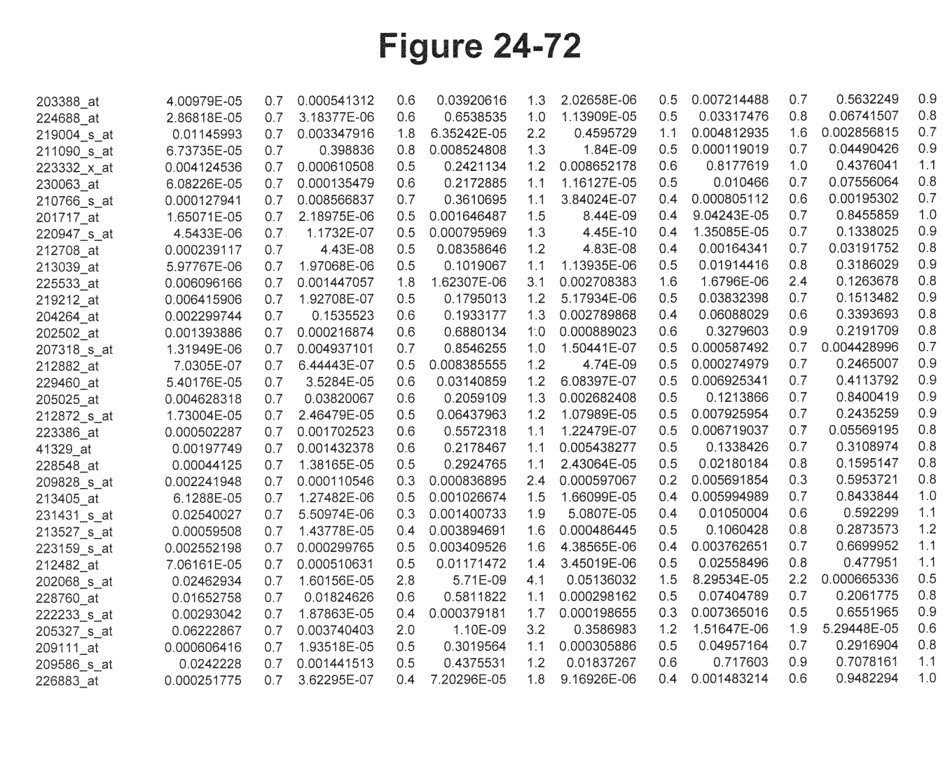

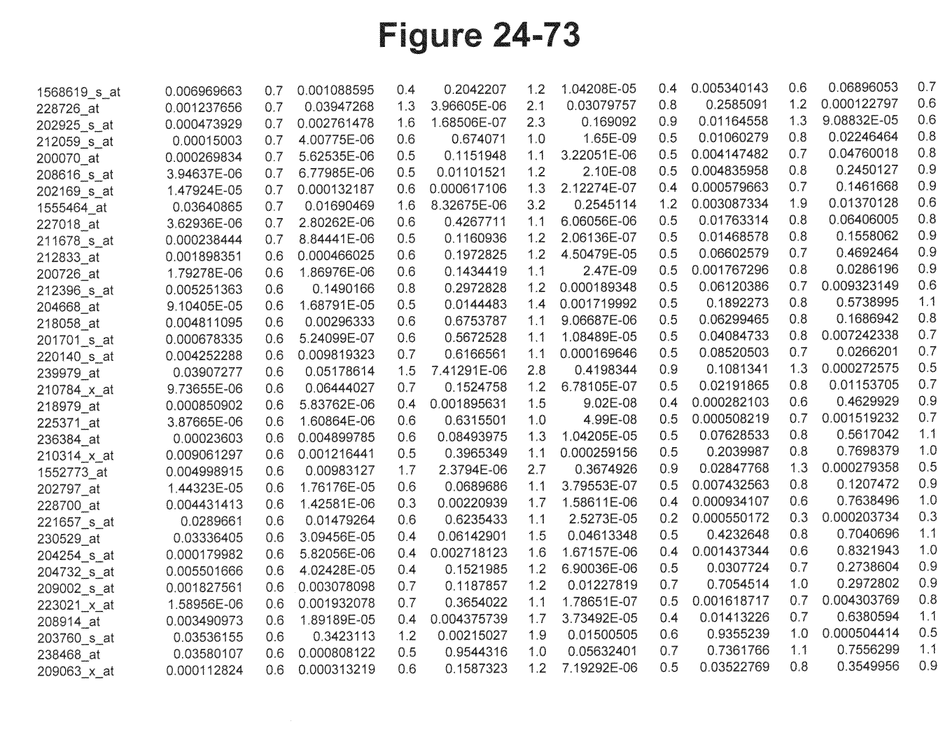

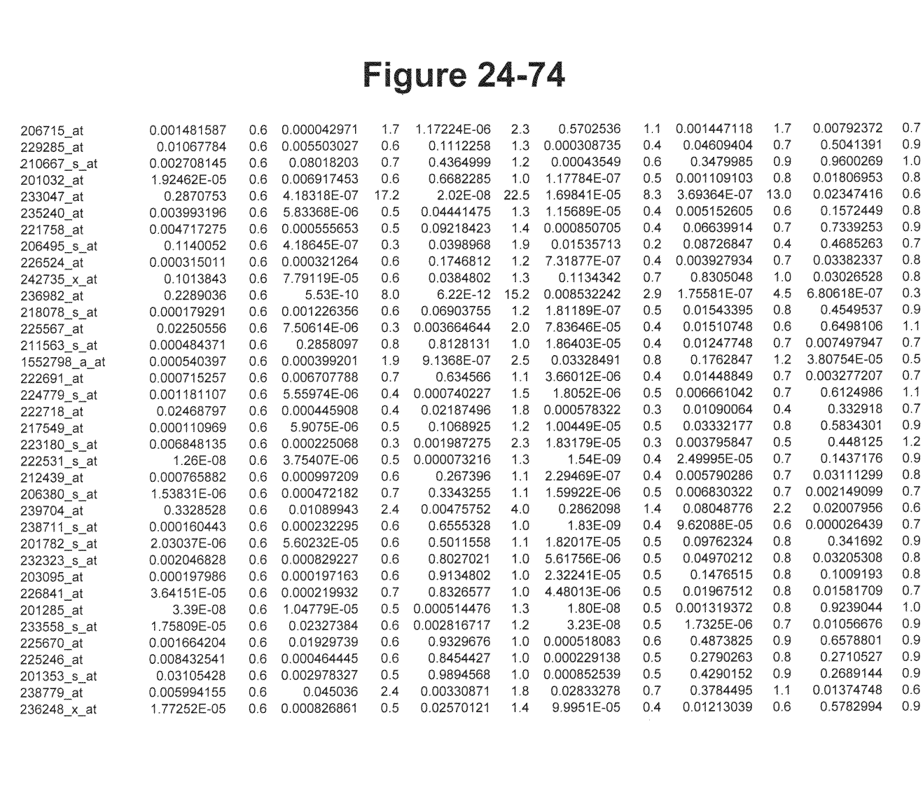

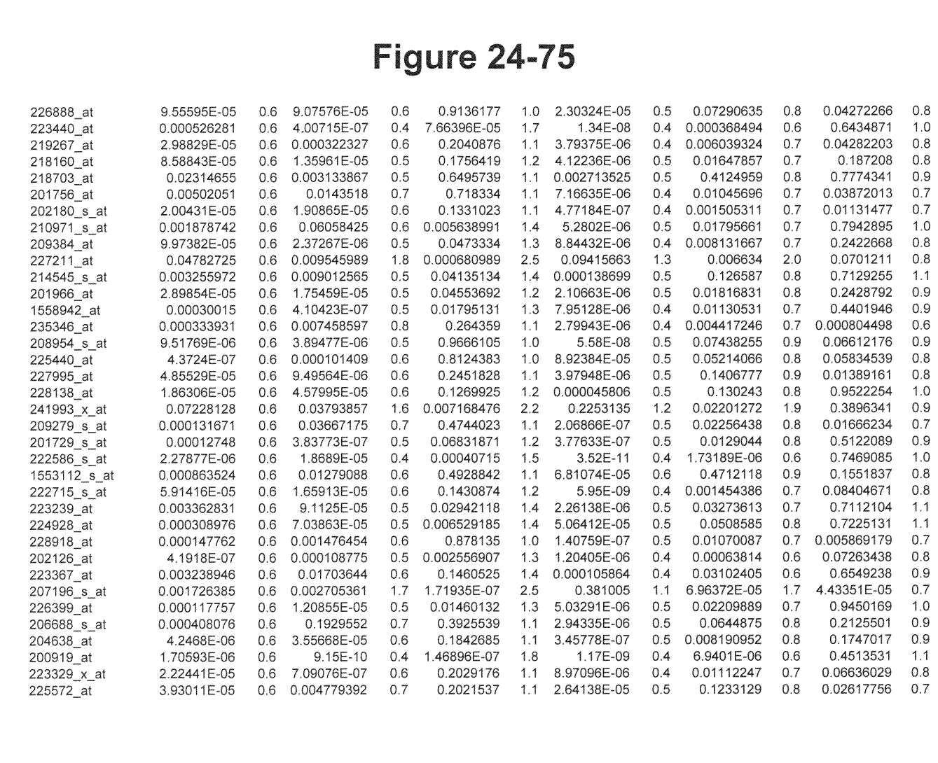

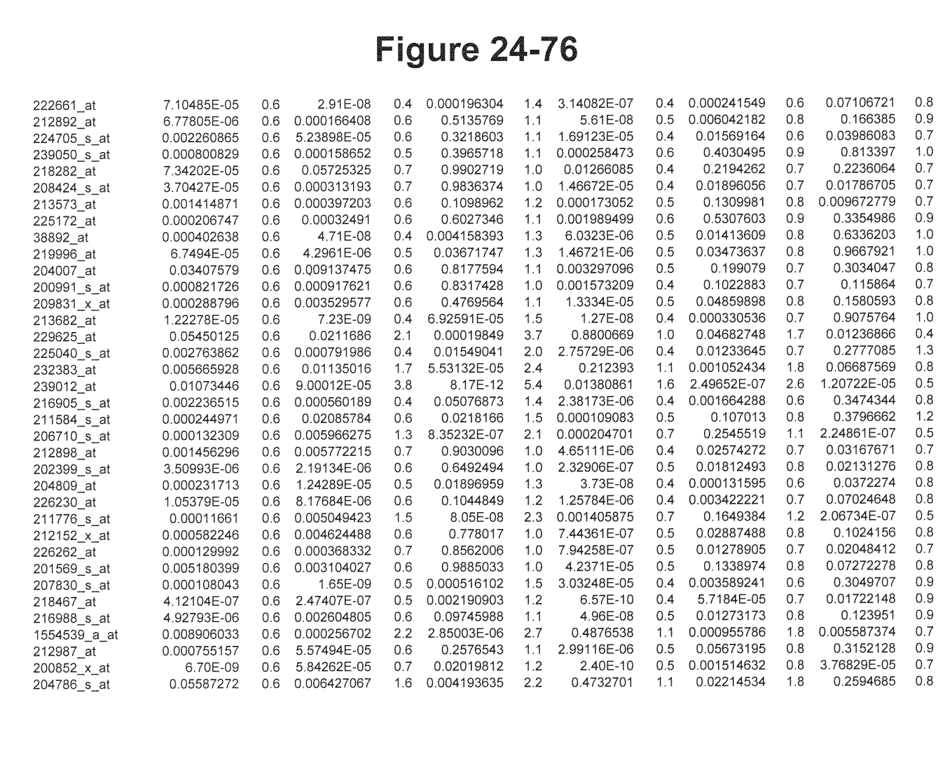

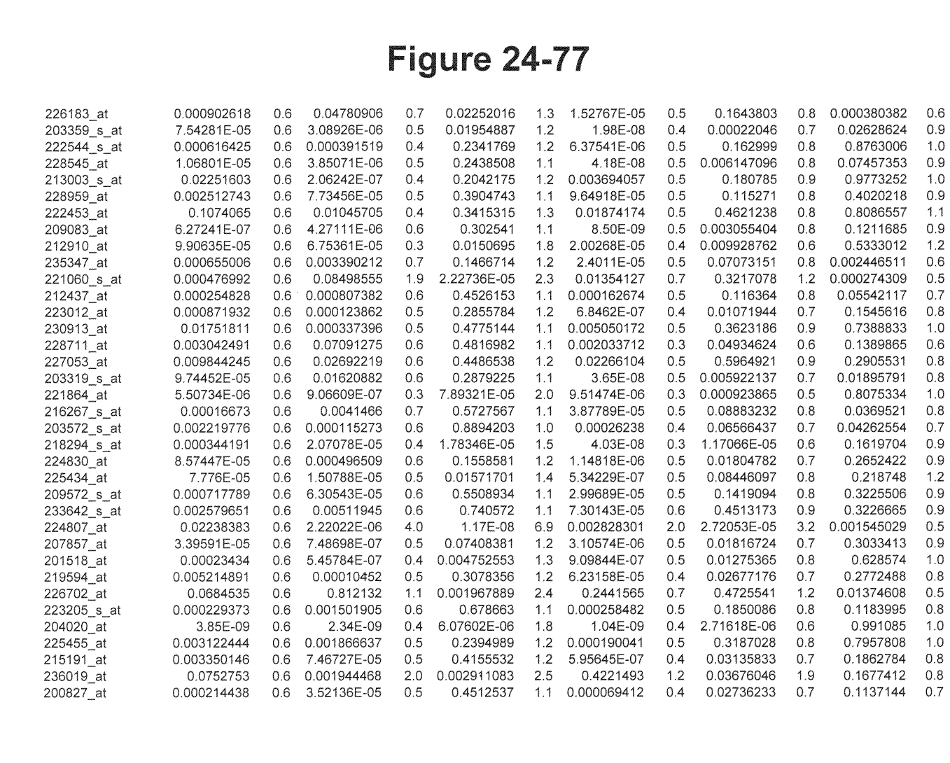

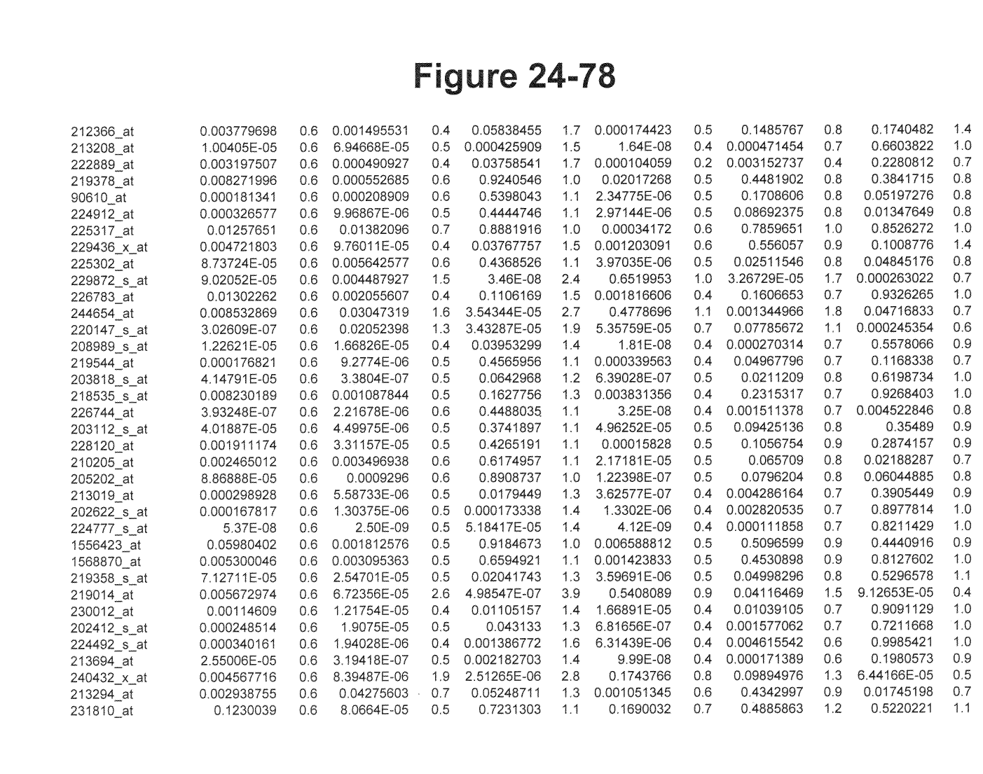

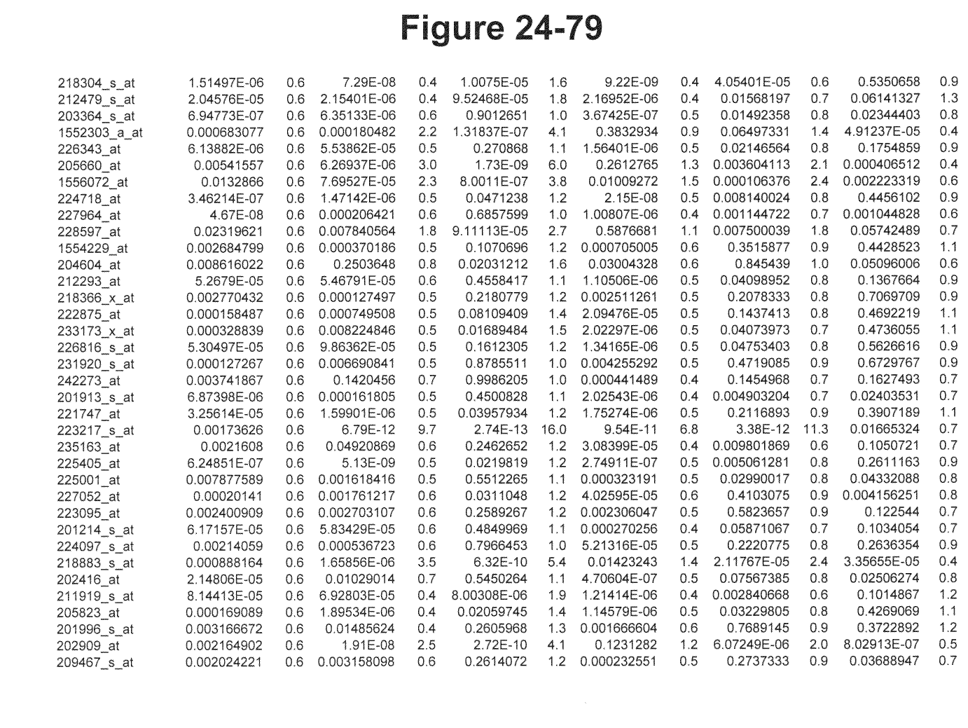

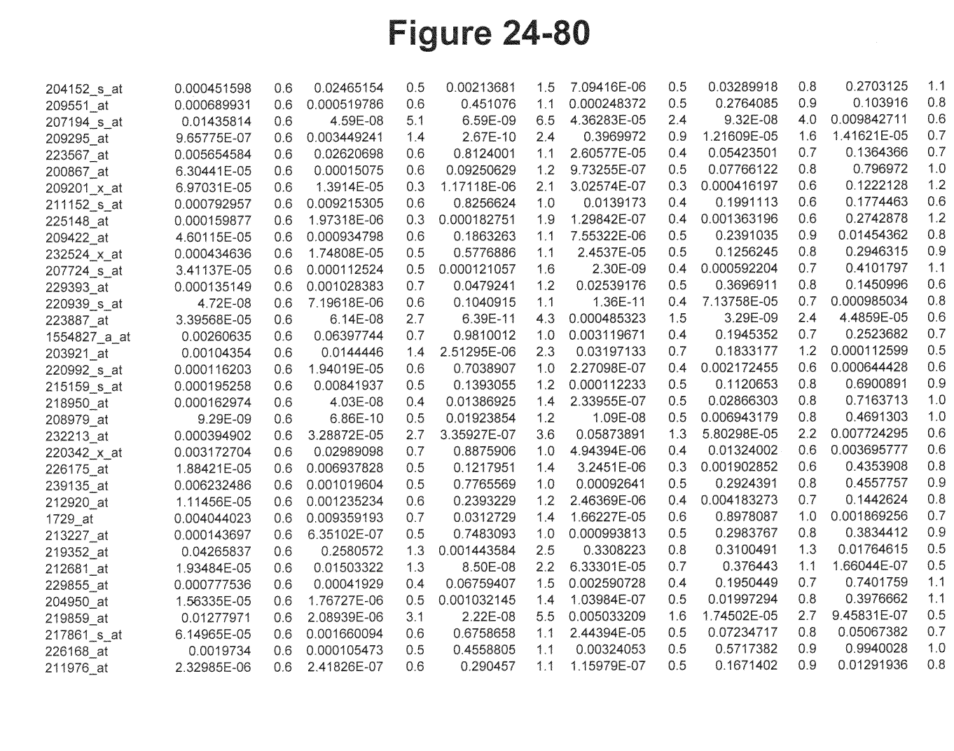

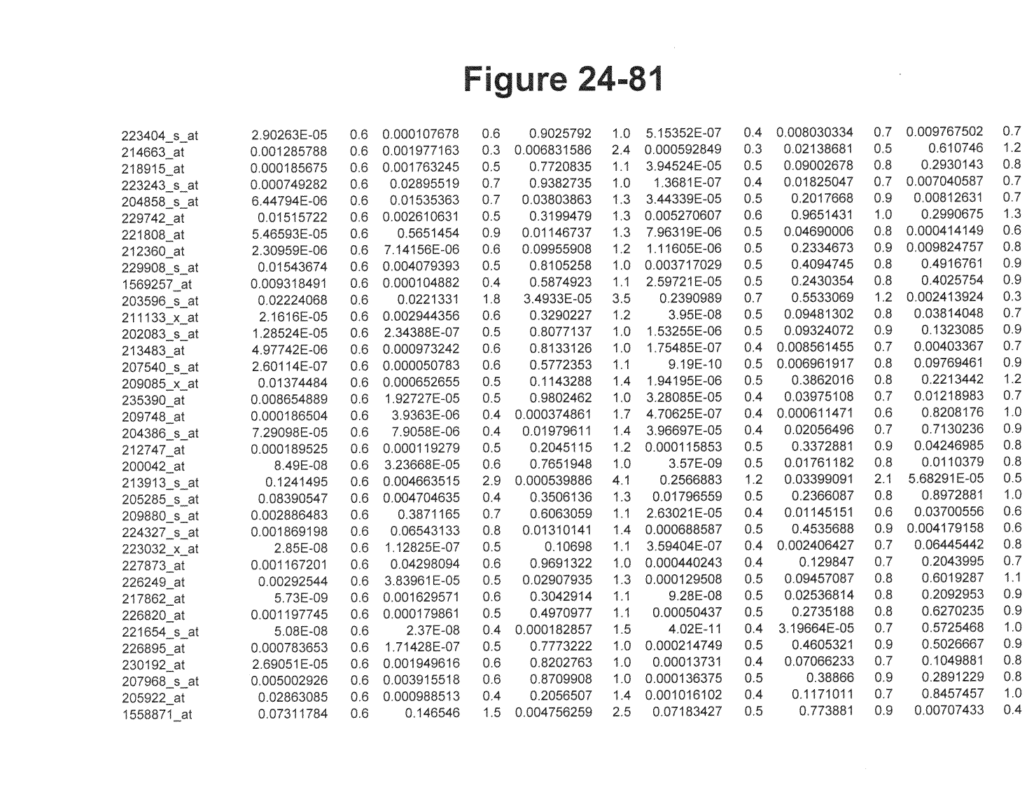

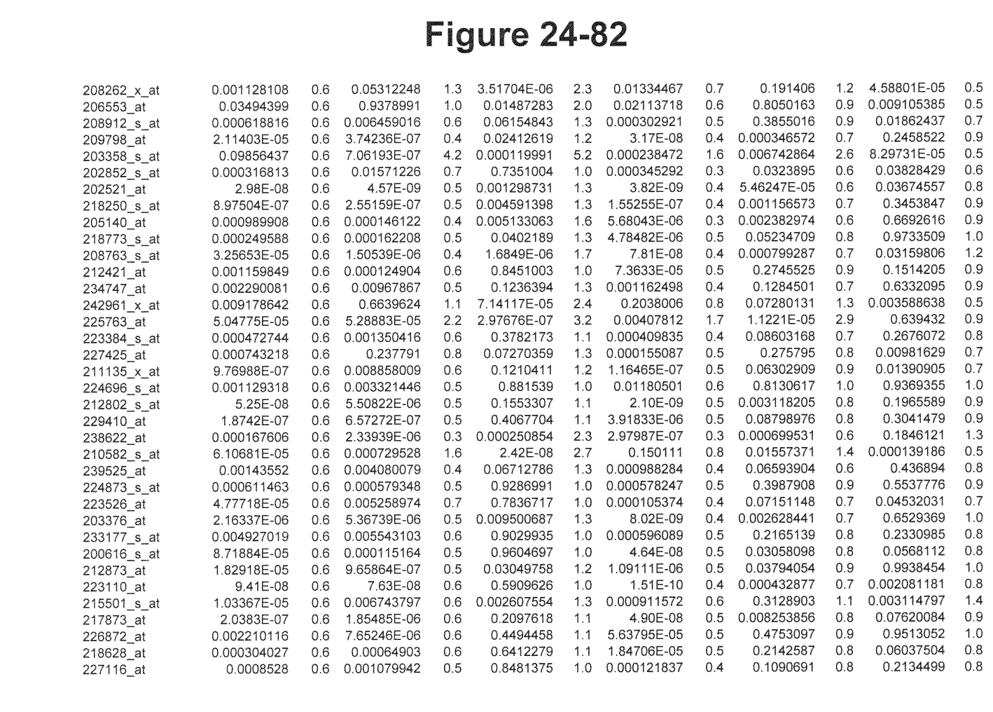

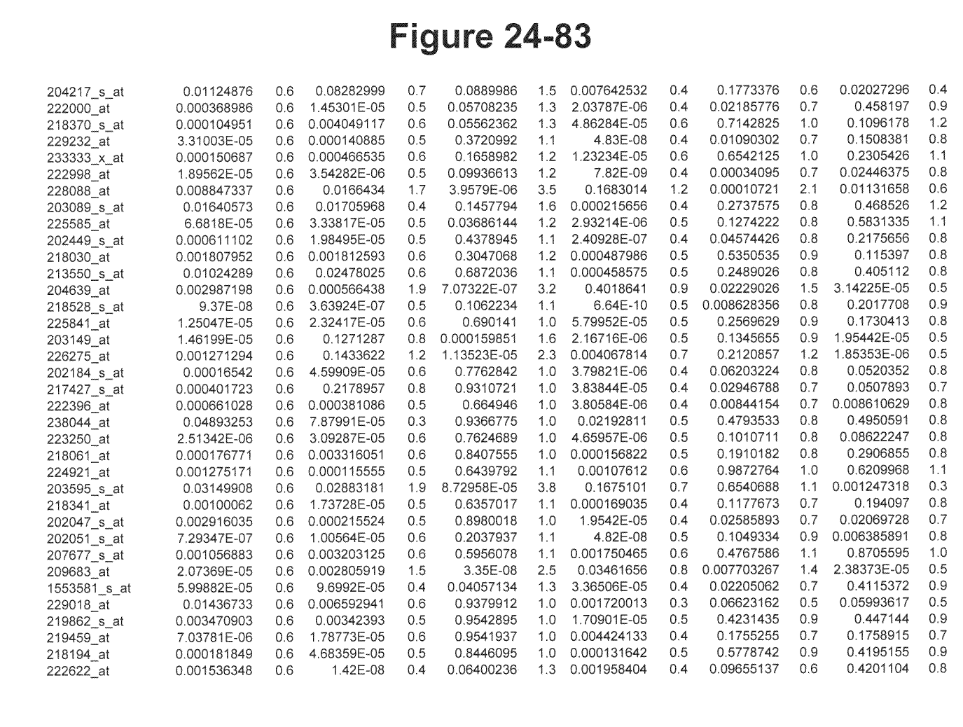

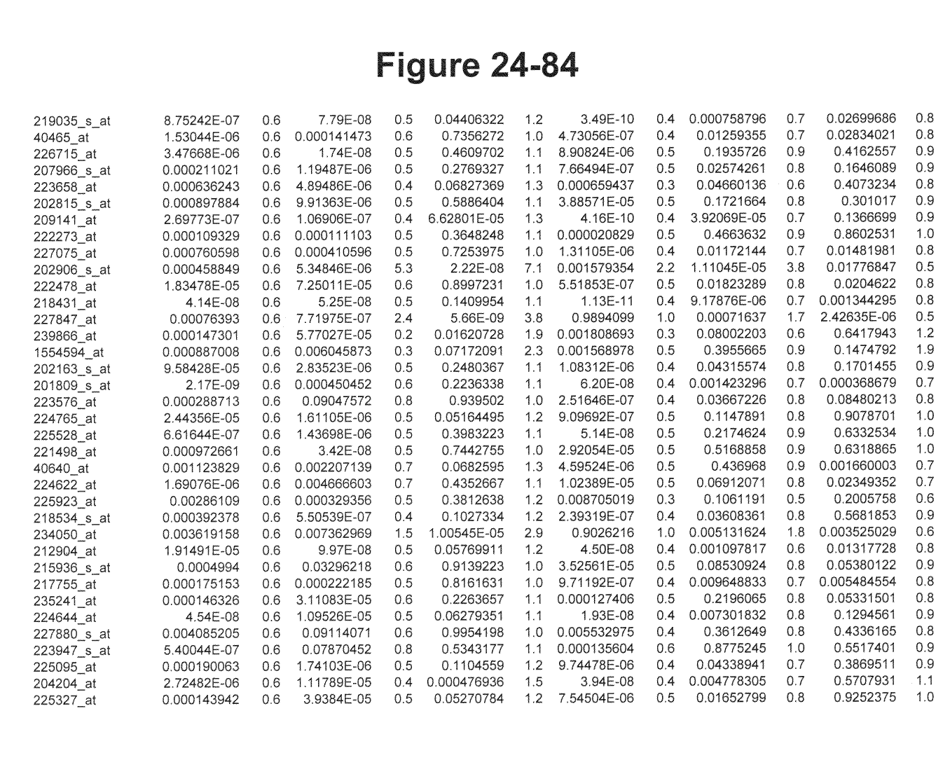

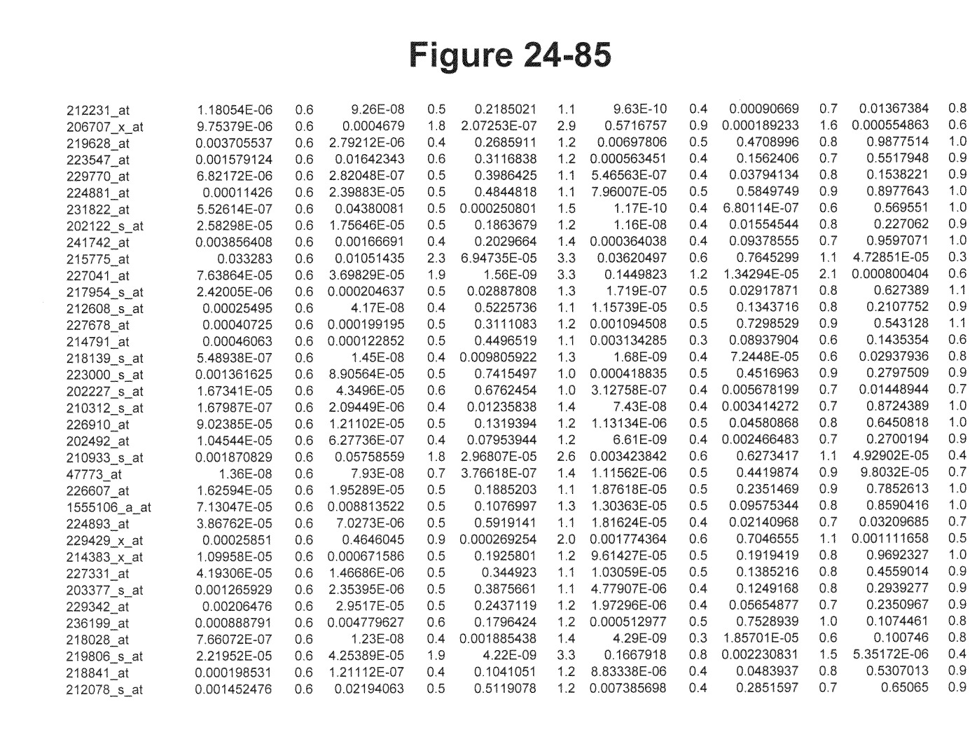

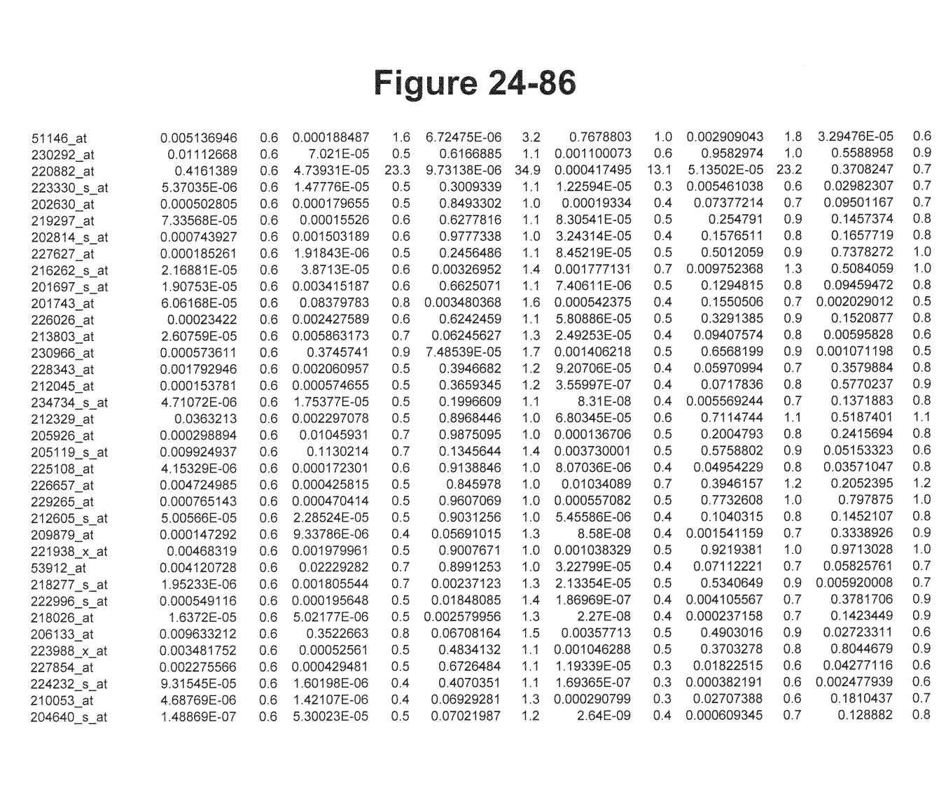

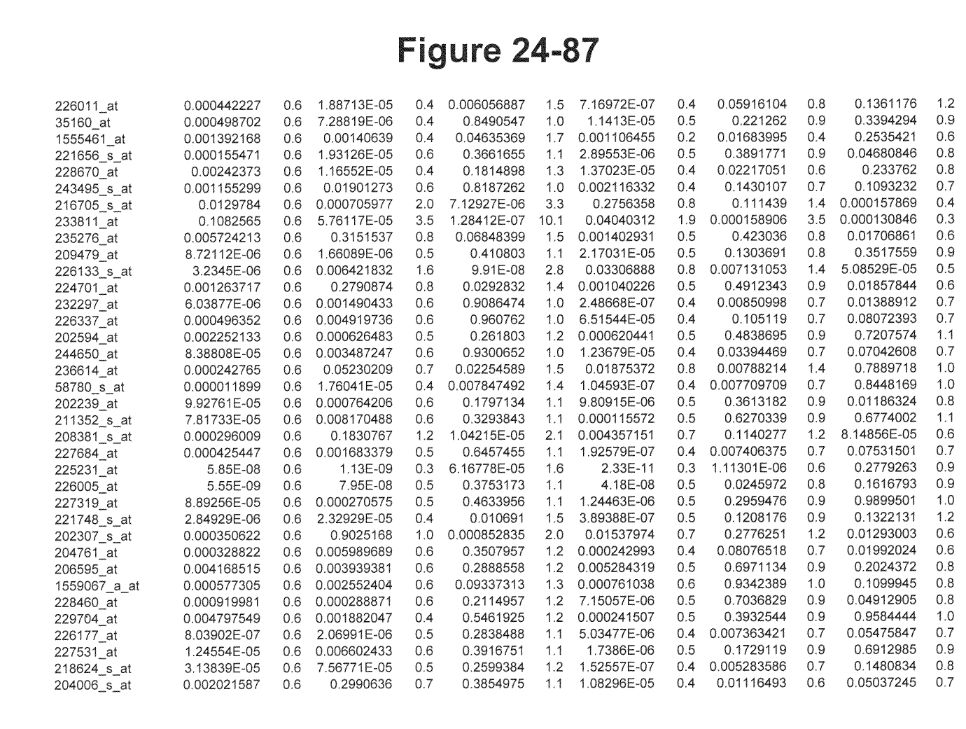

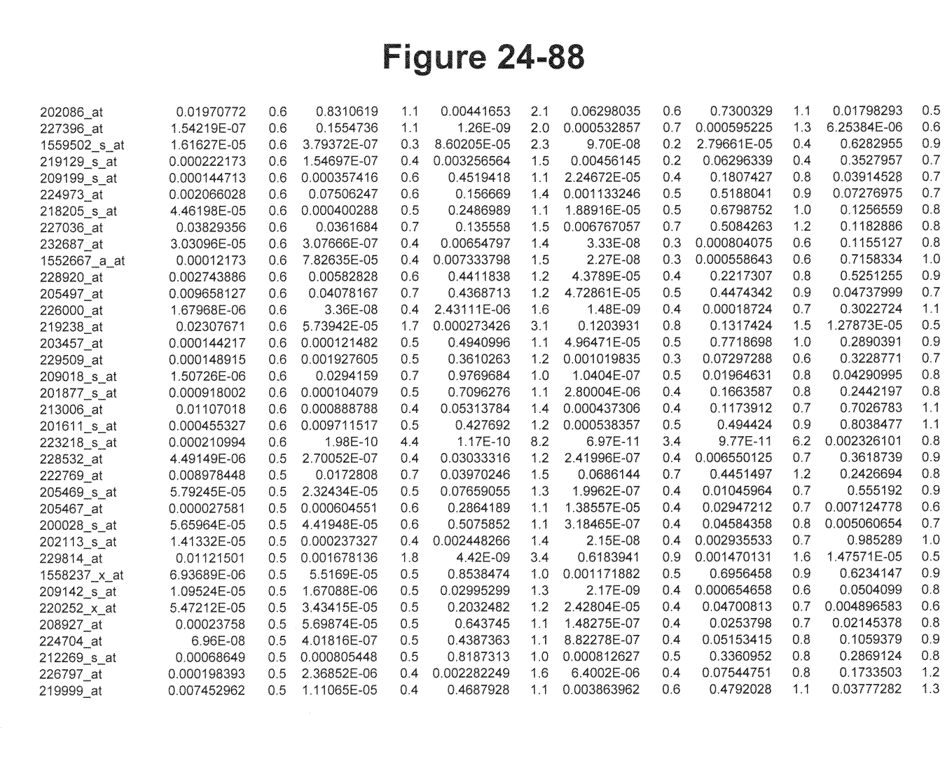

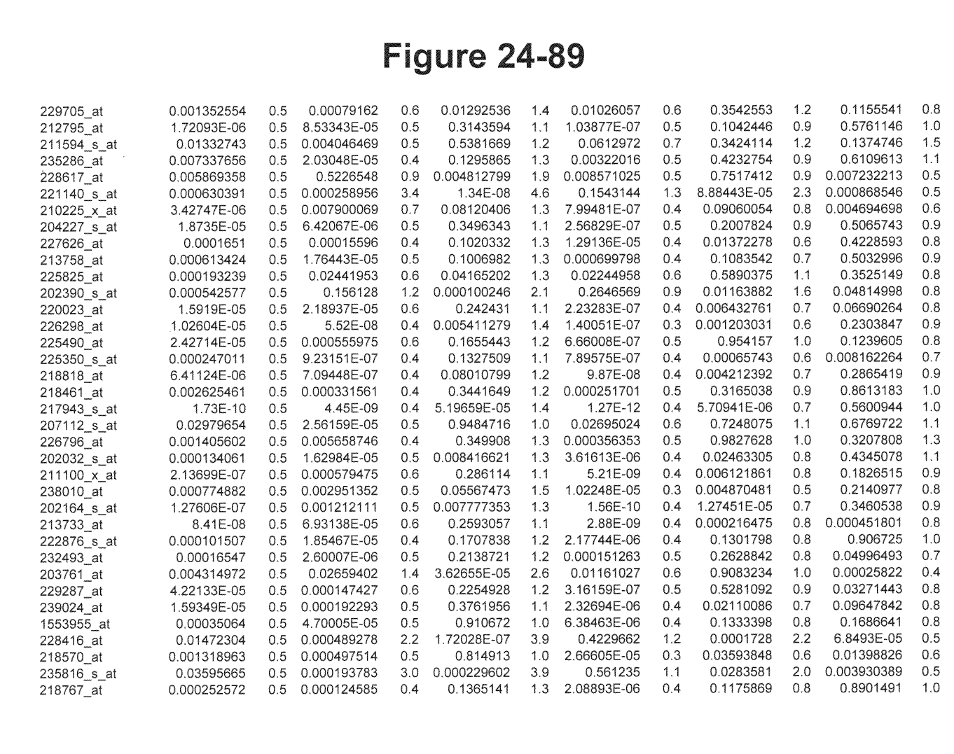

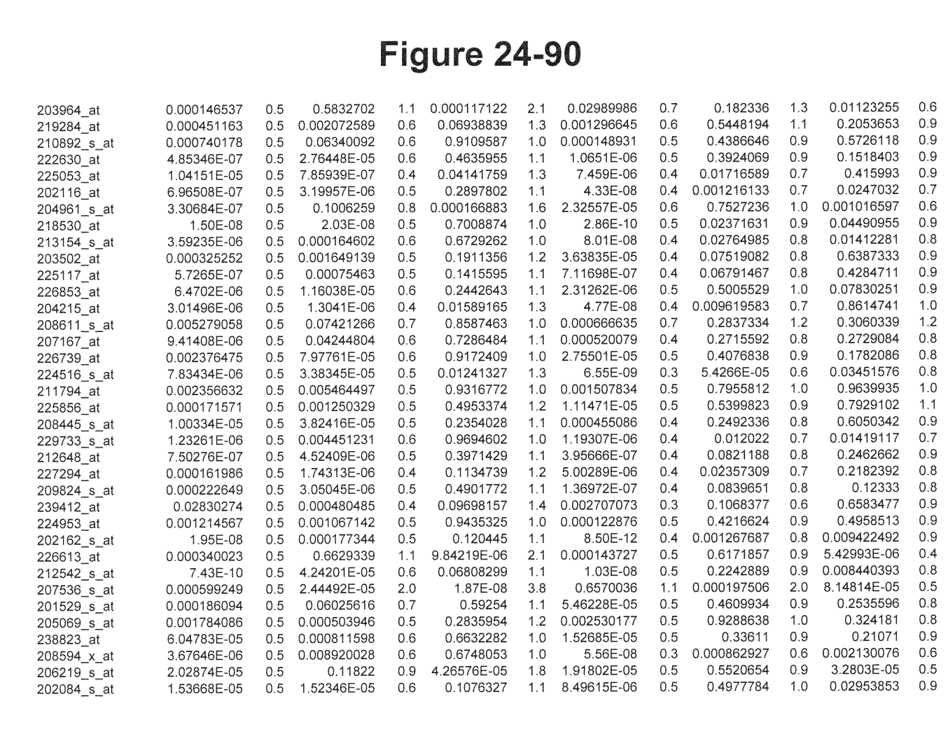

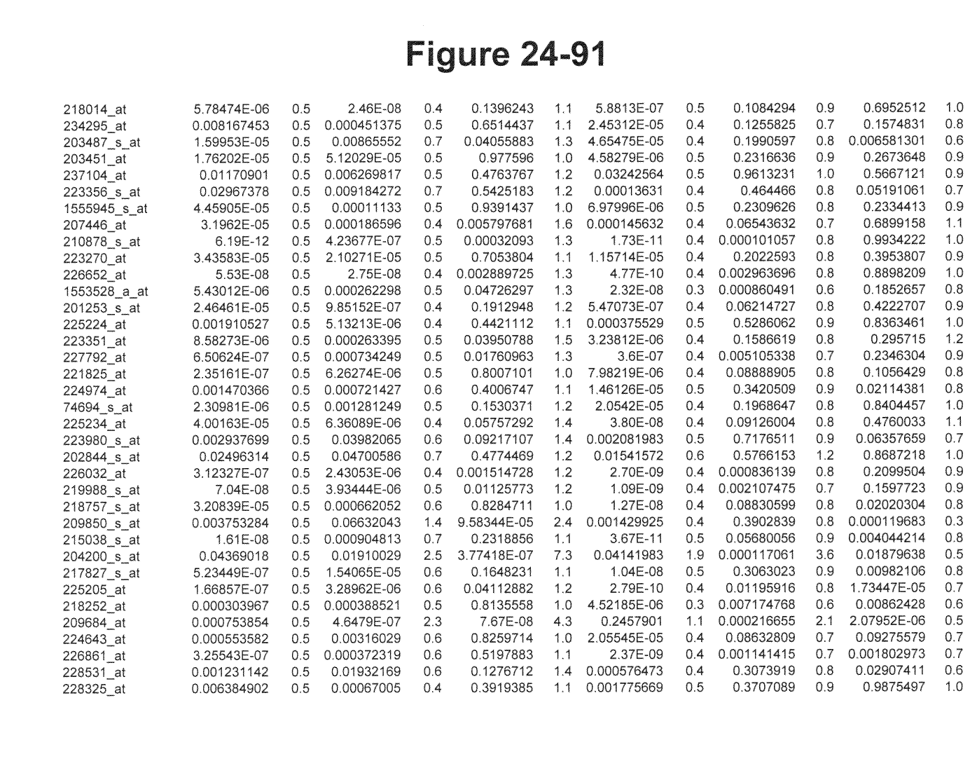

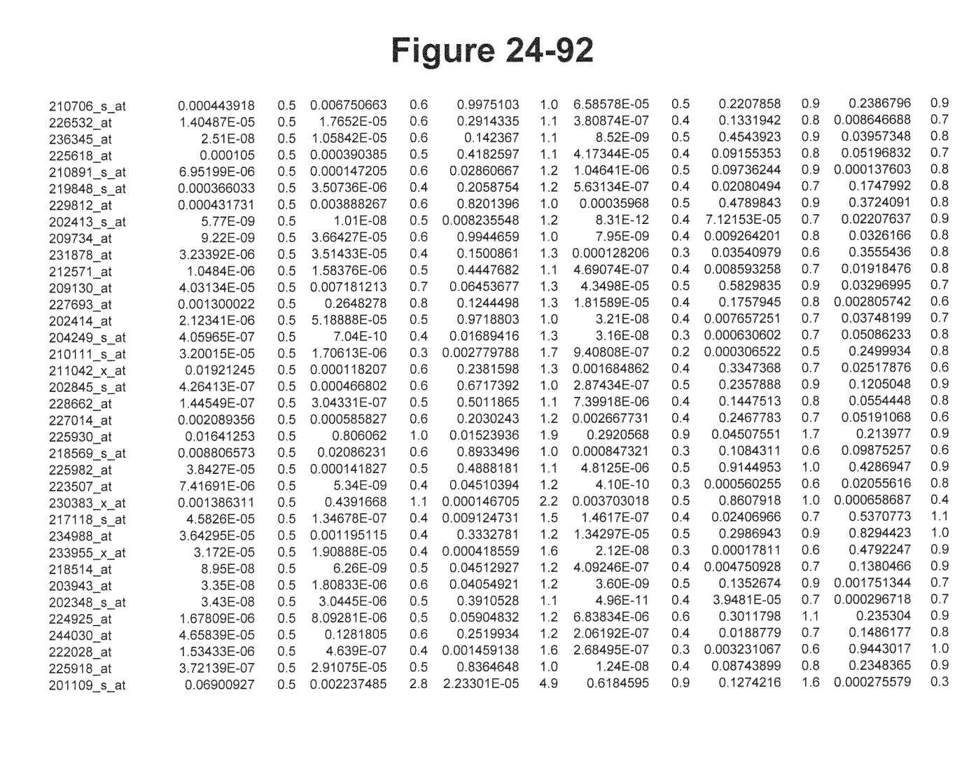

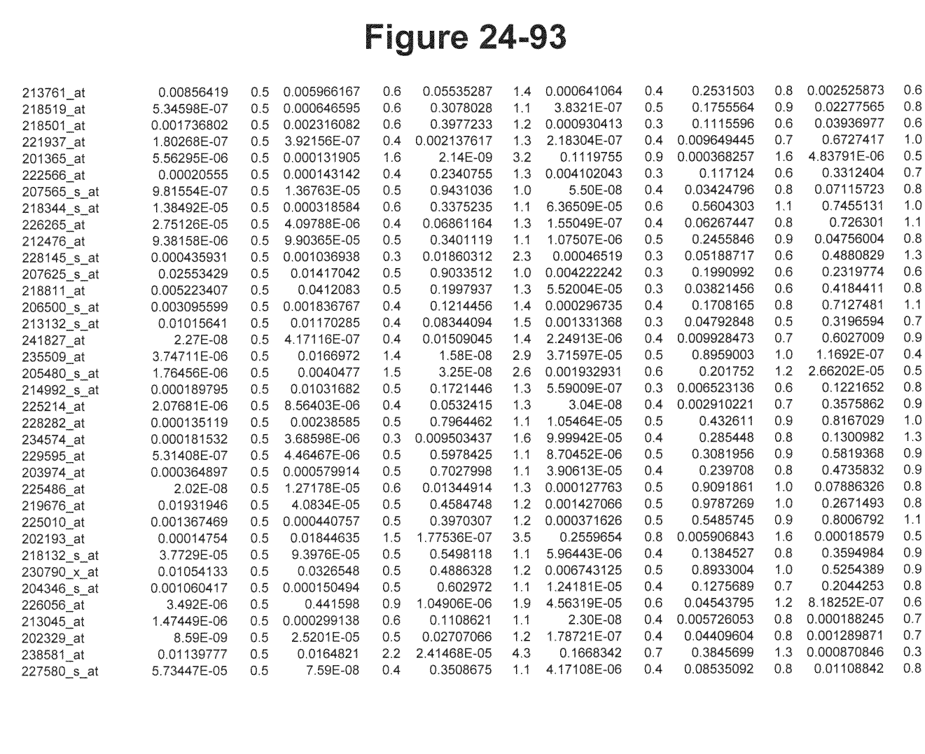

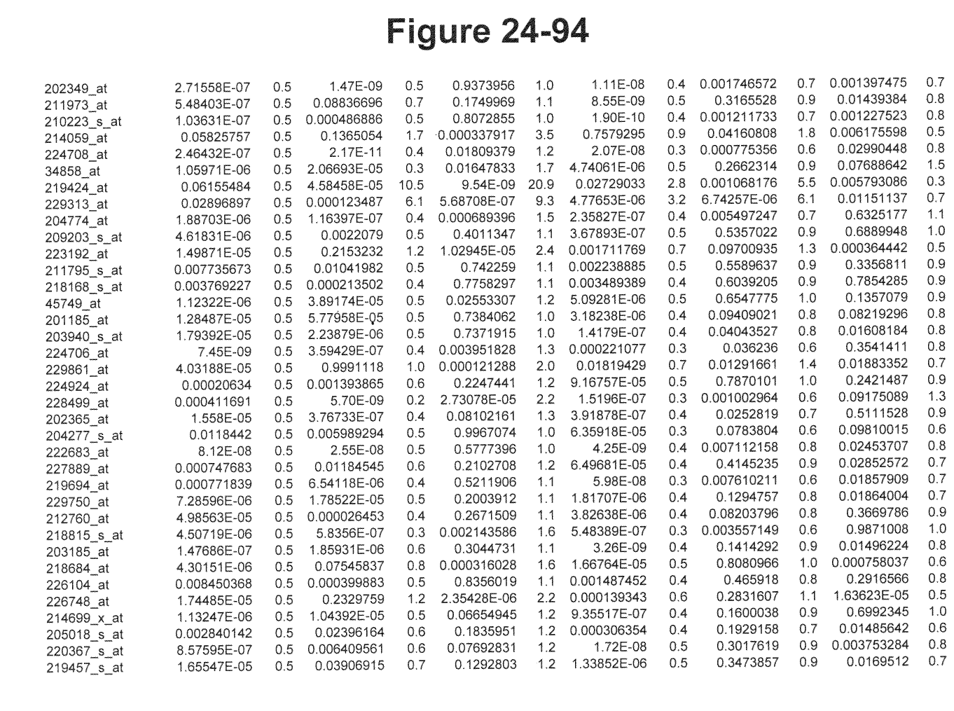

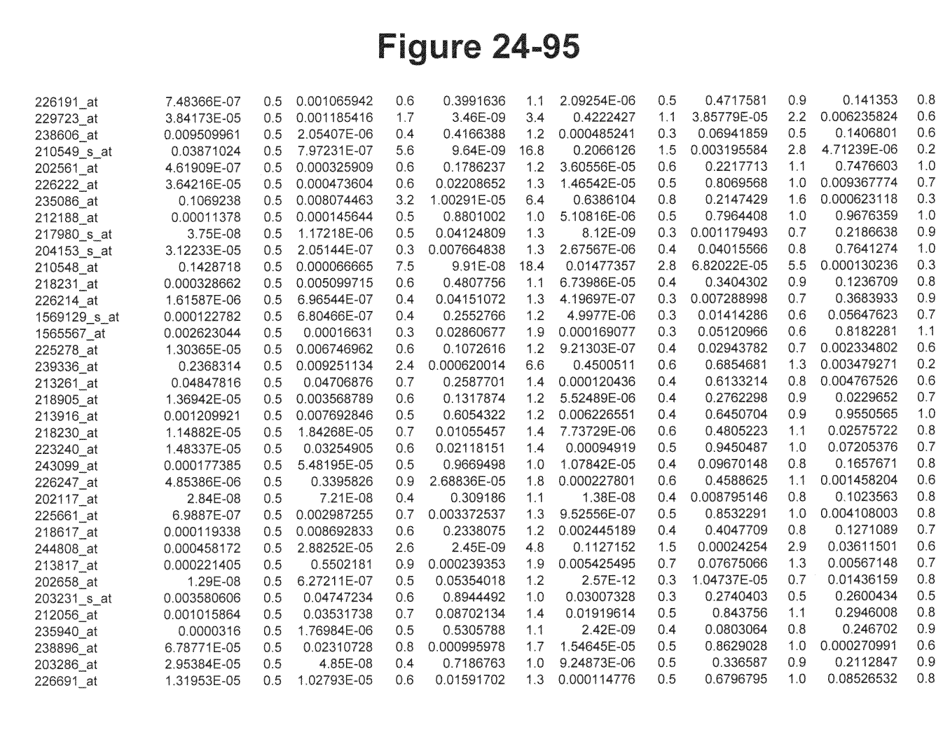

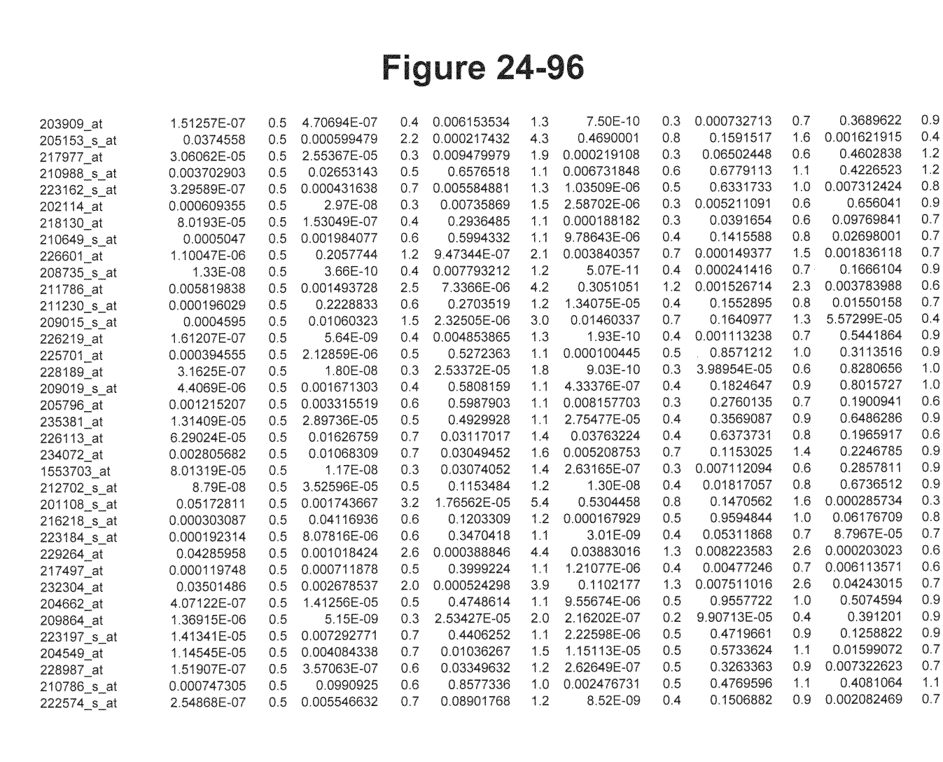

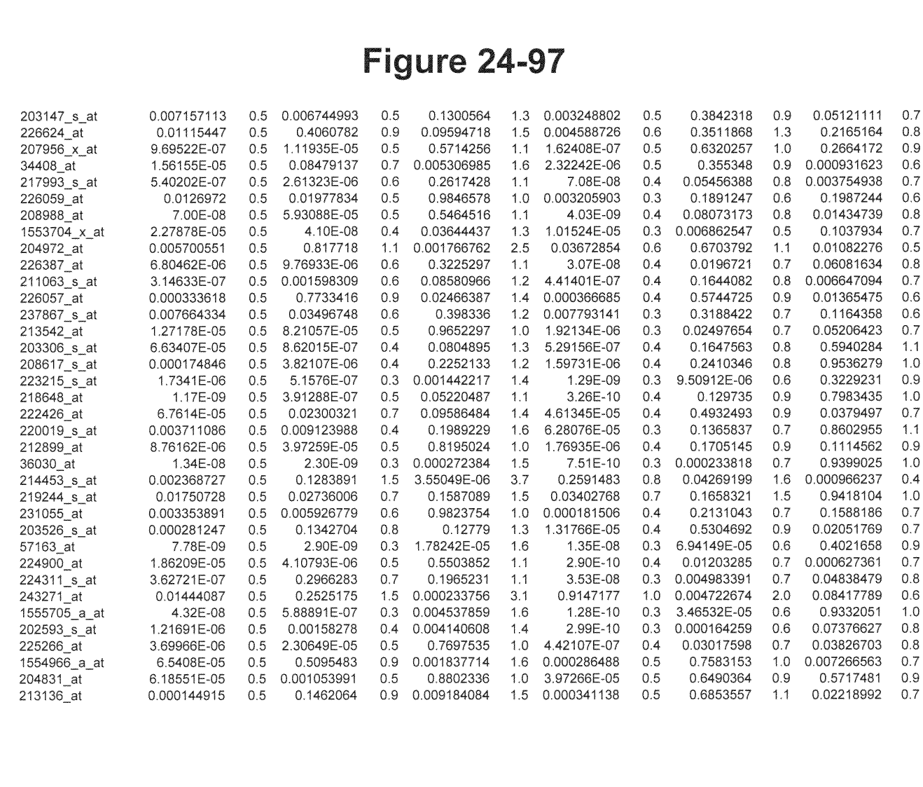

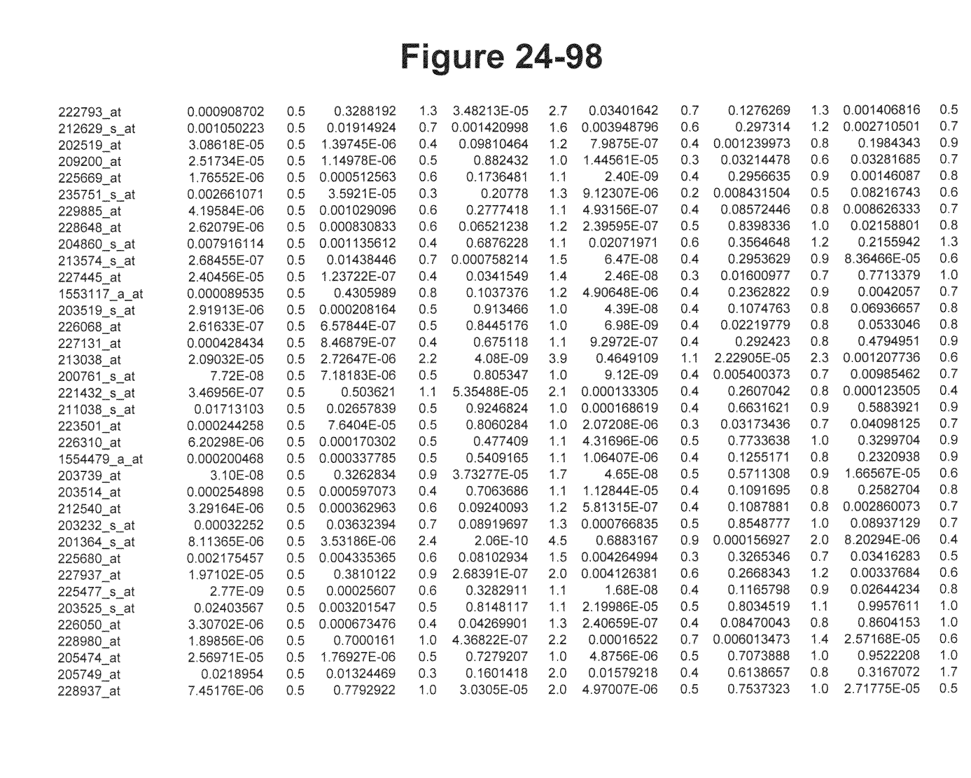

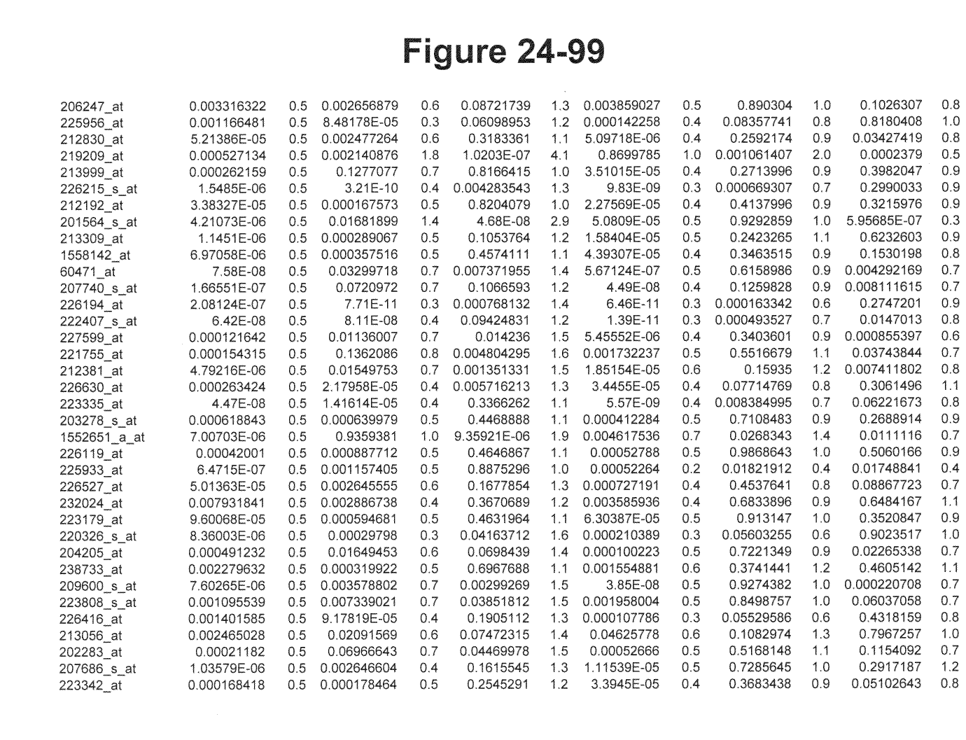

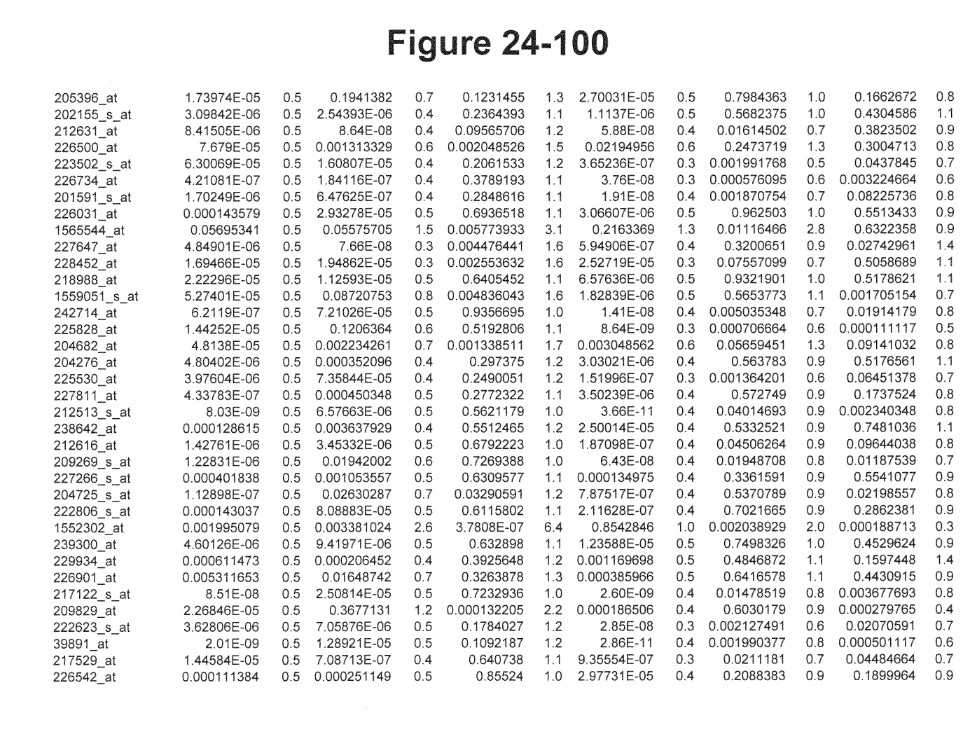

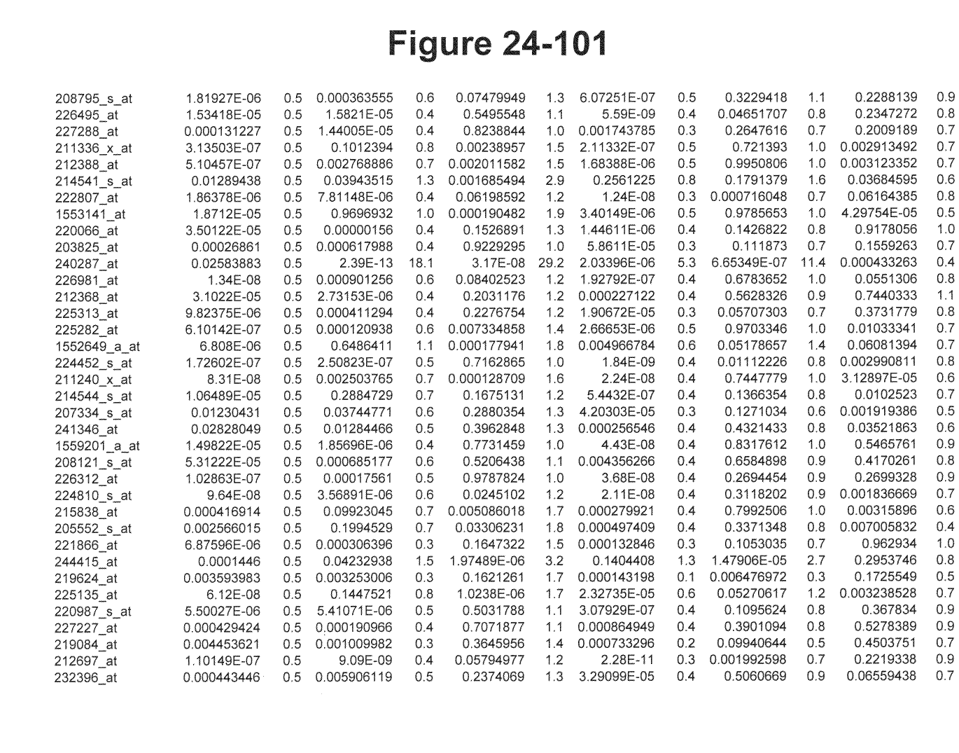

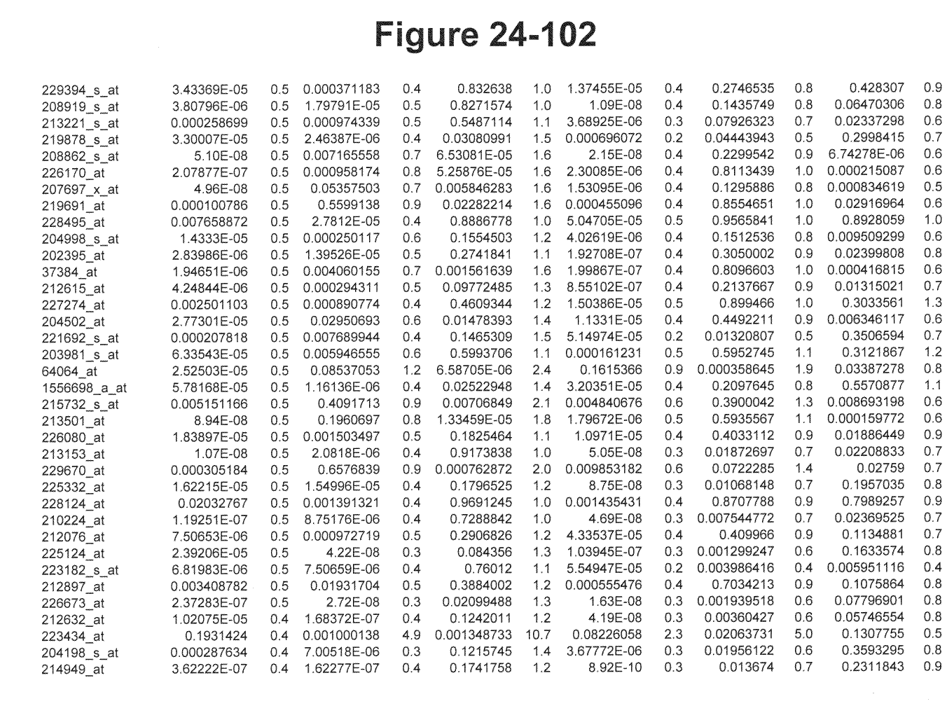

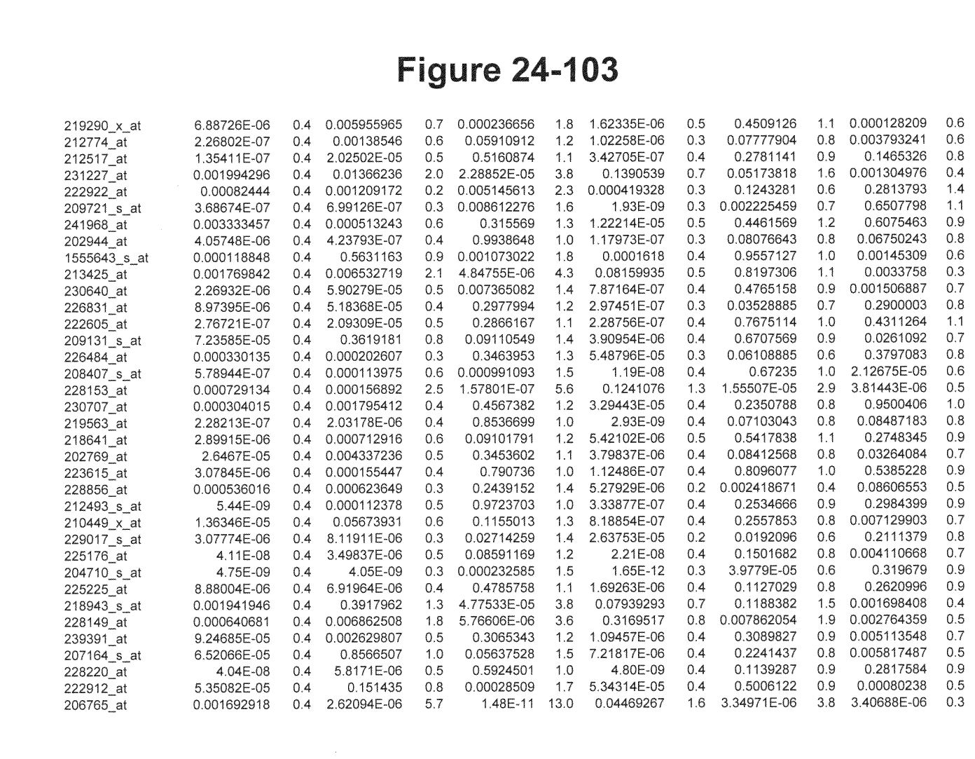

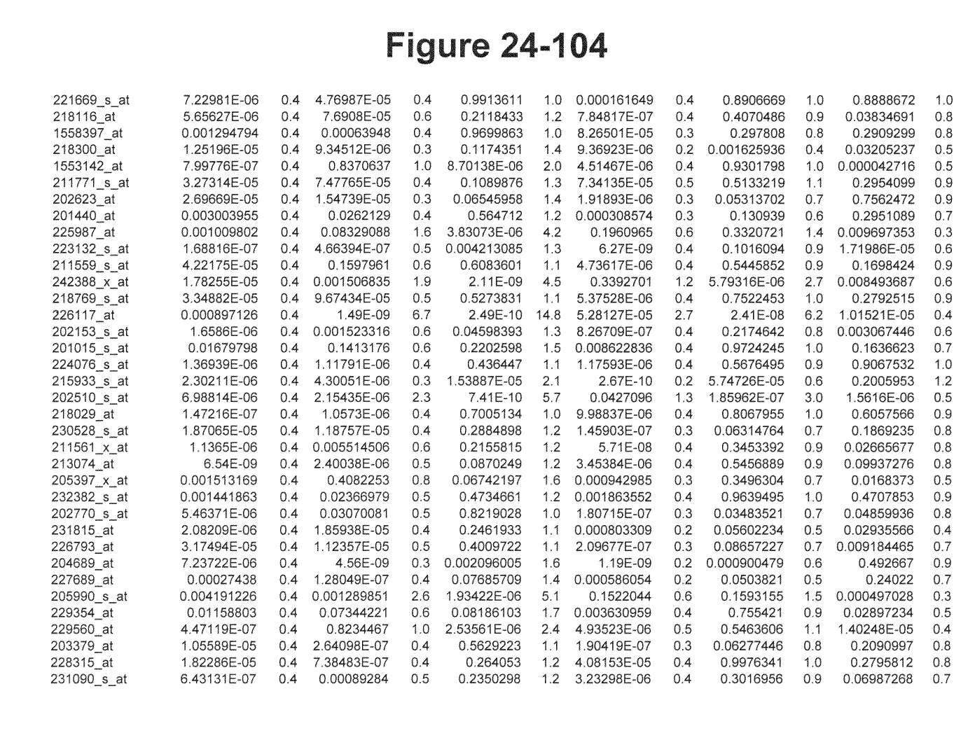

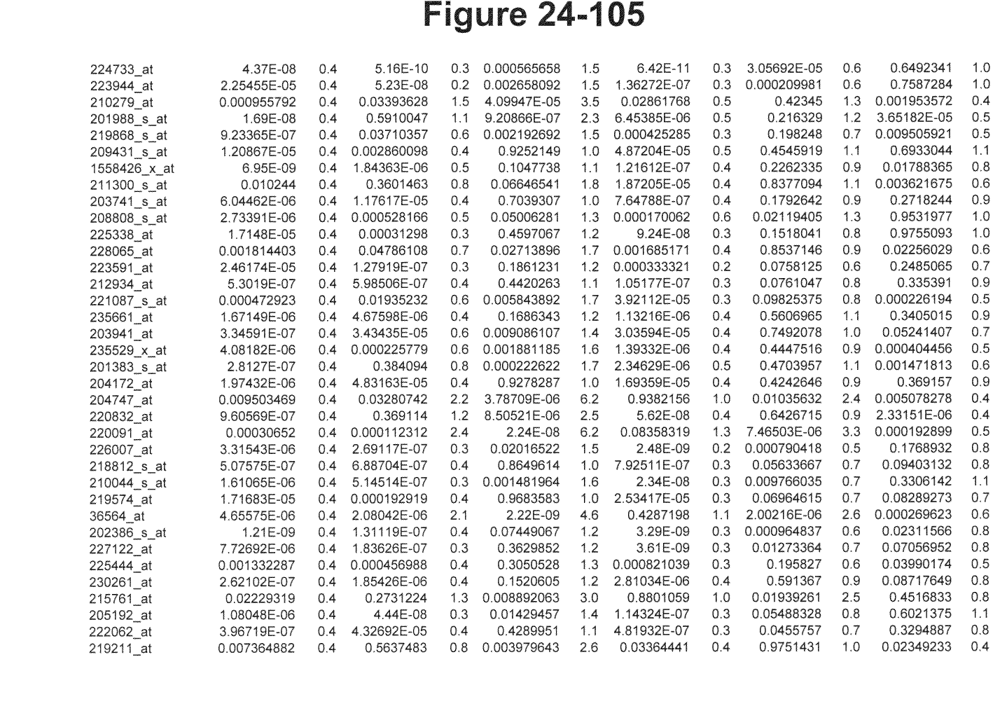

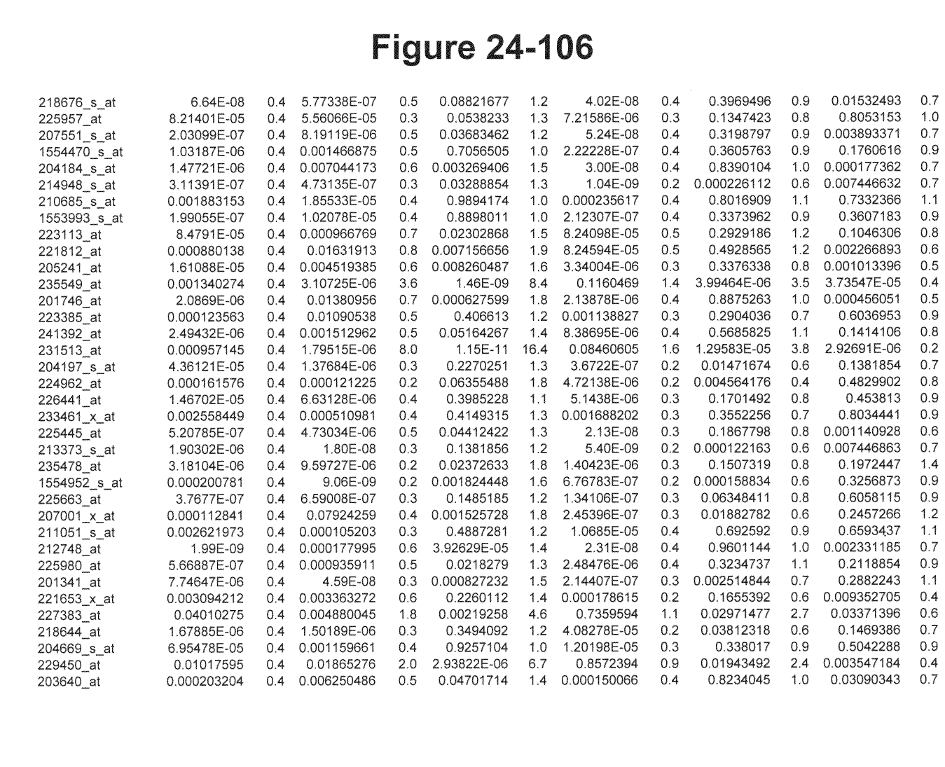

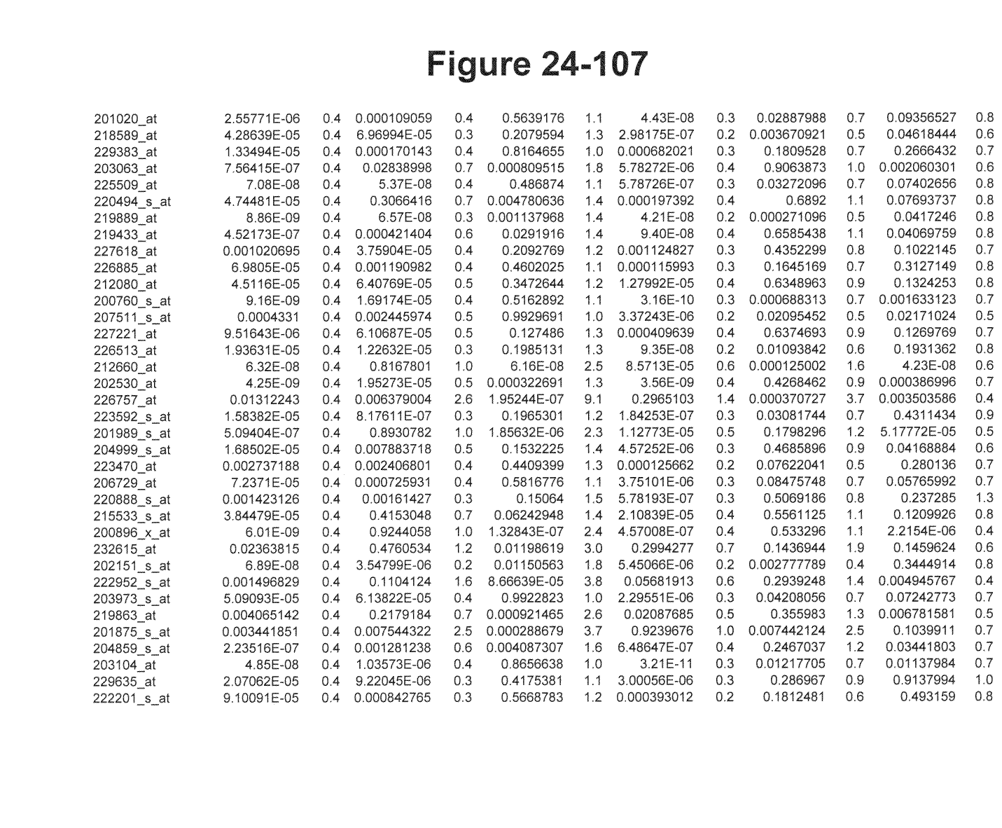

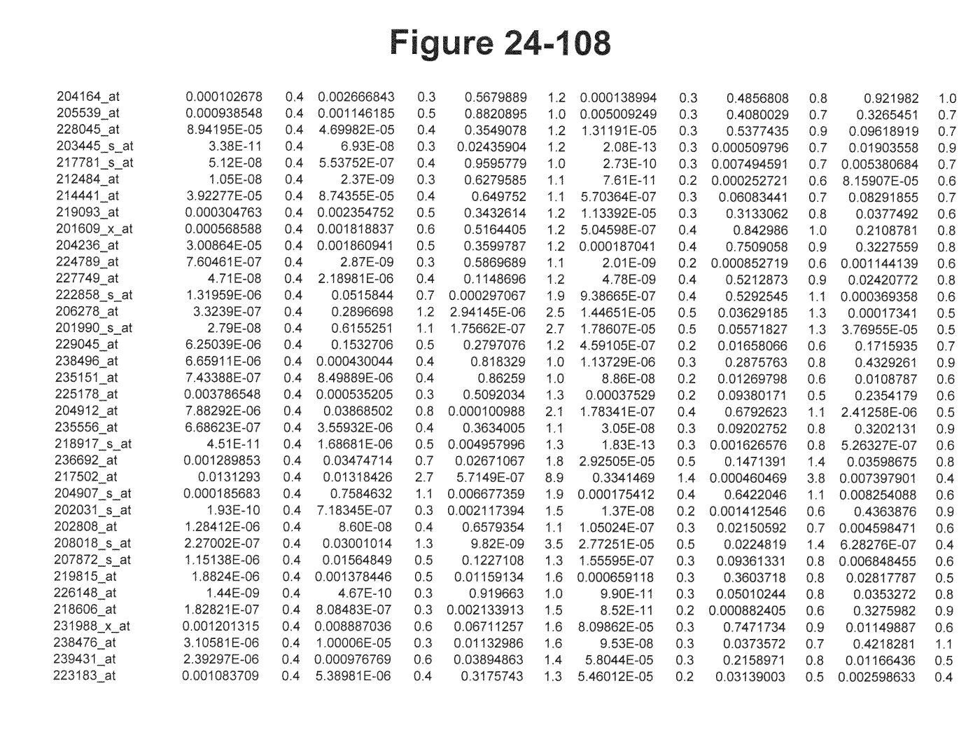

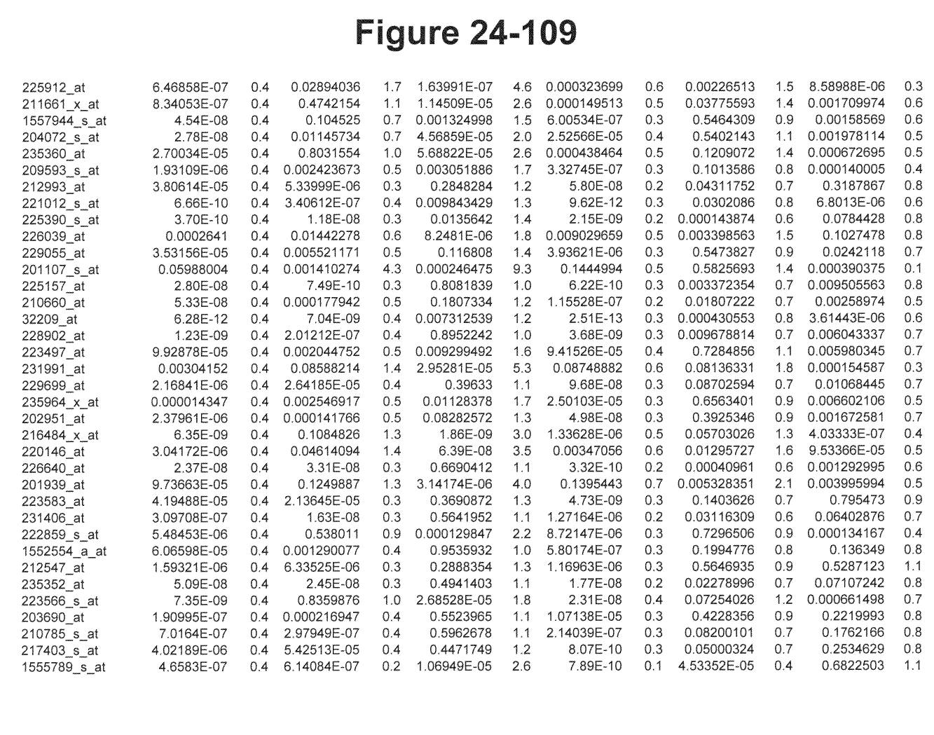

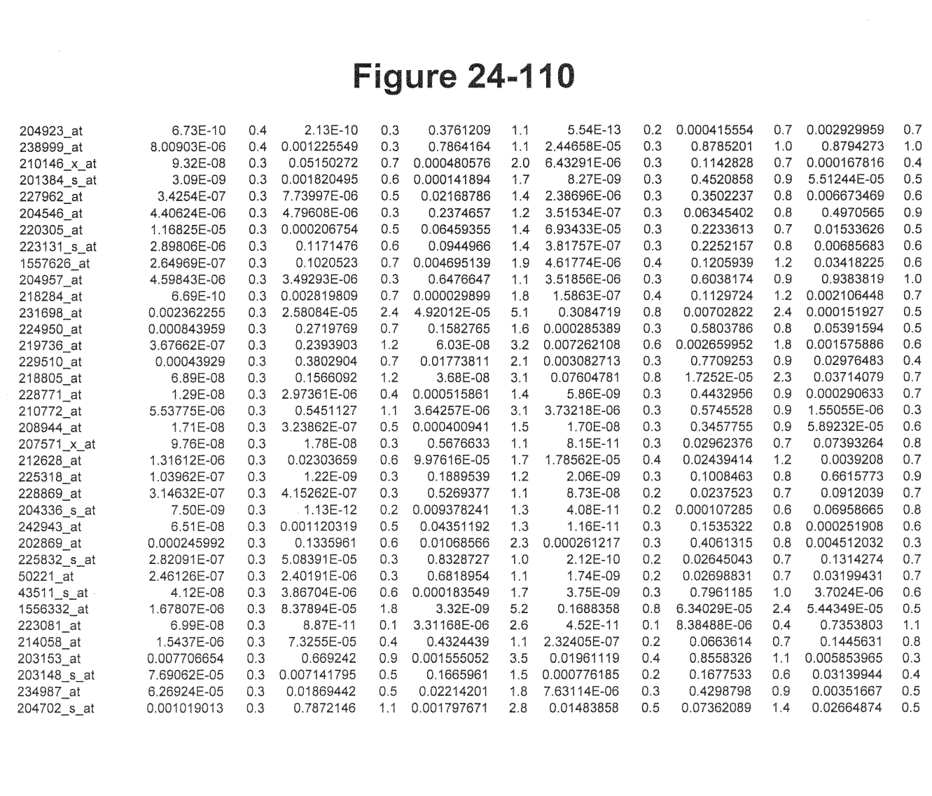

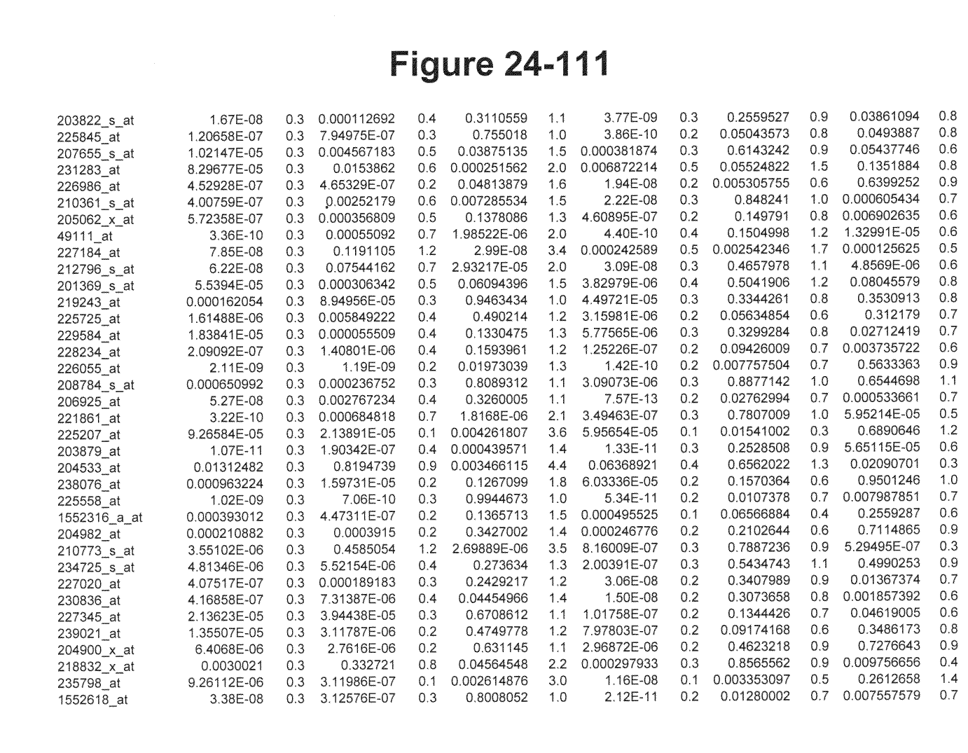

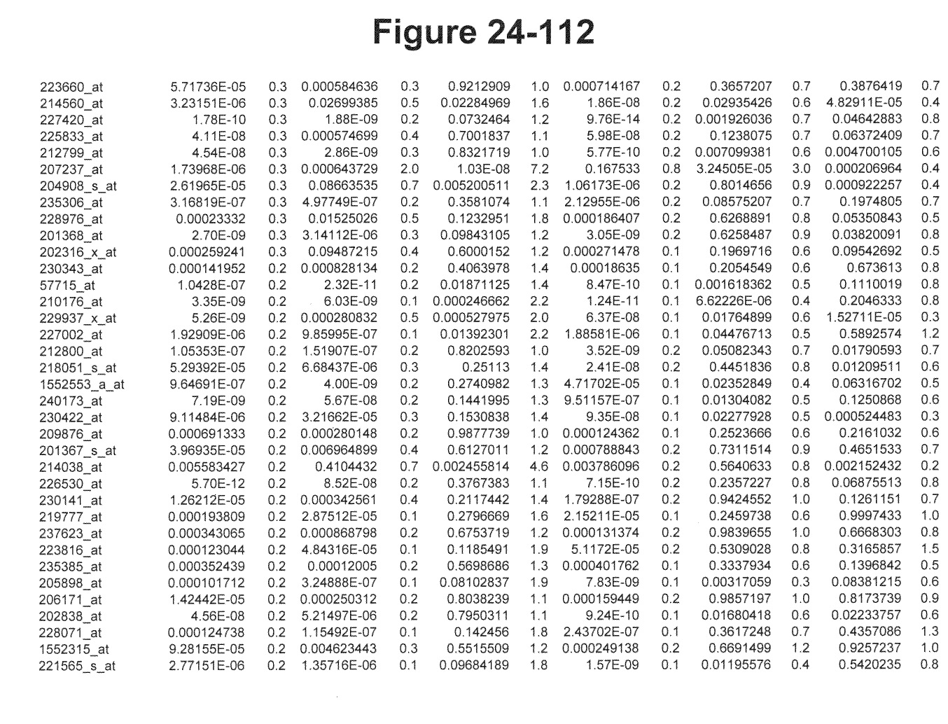

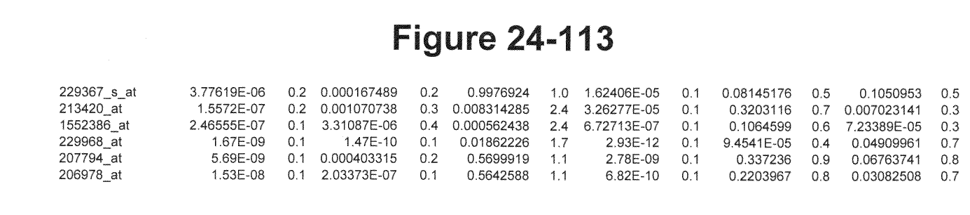

FIGS. 23 and 24 are tables listing genes which were determined to be responsive to TREM-1 activation and/or treatment with LPS. FIG. 23 shows the qualifier, gene name, gene description, and average intensity of identified genes with various treatments. Treatments included: untreated (control), isotype antibody (control), α-TREM-1 antibody, LPS, isotype antibody plus α-TREM-1 antibody, and α-TREM-1 antibody plus LPS. FIG. 24 shows the qualifier of identified genes as well as the p-value and ratio for comparisons between different treatments. The following treatments were compared: α-TREM-1 v. isotype; LPS v. untreated; α-TREM-1 v. LPS; α-TREM-1 plus LPS v. isotype; α-TREM-1 v. α-TREM-1 plus LPS; and LPS v. α-TREM-1 plus LPS.

Results

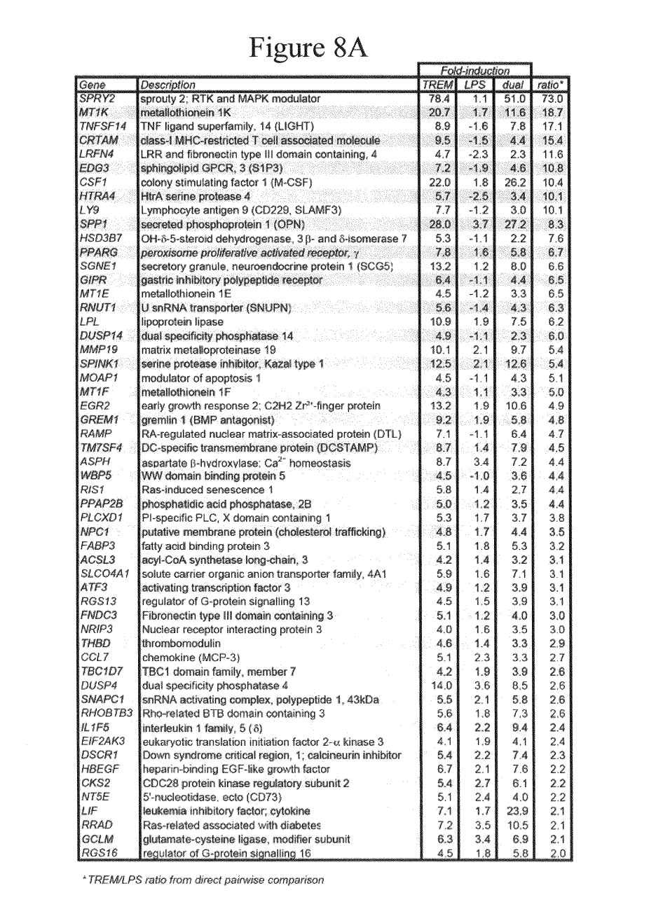

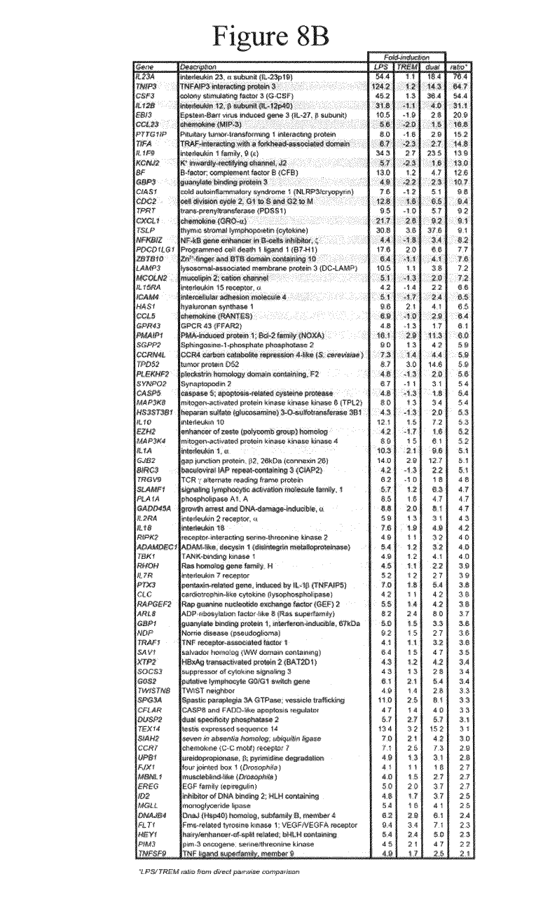

Multiple genes were identified as being differentially expressed in response to α-TREM-1 treatment and/or LPS treatment (see FIGS. 7, 8A-B, 9A-B, and 23-34) and can therefore be used as biomarkers to evaluate agents that modulate TREM-1 and/or LPS signaling. Differentially expressed genes fell into three main categories: TREM-1 biased genes, LPS biased genes, and genes which were comparably expressed in response to both α-TREM-1 and LPS treatments. The genes listed in FIGS. 8A-B are ranked by TREM-1/LPS ratio or LPS/TREM-1 ratio, respectively. The TREM-1/LPS ratio (see FIG. 8A) and LPS/TREM-1 ratio (see FIG. 8B) were calculated from a direct pairwise comparison, which accounts for any variation with respect to fold-changes in individual treatments. Fold-changes in gene expression resulting from dual treatment, i.e., treatment with both α-TREM-1 antibody and LPS, are also shown in FIGS. 8A-B, 9A-B, 23 and 24.

Genes which passed the above filtering criteria and which demonstrated >4-fold changes in expression were considered for further analysis. By these criteria, 238 genes were up-regulated >4-fold with either TREM-1 activation or LPS. Of these, 69 genes were induced >4-fold in both treatments, or >4-fold in only one treatment but within 2-fold in a direct pairwise comparison between the two treatments; these genes have been categorized as commonly up-regulated. The remainder of genes up-regulated >4-fold with either TREM-1 activation (62 genes) or LPS treatment (101 genes) have been categorized as treatment-specific (i.e., treatment-biased).

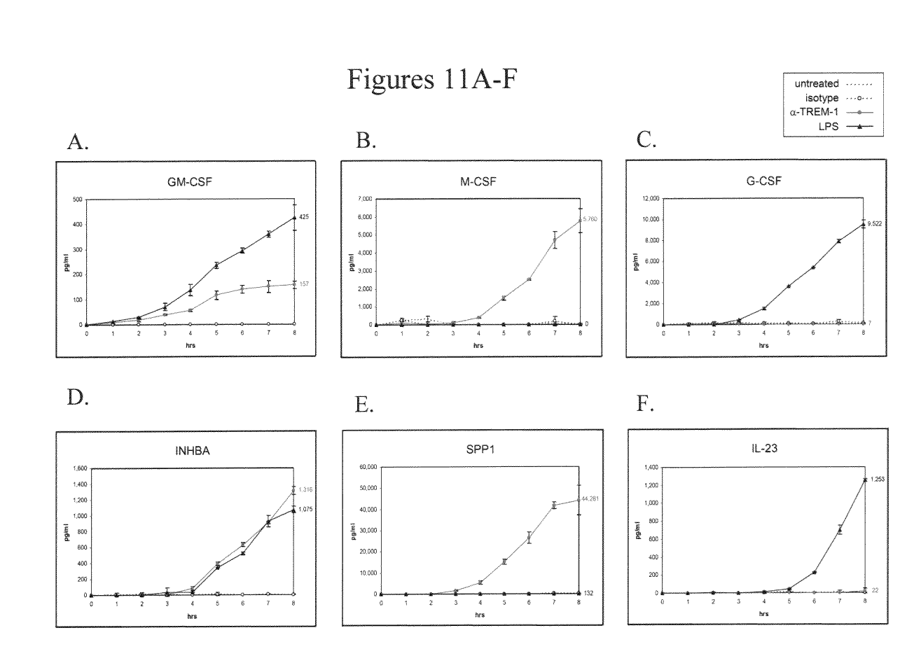

A summary of TREM-1 biased genes that are up-regulated >4-fold in response to TREM-1 activation is shown in FIG. 8A. Provided are fold-changes with TREM-1 activation (TREM), LPS (LPS), and combined TREM-1 activation plus LPS (dual), ranked by the ratio of TREM-1/LPS. p-values for genes up-regulated >4-fold with TREM-1 activation ranged from 7.7×10⁻⁴ to 2.6×10⁻¹². Genes identified as preferentially induced by TREM-1 activation include SPRY2, cytokines and related molecules (TNFSF14, CSF1, SPP1, CCL7, IL1F5, LIF), metallothioneins (MT1K, MT1E, MT1F), phosphatases (DUSP14, DUSP4), transcription factors (EGR2, ATF3), factors involved in lipid metabolism and/or signaling (EDG3, LPL, PPAP2B, PLCXD1, NPC1, FABP3, ACSL3), MMP19, and PPARG. Of these genes, SPP1 is up-regulated 28.0 fold (p=1.2×10⁻⁰⁷) in response to TREM-1 activation (see FIGS. 8A and 11E), but is not appreciably up-regulated after LPS treatment. Thus, SPP1 is not an obligate pro-inflammatory readout and can serve as a marker for TREM-1 activity in a patient (or patient sample) and in screening assays for identifying TREM-1 modulating agents. Moreover, SPP1 can also serve as an indicator of the efficacy or potential efficacy of a TREM-1 therapy for the treatment of inflammation or chronic inflammation, such as RA, in a patient. Additional genes which may be used as markers for TREM-1 activity are listed in FIGS. 8A, 23 and 24. Genes that met the filtering criteria but which are not listed in FIG. 8A include C6orf114, C6org128, C9orf47, KIAA1199, KIAA1393, LOC440995, and MGC33212. In general, genes preferentially induced by TREM-1 activation were largely unaffected by LPS treatment.

A summary of LPS biased genes that are up-regulated >4-fold in response to LPS treatment is shown in FIG. 8B. Provided are fold-changes with TREM-1 activation (TREM), LPS (LPS), and combined TREM-1 activation plus LPS (dual), ranked by the ratio of LPS/TREM-1. p-values for genes up-regulated >4-fold with LPS in this Table ranged from 1.2×10⁻³ to 3.6×10⁻¹⁴. Genes identified as preferentially induced by LPS include interleukins (IL23A, IL12B, EBI3, IL1F9, IL10, IL1A, IL18), interleukin receptors (IL15RA, IL2RA, IL7R), cytokines and related molecules (CSF3, CCL23, CXCL1, TSLP, CCL5, CLC, EREG, TNFSF9), factors involved in lipid metabolism and/or signaling (SGPP2, PLA1A, MGLL), kinases (MAP3K8, RIPK2, MAP3K4, TBK1, PIM3), regulators of NF-κB signaling (TNIP3, NFKBIZ), CCR7, and CIAS1. Genes that met the filtering criteria but which are not listed in FIG. 8B include C10orf78, C21orf71, FLJ14490, FLJ23231, FLJ25590, FLJ32499, KIAA0286, KIAA0376, LOC90167, LOC123872, LOC285628, LOC338758, LOC341720, LOCLOC374443, LOC387763, LOC400581, LOC441366, MGC10744, and MGC11082.

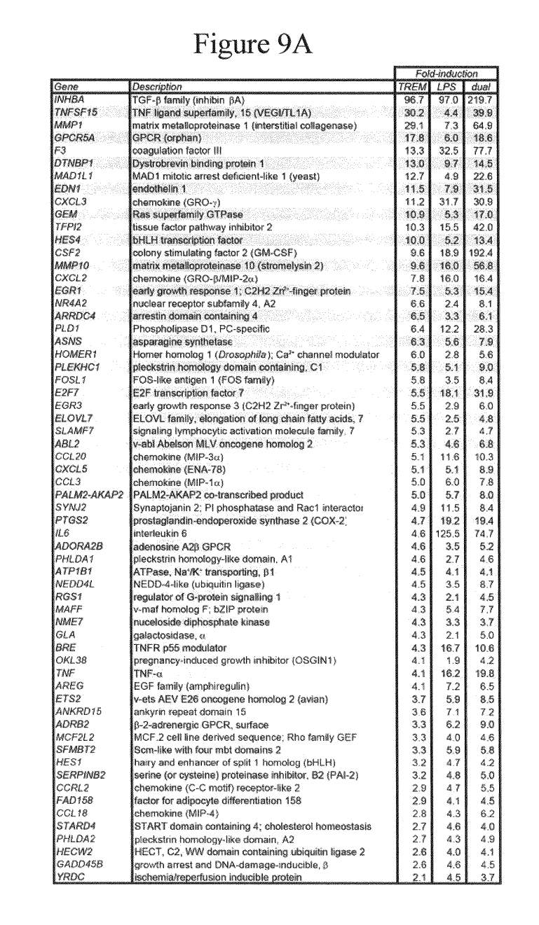

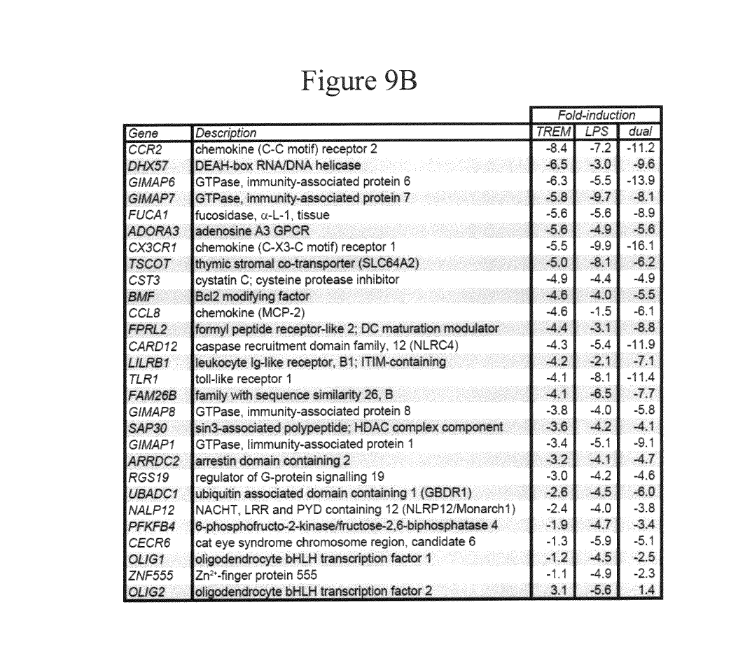

FIG. 9A shows a summary of common up-regulated genes (i.e., genes up-regulated by and TREM-1 activation and LPS treatment). Provided are fold-changes with TREM-1 activation (TREM), LPS (LPS), and combined TREM-1 activation plus LPS (dual), ranked by fold-induction with TREM-1 activation. p-values for genes up-regulated >4-fold with TREM-1 activation ranged from 1.7×10⁻³ to 1.5×10⁻¹⁰, and those for LPS ranged from 4.1×10⁻³ to 2.0×10⁻¹⁴. Genes identified as being commonly up-regulated include TNF superfamily members and modulators (TNFSF15, BRE, TNF), chemokines (CXCL3, CXCL2, CCL20, CXCL5, CCL3), other cytokines and mitogenic factors (CSF2, IL-6, AREG), matrix metalloproteinases (MMP1, MMP10), and PTGS2/COX2. These results are consistent with both TREM-1 activation and LPS eliciting pro-inflammatory responses. Also present are INHBA, coagulation and vascularization factors (F3, EDN1, TFPI2, SERPINB2), transcription and DNA binding factors (HES4, EGR1, FOSL1, E2F7, EGR3, MAFF, ETS2, HES1), and factors involved in lipid metabolism and/or signaling (PLD1, ELOVL7, SYNJ2, GLA, STARD4). Genes that met the filtering criteria but which are not listed in FIG. 9A include C20orf139, KIAA1718, LOC348938, LOC401151, LOC401588, LOC92162, and MGC4504.

In the combined (dual) treatment, the expression change for the majority of the genes in FIG. 9A was within 2-fold of the sum of those in individual treatments. One exception was CSF2 (i.e., GM-CSF), whose mRNA induction was significantly increased in combined treatment with respect to individual treatments (9.6-, 18.9-, and 192.4-fold with TREM-1 activation, LPS, and combined treatment, respectively).

A summary of genes with fold-changes <−4 (i.e., down-regulated >4-fold) with either TREM-1 activation or LPS treatment is shown in FIG. 9B. Provided are fold-changes with TREM-1 activation (TREM), LPS (LPS), and combined TREM-1 activation plus LPS (dual), ranked by fold-induction with TREM-1 activation. p-values for genes down-regulated >4-fold with TREM-1 activation ranged from 5.6×10⁻³ to 5.7×10⁻¹², and those for LPS ranged from 2.4×10⁻³ to 1.1×10⁻¹⁴. As seen in FIG. 6, a comparable number of genes were down-regulated in our analysis as were up-regulated, although there was less treatment specificity among these genes. Genes identified as being down-regulated include chemokine receptors (CCR2, CX3CR1), transcription factors (OLIG1, ZNF555, OLIG2), GTPases of immunity-associated proteins (GIMAP6, GIMAP7, GIMAP8, GIMAP1), and CCL8. CCR2, the down-regulation of which is a marker for monocyte-to-macrophage differentiation, is down-regulated in both α-TREM-1 and LPS treatments (see FIG. 9B). In addition, TLR1 and NOD-like receptors (CARD12, NALP12) are also down-regulated. Genes that met the filtering criteria but which are not listed in FIG. 9B include C9orf59, FLJ12442, FLJ33641, LOC90120, MGC2941, and MGC17791. The dynamic range in down-regulation was lower than that for up-regulation, as expected, given the limiting kinetic contribution of mRNA half-lives to the analysis.

In general, relatively few genes were down-regulated in one treatment but not the other. One gene that was not commonly down-regulated is the oligodendrocyte transcription factor OLIG2, which was up-regulated 3.1-fold by TREM-1 activation and down-regulated 5.6-fold by LPS (see FIG. 9B).

In addition, TREM-1 and LPS differentially regulate CSF production, with M-CSF being TREM-1-biased and G-CSF being LPS-specific (see FIGS. 7, 8A-B).

### Example 7

### Phagocytosis Assay

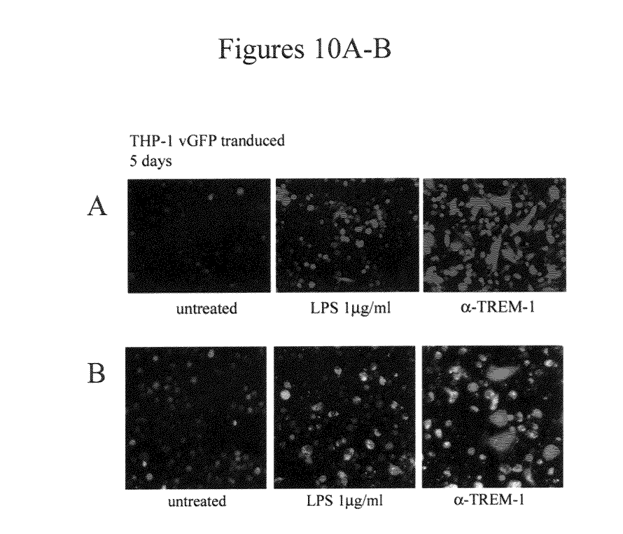

Human THP-1 cells were treated with α-TREM-1 and LPS to compare the effects of α-TREM-1 treatment and LPS treatment on THP-1 cell morphology and behavior. Human THP-1 cells (ATCC; TIB-202) were maintained according to the recommended propagation guidelines. For enhanced visualization, THP-1 cells were transduced with a GFP-expressing lentivirus prior to the indicated treatments in tissue culture-treated wells for 5 days. Phagocytosis assays were performed by adding Fluoresbrite™ polychromatic red 1.0 micron microspheres (Polysciences, Inc., Warrington, Pa.; 18660), incubating in a tissue culture incubator for 30 minutes, and washing with growth medium to remove un-opsonized beads prior to imaging. α-TREM-1 treatment induced morphological changes in THP-1 cells (FIG. 10A). Moreover, both α-TREM-1 treatment and LPS treatment induced phagocytosis of labeled microspheres (1 μM beads appear in red), consistent with a role for TREM-1 activation in stimulating an immune response (FIG. 10B).

### Example 8

### ELISA Profiling of Gene Expression

Differential expression of selected genes in response to α-TREM-1 and LPS treatments were confirmed by ELISA. ELISAs were performed on conditioned media as per the manufacturers' protocols. The levels of proteins secreted into cell-culture media following either TREM-1 activation or LPS were measured over an 8 hour time-course. GM-CSF was an analyte on custom-coated plates ordered from Meso Scale Discovery (Gaithersburg, Md.). M-CSF, G-CSF, INHBA, and SPP1 detection kits were purchased from R&D Systems (Minneapolis, Minn.; DMC00, DCS50, DY338, and DOST00, respectively). The IL-23 detection kit was purchased from eBioscience (San Diego, Calif.; 88-7237).

Detection of target protein levels (with fold changes in transcript levels for TREM-1 and LPS, respectively, in parentheses) confirmed that SPP1 (28.0 and 3.7; FIG. 11E) and M-CSF (22.0 and 1.8; FIG. 11B) are up-regulated in response to TREM-1 activation, but not in response to LPS treatment. These results also demonstrate that secreted SPP1 protein is detectable in extracellular fluids, such as tissue culture medium. Moreover, protein levels of G-CSF (1.3 and 45.2; FIG. 11C) and IL-23 (−1.1 and 31.8; FIG. 11F) confirmed that these genes are up-regulated in response to LPS treatment. Finally, protein levels of INHBA (96.7 and 97.0; FIG. 11D) and GM-CSF (9.6 and 18.9; FIG. 11A) confirmed that these genes are comparably up-regulated in response to both TREM-1 activation and treatment with LPS.

These ELISA results validate the use of transcriptional profiling analysis to identify genes responsive to TREM-1 activation and LPS treatment. These results also identify, for the first time, cytokines or related factors which are induced by TREM-1 activation but which are not also induced by LPS. Moreover, the ELISA results show that SPP1 is up-regulated at the protein level in response to TREM-1 activation and that SPP1 can be used as a marker for TREM-1 activation. Additional genes which may be used as markers for TREM-1 activity are listed in FIGS. 8A, 23 and 24.

### Example 9

### Analysis of Cytokine Production from Synovium of RA Patients Upon TREM-1 Activation

Activation of TREM-1 with a crosslinking antibody has been shown to trigger the production of pro-inflammatory factors in both human monocytes and neutrophils. We therefore tested whether TREM-1 activation had a similar pro-inflammatory effect in RA positive synovial cultures. The synovium culture assay was performed as first described by Brennan et al. (1989) J. Autoimmunity 2 Supp: 177-86. Briefly, synovial tissues were obtained during arthroscopic knee surgery of three different RA patients (Arthritis and Osteoporosis Center of Maryland in Frederick, Md.). Samples were placed in RPMI with 5% fetal calf serum (FCS) for transport. To disrupt tissue and release cells, tissues from Donor 1 and Donor 2 were treated with 50 ml of RPMI with 5% FCS containing 5 mg/ml collagenase IV (Invitrogen, Carlsbad, Calif.) and 0.15 mg/ml DNase I (Sigma, St. Louis, Mo.) and rotated at 37° C. for 90 min. Tissue from Donor 3 was prepared similarly, except that Liberase Blendzyme 4 (Roche) was substituted for the collagenase/DNase, and was used according to the manufacturer's suggested protocol. Liberase Blendzyme 4 is promoted as being virtually endotoxin-free. Debris was removed by passing the sample over 100 μm nylon mesh. Cells were washed and resuspended in RPMI with 0.5% FCS for plating. For antibody activation of TREM-1, tissue culture treated plates were coated with 100 μl of antibody solution containing either anti-hTREM-1 antibody (MAB1278, R&D Systems, Minneapolis, Minn.) or an isotype-matched control antibody, anti-E. tenella (Wyeth, Madison, N.J.), at indicated concentrations for 3 hours prior to cell addition. Anti-hTREM-1 antibody was assayed at concentrations of 0.12, 0.37, 1.11, 3.33, and 10 μg/ml; control antibody was assayed at concentrations of 0.12, 1.11, and 10 μg/ml. Wells were washed twice with PBS prior to adding 100 μl of cell suspension at a cell density of 6×10⁵ cells/ml. After 24 hours, supernatants were assayed for the indicated factors using multiplex ELISA plates (Meso-Scale Discovery, Gaithersburg, Md.).

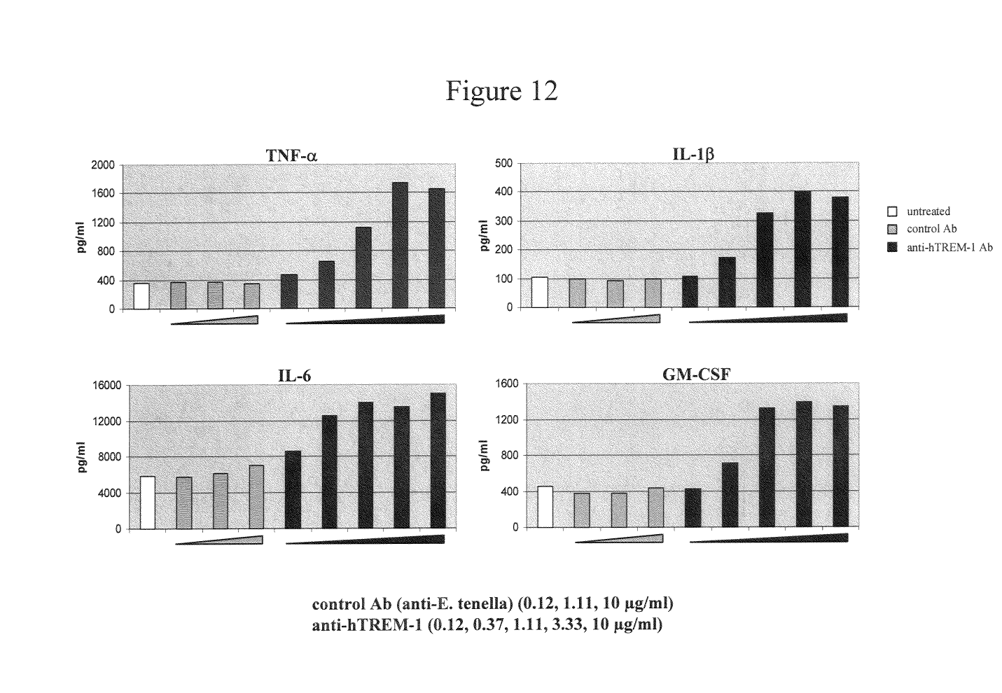

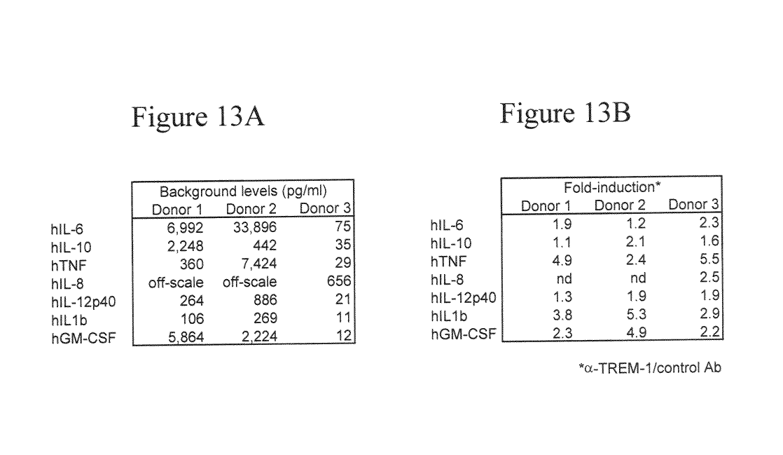

As seen in FIG. 12, which represents data from one individual, activation of TREM-1 in these cultures using a cross-linking antibody induced the production of TNF-α, IL-6, IL-1β and GM-CSF in a dose dependent manner. Similar results were obtained from all three donor samples. Moreover, FIG. 13A shows a comparison of spontaneous cytokine production in each of the three donor samples, and FIG. 13B shows a comparison of cytokine production upon crosslinking of TREM-1 in each of the three donor samples. As shown in FIG. 13A, Donor 3 spontaneous cytokine levels are considerably lower than those for Donors 1 and 2, which is consistent with less endotoxin contamination, but could also be due to donor variability. The results from all three donors indicate that TREM-1 is functionally present in RA cultures and that TREM-1 is capable amplifying the inflammatory response in RA synovium.

### Example 10

### mTREM-1-hFc Transgenic Mouse

Transgenic mice were generated to constitutively express a fusion protein comprising extracellular domain of mouse TREM-1 and the Fc portion of a human IgG1 (“mTREM-1-hFc”). The nucleotide and protein sequences of the fusion protein construct are shown in SEQ ID NO:7 and SEQ ID NO:8, respectively. Alternatively, transgenic mice can be generated where the TREM-1-hFc construct is under the control of an inducible promoter rather than being constitutively expressed. Soluble TREM-1-Fc fusion proteins are also well known in the art, and have been shown to protect against LPS and septic shock as well as zymosan-A induced granuloma formation.

K/BxN Transfer in mTREM-1-hFc Transgenic Mice

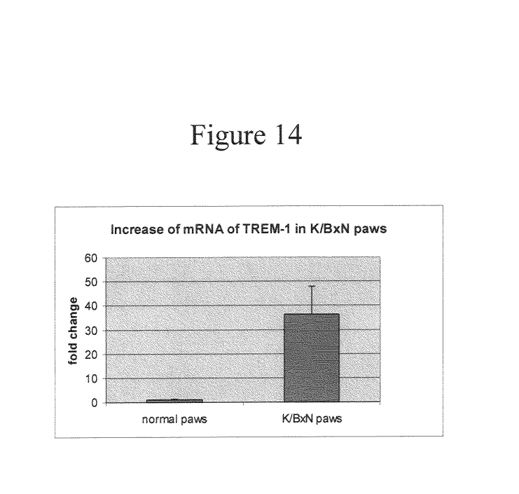

A murine K/BxN model is a mouse model that resembles many forms of human inflammatory arthritis, including RA (Ditzel (2004) Trends Mol. Med. 10(1):40-45). As shown in FIG. 14, TREM-1 mRNA expression was markedly increased in K/BxN paws as compared to normal paws. Therefore, serum or antibody from arthritic K/BxN mice can be transferred to experimental animals to determine if the mTREM-1-hFc construct inhibits the inflammatory response to K/BxN serum or antibody.

In one experiment, transgenic mice expressing a soluble mTREM-1-hFc fusion protein were challenged with K/BxN serum to assess whether soluble TREM-1 reduces arthritic inflammation. Briefly, TREM-1 transgenic (“Tg) mice were generated on a C57BL/6 background to express a soluble mTREM-1-hFc fusion protein under the control of a CAGGS promoter, which is a ubiquitously strong fusion promoter that is comprised of the CMV enhancer and the β-actin promoter. The overall construct was CAGGS/mTREM-1-hFc/rabbit β-globulin poly A. The soluble mTREM-1-hFc protein level in the blood plasma of transgenic mice was about 1-2 mg/ml. TREM-1 transgenic male mice (n=7) and wildtype male mice (n=7) were injected with 150 μl of K/BxN serum intraperitoneally (ip) on day 0 and day 2. Ankle diameter was measured periodically until day 14.

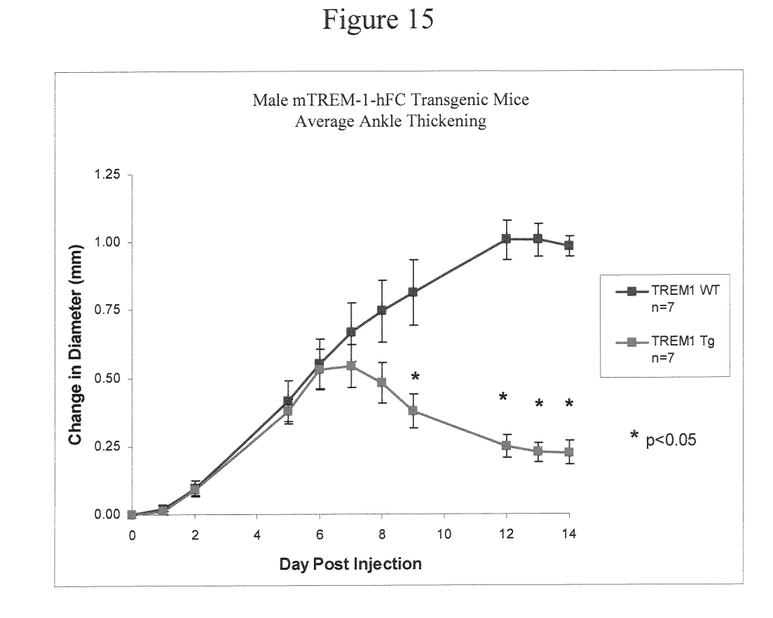

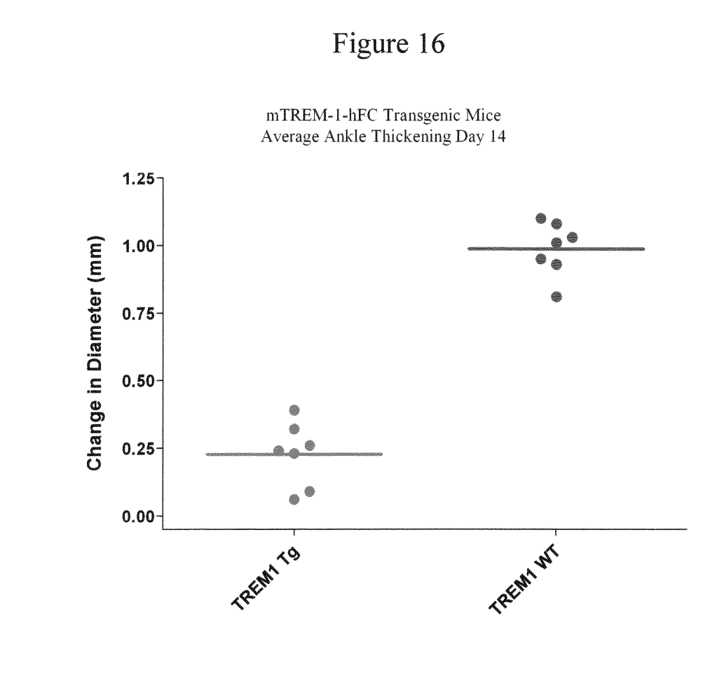

FIG. 15 shows the average ankle thickening of C57BL/6-TREM-1 transgenic mice compared to wildtype controls. As shown in FIG. 15, TREM-1 transgenic mice developed a similar phenotype as wildtype mice until day 6. Starting at day 7, ankle swelling subsided in TREM-1 transgenic mice while swelling continued in wildtype controls. Subsequently, a significant reduction in ankle swelling was observed from days 9-14 (p<0.05) in TREM-1 transgenic mice compared to wildtype controls. Moreover, peak swelling in TREM-1 transgenic mice was about half the peak swelling observed in the wildtype controls. By day 14, ankle swelling in TREM-1 transgenic mice was about a quarter of the amount of swelling observed in wildtype controls (FIG. 16). Thus, soluble TREM-1 is effective at significantly reducing the amount of inflammation associated with inflammatory arthritis, demonstrating that the use of TREM-1 antagonists, for example, TREM-1 fusion proteins and/or anti-TREM-1 antibodies, to modulate, reduce and/or inhibit TREM-1 and/or TREM-1 signaling is an effective method for treating inflammatory disorders, including, for example, RA.

Transcriptional and translational regulatory sequences used for generating fusion proteins of the invention may include, but are not limited to, promoter sequences, ribosomal binding sites, transcriptional start and stop sequences, translational start and stop sequences, and enhancer or activator sequences. In one embodiment, the regulatory sequences include a promoter and transcriptional start and stop sequences.

Promoter sequences may encode constitutive or inducible promoters. The promoters may be either naturally occurring promoters or hybrid promoters. Hybrid promoters, which combine elements of more than one promoter, are also known in the art, and may be used in the present invention.

Additional experimental animals can be generated by backcrossing a mTREM-1-hFc heterozygous mouse to a wildtype mouse, and wildtype offspring can serve as in-litter controls. Experimental animals can be tested in various animal models of inflammatory disease known in the art, such as, for example, LPS, and CIA, to determine if various mTREM-1-hFc constructs are protective of inflammatory disease. The mTREM-1-hFc construct can be constitutively expressed, or expression of the mTREM-1-hFc construct can be induced prior to, concurrently with, and/or at one or more time points after challenge with LPS and CIA. Transgenic mice expressing a soluble form of the TREM-1 receptor can also be generated to screen for putative inhibitors of inflammatory disease.

In a lipopolysaccharide (LPS) model of endotoxic shock, experimental animals are injected with LPS to determine the effectiveness of the mTREM-1-hFc construct in reducing the inflammatory response to LPS-induced shock. Further, experimental animals can be tested in a CIA model, such as in Example 2, to determine the effectiveness of the mTREM-1-hFc construct in reducing the inflammatory response to CIA.

Based on the association of TREM-1 and DAP12/TyroBP with RA, the mTREM-1-hFc construct is expected to be protective of LPS and CIA challenges in mice. Likewise, administration of a suitable TREM-1 construct and/or a suitable TREM-1 protein to a human subject afflicted with an inflammatory disease, such as RA, can reduce the severity of the inflammatory disease.

### Example 11

### Anti-hTREM-1 Antibodies

Anti-hTREM-1 antibodies are screened for the ability to inhibit production of pro-inflammatory cytokines in human RA synovium culture assays. RA and asthma models, such as in Example 9 and Example 12, have been used successfully as models of inflammatory disease to develop therapeutic antibodies which neutralize one or more aspects of the inflammatory response. Based, in part, upon the association of TREM-1 with inflammatory diseases, such as RA and asthma, anti-hTREM-1 antibodies are expected to inhibit production of pro-inflammatory cytokines in RA synovium culture assays and asthma models. Likewise, administration of suitable antibodies to a human subject afflicted with an inflammatory disease, such as RA or asthma, should reduce the severity of the inflammatory disease and/or lessen the symptoms of the disease.

### Example 12

### TREM-1 and Challenge with Anti-IgE Antibodies

Mast cells and IgE are well established players in allergic reactions, for example, acute respiratory disorders such as asthma or anaphylaxis, since crosslinkage of IgE on the surface of mast cells will induce signaling events that lead to mast cell activation and degranulation. This signaling cascade and the downstream consequences of mast cell activation and degranulation can be investigated in vivo in the mouse using a passive cutaneous anaphylaxis (PCA) model in which rat anti-mouse IgE is injected intradermally (id) into the ear. Anti-IgE antibody will bind and crosslink the IgE that is bound to the FεFRI receptors on the surface of mast cells to induce mast cell activation and degranulation. The ensuing inflammatory/edematous reaction results in a measurable swelling within the ear that can be calculated using an engineer's micrometer. Inagaki et al., “Mouse ear PCA as a model for evaluating antianaphylactic agents,” Int Arch Allergy Appl Immunol., 74(1):91-2 (1984).

Anti-IgE Challenge in Transgenic TREM-1 Mice

Transgenic TREM-1 mice and wildtype mice were challenged with anti-IgE antibodies using the ear swelling model. Transgenic mice were produced as in Example 10. The transgenic mouse strain used in this experiment contained a blood plasma level of mTREM-1-hFc protein of about 200 μg/ml. While under isofluorane anesthesia, ears of TREM-1 wildtype mice and transgenic heterozygous mTREM-1-hFc mice were measured for ear thickness. Anti-mouse IgE was diluted to 10 ng/20 ul in 0.9% saline. Transgenic and wildtype mice were challenged with anti-IgE antibody (BD PharMingen, San Diego, Calif.; catalog 553413) at time 0 in the left ear, while a separate group of transgenic and wildtype mice were challenged with endotoxin free 0.9% normal saline vehicle, as indicated in Table 2. Ear measurements were taken at +1 hour, +2 hours, +4 hours, and +6 hours following challenge.

TABLE 2
Anti-IgE antibody injections.

| | |
|---|---|
| WT females | N = 3 |
| Anti-IgE, 10 ng/20 ul id left ear | |
| WT females | N = 3 |
| vehicle, 20 ul id left ear | |
| WT males | N = 4 |
| Anti-IgE, 10 ng/20 ul id left ear | |
| WT males | N = 3 |
| vehicle, 20 ul id left ear | |
| mTREM-1-hFC heterozygous +/− females | N = 4 |
| Anti-IgE, 10 ng/20 ul id left ear | |
| mTREM-1-hFC heterozygous +/− females | N = 3 |
| vehicle, 20 ul id left ear | |
| mTREM-1-hFC heterozygous +/− males | N = 3 |
| Anti-IgE, 10 ng/20 ul id left ear | |

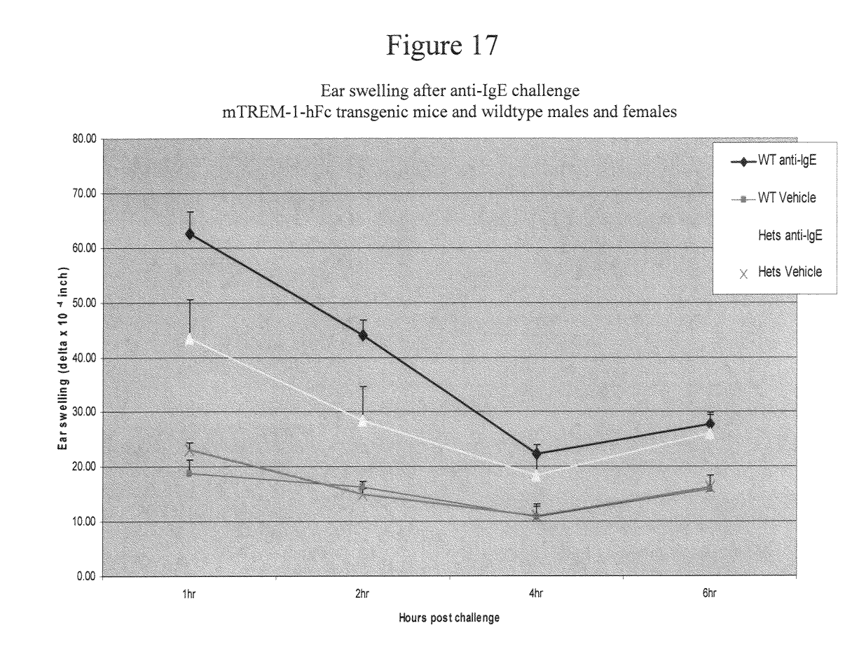

As shown in FIG. 17, reduced ear swelling was observed in TREM-1 transgenic mice as compared to wildtype controls. TREM-1 may therefore play a role in the allergic response in vivo since C57BL/6 mice overexpressing a mTREM-1-hFC chimeric protein have reduced cutaneous ear swelling. Thus, soluble TREM-1 is effective at reducing the inflammation associated with anti-IgE challenge. For example, soluble TREM-1 is expected to be effective at modulating asthma, anaphylaxis, acute and chronic urticaria (hives), angioedema, allergic rhinitis, insect sting allergies, and atopy.

Anti-IgE Challenge in Wildtype Mice Pretreated with Soluble TREM-1

Mice were pretreated with a soluble TREM-1 fusion protein to assess whether administration of soluble TREM-1 is protective of inflammation in an ear swelling model. The day prior to study, mice were either injected intraperitoneally with 0.9% saline, mTREM-1-mFc (500 ug/400 ul, 250 ug/400 ul, or 100 ug/400 ul) or anti-E. tenella-IgG 2a (500 ug/400 ul), as indicated in Table 3. Anti-mouse IgE was diluted to 10 ng/20 ul in 0.9% saline. Recombinant mTREM-1-mFc was generated comprising the extracellular domain of mouse TREM-1 and the Fc portion of a mutated mouse IgG2a (“mTREM-1-mFc”) (SEQ ID NO:27). The Fc region was mutated to reduce complement and Fc receptor binding. mTREM-1-mFc and anti-E. tenella-IgG 2a (Wyeth, Madison N.J.) were diluted in PBS to the desired dose level. Prior to challenge, ears of all the mice were measured to determine baseline ear thickness. Mice were challenged with anti-IgE (10 ng/20 ul/id) at time 0 in the left ear, while the right ear was challenged with 0.9% normal saline (20 ul/id). Ear measurements were taken at +1 hour, +2 hour, +4 hour, and +5 hour following challenge.

TABLE 3
Treatment schedule.

| | |
|---|---|
| C57BL/6 females | N = 7 |
| 500 ug/400 ul ip, mTREM-1-mFc (+17 hours pre-challenge) | |
| Anti-IgE, 10 ng/20 ul id left ear | |
| 0.9% normal saline, 20 ul, id right ear | |
| C57BL/6 females | N = 7 |
| 250 ug/400 ul ip, mTREM-1-mFc (+17 hours pre-challenge) | |
| Anti-IgE, 10 ng/20 ul id left ear | |
| 0.9% normal saline, 20 ul, id right ear | |
| C57BL/6 females | N = 7 |
| 100 ug/400 ul ip, mTREM-1-mFc (+17 hours pre-challenge) | |
| Anti-IgE, 10 ng/20 ul id left ear | |
| 0.9% normal saline, 20 ul, id right ear | |
| C57BL/6 females | N = 7 |
| 500 ug/400 ul ip, IgG 2a (+17 hours pre-challenge) | |
| Anti-IgE, 10 ng/20 ul id left ear | |
| 0.9% normal saline, 20 ul, id right ear | |
| C57BL/6 females | N = 7 |
| PBS/400 ul ip, (+17 hours pre-challenge) | |
| Anti-IgE, 10 ng/20 ul id left ear | |
| 0.9% normal saline, 20 ul, id right ear | |

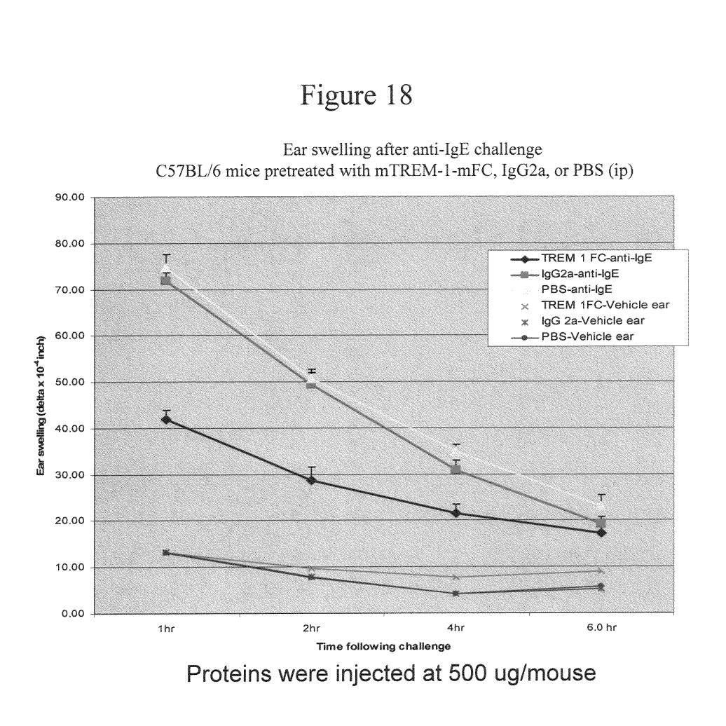

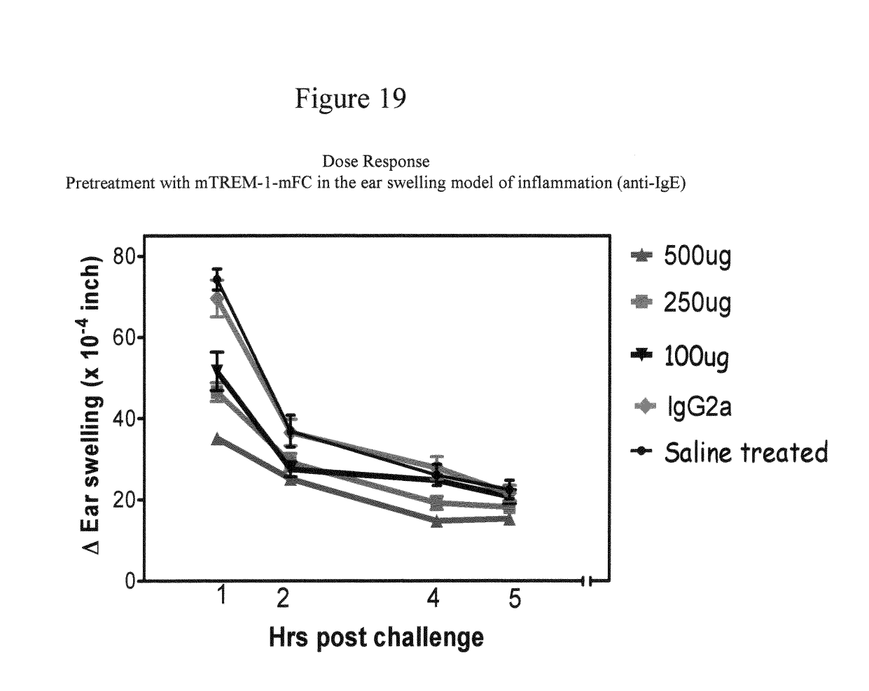

As shown in FIG. 18, pretreatment of mice with soluble mTREM-1-mFc protein reduced ear-swelling as compared to controls. Moreover, as shown in FIG. 19, the reduction in ear swelling is dose dependent. These data further demonstrate that soluble TREM-1 reduces the inflammation associated with anti-IgE challenge and that antagonists of TREM-1 and/or TREM-1 signaling, such as, for example, soluble TREM-1 fusion proteins and/or anti-TREM-1 antibodies, can be administered to a patient for treatment of inflammation associated with anti-IgE challenge. For example, soluble TREM-1 and/or anti-TREM-1 antibodies, are expected to be effective at modulating asthma, anaphylaxis, acute and chronic urticaria (hives), angioedema, allergic rhinitis, insect sting allergies, and atopy.

Anti-IgE Challenge in TREM-1 Knockout Mice

TREM-1 heterozygous (+/−) and homozygous (−/−) knockout mice were generated to assess whether ear swelling is reduced in the absence of functional TREM-1. Straight TREM-1 knockout mice were generated in which exon 1 and exon 2 of the TREM-1 gene were replaced by a lox P-flanked dual promoter driven Neo resistance gene, resulting in a reading frame shift in the TREM-1 gene. Gene targeting was conducted in C57BL/6 embryonic stem cells. TREM-1 knockout mice were bred with Protamine-Cre mice to generate Neo deleted TREM-1 knockout mice. On day 0, while under isofluorane anesthesia, ears of all of the mice were measured to determine baseline ear thickness. Mice were challenged with anti-IgE (10 ng/20 ul/id) at time 0 in the left ear, while the right ear was challenged with 0.9% normal saline (20 ul/id), as indicated in Table 4. Anti-mouse IgE was diluted to 10 ng/20 ul in 0.9% saline. Ear measurements were taken at +1 hour following challenge.

TABLE 4
Anti-IgE challenge in TREM-1 knockout mice

| | |
|---|---|
| Wild Type | N = 8 |
| Anti-IgE, 10 ng/20 ul id left ear | |
| 0.9% normal saline, 20 ul, id right ear | |
| 5 males and 3 females | |
| TREM-1 Heterozygous Knockout | N = 9 |
| Anti-IgE, 10 ng/20 ul id left ear | |
| 0.9% normal saline, 20 ul, id right ear | |
| 5 males and 4 females | |
| TREM-1 Homozygous Knockout | N = 6 |
| Anti-IgE, 10 ng/20 ul id left ear | |
| 0.9% normal saline, 20 ul, id right ear | |
| 2 males and 4 females | |

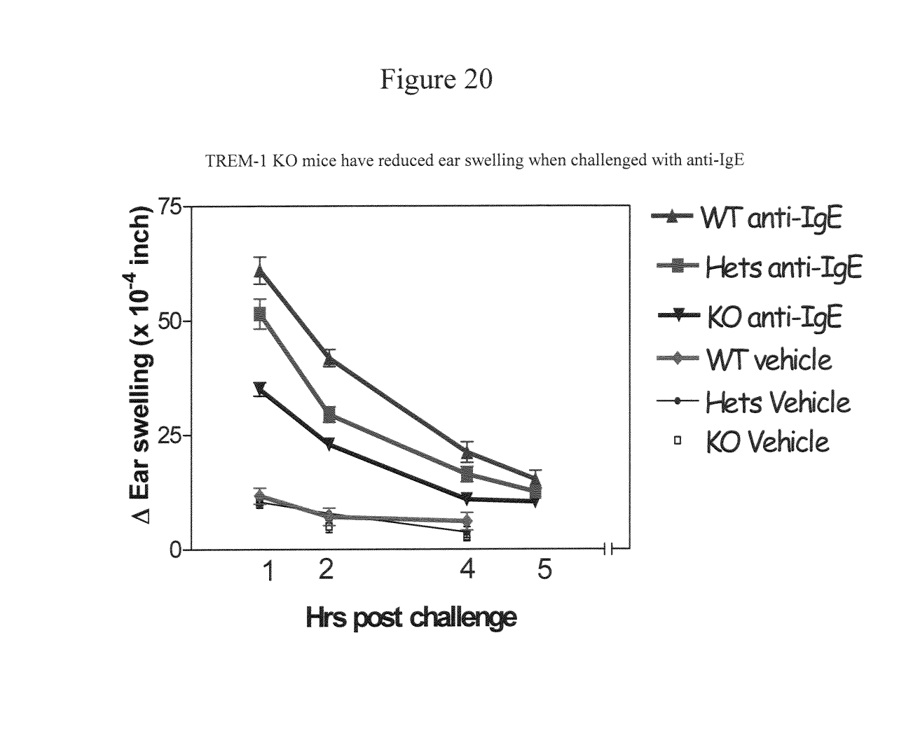

As shown in FIG. 20, mice that are heterozygous (+/−) for the TREM-1 gene and TREM-1 homozygous (−/−) knockout mice have a reduced ear swelling response following intradermal challenge with anti-IgE compared with wildtype (+/+) counterparts. This further demonstrates that TREM-1 is involved in the inflammatory response, and that TREM-1 is a therapeutic target for IgE-mediated inflammatory diseases/disorders, such as, for example, asthma, anaphylaxis, acute and chronic urticaria (hives), angioedema, allergic rhinitis, insect sting allergies, and atopy. Thus, antagonizing and/or inhibiting TREM-1 and/or TREM-1 signaling is effective at significantly reducing the amount of inflammation associated with IgE-mediated inflammatory diseases/disorders, demonstrating that the use of TREM-1 antagonists, for example, TREM-1 fusion proteins and/or anti-TREM-1 antibodies, to modulate, reduce and/or inhibit TREM-1 and/or TREM-1 signaling, is an effective method for treating IgE-mediated inflammatory diseases/disorders.

### Example 13

### shRNA and siRNA Knockdown of TREM-1

To demonstrate the utility of interfering RNA-based treatments for inflammatory disease, TREM-1 expression in THP-1 monocytes was measured after shRNA and siRNA knockdown. Briefly, various human TREM-1 and mouse TREM-1 shRNA sequences were generated and individually tested for the ability to reduce TREM-1 expression. Representative shRNA sequences are shown in Table 5. shRNAs were expressed in THP-1 monocytes by lentivirus transduction. Human TREM-1 siRNAs are commercially available from Dharmacon (Lafayette, Colo.), and were introduced into THP-1 monocytes by nucleofection. Representative siRNA sequences are shown in Table 6. After knockdown, TREM-1 expression was measured by TaqMan® RT-PCR at 72 hours post-transduction (in the case of shRNA) or 48 hours post-nucleofection (in the case of siRNA).

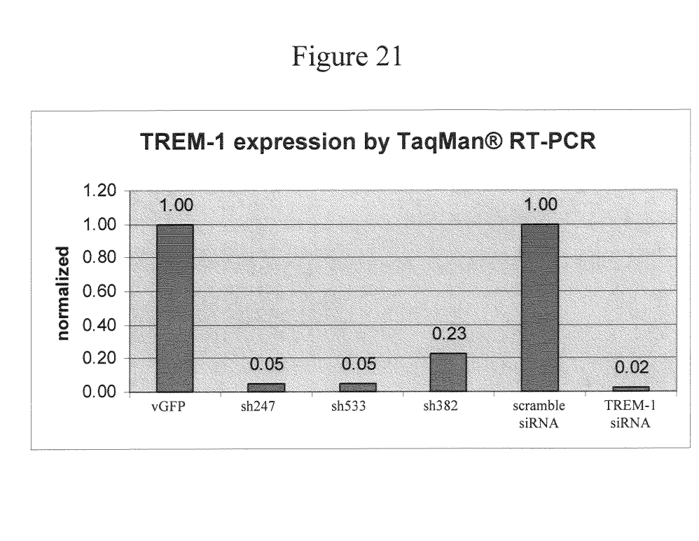

FIG. 21 is a bar graph showing TREM-1 expression by RT-PCR after shRNA or siRNA knockdown. As shown in FIG. 21, sh247, sh533, sh382, and pooled TREM-1 siRNAs effectively knocked-down endogenous TREM-1 expression in THP-1 monocytes as compared to vGFP and scramble siRNA controls. Thus, shRNA and siRNA knockdown are an effective means for reducing TREM-1 expression and can therefore be used in treating inflammatory disease.

To demonstrate that RNA-based treatments can effectively knock-down TREM-1 over-expression, TREM-1 was over-expressed in CHO cells prior to lentivirus shRNA knockdown. In one experiment, a human TREM-1-FLAG fusion protein was stably over-expressed in a CHO cell line. In another experiment, a mouse TREM-1-FLAG fusion protein was stably over-expressed in a CHO cell line. Subsequent to TREM-1-FLAG over-expression, shRNAs were expressed in each CHO cell line using a lentivirus. Various human and mouse TREM-1 shRNA sequences were generated and individually tested for the ability to reduce TREM-1 over-expression. Representative shRNA sequences are shown in Table 5. After exposure lentiviral shRNA, TREM-1 expression levels were assayed by Western blot using anti-FLAG antibodies as a probe.

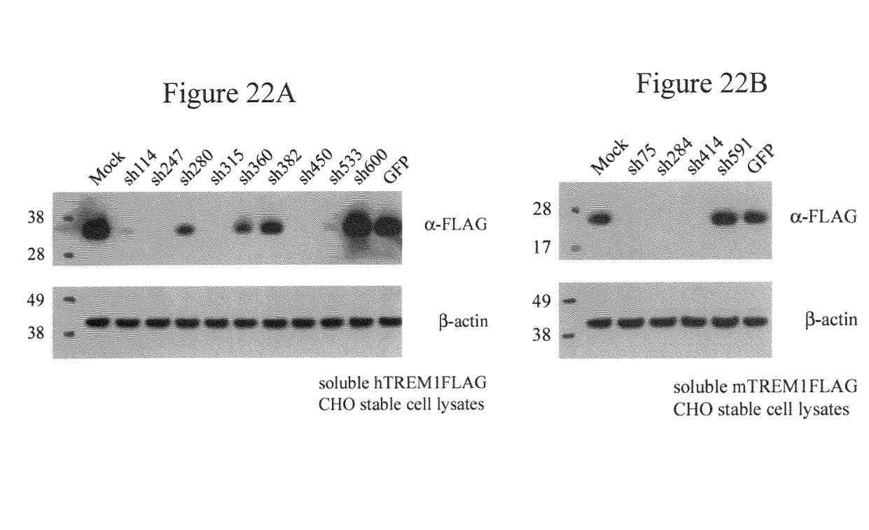

FIGS. 22A-B show representative Western blots depicting TREM-1 expression after lentiviral shRNA knockdown of TREM-1 in TREM-1 over-expressing cell lines. As shown in FIG. 22A, sh114, sh247, sh247, sh280, sh315, sh360, sh450, and sh533 effectively knocked-down human TREM-1-FLAG over-expression as compared to controls, while sh382 and sh600 were ineffective at knocking-down human TREM-1-FLAG over-expression. As shown in FIG. 22B, sh75, sh284, and sh414 effectively knocked-down mouse TREM-1-FLAG over-expression as compared to controls, while sh591 was ineffective at knocking-down mouse TREM-1-FLAG over-expression. Thus, shRNA knockdown is an effective means for reducing TREM-1 over-expression and therefore treating TREM-1 associated inflammatory disease.

TABLE 5

Lentiviral shRNA sequences. The sequence structure is
sense-loop-antisense.

| | | |
|---|---|---|
| | | SEQ |
| | | ID |
| shRNA | Sequence | NO: |

| | | |
|---|---|---|
| hTREM-1 sh114 | GTGAAATGTGACTACACGCTTCAAGAGAGCGTGTAGTCACATTTCAC | 9 |
| hTREM-1 sh155 | GAAAGCTTGGCAGATAATATTCAAGAGATATTATCTGCCAAGCTTTC | 10 |
| hTREM-1 sh247 | GGAGGATCATACTAGAAGATTCAAGAGATCTTCTAGTATGATCCTCC | 11 |
| hTREM-1 sh280 | GTTTACTGCGCGTCCGAATTTCAAGAGAATTCGGACGCGCAGTAAAC | 12 |
| hTREM-1 sb315 | GAAGATTCTGGACTGTATCTTCAAGAGAGATACAGTCCAGAATCTTC | 13 |
| hTREM-1 sh360 | GAGCCTCACATGCTGTTCGTTCAAGAGACGAACAGCATGTGAGGCTC | 14 |
| hTREM-1 sh382 | GCATCCGCTTGGTGGTGACTTCAAGAGAGTCACCACCAAGCGGATGC | 15 |
| hTREM-1 sh450 | GTGTATAAGATTCCTCCTATTCAAGAGATAGGAGGAATCTTATACAC | 16 |
| hTREM-1 sh533 | GTCAACTGCCGATGTCTCCTTCAAGAGAGGAGACATCGGCAGTTGAC | 17 |
| hTREM-1 sh600 | GTTCCGGTGTTCAACATTGTTCAAGAGACAATGTTGAACACCGGAAC | 18 |
| mTREM-1 sh75 | GAAGAAAGGTATGACCTAGTTCAAGAGACTAGGTCATACCTTTCTTC | 19 |
| mTREM-1 sh284 | GCTACAAGTTCAAATGACTTTCAAGAGAAGTCATTTGAACTTGTAGC | 20 |
| mTREM-1 sh414 | GATGTGTTCACTCCTGTCATTCAAGAGATGACAGGAGTGAACACATC | 21 |
| mTREM-1 sh591 | GTCTCCACATCCAGTGTTATTCAAGAGATAACACTGGATGTGGAGAC | 22 |

TABLE 6
Dharmacon ® human TREM-1 siRNA sequences.

| | | SEQ |
|---|---|---|
| | | ID |
| siRNA | Sequence | NO: |
| siRNA153 | CCAGAAAGCUUGGCAGAUAAUAA | 23 |
| siRNA206 | GCACAGAGAGGCCUUCAAAUU | 24 |
| siRNA49 | GAACUCCGAGCUGCAACUAUU | 25 |
| siRNA251 | GGAUCAUACUAGAAGACUAUU | 26 |

### Incorporation by Reference

All publications and patent documents cited in this application are incorporated by reference in their entirety for all purposes to the same extent as if the contents of each individual publication or patent document was incorporated herein.

What is claimed is:

1. A method of treating inflammatory disease in a subject, the method comprising the step: of reducing TREM-1-mediated signal transduction.

2. The method of claim 1, wherein the inflammatory disease is mediated by IgE.

3. The method of claim 1, wherein the inflammatory disease is a respiratory disease.

4. The method of claim 1, wherein the disease is asthma.

5. The method of claim 1 wherein the inflammatory disease is rheumatoid arthritis.

6. The method of claim 1, wherein the reducing step comprises reducing TREM-1 expression.

7. The method of claim 6, wherein the reducing step comprises administering an interfering RNA to the subject.

8. The method of claim 7, wherein the interfering RNA is an shRNA.

9. The method of claim 8, wherein the shRNA comprises an RNA encoded by any of SEQ ID NOs:9-18.

10. The method of claim 7, wherein the interfering RNA is an siRNA.

11. The method of claim 10, wherein the siRNA comprises any of SEQ ID NOs:23-26.

12. The method of claim 1, wherein the reducing step comprises inhibiting TREM-1 activation.

13. The method of claim 12, wherein TREM-1 activation is inhibited by administering a compound selected from the group consisting of a small molecule, a peptide mimetic, a peptide inhibitor, a ligand fusion protein, an antibody or fragment thereof that specifically binds TREM-1, an antibody or fragment thereof that specifically binds TREM-1 ligand, a soluble TREM-1 receptor, a soluble TREM-1 receptor fusion protein, and combinations thereof.

14. The method of claim 1, wherein the reducing step comprises directly inhibiting expression or activity of a non-TREM-1 protein involved in TREM-1-mediated signal transduction.

15. The method of claim 14, wherein the non-TREM-1 protein is DAP12/TyroBP.

16. The method of claim 1, wherein the reducing step comprises inducing an immune response to endogenous TREM-1 or DAP12/TyroBP protein in the subject.

17. The method of claim 16, wherein the reducing step comprises administering to the subject an immunogenic composition comprising an adjuvant and TREM-1 or DAP12/TyroBP protein or an immunogenic fragment thereof.

18. An antibody or fragment thereof that specifically binds TREM-1.

19. The antibody or fragment thereof of claim 18 wherein the antibody or fragment thereof is monoclonal.

20. The antibody or fragment thereof of claim 18, wherein the antibody or fragment thereof is a single domain antibody

21. A method of treating a subject, the method comprising the step of administering to the subject a therapeutically effective quantity of the antibody or fragment thereof of claim 19.

22. An shRNA comprising an RNA encoded by any of SEQ ID NOs:9-22.

23. A method of treating inflammatory disease in a subject in need thereof, the method comprising the step of reducing TREM-1-mediated signal transduction by administering a compound selected from the group consisting of a small molecule, a peptide mimetic, a peptide inhibitor, a ligand fusion protein, an antibody or fragment thereof that specifically binds TREM-1, an antibody or fragment thereof that specifically binds TREM-1 ligand, a soluble TREM-1 receptor, a soluble TREM-1 receptor fusion protein, and combinations thereof.

24. A method for assessing the efficacy of a TREM-1-modulating agent administered to a patient in need thereof, the method comprising detecting secreted phosphoprotein 1 (SPP1) levels in the patient or in a sample from the patient.

25. The method of claim 24, wherein the SPP1 levels are detected in a body fluid sample from the patient.

26. The method of claim 24, further comprising the step of comparing SPP1 levels to a reference, wherein an increase in SPP1 levels as compared to the reference is indicative of an increase in TREM-1 activity, and wherein a decrease SPP1 levels as compared to the reference is indicative of a decrease in TREM-1 activity.

27. The method of claim 26, wherein the reference corresponds to SPP1 levels detected in the patient or in a sample from the patient at a time prior to administration of the TREM-1-modulating agent.

28. A method of screening for candidate agents capable of modulating TREM-1 signaling, the method comprising the steps of:
contacting a TREM-1-expressing cell with a candidate agent; and
assessing secreted phosphoprotein 1 (SPP1) levels of the TREM-1-expressing cell to determine whether the candidate agent modulates TREM-1 activation.

29. A method of monitoring a patient treated for chronic inflammation, the method comprising the steps of:
administering a TREM-1 modulating agent to a patient in need thereof;
detecting secreted phosphoprotein 1 (SPP1) levels in the patient or in a sample from the patient; and
comparing the detected SPP1 levels with a reference, thereby monitoring the patient.

30. The method of claim 29, wherein the SPP1 levels are detected in a body fluid sample from the patient.

31. The method of claim 29, wherein a reduction in SPP1 levels as compared to the reference is indicative of a reduction in TREM-1 mediated inflammation.

32. The method of claim 29, wherein no change in SPP1 levels as compared to the reference is indicative of no change in TREM-1 mediated inflammation.

33. The method of claim 29, wherein an increase in SPP1 levels as compared to the reference is indicative of an increase in TREM-1 mediated inflammation.

34. The method of claim 29, wherein the reference corresponds to SPP1 levels detected in the patient or in a sample from the patient at a time prior to or concurrent with administration of the TREM-1-modulating agent.

35. The method of claim 29, wherein the reference corresponds to SPP1 levels in a control subject known not to have chronic inflammation.

36. A method of detecting the presence of inflammatory disease in a subject, the method comprising the step of:
detecting TREM-1 or DAP12/TyroBP expression or activity in the subject or a sample obtained therefrom, wherein increased expression or activity is indicative of the inflammatory disease.

37. A method of monitoring inflammatory disease in a subject, the method comprising the steps of:
(a) detecting TREM-1 or DAP12/TyroBP expression or activity in the subject at a first time or in a first sample obtained therefrom;
(b) detecting TREM-1 or DAP12/TyroBP expression or activity in the subject at a second, later time or in a second, later sample obtained therefrom; and
(c) comparing the expression or activity of (a) and (b), wherein a change in expression or activity is indicative of a change in disease status.

38. The method of claim 37, wherein the inflammatory disease is rheumatoid arthritis.

39. A method of evaluating a treatment for inflammatory disease in a subject, the method comprising the steps of:
(a) detecting TREM-1 or DAP12/TyroBP expression or activity in the subject at a first time or in a first sample obtained therefrom;
(b) detecting TREM-1 or DAP12/TyroBP expression or activity in the subject at a second, later time or in a second, later sample obtained therefrom;
(c) administering a treatment prior to the second, later time or the second, later sample; and
(d) comparing the expression or activity of (a) and (b), wherein a change in expression or activity is indicative of a change in disease status.

40. The method of claim 39, wherein the treatment is administered after the first time or first sample.

41. The method of claim 39, further comprising modifying a course of treatment for the subject based on the outcome of the comparison.


SEQUENCE LISTING (27 sequences)

SEQ ID NO: 1 - DNA, 24 bp, Artificial Sequence
  synthetic TREM-1 forward primer
cagatgtgttcactcctgtcatca

SEQ ID NO: 2 - DNA, 27 bp, Artificial Sequence
  synthetic TREM-1 reverse primer
ctgggtgagtattttgtggtaataagg

SEQ ID NO: 3 - DNA, 28 bp, Artificial Sequence
  synthetic TREM-1 probe
cctattacaaggctgacagagcgtccca

SEQ ID NO: 4 - DNA, 19 bp, Artificial Sequence
  synthetic DAP12/TyroBP forward primer
cctggtctcccgaggtcaa

SEQ ID NO: 5 - DNA, 21 bp, Artificial Sequence
  synthetic DAP12/TyroBP reverse primer
ggcgactcagtctcagcaatg

SEQ ID NO: 6 - DNA, 22 bp, Artificial Sequence
  synthetic DAP12/TyroBP probe
ttgtttccgggtcccttccgct

SEQ ID NO: 7 - DNA, 1311 bp, Artificial Sequence
  sequence encoding mouse TREM-1/human Fc fusion protein
atgaggaaggctgggctctggggactgctgtgcgtgttctttgtctcagaagtcaaagctgccattgttctagaggaagaaaggtatgacctagtggagggccagactttgacagtgaagtgtcccttcaacatcatgaagtatgccaacagccagaaggcttggcagagactaccagacgggaaggaacccttgaccctggtggtcacacagaggccctttacaagacccagtgaagtccacatggggaagttcaccctgaaacatgaccctagtgaggccatgctacaagttcaaatgactgaccttcaagtgacagactctggattgtatcgttgtgtgatttaccatcctccgaatgaccctgttgtgctcttccatcctgtccgcctggtggtgaccaagggttcttcagatgtgttcactcctgtcatcattcctattacaaggctgacagagcgtcccatccttattaccacaaaatactcacccagtgacacaactacaacccgatccctacccaagcccactgcggttgtttcctctcctggtcttggagtcactatcataaatgggacagatgctgacagtggtagcggctccggaagtggggagcccaaatcttgtgacaaaactcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 8 - protein, 436 aa, Artificial Sequence
  mouse TREM-1/human Fc fusion protein
MRKAGLWGLLCVFFVSEVKAAIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKGSSDVFTPVIIPITRLTERPILITTKYSPSDTTTTRSLPKPTAVVSSPGLGVTIINGTDADSGSGSGSGEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 47 bp, Artificial Sequence
  synthetic sequence encoding shRNA114
gtgaaatgtgactacacgcttcaagagagcgtgtagtcacatttcac

SEQ ID NO: 10 - DNA, 47 bp, Artificial Sequence
  synthetic sequence encoding shRNA155
gaaagcttggcagataatattcaagagatattatctgccaagctttc

SEQ ID NO: 11 - DNA, 47 bp, Artificial Sequence
  synthetic sequence encoding shRNA247
ggaggatcatactagaagattcaagagatcttctagtatgatcctcc

SEQ ID NO: 12 - DNA, 47 bp, Artificial Sequence
  synthetic sequence encoding shRNA280
gtttactgcgcgtccgaatttcaagagaattcggacgcgcagtaaac

SEQ ID NO: 13 - DNA, 47 bp, Artificial Sequence
  synthetic sequence encoding shRNA315
gaagattctggactgtatcttcaagagagatacagtccagaatcttc

SEQ ID NO: 14 - DNA, 47 bp, Artificial Sequence
  synthetic sequence encoding shRNA360
gagcctcacatgctgttcgttcaagagacgaacagcatgtgaggctc

SEQ ID NO: 15 - DNA, 47 bp, Artificial Sequence
  synthetic sequence encoding shRNA382
gcatccgcttggtggtgacttcaagagagtcaccaccaagcggatgc

SEQ ID NO: 16 - DNA, 47 bp, Artificial Sequence
  synthetic sequence encoding shRNA450
gtgtataagattcctcctattcaagagataggaggaatcttatacac

SEQ ID NO: 17 - DNA, 47 bp, Artificial Sequence
  synthetic sequence encoding shRNA533
gtcaactgccgatgtctccttcaagagaggagacatcggcagttgac

SEQ ID NO: 18 - DNA, 47 bp, Artificial Sequence
  synthetic sequence encoding shRNA600
gttccggtgttcaacattgttcaagagacaatgttgaacaccggaac

SEQ ID NO: 19 - DNA, 47 bp, Artificial Sequence
  synthetic sequence encoding shRNA75
gaagaaaggtatgacctagttcaagagactaggtcatacctttcttc

SEQ ID NO: 20 - DNA, 47 bp, Artificial Sequence
  synthetic sequence encoding shRNA284
gctacaagttcaaatgactttcaagagaagtcatttgaacttgtagc

SEQ ID NO: 21 - DNA, 47 bp, Artificial Sequence
  synthetic sequence encoding shRNA414
gatgtgttcactcctgtcattcaagagatgacaggagtgaacacatc

SEQ ID NO: 22 - DNA, 47 bp, Artificial Sequence
  synthetic sequence encoding shRNA591
gtctccacatccagtgttattcaagagataacactggatgtggagac

SEQ ID NO: 23 - RNA, 23 nt, Artificial Sequence
  synthetic DHARMACON hTREM-1 siRNA153
ccagaaagcuuggcagauaauaa

SEQ ID NO: 24 - RNA, 21 nt, Artificial Sequence
  synthetic DHARMACON hTREM-1 siRNA206
gcacagagaggccuucaaauu

SEQ ID NO: 25 - RNA, 21 nt, Artificial Sequence
  synthetic DHARMACON hTREM-1 siRNA49
gaacuccgagcugcaacuauu

SEQ ID NO: 26 - RNA, 21 nt, Artificial Sequence
  synthetic DHARMACON hTREM-1 siRNA251
ggaucauacuagaagacuauu

SEQ ID NO: 27 - protein, 436 aa, Artificial Sequence
  mouse TREM-1/mouse IgG2a Fc fusion protein
MRKAGLWGLLCVFFVSEVKAAIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKGSSDVFTPVIIPITRLTERPILITTKYSPSDTTTTRSLPKPTAVVSSPGLGVTIINGTDADSCAGSGSEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK